SEQUENCE LISTING (130 sequences)

SEQ ID NO: 1 - DNA, 21 bp, Artificial Sequence
  gRNA 1
atagcggacattcaacccagg

SEQ ID NO: 2 - DNA, 21 bp, Artificial Sequence
  gRNA 2
atttcctccactcaacctggg

SEQ ID NO: 3 - DNA, 21 bp, Artificial Sequence
  gRNA 3
cagacagctgttctatgtaca

SEQ ID NO: 4 - DNA, 21 bp, Artificial Sequence
  gRNA 4
tgtaagcacaagagaactggt

SEQ ID NO: 5 - DNA, 21 bp, Artificial Sequence
  gRNA 5
tgctgcaccttcggtataaaa

SEQ ID NO: 6 - DNA, 21 bp, Artificial Sequence
  gRNA 6
gtataaaagggggacaccggg

SEQ ID NO: 7 - DNA, 21 bp, Artificial Sequence
  gRNA 7
catagccacaacgcagccttg

SEQ ID NO: 8 - DNA, 20 bp, Artificial Sequence
  gRNA 8
aaatagcggacattcaaccc

SEQ ID NO: 9 - DNA, 21 bp, Artificial Sequence
  gRNA 9
gaacgacagcagcgcaggctg

SEQ ID NO: 10 - DNA, 21 bp, Artificial Sequence
  gRNA 10
gtggggatcagcaaatagact

SEQ ID NO: 11 - DNA, 21 bp, Artificial Sequence
  Mock
agcgaggtattcggctccgcg

SEQ ID NO: 12 - DNA, 21 bp, Artificial Sequence
  gRNA 21
ctcggatctggtttgcgagga

SEQ ID NO: 13 - DNA, 21 bp, Artificial Sequence
  gRNA 22
tctggtttgcgaggagcgggt

SEQ ID NO: 14 - DNA, 21 bp, Artificial Sequence
  gRNA 23
cccgctcctcgcaaaccagat

SEQ ID NO: 15 - DNA, 21 bp, Artificial Sequence
  gRNA 24
agaagaaacagttgctagact

SEQ ID NO: 16 - DNA, 21 bp, Artificial Sequence
  gRNA 26
aacacaccattgcgacaccct

SEQ ID NO: 17 - DNA, 21 bp, Artificial Sequence
  gRNA 28
ctcatttgcatgacaggtact

SEQ ID NO: 18 - DNA, 21 bp, Artificial Sequence
  gRNA 29
gctgttgtcactgtcaaccct

SEQ ID NO: 19 - DNA, 21 bp, Artificial Sequence
  gRNA 31
gtcattgtgacctaggaacac

SEQ ID NO: 20 - DNA, 21 bp, Artificial Sequence
  gRNA 32
cccaacctgggttgagtactc

SEQ ID NO: 21 - DNA, 21 bp, Artificial Sequence
  gRNA 33
agtactcaagggttctccttg

SEQ ID NO: 22 - DNA, 21 bp, Artificial Sequence
  gRNA 34
acgtggacaaattggccattc

SEQ ID NO: 23 - DNA, 21 bp, Artificial Sequence
  gRNA 35
tgtgcgcagagccccacagtt

SEQ ID NO: 24 - DNA, 21 bp, Artificial Sequence
  gRNA 36
attggccattccagaatacac

SEQ ID NO: 25 - DNA, 21 bp, Artificial Sequence
  gRNA 37
ttggagaggtctagggaaggg

SEQ ID NO: 26 - DNA, 20 bp, Artificial Sequence
  gRNA 71
ttttataccgaaggtgcagc

SEQ ID NO: 27 - DNA, 20 bp, Artificial Sequence
  gRNA 72
tattgtcaccccggatggaa

SEQ ID NO: 28 - DNA, 20 bp, Artificial Sequence
  gRNA 73
gctgcaccttcggtataaaa

SEQ ID NO: 29 - DNA, 20 bp, Artificial Sequence
  gRNA 74
aaatagcggacattcaaccc

SEQ ID NO: 30 - DNA, 20 bp, Artificial Sequence
  gRNA 75
gtgtcccccttttataccga

SEQ ID NO: 31 - DNA, 20 bp, Artificial Sequence
  gRNA 76
gtgacttacgagtgcgtgaa

SEQ ID NO: 32 - DNA, 20 bp, Artificial Sequence
  gRNA 77
ctgcaccttcggtataaaag

SEQ ID NO: 33 - DNA, 21 bp, Artificial Sequence
  gRNA 81
aataattgggtccccccaacc

SEQ ID NO: 34 - DNA, 21 bp, Artificial Sequence
  gRNA 82
acgtggacaaattgcccattc

SEQ ID NO: 35 - DNA, 21 bp, Artificial Sequence
  gRNA 83
tttaggatgggctacctctct

SEQ ID NO: 36 - DNA, 21 bp, Artificial Sequence
  gRNA 84
cacaatcaaggtttcccggca

SEQ ID NO: 37 - DNA, 21 bp, Artificial Sequence
  gRNA 85
gtcactgtgacctaggaacac

SEQ ID NO: 38 - DNA, 21 bp, Artificial Sequence
  gRNA 86
tgtgcgcagaggccctcagct

SEQ ID NO: 39 - DNA, 21 bp, Artificial Sequence
  gRNA 87
tttctcaatcccattgtattc

SEQ ID NO: 40 - DNA, 21 bp, Artificial Sequence
  gRNA 88
cctacagtacaggcggggtca

SEQ ID NO: 41 - DNA, 21 bp, Artificial Sequence
  gRNA 89
actggcttcctacagtacagg

SEQ ID NO: 42 - DNA, 21 bp, Artificial Sequence
  gRNA 90
aagaggtggaaacatgtgcct

SEQ ID NO: 43 - DNA, 21 bp, Artificial Sequence
  Mock 68
tcgataagccagtaagcagtt

SEQ ID NO: 44 - DNA, 20 bp, Artificial Sequence
  gRNA 91
caatcaaggtttcccggcac

SEQ ID NO: 45 - DNA, 20 bp, Artificial Sequence
  gRNA 92
gtgccgggaaaccttgattg

SEQ ID NO: 46 - DNA, 20 bp, Artificial Sequence
  gRNA 93
ggtgactgcctaagttagca

SEQ ID NO: 47 - DNA, 20 bp, Artificial Sequence
  gRNA 94
gttcctaggtcacagtgacg

SEQ ID NO: 48 - DNA, 20 bp, Artificial Sequence
  gRNA 95
ggtgaaggtctacacatcag

SEQ ID NO: 49 - DNA, 20 bp, Artificial Sequence
  gRNA 96
aacagctgttctgtgcgcag

SEQ ID NO: 50 - DNA, 20 bp, Artificial Sequence
  gRNA 97
gttctcattcgcgtgttcct

SEQ ID NO: 51 - DNA, 20 bp, Artificial Sequence
  gRNA 98
tgcaagtctgtcttgtggtt

SEQ ID NO: 52 - DNA, 20 bp, Artificial Sequence
  gRNA 99
tcttttgcaagtctgtcttg

SEQ ID NO: 53 - DNA, 20 bp, Artificial Sequence
  gRNA 100
tcattaaaaagccacaatca

SEQ ID NO: 54 - DNA, 20 bp, Artificial Sequence
  gRNA 110
caatcaaggtttcccggcac

SEQ ID NO: 55 - DNA, 20 bp, Artificial Sequence
  gRNA 111
gtgccgggaaaccttgattg

SEQ ID NO: 56 - DNA, 20 bp, Artificial Sequence
  gRNA 112
attagagaggttagagaagg

SEQ ID NO: 57 - DNA, 20 bp, Artificial Sequence
  gRNA 113
tttattagagaggttagaga

SEQ ID NO: 58 - DNA, 20 bp, Artificial Sequence
  gRNA 114
gaaaactaagtttattagag

SEQ ID NO: 59 - DNA, 20 bp, Artificial Sequence
  gRNA 115
gcatcgtaagcaaaaatgat

SEQ ID NO: 60 - DNA, 20 bp, Artificial Sequence
  Mock 116
tcgataagccagtaagcagt

SEQ ID NO: 61 - DNA, 20 bp, Artificial Sequence
  gRNA 117
ggccctcagctgtgggttct

SEQ ID NO: 62 - DNA, 20 bp, Artificial Sequence
  gRNA 118
ctcagctgtgggttctcatt

SEQ ID NO: 63 - DNA, 20 bp, Artificial Sequence
  gRNA 119
aacccacagctgagggcctc

SEQ ID NO: 64 - DNA, 20 bp, Artificial Sequence
  gRNA 120
cccacagctgagggcctctg

SEQ ID NO: 65 - DNA, 20 bp, Artificial Sequence
  gRNA 123
ggcacatgtttccacctctt

SEQ ID NO: 66 - DNA, 20 bp, Artificial Sequence
  gRNA 124
gcacatgtttccacctcttc

SEQ ID NO: 67 - DNA, 20 bp, Artificial Sequence
  gRNA 125
gaggtggaaacatgtgcctg

SEQ ID NO: 68 - DNA, 20 bp, Artificial Sequence
  gRNA 126
aggtggaaacatgtgcctga

SEQ ID NO: 69 - DNA, 20 bp, Artificial Sequence
  gRNA 127
aaacatgtgcctgagaatcc

SEQ ID NO: 70 - DNA, 20 bp, Artificial Sequence
  gRNA 128
catgtgcctgagaatcctaa

SEQ ID NO: 71 - DNA, 20 bp, Artificial Sequence
  gRNA 129
cagctgtcccccaccccaac

SEQ ID NO: 72 - DNA, 20 bp, Artificial Sequence
  gRNA 130
gtgggggacagctgaagagg

SEQ ID NO: 73 - DNA, 20 bp, Artificial Sequence
  gRNA 131
tgggggacagctgaagaggt

SEQ ID NO: 74 - DNA, 20 bp, Artificial Sequence
  gRNA 132
gggggacagctgaagaggtg

SEQ ID NO: 75 - DNA, 20 bp, Artificial Sequence
  gRNA 133
ggggacagctgaagaggtgg

SEQ ID NO: 76 - DNA, 20 bp, Artificial Sequence
  gRNA 134
cacgtgtcttcttttataaa

SEQ ID NO: 77 - DNA, 20 bp, Artificial Sequence
  gRNA 135
gaagacacgtgaatttgata

SEQ ID NO: 78 - DNA, 20 bp, Artificial Sequence
  gRNA 136
aagccacaatcaaggtttcc

SEQ ID NO: 79 - DNA, 20 bp, Artificial Sequence
  gRNA 137
tcattaaaaagccacaatca

SEQ ID NO: 80 - DNA, 201 bp, Rattus norvegicus
aagagcaagtccctaatgtgtagatctcctgcccggtgtcccccttttataccgaaggtgcagcagggcctctcaatttacaaacttcctggcaaaggctcctccctcttctagaccatgcacttttttgagcttaatttttttttcttttttcttttttttgcattgtaagcacaagagaactggtaagaatgaacttct

SEQ ID NO: 81 - DNA, 201 bp, Rattus norvegicus
gagcaagtccctaatgtgtagatctcctgcccggtgtcccccttttatacsaargkgsmgcmgggccycycaatttacaaacttccyggsaaaggcycccccctcttcyaraccawgsmcttttttgarcytaatttttttttcytttttcytttttttgsmttgkaarcmcmaraaaacyggkaaraawgaacttcygcm

SEQ ID NO: 82 - DNA, 57 bp, Homo sapiens
agaaaaaatcctgtgccgggaaaccttgattgtggctttttaatgaatgaagaggcc

SEQ ID NO: 83 - DNA, 47 bp, Homo sapiens
agaaaaaatcctgtgccgggagtggctttttaatgaatgaagaggcc

SEQ ID NO: 84 - DNA, 20 bp, Homo sapiens
gtgccgggaaaccttgattg

SEQ ID NO: 85 - DNA, 5831 bp, Artificial Sequence
  Sequence of the insert in the single gRNA adenovirus
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgtaagcacaagagaactggtgttttagtactctggaaacagaatctactaaaacaaggcaaaatgccgtgtttatctcgtcaacttgttggcgagatttttttcctcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttga

SEQ ID NO: 86 - DNA, 6199 bp, Artificial Sequence
  Sequence of the insert in the two gRNAs adenovirus
gcggccgcgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatagcggacattcaacccagggttttagtactctggaaacagaatctactaaaacaaggcaaaatgccgtgtttatctcgtcaacttgttggcgagatttttttcggccggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgtaagcacaagagaactggtgttttagtactctggaaacagaatctactaaaacaaggcaaaatgccgtgtttatctcgtcaacttgttggcgagatttttttcctcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttga

SEQ ID NO: 87 - DNA, 21 bp, Artificial Sequence
  Rat IL-10 forward
cgacgctgtcatcgatttctc

SEQ ID NO: 88 - DNA, 21 bp, Artificial Sequence
  Rat IL-10 reverse
tgccgggtggttcaatttttc

SEQ ID NO: 89 - DNA, 20 bp, Artificial Sequence
  Rat ppia forward
gacgccgctgtctcttttcg

SEQ ID NO: 90 - DNA, 25 bp, Artificial Sequence
  Rat ppia reverse
aaagttttctgctgtctttggaact

SEQ ID NO: 91 - DNA, 21 bp, Artificial Sequence
  gRNA1 forward
atagcggacattcaacccagg

SEQ ID NO: 92 - DNA, 21 bp, Artificial Sequence
  gRNA4 forward
accgtgtaagcacaagagaac

SEQ ID NO: 93 - DNA, 21 bp, Artificial Sequence
  gRNA cassette reverse
cgccaacaagttgacgagata

SEQ ID NO: 94 - DNA, 21 bp, Artificial Sequence
  SaCas9 forward
aactgaccaatctgaactccg

SEQ ID NO: 95 - DNA, 19 bp, Artificial Sequence
  SaCas9 reverse
tctggttgtcgttggtgtg

SEQ ID NO: 96 - DNA, 21 bp, Artificial Sequence
  IL1RN forward
ggggaccttacagtcacctaa

SEQ ID NO: 97 - DNA, 24 bp, Artificial Sequence
  IL1RN reverse
gttagtatcccagattctgaaggc

SEQ ID NO: 98 - DNA, 21 bp, Artificial Sequence
  RPLP1 forward
ctaatgtacgatttccaccgc

SEQ ID NO: 99 - DNA, 23 bp, Artificial Sequence
  RPLP1 reverse
gcaaccaaacttatcttcacacc

SEQ ID NO: 100 - DNA, 21 bp, Artificial Sequence
  gRNA1 on-target 1 forward
cctggtcagcaagaaatagcg

SEQ ID NO: 101 - DNA, 21 bp, Artificial Sequence
  gRNA1 on-target 1 reverse
ggttccattcacgcactcgta

SEQ ID NO: 102 - DNA, 37 bp, Artificial Sequence
  gRNA4 on-target 2 forward
tcgtcggcagcgtcaaatagccgtaatgcagaagttc

SEQ ID NO: 103 - DNA, 35 bp, Artificial Sequence
  gRNA4 on-target 2 reverse
gtctcgtgggctcgggcaaaggctcctccctcttc

SEQ ID NO: 104 - DNA, 25 bp, Artificial Sequence
  gRNA1 off-target 1 forward
tcaatgggaaacacaaaaagtttga

SEQ ID NO: 105 - DNA, 22 bp, Artificial Sequence
  gRNA1 off-target 1 reverse
cctctgtttttcaaagccctca

SEQ ID NO: 106 - DNA, 20 bp, Artificial Sequence
  gRNA4 off-target 2 forward
gcgtaggactcaccctaacg

SEQ ID NO: 107 - DNA, 20 bp, Artificial Sequence
  gRNA4 off-target 2 reverse
cgcagacaatggttggcatc

SEQ ID NO: 108 - DNA, 537 bp, Artificial Sequence
  Human IL-10 cDNA
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactaa

SEQ ID NO: 109 - DNA, 537 bp, Artificial Sequence
  Rat IL-10 cDNA
atgcctggctcagcactgctatgttgcctgctcttactggctggagtgaagaccagcaaaggccattccatccggggtgacaataactgcacccacttcccagtcagccagacccacatgctccgagagctgagggctgccttcagtcaagtgaagactttctttcaaaagaaggaccagctggacaacatactgctgacagattccttactgcaggactttaagggttacttgggttgccaagccttgtcagaaatgatcaagttttacctggtagaagtgatgccccaggcagagaaccatggcccagaaatcaaggagcatttgaattccctgggagagaagctgaagaccctctggatacagctgcgacgctgtcatcgatttctcccctgtgagaataaaagcaaggcagtggagcaggtgaagaatgattttaataagctccaagacaaaggtgtctacaaggccatgaatgagtttgacatcttcatcaactgcatagaagcctacgtgacactcaaaatgaaaaattaa

SEQ ID NO: 110 - DNA, 528 bp, Artificial Sequence
  Pig IL-10 cDNA
atgcccagctcagcactgctctattgcctgatcttcctggcaggggtggcagccagcattaagtctgagaacagctgcatccacttcccaaccagcctgccccacatgctccgggaactccgagctgccttcggcccagtgaagagtttctttcaaacgaaggaccagatgggcgacttgttgctgaccgggtctctgctggaggactttaagggttacctgggttgccaagccttgtcagagatgatccagttttacctggaagacgtaatgccgaaggcagagagtgatggggaggatatcaaggagcacgtgaactccctgggggagaagctgaagaccctcaggctgaggctgcggcgctgtcatcaatttctgccctgtgaaaacaagagcaaggccgtggaggaggtgaagagtgcctttagcaagctccaagagaggggtgtctacaaagccatgggtgagtttgacatcttcatcaactacatagaagcctacatgacgatgaagatgaggaagaactga

SEQ ID NO: 111 - DNA, 19001 bp, Homo sapiens
cacccctccctaacacacagtttacatcctgtgacccctttgaccagatatatgtttatacacttcggtgcaggtagctttccaattttgtacagatgtctgtaagtcacttttttggaaactggagcaacatctctgtagaaactctacttataaatgttgattcattcacagcctacaaagcaggggttacactataaataggaccaaacttagttcaggttcttgggagctgggggtctatgagatttacaaaggacagcttctttctcctactggacatatgaacagcctcaggcaacatctgaaatagtcgaggtcgtcttgggtcaacttctgtcacactctgctccctctccctggctgccctctctggaggtccctctgcactttcccctctgaggggctgtaagccctccattgaaatgctaaccttctgctctgcttaaccacatgctcccagatgacctactctcctccccacctgtcccagctcacagatcagccccacaggaactcagcttccaggattagaaccacccagggatttcagtgttattttcatggcttctttcccaccactgtatttgacctcctgactctctagaagttgctggtgatttcgtaaaggatttctcacaagcacggttaaggaagtgccaatggttgccactcaagtccatagaaaacaggcacttctaaattgggggtggtgtatgtgttcttttgcattatatatatgtttgtgttccatgggtcttcagtctacctgaagtctacctgcatggaccctccttcaaggatccaagaatctgctgatccagagcctgcatgacccgagtattcatccacagtggttttcacaggggtcagatatcctgtttctagcactggccattcctccctgcctgctcgccttttcccatgggatacaatggaggcattgggggactttaatacttcaaagcccgtagcagctggcaactcctgggactgaaccagcacccagactggcctcactgtgctctgccacgggaatccaggatcccatggtcacctctctcaagggaatttattatctgagtcccacgtcaggagccaagtacaccaacaacacttcctcctcggcataggcctgcgtcgcgcaggcatcaacgcaacacagacattttactgcgtggtgcagataattgcttgagaacgcaaagttctttctgaaagagatgtgaagcgctttctagggctttccagtaatcttcttttctccctgaaacagtcaatgtgagaaatccacttccggaaggtgaccacctcccttccacacccccgccgcccccggggccatgctcttgaggaaaaggcagcatcacgtcctcccctaccaagtggaggctcttctgatttccactgctctcagcaggcctgactcacaacgaaactgcaacctcaaagtgctgggggccaggattgcgagaaggaaatacaggtgaacacacgcaaaagctgccggaggaaaaggttttgtttctcttacagaatggcacttccagggctgggcagaagctatgagctagaagaggctgacagggcctggtcttgtgctgacctggtggatttggaaaggaaggaaaatatgtatcagaaccactcatgttcgctcgctcctgccgaggggaacagaaggggcaggctgggaggagagataggggaatcacaaaatttaagggcattagctttggaaatcttcttagaaaaacaactctggcaagcccttggattattgatgcggaaagcaaggccaagggatgaaaaggaacttgtttgggatctcatagtagaaggtccagggctggaaactaggcctcctaaatgcctgagccagtcagtctttctactatgagactgacttctctgtctccctgtaacatcctcagaatattggagttgaaattggtctgttccaagtcttcagatccacgaagagacatacttagcttttagcagctgagtattcccaaggaaaaacatcaaccactacacttcaggtgggctggctacttgctgtatgtcaagtattcatccaagcattgtgtacagtcactcattccgcccttgcaacactacgacagcaccaattatcatctccaattgtatatagatactgccgcacagggaggctcagaaacttgcccaaggtcacacagatgggaaagggcagagctgggttgcaaacccagtccgactccagggcccgcacacttaaccaccatgctctgctgcaaagacaagcccaaagacagagcacatcttggggtttccaataacagaacacttggacccagggtagggctgggggttgagcaaagtggggaaggcacttccagcctttgctgtcagaggtatataaggtctgccacataagagaggctcaagtgcacaacccttagccgctttcgtgaaaccagttgactacctgataccaatttatatccaacccactgcttgaggcacctgaattcaaggttcttgctgggaacagtctcagatcacccaaaagccaaggtacaaacagattctccttaattacttgaaagaataatactgtttagaaagtattccttgtattttaagtattttaatcttaaaaagaatacttttaaaatcatcaaaagagtatcagaaccttgttgggcctccttaaactattttgaactacatctgaaggtcgagacaacttagggcctctggagttcttagtccagcaccctgggaaaaggagtttctttttcccctccttctatagatggtctcccacaatcaggtgggcaaccagggattgagctttaatatgaccacacaatgtctgacccagttcctaaaagggtgccctccttataaaatgatttctaagttcaccagagcacttagagctcacctactgtgtgtgccaggcaaatctgcatgggatgagacgagaatggcaagccaatgatattgatttaattggacaaattctggccagtggggctaggaaatgtctgtttttattttcaatgttgttctaaaaactgagaaagcacaagagttaccaaagactaagcagggtttttaatgccccttagaattaaagaggagggagagaggagaataaaaggcagaagaagaaagcagagcccaaaataagcctgcagtagagcctgtccaacagctctgtgccccttgaccacacccagcctctagaacctagtcctaggcctggggccccttggagaaggggtttacagtaatgacaggtcactggcaaatcaaccccagaactcccatgggttttgacttgaaaccacaaacaccgcttgtgaggcactgtagcttccgtgacatgtgagaggctcacaggagctgctaaactgaaagaccaccctggctatttggaaactctgaataaagaatacagcagtaggcttctcccttgtctacccaagccaggtgccctttgggtacatcctctgcacctgcctctgaggccacactttgatcattctcctcctacttgtcctcacccagagatttctgtgaaacacttacagcattcattctctaagggtggtctgagaaaccctgagaggtcttcccttttccaggggaaaattcttatgctccaactaaaacggcatattacaacagaccgcatgtaagagcagttgtgaggatcctgcttctatcaagccagagagttaaaaatgcttggaaaaaaatgcagaacactgccactcttctcactaaatgtgttttggtttggaaaatagttacatctcatttaaaaatgccatgttaacatgtaagggtttattattgtttatgactttaaaaagatgtcataggaggtatttttaaaaatttatcagttttactttctaatatgataaatgtttacacatatatgtatatataactatatacatatatatgtatatataactatatacatatatatgtatatataacatatacatatatatgtatatataacatatacatatatatgtatatataactatatacatatatatgtatatataactatatacatatatatgtatatataactatatacatatatatgtatatataactatatacatatatatgtatatataacccacataagtgaaagctctttgggggtcctctataactttgaagatgtaaaagagtcctggatccacatagttcgccaacagctgacttagggaaccgggggcaaggctggacgcagttgctcgcacccactccctttcccttctcccttagtctatgagctaagaggggcacatgtaaatgacagtcatagtggaagtagcatgaatccagggtgggaggaagcaactgcatcttcatgtgtgatgcagctgtgcatccaaaatgcacaaaacacacggagtatggggaggattgagagtcagtggcacgttttagaggtaaggaccagaatgggcaggatgatgtccttctgagcaaagtgctcaacttcctcctctctctcttttttttctttttttttcttttttttttttttttttttttttgagacagagtcttgctctgtcgcccaggctggagcacagtggcgcgatctctgctcactgcagcctcagcctcccagattcaagtgattctcctgcctcagcctcaccacgcccagctaatttttgtatttttagtagagacagggtttcaccatgttggccaggctggtcttgaactactgacctagtgatccgcccatctcggcctcccaaagtgctgggattacaggcgtgagccaccgcgcctggcccctctcctctcttttataaggtttctggaaggtgttgctcctgtaggggtactgcagtgggaaggcaggccaaactatgctttataacactgtgacactcatggatcagagacaaaatagaaacaccattctgtctgaaaacactccaaggcagaaatcttcccctaagacaaaaatatctaagccaccctcactatcattcccaattcctaaacttaaggtacccaaggagtacaaaatagattcataactgtttaatggatggaaacttattgtcacagactttcaacttcttaccaccacaacctcacacaccgaacacatcttagacagctgcccctaattgttgctgcaggcccagagaacccagagtgatttgatacttacatgagtaaaggaattaggtactgatctgaaaaagaattgcattcagattctacctttgccattaactacctctaggacctgggaaagtcttcttctctaggcctctgtttgcccatttataaaagaagacacgtgaatttgataagaccccctcttcttctgacgtccatatagaattttaacattccagcagccatggaggagttaacatccgcctggaagagcactaattagtaatcagatcaattcttttaaaaactggaaaagctataataggtataggcatatagtttgtaaggacagtggtttcttcagatgaatccattctctacttctactttataagagatggaagaacagacatatagccatgtagcactacaaaaagtttaggaagcacatgctcagccgcccagtcagccaacaaatatttagtgagaaccttggatgtgcccagaaccatgctgagtactgagcaaacaacactcatcaaaaacagatacaggccctgccctcgaagtggtcacagtctcacaggagagacagaggtcaatcaaatgatcacatagccacatgtctaattacaaactgacataaatactacagaggggtgggagcaaggtgttctgagaagatacacccagggggcctgacctagactggggaatgatgaactcatttaaggaaggaacatctgagctgagagctgaaggatgaagacaggaggagagcaggaagggagaactaaatgtgcaaaggccctgtgataggagagagaggggataaatcactgggcatagtggctggtgcaatgtaagcccagccacttggaggctgaggcaagaggattacttgagcctaggagttcaaaaacagcctgggcaacatagcaagaccctgtctctttaaaaaaaaaaaaagagagagagagagcatgcggatcatttgaaaaaaaagaagaagaaagtcaatgaggctggagtgcagagggaagtagaaagtgaagagtgccgcaagttgaagcagaagagataggcagtgtcagatgataaaggaagttatagaaaacattagaatatgagtccttatttgaccaagagagggaaagtgggaagccatggaagggttcaagtagcaacagagtcatgatcagattggtgcttggaaataattactcttgctacaacgtggagaacggtctagagaatggatattagaataacaatttggcatttattttggagtccaggagagatgagacagtgttggggtgattagacccaacactaggccatgggggctacaaagtccagcagagtcaaaggaatgagaaaagacaagttaagggtgcataaaatgggtccagtgggccaacactggtatggaggctgcaaaggccctgagctctgggagcccacactatttattggtgatcaaacaaagaagcaggtggtgaggacgtgggggtaaacaggtgagggcgtgaggatgtgggggtagaaaggtagtggtgcatcaagcatagctgtgatggtttagcattttctttgacacatatagaatatgctctgctgctcaagataatggagagcatgtttacgagcctgggagagcaaccaacaagtctgtgcacattccatccaagccacgagggattttatgccctgggcttagatttgtggtgcagcagggcagccttctaccctttggcacagagcttggtgttccaaaggccacgagcggttttagaccctggaccccggacatctcccaagactcttttatattatgacagacaagccagtcctgcctcagctcttctaccaacagatagtagcttgaaccagacagtcattgctggtgatgatggaaatggagacaaatcagtagattagagaaatgtttatgaagaaaaatgtggaggttctggtgatagattgaacatagattggcaaggaagagggagggaccaacaaatcatttctagagttaattgatagttccatttactgagcgagggtgcactggatgaaggtgaaaccagagagagatgattttactctcagatacagaatcaactacataaattggcaggggaagacagtacaaaataaaaatgtggaaccccctgctcaacagttattgagaatttcaagatggtggtagcagagtaaggggaggacttttttagtcatgggaccctgtgtgactgcagaagtcacttatctacaaacccagccctgcttggatatgttgggttggaggtgccagccatccagtggagactggagcaaactaaagcgcaccagagggacagtcctggagctaaaagaatccatttgtgccccacactcaggagcccactagttatcctaggtagtcatgcagacagagcagaaacaactatggtcctctgggtggccaaggctcaaacacctggtgtcctggtccctgataagccaaatttctcatatgagaagttacagagcacagtgtccatcccctaaggaaaatccaaattgcctgatttggtgccatagttgcacaaaggggaattccacattggctgcccaaagatcagcccttccgctcatctgtggtttgcagtgtctgccggccatgatcctcggaagggcgcttaatccggtttgcataacccttgttccattccacctaagggccagccccagcccgggctcctcttaaacttctggcccaccaggcgtttattctgaagtaccccagacactatcaaagccaatgtcaggaaagcccagaggaatacagtgctcacagactcctgtctccttgcaccccaaactggccacactttgggtgtgtgataggcattggggtctgttgcagcacagcttctgaggaagcctctgtcctaggagctgaggaagcttattcgagggaatactagaaatttcaagctggcaaagctattttacacactaaatgtcctggataagaagaaatgaaactcccgttatcagctttaaaaaataactactaatggcaaataggagtcaagcccggaggggcttcttcatcaggacagttgtcacccatccccaagatgggcacggtctcaccttctctgcctgcatcgtcaacctgcctgctctctgccggctgaccgtgttgcccaacaagctgtctaaacccagagtccagcaagcccggatccctgaaccttccctctgcagctccagagccaccctcacaaggctgctagcctgctccccactcttgggtttgagctgcagaattgcagtaaggcatggggtaactacaccaagaaggctgccattttgcttagcatgagagtagctgggaggcagattctttttttttttttttttttgagacggagtctcgctctgtcgcccaggctggagtgcagtgacgcgatctcggctcactgcaacctccgcctcccaggttcacgccattcttctgcctcagcctcccaagtagctgggactacaggcgccaaccaccacacctagctaaattttttttttttttttttttttttttgagacggagtctcgctctgtcgcccaggctggagtgcagtgacgcgatctcggctcactgcaagctccgcctcccaggttcacgccattctcctgcctcagcctcccgagtagctgggactacaggcacctgccaccacgcccagctaattttttctatttttagtagagacagggtttcaccgtgttagccagggtggtctcaatctcctgacctcgtgatccgtccacctcggcctcccaaagtgttgggattacaggcgtgagccaccgtgcccgacaagggaggcagattcttttttttttttttttttttttttttgtagttgcaagatttaatacagtgaaaacagagctcccatacaaagggaagggacccaaagagggtagccattgccggcttggatgcccgggtttatgtcccgatcattgtccctcccgctgtgctctcaggcgatagatgattggctatttctttacctcccgtttttgcctaattagcattttagtgagctctctttaccacctgattggtcgggtgtgagctaagttgcaagccccgtgtttaaaggtatatgcggtcaccttcccagctaggcttagggattcttagttggtctaggaaatccagctagtcctgtctctcagtccccgctctcaacaggaaaacccaagtgctgttggggaggttggccgatgactgctctaatgggaggcagattctttataagatcttgtaaactgtagaatgcaccctccaaaatctatttgcataagcacacacacacacacacacacacacaccccagcagttcttgcctgcccagattcctctgcagctaaagtgatgaaacttactgggcggagcttcctaaaaagattattagggtctcctgggttggtgtgcctttaaacctttggactttaccacctcctatctctcctatctccttgcaacaaaggttaggagaacaagaatgcagaaaaaacgggtcctggatgacatctgagtgcctgctttgggcttcttgatgagtgagacagaaaataaaatacaaccccctcttttaaaagccatgcttactcaggttttccttcatttgcagctaaatacagaaatgagagaatattttggagcagggatggaagaagagaggtattccccttcccacaaccttctgatttcccagtacatcccccactggaaaaattcatttaaaatcagtataataagcattgattagatgcctactatgcatctgggcttgagggcaaactggactcaggccttttggcctcaagaagctcacagtgtgagagtggcatttgtgtcctcttgaaattcacaggactaaattgtgcccaggctgacattctatccatccataggtgcctgccttctcacttccctctcttcatgggctcttgccttgtaccaaaatccaaacccaaatctcctcacatgtgagtgttggcattcatgtctcagacatgacctatgggcttgggacttttccccgtggaccccagtgacttttcagatgaacaggtatcttcaaaaacttgagaaataggagtcctgtttgttgttcttgttgctttgtcaatataaggacacagggtctttattcaaatgttcatatctatctcttgacagaaatactatgagacatattgatggagaagccgttatctccatatgctaaatgaggacttgcaccagggaacttgcccatggttctctccaaccacttaaattctgaaattttgaaatgagagtggacagtaatttcaaatcaatggggaaagaatcaaatcttcagcaaatggcttgagataattagctacacatttcagaacaaataaagaagtcagatccgggccgggcacagtggctcatgcctgtaatctcagcactctgggaggccaaggcgggcggatcataaggtcaggagatcgagaccatcctggttaacacagtgaaaccccgtctctaataaaaatacaaaaaaaaataaaaaaacttagccgggcgtggtgccaggcgcctgtagtcccagctactcgggacgctgaggcaggagaatggcttgaactcgggaggcagagcttgcagtgagctgagatcatgccactgcactccagcctgggcaacagagcgagactctgtctcaaaaaaaaaaaagaagtcagatcctaacctcaaccctatttaacagattatagatgaaagaaaggtacaaatggcttttacatacctcccttctccctgacattttgtatgtgtgtgtgtgtgtatttacacacacatctcatataaggaaattgaagggaggctgcctgcatccctgagtcactctccctctccttctgaatgcttacctgtgcccagaccacctccttagcctcgcaccctccaggcttacagggcactcttctatgcccatcccaagtatagctgataccttccaagggccagacttggtgctaagtaccaagtacgcaaagattaataaaacaatgtcctgtttcagggagctcaaagctgattcggcagggcatggtgtgtacatgaatgataaccacgtagggttgcaggtttcctagtgaggtaagcacaaggcaagatgggaaacaaaggaaggaggggttcacagcctcacccagagtccagaacccctggcctgcctggtgcccatgctgagtccacttctggaacacccagctcagagagggggttagacctgcaggctaacacagacacagcccagaaaacccaggagccgagggggaaggagaaaggtgcaagaaggggaaacccaggtcctggtccccttctctctgcttcctggcagcagaactcagacagaacccttaagccagtctaagtctggcaggaccagtaagttctgagttagctccatactagtttctagcaggctctttctcacttcctgattcttaggtttctacattgacactccctgaagagttgggaagagacaccacagtcccctgaccctgatccataggtcacacagcagggacatccacagggtgggcgtgggccctctcatccctccctcccactcacttcacgctggctgggccccaaggtgtttgcaccccttgcagtgagtgaccttctctagtgcagcaagctcagaacctgctgccactggagttgtcccattgctgatgcagaaaggtgaagaactagcagaacactggaaatgccctccatctgggtccatggctacttaagctcaatgctccctggcaggcaggaggacaggtgctattgccctgttgggacagatgaaaaacagacacagggaggatgagtgatttgccctgactatagagtggcagggccaaggcagagcccaggcctcctgcacctaggtcagtgttcctcccagttacagtctaaactggaatggcaggcaaagcccctgtggaaggggaaggtgaaggctcaatcaaaggatccccagagactttccagatatctgaagaagtcctgatgtcactgccccggtccttccccaggtagagcaacactcctcgccgcaacccaactggctccccttaccttctacacacacacacacacacacacacacacacacacacacacacacaaatccaagacaacactactaaggcttctttgggaaggggaagtagggataggtaagaggaaagtaagggacctcctatccagcctccatggaatcctgacttcttttccttgttatttcaacttcttccaccccatcttttaaactttagactccagccacagaagcttacaactaaaagaaactctaaggccaatttaatccaaggtttcattctatgtgctggagatggtgtacagtagggtgaggaaaccaaattctcagttggcactggtgtacccttgtacaggtgatgtaatatctctgtgcctcagtttgctcactataaaatagagacggtaggggtcatggtgagcactacctgactagcatataagaagctttcagcaagtgcagactactcttacccacttcccccaagcacagttggggtgggggacagctgaagaggtggaaacatgtgcctgagaatcctaatgaaatcggggtaaaggagcctggaacacatcctgtgaccccgcctgtactgtaggaagccagtctctggaaagtaaaatggaagggctgcttgggaactttgaggatatttagcccaccccctcatttttacttggggaaactaaggcccagagacctaaggtgactgcctaagttagcaaggagaagtcttgggtattcatcccaggttggggggacccaattatttctcaatcccattgtattctggaatgggcaatttgtccacgtcactgtgacctaggaacacgcgaatgagaacccacagctgagggcctctgcgcacagaacagctgttctccccaggaaatcaactttttttaattgagaagctaaaaaattattctaagagaggtagcccatcctaaaaatagctgtaatgcagaagttcatgttcaaccaatcatttttgcttacgatgcaaaaattgaaaactaagtttattagagaggttagagaaggaggagctctaagcagaaaaaatcctgtgccgggaaaccttgattgtggctttttaatgaatgaagaggcctccctgagcttacaatataaaagggggacagagaggtgaaggtctacacatcaggggcttgctcttgcaaaaccaaaccacaagacagacttgcaaaagaaggcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttgtgagtatgattccttcctgtcctttctctcttcctgggactgcctgaactagacattctcctggaactataagaaccctcctcctgcgcctccacctccatccccaacacctattcccccaaacttaaattcttaagagaatcctagatcaagccatgggtttggtgagttaagctaagccagatgatacagtaaatgtgcaggaaacctgccttataaagtaaatgcgttctctctcgtgctgagaaacttataagatcctgctggcgctctatactttattggctaggagaagtaaagaaatgtctgattcgaggtgaagatgctccccatgccttgcagcagggaaatttaaattgcctctgcttagagcgtttccagacctgaaagaccagtggtttagggaagcactctacatgagggaaacctgcattagaaggagcttcttaatccctgggatctttccaagctaaactggatgtctacagtggggagaaagaaaagcagagaacaggacatgaggggggctcaaggccccgaagggttgacataggtgtcccttaaagccgaatgtagctccgcagaaagaagaccaggactgagtcaagcttctgctttcccttcaaaatcggccagattttttaaataacttgactctgaggaggaggacctgatttaagtgatggtcccatcactgttgaatcctctgtttttaaaactccccttttgatttttttgggccagagccaattttatttaaaaaaaaaaatctctaaatgaaagggcatcaaaaagaccgcatttcagttatttccccaaacctcaagttcattctccttttgttcttcctgcagcaaatgaaggatcagctggacaacttgttgttaaaggagtccttgctggaggactttaaggtgagagcaggggcggggtgctgggggagtgtgcagcatgattaagggaagggagactctgcttcctgattgcagggaattgggtttgtttccttcgctttgaaaaggagaagtgggaagatgttaactcagcacatccagcagccagagggtttacaaagggctcagtcccttcggggaggcttctggtgaaggaggatcgctagaaccaagctgtcctcttaagctagttgcagcagcccctcctcccagccacctccgccaatctctcactcacctttggctcctgcccttagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtgtaagtagcagatcagttttttcccttgcagctgcccccaaaataccatctcctacagaccagcagggacactcacatccacagacacagcaaagacacagactggcagagctagctgtaaatgaggaaagactcctggagtcagatctcttgctcatttctctttgagcaggcgttgggggtggctgctaggcatttacatgtgaaatttgcaaacagctttcctgttatttgtgagtcatttgtgggttattaactactcccctctctcttcataaaaggagcccagagcttcagtcaggcctccactgcctctttgtaactagaccctgggcggggagctaaggttcccaagcagaggaaacatcattcacctcttttaatctcaatgttttgaaagcaaagctctaagaagggcccaattgactgacaggatttcccctggcattttagaagggacaagggggctattcatccccaggctagtgtctatgagtaattcctccaggtaatttatttctccaactgaaatgatgccctcactactaatggtttcccctgttctgtcaccaatattggaaaatcagttggtgtctatttgtaggacaaggctatgtgaagggtttggtcccagtagcttccctcctcagatgcttagaagtgttcctcggtggctgtgactgacggggaggaacaggagagagaggcagaaaaggacaggctgaagaatgcctcgctcagcactgcaggagatactgtagagttctgggggaggaaggaatcccaagacctgggttgtcatccaagccttgcaaacatcttggagtgagtcctggagaaatacatttaactcccagggccatggaagcagggctcagttctctctgggagctgtgaggcaaggcatttggataaatctggcctcctcatgatgccaccagcttgtcccctaagtgtgatggacatggagctggaagccaggatcaccaacactttctcttttcttccacagcatcgatttcttccctgtgaaaacaagagcaaggccgtggagcaggtgaagaatgcctttaataaggtgagcttggatggtggcagagagggtctgcagagcacaacccatgcccactccccaaccccaaagcatggaaggtggtggggactcaataggccccattcttcattggagagagtgtgggaacctgacagatggtatgacctgctcagccagtgaggagctgctgccttgattgtatttgttttctgttaagtgtctttgggggtttctaaatgactgctcgctgcctttgcaggcttgcgggttaggctggccggccagcctgtgaacacagtgagctgcatgctggggagagtgacaaaggaaacagaaagtacagaaagtagcttgttgggaatctaggctgaacccacacgtgcaggaagctggcacataaatgtgcacatacaaatacacctgggggttcagcccagactccccagaactcagaatgagcaggaagctggattctcacttaacctggagttggttcaagcccgctttccatctgcccttcgcacctgcggaggtgcctgagaatgtcagttcccaaacgaaatggggtttcacacttccaactgtgcgtgaactttttcagtctgatttcccagaaaccgtgcggcctatgtcctcctcgtgggctggggacagacactgcacagagtgccaacatcagggggtgtgaatttctcatagtaggtcagggcggcagggcagggcctgctcagtgtgttggtgggagaacacagacatttaaaaggctccctcctctcctctcaccgtcttgctttcgaagcgcttcctctaatgtcttttcatcaaactctgcataatcatcatgtgaatacgtgacctttaaaattgttgaaaaggcatcattttgaagacagtgctttgcaaaatgaatgctccccttgctagggggaggcctggaggagatgaaggtcaatgcacagcctttcccaaggcagctaggcctatcctctggtttacttcccagcgtgagggagaacaagcaacctctgcactcaaggtcatgcccatccatgagcatgagggaggggagcctatttagtccccagaaaggattttaactgtatgtttcttatctctctgcacagctccaagagaaaggcatctacaaagccatgagtgagtttgacatcttcatcaactacatagaagcctacatgacaatgaagatacgaaactgagacatcagggtggcgactctatagactctaggacataaattagaggtctccaaaatcggatctggggctctgggatagctgacccagccccttgagaaaccttattgtacctctcttatagaatatttattacctctgatacctcaacccccatttctatttatttactgagcttctctgtgaacgatttagaaagaagcccaatattataatttttttcaatatttattattttcacctgtttttaagctgtttccatagggtgacacactatggtatttgagtgttttaagataaattataagttacataagggaggaaaaaaaatgttctttggggagccaacagaagcttccattccaagcctgaccacgctttctagctgttgagctgttttccctgacctccctctaatttatcttgtctctgggcttggggcttcctaactgctacaaatactcttaggaagagaaaccagggagcccctttgatgattaattcaccttccagtgtctcggagggattcccctaacctcattccccaaccacttcattcttgaaagctgtggccagcttgttatttataacaacctaaatttggttctaggccgggcgcggtggctcacgcctgtaatcccagcactttgggaggctgaggcgggtggatcacttgaggtcaggagttcctaaccagcctggtcaacatggtgaaaccccgtctctactaaaaatacaaaaattagccgggcatggtggcgcgcacctgtaatcccagctacttgggaggctgaggcaagagaattgcttgaacccaggagatggaagttgcagtgagctgatatcatgcccctgtactccagcctgggtgacagagcaagactctgtctcaaaaaataaaaataaaaataaatttggttctaatagaactcagttttaactagaatttattcaattcctctgggaatgttacattgtttgtctgtcttcatagcagattttaattttgaataaataaatgtatcttattcacatcatgttctggtgtcagttgactcttgtctgactaaaattgttctctcaaagctcccaagacaaaagagagtgggtgaatgaattctgcatttcagagaaaggaagccagctaccccctacccccaggagagagggaaggctccaaacctgatcagagaaaaagctattctcactgcaaaagccagtagcaaagacatcatggtcttcccacaactcaatcctttaaagatttgctcctaaatccttaaagatcatgctgattttcaaggctaacggggagattctgtaggtaccaaaattagaaaggtaaacaggggaaggttttttggaggttgtttttgtttttgttgttgcttttcgagacggagtctcgccctgtctccaggctggagtgcaatggcatgatctcagctcactgcaacctccgcctcccaggttcaagtgattctcctgcctcagcctcccaagtagctgggactacaggcatgtgccaccacgcccggctaattttttgcatttttagtacagatggggtttcaccatgttggccaggctggttaggaactcctggcctcaagtgatccgcctgcctcagtctcccaaa

SEQ ID NO: 112 - DNA, 11892 bp, Homo sapiens
aggcatggggtaactacaccaagaaggctgccattttgcttagcatgagagtagctgggaggcagattctttttttttttttttttttgagacggagtctcgctctgtcgcccaggctggagtgcagtgacgcgatctcggctcactgcaacctccgcctcccaggttcacgccattcttctgcctcagcctcccaagtagctgggactacaggcgccaaccaccacacctagctaaattttttttttttttttttttttttttgagacggagtctcgctctgtcgcccaggctggagtgcagtgacgcgatctcggctcactgcaagctccgcctcccaggttcacgccattctcctgcctcagcctcccgagtagctgggactacaggcacctgccaccacgcccagctaattttttctatttttagtagagacagggtttcaccgtgttagccagggtggtctcaatctcctgacctcgtgatccgtccacctcggcctcccaaagtgttgggattacaggcgtgagccaccgtgcccgacaagggaggcagattcttttttttttttttttttttttttttgtagttgcaagatttaatacagtgaaaacagagctcccatacaaagggaagggacccaaagagggtagccattgccggcttggatgcccgggtttatgtcccgatcattgtccctcccgctgtgctctcaggcgatagatgattggctatttctttacctcccgtttttgcctaattagcattttagtgagctctctttaccacctgattggtcgggtgtgagctaagttgcaagccccgtgtttaaaggtatatgcggtcaccttcccagctaggcttagggattcttagttggtctaggaaatccagctagtcctgtctctcagtccccgctctcaacaggaaaacccaagtgctgttggggaggttggccgatgactgctctaatgggaggcagattctttataagatcttgtaaactgtagaatgcaccctccaaaatctatttgcataagcacacacacacacacacacacacacaccccagcagttcttgcctgcccagattcctctgcagctaaagtgatgaaacttactgggcggagcttcctaaaaagattattagggtctcctgggttggtgtgcctttaaacctttggactttaccacctcctatctctcctatctccttgcaacaaaggttaggagaacaagaatgcagaaaaaacgggtcctggatgacatctgagtgcctgctttgggcttcttgatgagtgagacagaaaataaaatacaaccccctcttttaaaagccatgcttactcaggttttccttcatttgcagctaaatacagaaatgagagaatattttggagcagggatggaagaagagaggtattccccttcccacaaccttctgatttcccagtacatcccccactggaaaaattcatttaaaatcagtataataagcattgattagatgcctactatgcatctgggcttgagggcaaactggactcaggccttttggcctcaagaagctcacagtgtgagagtggcatttgtgtcctcttgaaattcacaggactaaattgtgcccaggctgacattctatccatccataggtgcctgccttctcacttccctctcttcatgggctcttgccttgtaccaaaatccaaacccaaatctcctcacatgtgagtgttggcattcatgtctcagacatgacctatgggcttgggacttttccccgtggaccccagtgacttttcagatgaacaggtatcttcaaaaacttgagaaataggagtcctgtttgttgttcttgttgctttgtcaatataaggacacagggtctttattcaaatgttcatatctatctcttgacagaaatactatgagacatattgatggagaagccgttatctccatatgctaaatgaggacttgcaccagggaacttgcccatggttctctccaaccacttaaattctgaaattttgaaatgagagtggacagtaatttcaaatcaatggggaaagaatcaaatcttcagcaaatggcttgagataattagctacacatttcagaacaaataaagaagtcagatccgggccgggcacagtggctcatgcctgtaatctcagcactctgggaggccaaggcgggcggatcataaggtcaggagatcgagaccatcctggttaacacagtgaaaccccgtctctaataaaaatacaaaaaaaaataaaaaaacttagccgggcgtggtgccaggcgcctgtagtcccagctactcgggacgctgaggcaggagaatggcttgaactcgggaggcagagcttgcagtgagctgagatcatgccactgcactccagcctgggcaacagagcgagactctgtctcaaaaaaaaaaaagaagtcagatcctaacctcaaccctatttaacagattatagatgaaagaaaggtacaaatggcttttacatacctcccttctccctgacattttgtatgtgtgtgtgtgtgtatttacacacacatctcatataaggaaattgaagggaggctgcctgcatccctgagtcactctccctctccttctgaatgcttacctgtgcccagaccacctccttagcctcgcaccctccaggcttacagggcactcttctatgcccatcccaagtatagctgataccttccaagggccagacttggtgctaagtaccaagtacgcaaagattaataaaacaatgtcctgtttcagggagctcaaagctgattcggcagggcatggtgtgtacatgaatgataaccacgtagggttgcaggtttcctagtgaggtaagcacaaggcaagatgggaaacaaaggaaggaggggttcacagcctcacccagagtccagaacccctggcctgcctggtgcccatgctgagtccacttctggaacacccagctcagagagggggttagacctgcaggctaacacagacacagcccagaaaacccaggagccgagggggaaggagaaaggtgcaagaaggggaaacccaggtcctggtccccttctctctgcttcctggcagcagaactcagacagaacccttaagccagtctaagtctggcaggaccagtaagttctgagttagctccatactagtttctagcaggctctttctcacttcctgattcttaggtttctacattgacactccctgaagagttgggaagagacaccacagtcccctgaccctgatccataggtcacacagcagggacatccacagggtgggcgtgggccctctcatccctccctcccactcacttcacgctggctgggccccaaggtgtttgcaccccttgcagtgagtgaccttctctagtgcagcaagctcagaacctgctgccactggagttgtcccattgctgatgcagaaaggtgaagaactagcagaacactggaaatgccctccatctgggtccatggctacttaagctcaatgctccctggcaggcaggaggacaggtgctattgccctgttgggacagatgaaaaacagacacagggaggatgagtgatttgccctgactatagagtggcagggccaaggcagagcccaggcctcctgcacctaggtcagtgttcctcccagttacagtctaaactggaatggcaggcaaagcccctgtggaaggggaaggtgaaggctcaatcaaaggatccccagagactttccagatatctgaagaagtcctgatgtcactgccccggtccttccccaggtagagcaacactcctcgccgcaacccaactggctccccttaccttctacacacacacacacacacacacacacacacacacacacacacacaaatccaagacaacactactaaggcttctttgggaaggggaagtagggataggtaagaggaaagtaagggacctcctatccagcctccatggaatcctgacttcttttccttgttatttcaacttcttccaccccatcttttaaactttagactccagccacagaagcttacaactaaaagaaactctaaggccaatttaatccaaggtttcattctatgtgctggagatggtgtacagtagggtgaggaaaccaaattctcagttggcactggtgtacccttgtacaggtgatgtaatatctctgtgcctcagtttgctcactataaaatagagacggtaggggtcatggtgagcactacctgactagcatataagaagctttcagcaagtgcagactactcttacccacttcccccaagcacagttggggtgggggacagctgaagaggtggaaacatgtgcctgagaatcctaatgaaatcggggtaaaggagcctggaacacatcctgtgaccccgcctgtactgtaggaagccagtctctggaaagtaaaatggaagggctgcttgggaactttgaggatatttagcccaccccctcatttttacttggggaaactaaggcccagagacctaaggtgactgcctaagttagcaaggagaagtcttgggtattcatcccaggttggggggacccaattatttctcaatcccattgtattctggaatgggcaatttgtccacgtcactgtgacctaggaacacgcgaatgagaacccacagctgagggcctctgcgcacagaacagctgttctccccaggaaatcaactttttttaattgagaagctaaaaaattattctaagagaggtagcccatcctaaaaatagctgtaatgcagaagttcatgttcaaccaatcatttttgcttacgatgcaaaaattgaaaactaagtttattagagaggttagagaaggaggagctctaagcagaaaaaatcctgtgccgggaaaccttgattgtggctttttaatgaatgaagaggcctccctgagcttacaatataaaagggggacagagaggtgaaggtctacacatcaggggcttgctcttgcaaaaccaaaccacaagacagacttgcaaaagaaggcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttgtgagtatgattccttcctgtcctttctctcttcctgggactgcctgaactagacattctcctggaactataagaaccctcctcctgcgcctccacctccatccccaacacctattcccccaaacttaaattcttaagagaatcctagatcaagccatgggtttggtgagttaagctaagccagatgatacagtaaatgtgcaggaaacctgccttataaagtaaatgcgttctctctcgtgctgagaaacttataagatcctgctggcgctctatactttattggctaggagaagtaaagaaatgtctgattcgaggtgaagatgctccccatgccttgcagcagggaaatttaaattgcctctgcttagagcgtttccagacctgaaagaccagtggtttagggaagcactctacatgagggaaacctgcattagaaggagcttcttaatccctgggatctttccaagctaaactggatgtctacagtggggagaaagaaaagcagagaacaggacatgaggggggctcaaggccccgaagggttgacataggtgtcccttaaagccgaatgtagctccgcagaaagaagaccaggactgagtcaagcttctgctttcccttcaaaatcggccagattttttaaataacttgactctgaggaggaggacctgatttaagtgatggtcccatcactgttgaatcctctgtttttaaaactccccttttgatttttttgggccagagccaattttatttaaaaaaaaaaatctctaaatgaaagggcatcaaaaagaccgcatttcagttatttccccaaacctcaagttcattctccttttgttcttcctgcagcaaatgaaggatcagctggacaacttgttgttaaaggagtccttgctggaggactttaaggtgagagcaggggcggggtgctgggggagtgtgcagcatgattaagggaagggagactctgcttcctgattgcagggaattgggtttgtttccttcgctttgaaaaggagaagtgggaagatgttaactcagcacatccagcagccagagggtttacaaagggctcagtcccttcggggaggcttctggtgaaggaggatcgctagaaccaagctgtcctcttaagctagttgcagcagcccctcctcccagccacctccgccaatctctcactcacctttggctcctgcccttagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtgtaagtagcagatcagttttttcccttgcagctgcccccaaaataccatctcctacagaccagcagggacactcacatccacagacacagcaaagacacagactggcagagctagctgtaaatgaggaaagactcctggagtcagatctcttgctcatttctctttgagcaggcgttgggggtggctgctaggcatttacatgtgaaatttgcaaacagctttcctgttatttgtgagtcatttgtgggttattaactactcccctctctcttcataaaaggagcccagagcttcagtcaggcctccactgcctctttgtaactagaccctgggcggggagctaaggttcccaagcagaggaaacatcattcacctcttttaatctcaatgttttgaaagcaaagctctaagaagggcccaattgactgacaggatttcccctggcattttagaagggacaagggggctattcatccccaggctagtgtctatgagtaattcctccaggtaatttatttctccaactgaaatgatgccctcactactaatggtttcccctgttctgtcaccaatattggaaaatcagttggtgtctatttgtaggacaaggctatgtgaagggtttggtcccagtagcttccctcctcagatgcttagaagtgttcctcggtggctgtgactgacggggaggaacaggagagagaggcagaaaaggacaggctgaagaatgcctcgctcagcactgcaggagatactgtagagttctgggggaggaaggaatcccaagacctgggttgtcatccaagccttgcaaacatcttggagtgagtcctggagaaatacatttaactcccagggccatggaagcagggctcagttctctctgggagctgtgaggcaaggcatttggataaatctggcctcctcatgatgccaccagcttgtcccctaagtgtgatggacatggagctggaagccaggatcaccaacactttctcttttcttccacagcatcgatttcttccctgtgaaaacaagagcaaggccgtggagcaggtgaagaatgcctttaataaggtgagcttggatggtggcagagagggtctgcagagcacaacccatgcccactccccaaccccaaagcatggaaggtggtggggactcaataggccccattcttcattggagagagtgtgggaacctgacagatggtatgacctgctcagccagtgaggagctgctgccttgattgtatttgttttctgttaagtgtctttgggggtttctaaatgactgctcgctgcctttgcaggcttgcgggttaggctggccggccagcctgtgaacacagtgagctgcatgctggggagagtgacaaaggaaacagaaagtacagaaagtagcttgttgggaatctaggctgaacccacacgtgcaggaagctggcacataaatgtgcacatacaaatacacctgggggttcagcccagactccccagaactcagaatgagcaggaagctggattctcacttaacctggagttggttcaagcccgctttccatctgcccttcgcacctgcggaggtgcctgagaatgtcagttcccaaacgaaatggggtttcacacttccaactgtgcgtgaactttttcagtctgatttcccagaaaccgtgcggcctatgtcctcctcgtgggctggggacagacactgcacagagtgccaacatcagggggtgtgaatttctcatagtaggtcagggcggcagggcagggcctgctcagtgtgttggtgggagaacacagacatttaaaaggctccctcctctcctctcaccgtcttgctttcgaagcgcttcctctaatgtcttttcatcaaactctgcataatcatcatgtgaatacgtgacctttaaaattgttgaaaaggcatcattttgaagacagtgctttgcaaaatgaatgctccccttgctagggggaggcctggaggagatgaaggtcaatgcacagcctttcccaaggcagctaggcctatcctctggtttacttcccagcgtgagggagaacaagcaacctctgcactcaaggtcatgcccatccatgagcatgagggaggggagcctatttagtccccagaaaggattttaactgtatgtttcttatctctctgcacagctccaagagaaaggcatctacaaagccatgagtgagtttgacatcttcatcaactacatagaagcctacatgacaatgaagatacgaaactgagacatcagggtggcgactctatagactctaggacataaattagaggtctccaaaatcggatctggggctctgggatagctgacccagccccttgagaaaccttattgtacctctcttatagaatatttattacctctgatacctcaacccccatttctatttatttactgagcttctctgtgaacgatttagaaagaagcccaatattataatttttttcaatatttattattttcacctgtttttaagctgtttccatagggtgacacactatggtatttgagtgttttaagataaattataagttacataagggaggaaaaaaaatgttctttggggagccaacagaagcttccattccaagcctgaccacgctttctagctgttgagctgttttccctgacctccctctaatttatcttgtctctgggcttggggcttcctaactgctacaaatactcttaggaagagaaaccagggagcccctttgatgattaattcaccttccagtgtctcggagggattcccctaacctcattccccaaccacttcattcttgaaagctgtggccagcttgttatttataacaacctaaatttggttctaggccgggcgcggtggctcacgcctgtaatcccagcactttgggaggctgaggcgggtggatcacttgaggtcaggagttcctaaccagcctggtcaacatggtgaaaccccgtctctactaaaaatacaaaaattagccgggcatggtggcgcgcacctgtaatcccagctacttgggaggctgaggcaagagaattgcttgaacccaggagatggaagttgcagtgagctgatatcatgcccctgtactccagcctgggtgacagagcaagactctgtctcaaaaaataaaaataaaaataaatttggttctaatagaactcagttttaactagaatttattcaattcctctgggaatgttacattgtttgtctgtcttcatagcagattttaattttgaataaataaatgtatcttattcacatcatgttctggtgtcagttgactcttgtctgactaaaattgttctctcaaagctcccaagacaaaagagagtgggtgaatgaattctgcatttcagagaaaggaagccagctaccccctacccccaggagagagggaaggctccaaacctgatcagagaaaaagctattctcactgcaaaagccagtagcaaagacatcatggtcttcccacaactcaatcctttaaagatttgctcctaaatccttaaagatcatgctgattttcaaggctaacggggagattctgtaggtaccaaaattagaaaggtaaacaggggaaggttttttggaggttgtttttgtttttgttgttgcttttcgagacggagtctcgccctgtctccaggctggagtgcaatggcatgatctcagctcactgcaacctccgcctcccaggttcaagtgattctcctgcctcagcctcccaagtagctgggactacaggcatgtgccaccacgcccggctaattttttgcatttttagtacagatggggtttcaccatgttggccaggctggttaggaactcctggcctcaagtgatccgcctgcctcagtctcccaaattgctgggattacaggcgtgagccaccacacccggccaggggaagtttttagggtgaatacaaattgcctgggttatcctgaggattaagataatgagctaacagggcttctggcactaggagatgttcgcagtgcgtatagttggttgttattggttatcacaaccaaaaactcccgccaaatcctctttgagctgagatcagaggcaacagggctggacccaacagagttgaaaataagcaaattagcccacttgagaggatagccaaggacataagctgctgctctttctgtcccctctctgagccaccagacaaatgtgagattggcttctgactgcggtctgctgggaggggcacggtggctggggaagccctactcaaagccccgtgacttagaggagagaggaggaaaaaaatgagctgagtaaacactagtccccctcacgctctgcctgggcagccctggtctggggaaggctgggagcggacaggctgtggtcagtttttcagtaagacctgactcacttcggttaaagtaaccataggaaggaagtgggaaacatcaaagcgaaaattctgagatgaaagggtaggagtaagaatggaggttagcagaagataaggagagggggcctgaagctctctactctgaggctgtccatcctgaactgaagaagacatcctgagtctgtgaaaggaaaatcttggggcctcaaaatcaccaagctaaagggaaaagtcaagctgggaactactcagggcaaacctgcctcccattctattcaaagtcatccctctgttcactgagatagatgcatatctgatagcctcctttggaaaagaatcagaaactcaaaagaatgcaaccatttgtctcttacctacctgtgacctggaagccccttccctgctttgagttgtccccatctttctggatggaaccaatgtacttcttacatatattgattgatgtctcatgtctccctaaaatgtataaaaccaaactgtgccccaaccaccttgggcacatgtcatcagaacttcctgaggctgtgtgacaaagctgtgtcacgggcacatgtcctcaaccttggcaaaataaactttctaaattaactgagacctgtcttaaattttcagggttcacaagtcccctggcctggaaggcttaggccattagccttccaggcaatgcatactgactttagtttgagcctggcatttcatctgagcgaagtgggcatctgaaacaaacagaaactcacctgaaaaaaagctgtacagctgaaaagatgttaatgatagctacaatttattaagcaactacagtttattaagctagtcacttattttaagccatttggtatgtattatgtagcaataagatggcatta

SEQ ID NO: 113 - DNA, 55001 bp, Homo sapiens
atataaacagtaaagttaacatcttggactgaagcactggcattcggtttacctgaccttatttctggttggattttacatttgcataactaaaagggttcaccagaattctaccaaaacacagtctatccccagagcagattccaatctccgccctcaaaggcacagactccacgctatctagaggcttgatccccctgcccatttctgaggctgggagcctgcagtgtggcctttgggctaaagatggcgacagagatcaatgttggcgctgcctttttggggctaggttctgaatgagctctaacatttaattcttcaaatacttagcagctactgtgtgtcagcccttgtgcctcaaatccccgcctcacttacagtagatgcatccttccacgcaatgcgtgagagtagaactggatcctgtgatgacttcaggaagtccgggtgtccattctgttgggtaacaatttttgttagctcccccttgtcacgagggacacagcccctgctctctaggagtttggagcttaggagggaaggtagatattagacacatactcatttcgacatttttcccacctatgtgaaggagcatcacacgaatgcttgatcccttctccccatgacagagacgttgctgtgaatctgtctcctgtacactcattccctccaattcccttggatgttcttgcccaggctctggtcacagaattgggtgacaaaggatgctgggagccttgagttctagcagaggctcttccatactggaatatttctcacagacattttctgaaggtccatagtcacaggacccatgatacaaaaaatagcactggttatgtaagtttgtctgcataaaggacagcagacgtaggtatcagctagttatgcattttgtagactggttccagtgcacacattccaatgagtgctcataggtttatgtgagaaaatgtgttatttttgataccttcttccacatgcctctccctgttcacaccctctgcttcagacactggtctcctcacaattctccaaagacacactctaggcacgatgctactccagagctccagcagtagccttgtgttttgaacacttgttccccaattacctgcctggcttgtttcctcagtgaggcctacgctggttacccccgtgaaaactgcaatatgcaccccctcaaatctcagcacccttagattacctatggtactatcacattctaatacactctataatttattttttatttactgtactttttttcctttatcctccactagaatgcaagatccacgggcagaggattttctttttgccttgtttatgtattaccagcatgtacaagcatttatgacgtgtcaagcactcagcaacatttgctgaatgaagaaaagaactcaaataggattcccaaaagggtcctacttagaggtcatgctctgcagagactagcaccagggcccctgtccagatttattaggaaagactgtggcatcaggtgggagtagtgggaagagggtctagtctccagaaggagggctggtgaggggtggacaggatatcagggtttccagtcaatatctgaatcactttgcatggtgtctcagttatctattgctgagtaacgaaccaacccacaatgcagtgatttaaaacatcagtgattcattattccttctgattctgtgagttgactaggtggttcctctgctgatcttgccagggctctgttgcactcagctggaggctcaactcagctggaaagtccaagatagtctctcccatgtctggctgttggtgctggctgttggttgggggccttggctattcatgtgtcctcccatcctccagtaaactggaatgactttcttacatggctatcttagtgttgcttctcaaaaagggaaaggtagaagctgcaaatgctcttgaggcctagggtccaaaactcacatttgtcaggaaggactgtggcagtgggtgggagtagtggagaaagggtctagtccccagaagtggagctggtgagggtggatgggatactggggctcccagccaatgtcagaagcactttgtcataacctgcctcatgttattcactcctagcatagcctgttggtctaagcaagtcccaaggccagccctgatttatggagacgtgggtagagaaatggattcttcctctgaaggggaagatctgcaaagaatctgtgcccaaagtcactgtaaacaaatggaagaggagggggtaaagcacaggctgactgattcgtgaatgtgcactcctgaccatcatgctatgctgcctccatgagaaactctgacatggctgtgtagccagcaattcattcattcattctttcatcaaatgttactgaccacctgcaatgttcaagtgcagaactgggcagggggcatacagaattgaatggggtccaagatttggtgctcctaaggaagatgagaaagccagccttagccaagctctctcaaatcttcttcatcttgctctgtggttttaatgctaactaggaacccaagggagagggtcagcccttggaatgtcttccagaggaaggtgaagcccagctggagtcccagtctgtggcaagtaaactgggagaagcaaggacaggcttaaggcttagcttctgtgccaggggctatgtgtaaggtttgacttcctagccttctcaggttcacacactactcctcattccttcatgccctcccaagccctgaattcaatgttgggcaacagtgagagcttaatcaatggctttgaatgattcttaggcaagctgagaggaatatcaacaaatgttttatccgatattcccaagagtagcctgacccaaatccaagattatctccatggcctgcccctttacatcaactcagaggatgctgctgacattatatatttgcatgcttactatgttaaaaatgtactgtcaggaaaccagaggctctcaaattatcttaaccacttatatcaggatcagctgaaatgctagttaaaaatgaagatttctgggactctaccccttactggctaatacaaaattgcaaaattgtggagctgagcctagaaagctgaatttctaggcagcctgtgggttattcttatgaagagtctgaggatctccagcctctacagaacgtcacttcttccacgtccatttctgccactgatgctgaataggcagaatccctccttactaccaccacctgtaacatgtgcttgtcacatgagttaaaaataaagccccctgttgtaagtcgcaagtcagacccacagaatttagatcccaatcagggaacgtgacgcttggtacgtttatgttcttatgacaaaaaagggaataactaaaacaggaagttgaaaatcaaactttgactttcctatatggaaccatgcgccaaattctgaccctctctgtcactctctgctcgtgtgcgttagtcataggaggtcatggaaaatcatcctcattcctcattatgagaggctgctcatcagtcattacccctacaccctggcggggaggcaaagtaggtgaccacgcatgcaaattcagggaagtttggctcagagagttcacatcccctcctaacacaagagacagagacaccaaaggtttggaatccagtaggggtgggagatgctttgcccagggctgtctagtatggagttaagaatatgttttttgggggtgagtgttcaaggtagatgatgcttccttccctgttctggaggttgggtcctcttccataaggtgttgtcctttggtacagctttgttgtaccatatagatggtaccttctgggcctggacaatcacttgaccaaagggatgggagctcttgctctgaagagatcctgacaagagaatataggtgtggcagagcccattcctactccctgcacaccctgctggcagtaggtagacatttacccctttatcggtgttcttatatggcaccagactgtgagctcctaaggggatggggccttggcatttttatttctagatagagtcttgcctcacaaaaggacagagatacagtctaagaaatgtgtcattaggcgattttgttgtgtgaatatcacagagtgtacttacacaaacctagatggtacagcctactacacacctagactatatggtataacctcttgcccctaggctataatcccgtacaacatgtgactgtactgaatactgcaggcaactgtaacatgatggtaatgatgtgtatatttaaacatatgcaagcatagaaaaatatagtaaaaatatgatattataatcttacagggccaccgttgtgtagacagttcatcgttgagtgaaacttcattatgtagcacgtgattgttctcataaatgaacatgaaatgattgggccaccttatatagaaatgcagggtttccactgtgtaactctagagggcaccactcacatgttgatatcatagaattgaatatttactataacaatattatgacagttggctataaagtgccttgtactaaaaaaaaaaaaaggccagcatttctaataactttctaactgatgggaataaaatcagaacaggcagcctgttgctaatccactccaaggagccacaagttgttaaagcaaagagcacagccagatcaggaaatccattaaggcaagctacaagccgggtctttatcaggagggcaatatggagtcaccatggatgatttcaagcaggggagtgacacgatcagattgacctcctaaaatagttgcttctggctgccaactggagcatggattattggcacagccactcaagtggtttccttcaagaattgttagacactcatcatctctcaactgcaggagatttgagagaagttaatagatttaaggcatctttgggaggtagagttaatagattttggtggtttattgtaggcagagtgagagagtcattgtgaaggtttcttgttttggtgtctattggtaggtgtctctactaatgagtggtggttctattgatgggtggtgattctattcactgacataagcaatgctggaaaagaaagtggtagtttgggagcagagagtgtggaaatcataagctctgcttttgcctatgttgaattttgaggtgtatgtgagcatccaggtaaaggatttcagtgggcagttacatatgcagatatggagtgcaggagagaaatttggaatggagatataaatttcagagttttcagcacaaagaagatgaacttatgaggtgaatgaaatgaagagagaggttagtttaagagaggagatggcccaggagagagagatatttgagaggcaggcagaagatgaaaagccttctatggaggtttagaaggagaggcctggagaggcagaaggaaaatcaggagaagtggaatcatggaagtctatataagagggctttagggcgagagccagcagtgtcggatgctaacagtagatcaagtccagaaaagtttgaaaagtgtccactggatctaggagctaaaaactccagagtgccttcagaagagtgattgtgcaaaagtgaagatgaagggtcttcaggagtgagtggaaagtgagaacacagagacagtgagtagggagaattattttaataactttggctgtgaaggggaggagaaagagaagggtgtagctagagggagtcatggggctaaggaaagaaactgtcaagatgtggcaggtttaaacaagtgtgaaagctgataggaaggaaggagccagtgaaggacaggttggaaaaaacagggcgaggttacagtatgagtacatgcatgagctctcgagcaaacctgcctggattcaaatttccactctgttatttctcatcagagaaaatagaatgaaattgatgtactcaaattactgaaaaaattataagtgtaggagagtcatgcttttcttattttctaatgaatccgggagcaaagaaaaatgtcagtgtctggctgggcgcagtggatcacacctgtaatcccagcactttgggaggctgaggcggtctggtcacctgaggtcaggagttcgagaccagcctggccatgttggtgaaaccccgtctctattaaaaataaaaagttagctgtgtatggtggcatgcacctgtagtcccagctactagggaggcagatgcaggagaatcacttgaacccaggaggcagaggttgtagtgagctgagatcatgccactgcaccccagcctgggtgacagagcaagacaccatctcaaaaaaaaaaaaaaaaaaaaaaaaggctgggcacggtggctcacacctgtaatcccagcactgtgggaggccgaggtgggcagatcatctgaggtggggatttcgagaccagcctgaccaacatggagaaaccctgtctctactaaaaatacaaaattaggtgggcacgttggcacatgcctgtaatcacagctactcaggaggctgaggcaggagaattacttgagctgggcaggtggagattgcagtgagccgagatcatgccattgcactccaacctggacaacaagagcaaaactctgtctcaaaaaaaaaaagtgtcagttaccttctttagaattagcaataaatcttttcagtatttataccattaaaaatttatatcattcaaaatatgaaaatactttttcagtattatttccatttatctatttatctatgtttcatgtatctattgctgtggaataaaccacccttaaacttaatggcttaaaaatagaaatcatttaatctgcgctcgcttcggcagcacatatactaaaattggaacaatacagagaggtttagcatggcccctgcacaaggatggcatgcaaattcataaaccgctccataagaaataacttttttttttttacaaaagaaaatacctattctcctttgtgactctgtaattgggcaggattcagtggggacatagggacatcttgtcttcactccactcaacatcaactgagaggagtctaagggtgggaactggactggtcagatgactgccttactcacaggtctggagattgatgctggatctctgctgggacctcagcgggactgttggccaaaccacctttgcttggcctcttcatgacacctgggcttccccacaatatggcaactgggtttcaagagtaagggctaggagagagagagagtcaggcagaaactatataacttttgatgacttcgcttcagaagtcattcagcataacttttgcctcgttctattcactaatgtcagtctgcattcaaagggaggaaaatcatactccacttcttaatgagaaaagtctcaaagaattcatgggcatgttttaaaatcagaacagcaacaagttgccaattaagataacaaattacaggtttcattttcagggttgtttctacagtactttgaaacattttctgaaatcttcagtgtcttacactgattaataaacataaggaatgtacgttcataatatatccctcttttgttcaaatctttgcaatgacatatttattttaactaggaacagaaaagttggagatggcagccaggtgcagtggctcatgcctgtaaccccagcacttcgggaggccaaggcgggtggatcacgaggtcaggagctcgagactagcctggccaacatggcaaaacactgtgtctactaaatatacaaaaattaacagggcttggtgtcatgttcctgtaatcccaactactcgggaggctgaggcttaagaatcacttgaacctgggaggcgaagtttgagccgagatcgtgccactgcactccagctgggcaataggatgaaactccgtctcaaaaaaaaaaaaaaaaaaaaaagaaaaacagaaaagttggtgacagcagtgggtatttgtacaggtctttataatgcacagaaaaaaagtgctagtcttgcggctgttctcagtttaatgcctcccagatgaattctttgaaaaagtttatgttgcaaaaccactgaagctgtgagggagtgggggcacctgtatcagagaactttttgtttgtttgttaattctgaagactagaagcactataagcgatattatttgtatgataagcaacagctcaggcctttgggctcttaagattttctcagccgaaatgaaataagaacgaaaagcagtttcatttaataatagtgggataaagaatatagagcatttttcttcccctttgtgtaaacatggttaagtaaagtgggtgtaacagtaataagaaccaccacttcacaggcttctatggggaagactgggttcactcatgcactccaagcccttagcacagagccaggcacactgaggtatgactaagtgactgaggatgactgatgatgtgagagccttgaggagcctgaatgggacacaaagcaaatgcagagggactggccttgggtaagagtgggctcctgcttctattgaatgagagagaagagagaaagcataaagaaagtttcaataggttttaatgtgggctaatggggaactgagaaagtgtccttctcctagcttctattactttgctataagtgaggaaaaaggagatgggctaggagattgaagtcagtggagtaggctggaaatattttctgtggagatgggtcagccagctcattatagaaacatgaaaggtttgctaagcaaacatggggccagttagttttggatatcatcagtgtggggcagtggctttgggttagtctgggagggtggaccttggacagatccagagctttccacacagtgcaatgggagggcaaaggagccaaacagtggagaatattgatgaaagaatggtccaagtgagcaactgaaggaagtgaagacctccaaggaggaggtggtagagcaaatttgagcaggttaaaggatggaaggtttctgtgtggttgaaatgcctgagcaatgggaataatgaagaatgagaaagagagagagagagaacaagactgctatcggttgtggtcagagggtagtatagtaatttttacagaaaggtcatggtgttagtcatccaaatggacattaagaccccttggggattaggacaaagaagagagctaggttccagtgacaaatatttaataaataggggagaatgactaggcagtcagtagctgaaagctacaaagagagatataggatggtaaaaacgttaaaggactgatgcttttgtttgtttttgtttttgttttgttttacaaaggctggagaagtcacaatctgaaaatgtcagtagagagatgaccccatgctggccctgtgatatccagatggtaggggcatgagcttcttctccttgagaaagatgcagggcaaacatggtcccagaggatagccaggtctccgccaaggcacaaattgagaaaacagttagaaaagagaatggaaaaaaatagataggtttattcatttaaaaagtaactatcaagcacctgctctgtgcatgatactgttttgggcgttgagatgcatctgtgaataacacatagtaaggaatttacattctagcggtaggagacagagaataaatgcctgagcaaatacatactcaatataaatttggatagtgatacaggctgtgcagaaaaaatatctgctattagatgaacagtgaaggtgagcagagaaagtggaggtttgctacaaattgtgttctccaaaggaaatggagagtgctgtgggagttgtgttgacagtaaccctagatagattttattgtattaatgcttgctagaataaatagctctactgaatagcagatggaggaaagcacactgggctttgcaaatcatcactgtgatgcatgtaattttgcagcactgccagggtttctcacagcaactcattcccattgtggccccttagggaaagctgtgaccagtgcaagatctctcctacctgcatgacctcagggccagcaacagacaattgcaactcctgcccacagactccatgggtcagggaaccactggaaagcaaagcagacacttgctgtatcagggttggaggaagcctttgaggccctttgcatgcagctcagagaggaatggcattctcaggttgagcaaagggaagtggctagcacttcagacatgacactcattgaagcttggaggagacttttttagggcaaagaaagggaagttttatttcactcatggttagtaaacataagacatgtacattcttaatacatctccaaataacattagaggaagaccaggaacaatgctgaggggatagagctataaccgactggggtgagaagtgggggcatttgggaagggtgagaagtgggggcatttgggaaagagtcctcattccaagtggcctcaaggtcagtatctgggctcctgtgattctctctgatgctctgattggccactgcattggaaatgccactcaggctcagattggtgccctccttgtgtgttttttctttatcacatatcctacagtccactttcctgattctctcctaacatgtgcctcttcttgagatcaagaggaagatattattttctagttacagttaaagtttttcctttttaaaatgaccaaattaattcattgatacctcatttattcccaccttttgagctctcattatattcaagacactattgtaaaaaatggagacttaaagataaaaataatgttataattcacccagcaggaagaagagataatttttttgaatgacgctacaaaataatgtaaatgttgggtttccctttactccacattctctccttcaggcatctccaagcttcaaccataactgacagatctttatctttagtgcatttttctcttctagcttccaggtttgtacatccagtttccaactggacactccatttagcatcccatctgcatctcaaacacaacacaactcaaatggaattaaccgtctccctgactacccttagaatcaatctgttcctccacctcttgtatcatctttctcaatgaatgatgctattgctgcccgccagttatctaagcctttaaataataatacccttgatcccttctacattcaatagtcacttcccatatttagtagttactaggtcctattgagtctacctcctaaacatttctagggttttctctccatcctcacggtcagcactgtgggttagactcttgtctccttatctgtaggtcactgaacaacctcccagctagtctccttaccacaaagtcttgcctccattttttttttgttttgttttgtttaggagcagaggtttaataggaaaaaggaagagaaaggagaatggctctctcttgcgagacagaggggctcccaaatgggaattcccagtcttgctttgttctaaagcaggtccacattgctacatagggatggcaccctgccttttcttcaatggctccccacatgtttgtagcacagaaaaggctcctcagattctgggccttgccaatcttcaggtcatttctccattgcccttccggattcttgtacatgcagcaaatttaataacagccattatgattatgaggttcttaaagattctccaggatgctgtcatctctcacaagttctgtctttgcacagaacttgtatctcaacttctgtttggaaaatccttcctcttctacttccctggttaattttctcacatttcagagatcagctgaaaactcaacccctcttggaaaccatgtgtagctctgcagctttttctcagcagccacgagacactggatcgcccccagcataacacttgccaaatggcatgttaattgtgagcctcttccatgaatctgtaggtagttcaagggcagaactgtgccctgtgtgtttgtatctccagtgactgacttgtactggatactcaataaatgtgtgttaaatgaatacatgaaaaaagggagaaagaatgaaaggaagaaagggaagaaagaagaaaggaaaagaaaaggaagggaagaaggaagggaggaagagggggaggaagagggaaagggagaaaaatgtagactatgggaatgaagaggagcaataactaactttaaatcctatactaaataggcagtacagtcttcaaagaagaggagatgtttgggccaactgttgaggtaaagggaagagttatccaggcagagtaagggcaaggggatttttagacacagccaagaacgcgcacaagcagagaggtatgcaaaagggtgaaatgttggttaatttgttgggattcagtgaaagactataggaagtaccctagctagtttaatgggaaaatgtttccatataaagaatttggttcttacagagtcagctggaggaccacaggaatgggcttttgactggacatccaggaataattcccagagggacactgtaaaactggcttgctggagagccatcatctctgtcacaatcaggaaaatgctgctacactgctggctccagcaacacggcactggggcccagtgcagggatcacacagctgttggctccagaaaaatacggctttacctataatccggagatcagaaagttacctcctgaagcatgctgtgcttgctaaaatatgcagtaaggaaatagatggctctaggtcaggcgtggtggctcacgcctgtaatcccagcactttgggaggccgaggtgggtggatcacctgaggtcgggagttctagaccagcctggccaacatggtgaaaccctgtctctactaaaaatacaaaaatgagccgggcatggtggcagatgcctataatcccagctactcggaggggctgaggcaggagaagcccttgaacctgggaggcaaaggttgcagtgagccaagattgcgctattgcatgccagcatggaggacaagagtgagacttcgtctgaaaaaaaaaaaaagaaaaaagaaaatagatggctctatttaaataatgtattccaaatgtcagtctgccaagaatgcatctgcttggcagaattgaagtcacatctggaactcttgctgtgagagagtctgagaaatgtgggttttagctttccaagcactgcaggaatgagttgggtgggtgctaagagagacattgagtagccaagccaccatctgtactacctcttccatgtcttctgaggtgtaggaaccagaaatggtctgtgtatgtcagtagggataagccagagctctttgtagagtggtttcccctcctgctccccggccatgctggggcactacgcaatttgtgtacctttatccccatggtggaaagttgagccaacactctcacattcttctcaagtgaagcctgtgctccttttctgggtttccaccgtgcagcttagagcccagcttagcatcaccatatctggggacagtgctcatagggtggctgactcacagagactaacttgatactcttctacaagtgaatgtagtacagagtatggggcttggaggagaggtcttgtcccaccatgagccctactcttcaatagctgtgtgaccctggacagactcctgcaatgttctggatcttgtttggtgctttgtgatatcaaaatgctcactcataaagtgagtggtcaggataggagctactgccacaggtaatccaatgccttttgtgtcttgcttcactccctcagattcaaagtcttcagtaagagcatccatgtgacctataagcaaaggctcatgtgcccacactttcctcactatcagggaggaggaagggagtagttggttctttctggttatgaaatggaaggcagagccctgtcttccaccaaaacgagaccagtgtggaattcatccccaaatggaaagcatgtttagttaatgggtagaaaatttaaaagccactacactgcgaatgcctgtataattgcacatatttttaaatagagattgctcacttttgcttcctcctgggcatgacactgatatccatgaaatcataagtttaatggactgtgtatttctattacatcttgaaataaacacataataatactattaaaacacagaaatacatcctgagatctgaaattaatcctgtgtaccaccagtgatatacatcacatactttaggaacccaaatgcaaagtgttgacaacaattagtgtggcaaattaatctttatgggtagatccatgtgcatcagtcaggtctctgtggaacaagcagtggaaatagacttcagactaatttaaacagaaatttatttatttttagaaaaaagtattcaaaaaaaatcaggttagttcccagaattaataggaaagctggataactggcttagaaaatggacagagatcaagggagatttttgtagggggtggggaatgtgccaatgaaagatagagaggggctattcacagaggaaggatcattctgctgtaacattctcttcttcctttaaaacaaatattttttgggtggtgctgggaatgaaaatcattttattttcagcattacttcgtttctgcgtgtgattacatttttttcaataggttgctgggaaacaggcatgtttggttacatgaataagttcttcagtggggagttttgagattttagtgcacccatcacacaagcagtgtacactgtacccgctgtgtagtcttttatccctcattcctcctcccaccctttccccctgagtccccaaagtccattgtatcattcttatgcctttgagtccttacagcttagctcccacttatgagtgatatatatatatatgtatatcacattttctttatcccctcattgattgatgggcttttgggctggcttcatatttttgcaattgtgaattgtgctgctataaacatgcgtgtgcaagtatctttttcatataatgacttcttttcctctggatagatacccaaaagtgggattgctggatcaaagagtagatatatttttagttctttaagaaatcttcacactgttttccatagtggttacaccagtttacgttcccaccaacagtgtaaaagtgttccctttttaccacacccatgccaacatctatttttttttattatggccattcttgtgggaataacttgttattgcattgcggttttgatttgcgtttccctgatagtgatattgaacatttttccataagtttgttggccattcctatatcttcttttgagaattgtctattcatgtccttagcccactttttgatgggtttgtttttttttcttgctgatttgtttgagtttcttgtagattctggatattagtccttttttggatgtatagattgtgaagatttgctctcactctatgggttgtcggtttactctgttgattatttcttttgttgtgcagaagctttttattttaattaagtgccgtttatttgtctttatttttgttgtgtttgcttttgcgttcttggtaatgaagactttgcctaagccaatgtctagaagggtttcccaatgtctagaagggttttctagaatttttatggtttttggtcttagatttaagttcttgatccatcttgagttgatttttgtataagatgagagatgaggatccagtttcatttttctacatgtggcttcccaattatctcagcaccatttgttgaatagggtgtccttttcccactttacgtttttgtttgctttgtcaaagatcagttgactgtaagtatttggctttatttctgggtcctctattctgttccattggtctatatgctgatttttataccagtactatgctgttttggtgactatggcctttttttttttaattaattaattaatttatttattgattgatcattcttgggtgtttctcgcagagggggatttggcagggtcataggacaatagtggagggaaggtcagcagataaacaagtgaacaaaggtctctggttttcctaggcagaggaccctgcggccttccgcagtgtttgtgtccctgggtacttaagattagggagtggtgatgactcttaacgagcttgctgccttcaagcatctgtttaacaaagcacatcttgcaccgcccttaatccatttaaccctgagtggacacagcacatgtttcagagagcacagggttgggggtaaggtcacagatcaacaggatcccaaggcagaataatttttcttagtacagaacaaaacgaaaagtctcccatgtctacctccctctacacagacacggcaaccatccgacttcttaatcttttctccacccttccccgctctctattccacaaaaccgccattgtcatcatggcccgttctcaatgaactgttgggtacacctcccagacggggtggtggccgggcagaggggctcctcacttcccagtaggggcggccgggcagaggtgcccctcacctcccggacggggtggctggctgggtggggggctgacccccccacctccctcccagacggggcggctggccgggcggggggactatggccttatagtatagtttgaagtcgagtaatgtaatgcttctagatttgctctttttgcttagtcacactttgactatgtgggctcttttttggttccttatgaattttaggattgttttttctagctctgtgaaaatgatggtggcattttgttgggaattgcattgaatttagattgcttttggcagcacggtcattttcacaatactgattctaccatccatgagcatgggatgtgtttccatttgtttgtgtcatctatgatttctttcagcagtgttttgtagttttccttgtagaggtctttcatctccttgggttaagtatattcctaagtatttatttttatttttatttttttgcagctgttgtaaaagggattgagttcttgatttgattctcagcttggttgctgttggtgtatagcagggctgttgattagtgtacactaattttgtatcctgaaagtttgccaaattcatttaccagttctaggaactttctggatgagtctttagggttttctaagtatacagtcatgtcatcagcaaacagcaacagtttgacttcctctttacttgtttggatgtcctttattgctttctcttgtctgatttttctggctaggacttccagtactatgttaaatagaagtggtgaaagtgggcattcttgtcttgttccagttctcaaggggaatgctttcaactttttcctgttcagtataatgttggctgtgggtttgtgatagataacttttattaccttaaggtatgtcccttcgatgccaattttgctgagggttttagatcataaaaggatgctggattttgtcacatgctttttctgcatctattgagatgatcatgtgatttttgtttttaattctttttatgtaatgtgtcacatttattgagttgtgaatgttaaactatccctgcattcctggtatgaaacccatttgatcatggtggattacctttttgatatactgctggattcgattagctagtattttggtgaggatttttgtgtcatgttcatcagggatattggtctatagttttcttttttgttatgtcctttcctagtttggcattagggtgatactggcttcatagaatgatttagtgaggattccctctttctctattttttggaatagtgtcaatagtattgataccaattcttctttgaatgtctgatagaattcagctgtgaatccatctggccctggacttttcttgttgttaccatttccattacaattacttctaactaccacttcaatctcactgcttgttattggtctgttcagagtttctatttcttcctggtttaatctaggagggctgtatatttccaggaaattatccatctcctataggtattctagtttatgcacatgaaggtgttcatagtaggcttgaatgaccttttgtatttctgtagtattggaatgtttcccatttcatttctaattgagcttatttggatcttctctctttttcttggttaatctcgctaatggtctatcacttttgtttatcttttcaaagaaccagtttttgtttcatttttcttttgtattttttttggtttcaatttcatttagttctgctctgatcttgtcatattaccagaatagctttactggttccttctcatttgggtagactacgtcagagtgaatatctgggactcaagggctgctgttcagtttcttttgtgcctcggggtgctcccttgatgtgctgctctcctttcccctaaggatggggcttccttagagctgaactgcagtgattgttatttatcttctggacctggccacccagtggagctactgggctctgggctagtactggggagtatctgcagaatcccgtgatatgatccgtcttcaggtctgtcagccatggattccagcacttgctttggtggaggcagcaggggagtgaagtagactctgtgagggttcttgtatttttgttaactgcactgattttgtgttggttggcctccagctaggagttggtgctctcaagaatgcatcagtatacggaggatcaggcagtgggtgagacctactcctggccttaaagctcccaagagattatgtattttgtatttggctgtcagggcgggtagagaaagatcatcaggtaggggcaggattgggtgtgtctgagctcatactctccttgggccaggcttgctaaggcggctatggggtctgggggtgtggttcccaggccaatcgagttctgttcccaggtggattatggctgcctccgctgtgtcacacaggttgctggggtgtggttcccaggccaatcaagttctgttcccaggtggattatggctgcctccgctgtgtcacacaggttgctggggtgtggttcccaggccaatcgagttctgttcccaggtggattatggctgcctccgctgtcacacaggctgctggggtgtggttcccaggccaattgagttctgttcccaggtggattatggctgcctcctctgtgtcacacaggttgctggggtgtggttcccaggccaatcgagttctgttcccaggtggattatggctgcctccgctgtgtcacacaggttgctgggggtgtggttcccaggccaatcgagttctgttcccaggtggattatggctgcctccactgtgtcacacaggttgctggggtgtggttcccaggccaatcgagttctgttcccaggtggattatggctgcctccgctgtgtcacacaggttgctgggaagtaggagaaagctggcagccacaggcctcacccagctcccatgcagcccacagccctaaaggccggtttcactcccaccatgccccaccaacagcaatgagtttacttccaggaagctggtgagcagggctgagaacttgctccaggctacaagcctcccagctgagaaaacgagcgaactcacagttcctcgactgtcccatggagcctgcagtggcaatccgcctccttcaaagggtctgtggactttctcggctttcctggtatgttcctgtggaagttcttggagcaaaagttcacgatgtgggtctccatacactgctctgttcatctgagtgggagctgcaaattagtcctgcctcctatccgccatttttcctcagcatcctcttctttcttttcttttttttttttttttttttttttgagacggagtctcgctctgtcgcccaggctggagtgcagtggcgggatctcggctcactgcaagctccgcctcccgggttcacgcccttctcctgcctcagcctcccgagtagctgggactacaggcacctgccaccacgcccggctaattttttgtatttttagtagagacggggtttcaccgtgttagccaggatggtctcgatctcctgacctcgtgatccgcccgcctcggcctcccaaagtgctgggattacaggcgtgagccaccgcgactggcccctcttccttcttaaaagctggaccacgcctgtaatcccagcactttgggaggccgaggcgggcagatcacaaggtcaggagatcaagaccatcctggctaacatggtgaaaccccgtctctactaaaaaagtacaaaaacaattagccgggcgtggtggcgggcgcctgtagtgccagctacttgggaggctgagacaggagaatggcatgaacctgggaggtggagcttgcagtgagccgagatcgcaccactgcactccagcccgggcgacagagcgagactccgtctcaaaaaaaaaaaaaaaaggctggaaaactggagattattttgaggccaggcgtattcatgttcaggccaacaggtactagctgagccttgtgctagtcacaagagaagcagcaaatgacaaggggaggacaggagtctgtcaggccaagcactggtcccttatggagacaaagaagaatgagccggaccacagctatgaggttgacctaatggtttctcttgcaggcacagtgccattgcatttcctacagggccccacttaggaggcacttctatcactctatcgcacagatgcagaaactgaggctcggagaggttaaggaacttatcccagttagtaagtggtgccgacaagaatcaaatttagacagttctgacagaaagccatgtttgtaatcaccacatattcatgctccttcttagcacagggtggctaaaaagctcaaacaagccaagatttaaataccacaaagaagatccaagtgacctgtgaggaggagactgccagtggatgggcgatcaggaagactttgtaggagaggaaaaatatttttttctcagttctcataagttcttagttggaatgggcagatgaataagagaaaaacaagcagatgttaattaacatgtatatttcttatatacatgggagacacccagggaataagtagctttcaaagaggtggctttgaattccagcttatatagtgtcttcaacaaagaacagtaaagttttagagacatgacaagggaattaaaaaggactttgagtctctaagagtagtgacttgtggaaaggcgaaatgtcagataaaggctagtttgtaaagcttgttaatgtagattcccctggtgccatctccagactgataagagtcttaaagttgtcttcgctggttgacctttgtcctctctggtagacgcagtgggggtgaggagtaggggttagggtagcgagggggtttccttccacctgtgggaagaggcattcacattttaagattgggagcaggaggcctgatgtgattacagctgtgatgtaaaaccgacattcaatgatattttaatatctcatacctgtaatctttttaaaaacttgggaaaacaacttataatttttacaagaacattaataaaaataacaagttgcacctactatagccactatttcataaaagaaaggccatttttacatttcctctccactcagtgagcatgatctcagaagaaaggactgcctttctcaagaatgaagagttaacaatagcatcccaacatatttaaatatcaagccctgggctctcgattttctaatttaatttagctcatcattaaaactggcactggtctaaggaccaacatgtagaaaccaccgctctgaaagatctggcagtagcatagaaggcagattggagctggggaagagagacttttttattctttttttatttttattttttgagatggagtttctctcttgttacccaggctggagtgcaatggcgtgatcttggctcactgcaacctccacctccccagttcaagtgattctcctgcctcagcctcccaagtagctgggattacaggtgtgcgttatcacacccggctaattttgtatttttagtagagacagggtttctccatgttggtcaggctggtctcgaactcccgaactcaggtgatctgcccacctcggtctcccaaagtgctgggattacaggtgtgagccaccgtgccccgcccgagactttttttcttaaggagattgtcttagtcacctcagactgccataacaaaataccacaggctgggtggcttaaccaacaggaagaatgtatttgctcccagttctggaggctgtgatcaagtctcagatcatccacttgctggctggcagacagctaccttctcactgtggcctttccttggtgtctgtgcaggcacggggacagtgagagctctctggtgtctctacctcttcttataaggacaccaattctattgtgttagggccccacccttatttaacctcaattactttcttataggctctatcttcaaatacagccacactgggagttaggactttgatatgtggattgtggagggattcagttcacacagagctattgcaatggtttggacaagcagctctaattggggaagggggaagcctgtaggcatgggaaggagaggtcaggcgtgtcacgtgcgtctgtgtgaaaagaccaccaaacaggctttgtgtgagcaacaaggctgtttatttcacctgggtgcaggtgggctgagtctgaaagagagtcagcgaagggagatggggtggggaggttttataggatttgggtaggtagtggaaaattacagtcaaagggggtttctctctttgggcaggcgtgggggtcacaaggtgctcagtgggggaggttctgagccaggaaaaggaatttcacaaggttaatcgctcagttaaggtggggcaggaataaatcacaatggtggaatgtcatcagttaaggcaggaactggccattttcacttcttttgtgattcttcacttgcttcaggccatctggatgtatatgtgcaggtcacagggcatacattggcttagcttgggctcagaggcctgacaaggcacagaaggagctgggatagaggtgcaaagtactcatgctgacctgacatcccatgcccacggcagacatgactacttgatcggacaatactttcttggaatcaaagacacaacttcacagtcctcagcatcatagttcaggcagcctgtatcagtagatcagagtgggcacatggcctggtgctatcactccatcctgtgaggggagatgggattggaatggaggtggaggagtcacaggtggtcctgatatcacaggcctgggagtctgaaagaagtgcagtaggtagtattatggccctcaaagacatctgtgtcctactgtctagagtcaattaaagtgttacctaacatagaaaaaaggattttgcaggtgggattaaactaaggatctcaagatagaaagactctcctggagtatccagttgtgtccaatgtaagaacaagcgttctcaaaatagggacacaggagattgaattgcaaaggagatgtgctgataaacggagaggttggagtgatgccgccatgaggcaaggaatgtgggtggcctctagcagctagcaatgattcggaaatggattctcccccagagtccagaaagatcacagcagtgctgacaccttgatttgagcccagtgtcacctctgtcagacttctgccctatagaactgtaagaggataaatttgtgttttaaggcacaaagtttatggtaatttgttatagcagcaatgggaaactaatacaagaagggttgtgttatcaacagaaatacagcaatcagaagaaagatggttggggagagcagagggtggggtgggaaggtgatggattctgtttgggacattctgggtgtcatatgaatattattggggaactgtccatcaaaagctcagacttcaaagaaagaaaggaaggagcaacaatattttgaaagggagaaagtaggtaatattcaatatgtgttgagtacctacaaggtttttagttctttcaccaagttattttatttaatttttatggcatcaaggggtgttttttttttttcttaaaccaaattttctcagttaagcatcccatgtaagtggccaggggttaagagatgtgactaagactagacagagtgattttcatcattacatggtactatggttctactgaagcactgaggaaggaagtcaagagagttcctgagaaatgtctggagtcttagtaacatgcaaagtgaggcagaggtattgtcagtgttagctggtcactttgaaaggttagaggtaaataaagttcagggcaatggtacttggagaggagtgaatctttggtaacctggaggagatgcttctcagaggaaaatttcttcacagcatgaagtcaccagctctaagactagtgggtgttctgggggatgttagcagtgactcactgactgttggcccagtgaggggagggatgaaaggctgggtagtgggtagagaatctgactcctcctgcccccacatgtctattcctgggccttctcatggtgggggcaaaaggtatgcaaagggatttccgcagtgtctgagtgctcccaggagacggaaaagtaactgatgacttggagttggagtcttgttggctcaggcagctggggcgttttgggatttcaacacctccctttagtttctatgtatggtgctgagcaagtagtcaaagactttggtcttgctttaccccagtagcccacaagcctgagtacatggctaacatttattgggcacttagtgtgtgccaaagagctttatgagacaggttctttttgagtcccattatactgataatgggactgataagaaacagaaggtcagaaaggcccagtaacttgggcaaggtcatgtagctagtaagtggtccagctgaaatttaaaaccaggtttatccaactccaaatcccacatctgatttattttgccatgaattaaaacatagttctgattttgagaaactcccagtcccagtgggaaaacagatggaatcacaaattaccataacacagtgtagtgatgtagatataaaaacaaagacatgggaaccctgaaaggaagttgtaacaccaatagaggaatgcacggaggagatctttcagaggaggtagcctcgtagctgggctcaggggtggatgagtttctggctagaagcgcattgtgggtagagggagaacatatatagaaaggcatgaagatggtggacatgtttggaaagagtgagtactagactgatgaacacgatgagtcaggagcttatgacgtcaggggctactgctagcctagctaatgactgaagattctgtgaaaagaaaaccctaacatttccagctgagtactagggccatggtgattatatatcatctctcatgacaacacaaatgtttgggcttacttatcatcctgctcagtaatcattatgtcactcattaaagatgtatcagggtaaattttaggtgagaattgattagaaatgtaaactctatatatgtagttacaggcttcacagtttgccactcaaacaatatggtcatccaatcatgaggcacaaccttctgctcagaacttttttataattgggagaaagtgacatccgagttccatctcttctgagaactgtgcagctcagaaatcttcttcccttttttgatcccccagttgggtgagatgacattaacagtgtgttataagcacttttcatgtttactaccccatattcaaaattaagcatttcaaacaactgtataattgtctataaatgtagcattattagttaattttccttttaacacattgaggttacctctaatgttttgctatttttaactatgttgaaataatcatgtggtttttattctttgaattattttatattgtaatgttccctgaaatgaaattagttgatcaaaagatgagtaatcttttcatgacttttgatacatgtcactcaatcaccttctggaagagttggaacaagagcctattgctggcatcccatttcatagccatctcaccaatgttggaaaatgggcttttcctattttctgctaattcattagttgttaatggcttattttaactattaatcccttgttattgatgaaagttaagcattttctgtatttttattttctgtctaaattctactttttgcacaatttcctttgctcacttcttcactggaatttagtatttttctttccaatttgtatgactccaaaatcagtgataaaataagcaaagggtctttcttagaggctaaacaagggaatgaataaggaacaagaacagcaaagggatagtgaataaagatacaaaataataacttatttttatttttgaagctttccccaggcagtaattcttgacctttttaagattataaattattttgagatatgcattcttatcctcaaaatgaattagtacataaaatttaaatgggttgatagatcccctgaagtgtatccacaaaatgctctcaggttaagcaacattgttctataataactgctttaaccaaaatgtttaattattgcttgtgtttacctgtcctagcaaagagctaattgttaaattttgtgttagagggtcaatattataaataacttaatgtggcctctctttcctttcctttttttgagagttttgaggctatcactaagtgggctttagagtaccaggtgggaatctgacctcactgctcaaactttcaacttcaggcatgaagtggtcccagtgatgtgggaacctcagagtctaaaacaaaaatagtagactgaggccctcagaaaccagacttatcagattcagaatttaatataactatgtttgaaagatttaaagaaaataaatcaaattaaaaagtgagcaagaagcaagaataacaaatgaactggcatatttgagaaagaattttgagcttttagaaataatacatacagttattgaagtaaaaactcaagtcaataggtttaaacagcaaaatgaatatagttaaagaaaactattgtgctgaaagatttaaaaaactattacacagaatgcaaaatgatgaaatagggtaatttaatatatggcagatatgttaagacactatgaggatagtatgagaagacctgatagacatctaatcacagcaccttaaggtgaaaatagagagagtgcaccaatattgaggagttaatgtctgagactattccagaactgataaatagatgaatctacagatctcagaaacacagcttttacaaaggataaatgaagagaaattcaccagagatacattgtaatgaatctgtaaatcaccaaagctaaagatattatttcacaagtggaatgacagctgacttctcaacaacaacgaaagcaaggagacagttgaaagacatcttgaaaatggtgagagaaaaactaactgttaatataaaattgtgtacctatcaaaaatatcttttggccaggcgtggtggctcacgcctataatcctagcactttgggaagccaaggaacgtggatcacttgaggtcaggagtttgagaccgtcctggccaacatggtgaaaccccatctctactaaaaatacaaaaaaaaaaaaaaatagccatgtgtggtggtgtgcacctgtaatcccagctacttgggaggctgaggcaggagaatcgcttgaacccaggaggtagaggttgcagtgagctgagatcatgctgctgcactccagtctgggtgacagagtgagactgcctcaaaaccaaaccaaaccaaaccaaaccaatctttcaagaagtacaccataaagacatttctggataaacagtgaatgagattacttcctaaacgaatatgtaaggatgtttctttttaaaattttttttgggtgtattcatcacctcaagcatttattcttttgtgttgttacaactgataagaatatctaaggatgtttcaaaaggaagaaaaatgatccctaaagtctggtatattagtttcttattagttgctctaagaaattactgcaaacttagtggctttaaaaaacacgaaattatctttcagttctgtaggtcagaaatttaacctcctgatttctggtcagaagtcttactgggctaaaatcagggtatcagcagggctgctggaggctctggagatgatgtttctctaatttctagaggcatttgcattccttagctagtggcttcttcccacatcttcaaagctaacagtggcagtcgagtccttctcttatcttttttttttggggggggttataaattttttttattattattatactttaagttttagggtacatgtgcacaacgtgcaggtttgttacatatgtatacatgtgccacattggtgtgctgcacccattaactcatcatttagcattaggtatatctcctaatgctatccgtcccccctcccccaccccacaacagtccccagtgtgcgatgttccccttcccttctcttatcttatcactctgacctcttctgcctcctttctctacttttaaggacccttgtgattagattgggtccacctaaataatccaggataatctctttattttaaggttagctgattagcaacctcaattccatctgtaaccttaatttcttttttgccatgtaacctaacatagtcacaggcccaagggaataataggatgtgggaattgcgggtggtggagtaggggcattattctgcttaccacaggggaccttggatgtcagtaaaaaatggtgaggaagaaaacaggaaacgtagaataatctaaacaaatctttgaaaaataagtcgaaatgtggggttagaaaaagggcagaactaaaatgttaagacaaattaaaatgttaagcctataagttgagagggagatgatcaaggttaaaaatactcataaatcatatttttttgggtagggggttagatatttattaattttagaccctgagaagtaaaatagtcatgacaaaaatttaatagtaaccactaaaacaagagaaacagagtatataacttctaaatcaatatgggaaggggaatggaataagaaaacaatagcaaccaccaacaccccacccaaaataaataataaggtgagagagaaaaagaagcattgaaaaggggctaaaataaaataaaaaattaagtcatgcccgtaatcccagcactttgagaggccaagatgggtggatcacctgaggtcaggagttcgagaccagcctgaccaacatggggaaaccccatctctactaaaaattcaaaattagctgggtgtgggggtgcatgcctgtaatcccagctactctggaggctgaggcaggagaatcgcttgaacccaggaggcagaggttgtggtgagcagagattgtgccattgcattccagcctgggcagcaagagcgaaacgccatctaaaaaaaaaaaaaaaaaaattaagatagtaaaacaaatccaaatacattaaagattacaaaaaatgaaaatgaactaaacttcctagttgaaagacatattgtcagattggattttaaaatctggcaatgatatttccaggacacagtcctaaaatattaaacacagaaaagttgaaagtaaagggatggaagaacacagtcaaactaactagaagagagctgggttcacccttttatatcagagcagaatttcactgagaaaacactaacaggaagggggaaaggcacaacatagtgatgaagcacttaattctcaacaaatgtatgacacttctaaactattatgactgcaaaaaaaaaaaaaacctcagcatatataaaattttggaagagccatggctatcagctgacatgcacttaccatatttgcattgtcctactacaaactttacccatatgaccaaggtgctgtcttcatccccattttacagatggggaaatttccccatagagaaaaagatttgggccatgccttggtttcttcttgactctgcttcctgctgattgatcctcacacctcacctggagacctacttcacattatccatcaggatgagaactggtgtgcctttaccaaaggaccacagtcaccttcaaaacgtgtttagatagatctggatggagacttatgtggttgcagatgaaaaaaaagtccccatggtatatctgactccatgtcattcactgcatgaacactcacacacatgcacacgtacacaggcaaacacaatcctgttctggtccggcagtagtgccaacactcaaatcccaggtcctactctgaagcctttgaggaaaccattgtcattctggggtttatgggaatatttcttcttggtgccccttgttcctcacagaagctttttttctgtgctgtgggcttgctatcctagctttccaagctatcctagttttgcaaatttctctgatcaatcacatcaggttcttggatcatcccaaaattaaagaggtacgttttgctgcaagagacaaagacattcacctctcacacagtttgaattcctgggtagaaacagatttcaatgctggccagcctctgtagctcccagtgccccctcaattatgactcccgaggcattggagcaggatcctcaccttgcgcaaagataattaggtgtgctgaagaaagccagacactgctctttcttagctctgcatccttaaaaagttaccactactccttctctttgaacctttgacttgtcaatggtaaagtgcctatgattcttaaagggataaaaacgcaacctatataaagcacctggcatagtgcctggcacaggtgggtgtttaacaaatgttaactcctttctatcccactgtgggtgtccatatcctcttgggtaggctgcctctggtttccattgtgtcatcagagccatactaaatcgatgtgttggacatggatcccaagaactaatttctggaatgagttatgcctaccaacttccctgatacttacttctgtctctccatttatcaaatggaaatgtttgcatttggcctctgccagttggagtaaccaaaaggcttagaggttttggagaggtcctacactgccatctgtttgaccacatactgcctttcacgtgtaataagcattgtttcttacatttgtctaggaattagcagttcacaaagcacattcgcatataagggcttgttttgaattgatcttggcagcaattctatgagacaagtaaaaggtaggtcaagcattataatcctcattttaaatctgggaaaactgaatctcaaaaaggttgaaagacttgtctaggggacagtgtgtgggtaaatgagaagttaagatttgctgaactggcatttcctgactacatatctagtgtttatttatggagaaggcactacggtggccaagtggctcagagtcacacagctctgtgcctcagtttatttgtctgtaaaatgaagataataatatattctgaatactgttgtggaatttcattgagatagttcacataatggcatggatactgtagtacaccgcctagattcacaaccccacgactatccatgagaatggcccatagctcaagaggtcacattctttccccaggtgaagcccgcatccaatgactgctcagtgtgggtatataaaggcctggctcccttgccagtgccaccctacctcctggcgaatggactgaggcttgcattatgactgcattgcagctcaactgttctttccattcagtctttcctttacactcacacaggtgaggacccagccattaaatgcctcacatgataacctctattgcatgctaggcttcgtgtgcacacacaaaaaccatcctctatcagtcaggtcccttacttgcagaggcaaagaggccagtctgggttagatgtgcaccctgtttttacaacagaacgagacaaacaggtcctccttaggtgtaagttcatggccttggccccacccctgaaactcagccatctgagacagttttaggcctgaggcaaccccagactttgggttgttgtttgctgggcacagcctccttttgccaatgttgcaatcctttgtaagagaccacaggttgatcacccgttcctgttactgagcacagagaggtttgtaggccagtctctccaggaattctgacgcactgcaaaatcccatggtctgaatgcttctaccttccctgtgaaagcccctggcctcagaaaaggaagctggtttaagtgaccaacatttggggcggagcttccgagggcccagccaccgtgttacatgcaacactcaacagagatattctttggctagagttgtttttctgggtataggaccctataattaaaatcagctctccaacttcccttcctccaaaaagtatacaatgtaaggaggaaaatgcaaattgaaaagttgctagtccttcccgccagaatgccacctcaagcctcccctgcaggaagttctggagtccccaactctgccccaagcctgaggcccttgataaggtaaagggatgtgagtgtggaggccggagcccccctcgccctgtaggctgctccccttgcttttccctttgaatgttacagtttagttctgtgacttacttataattgcctgaattccccgcccacgctttctcttactttggggcttctcaccgttcctcttcctccatcttcctcctctgaccccactcccaagcctgaatggggtgttcctcttccgtgctcccacaaaaccctgtgcctctttcatcgttgcatatattatgctgtaatgtagttttctggaggtgtgaagactatgaatcctatgaagacaaatacacacctaccttgctgaccgttgtatcctcagaatctaatactgcacttggcacacagtggttgggagtaaatattcatgaggcaaacacacaggaaggactcttttgttatggttgtttgttgttgttgcaagtaacataaccccaccttgatctagctgactgacggctctaccaccttgtcttttgcagcacaagaggaataggaactgcacctcttccttcagtttcagcttgaataatatcaggaagattcgtatcggtctgagttgggtcacgtacccgacgtgctatagctgaggatggggtaagctgattggagtttgcaacactgttcacatagccaagatatggaaagaacctaaatgtcaactggtggatgaatggataaagaaattgtggtatatacatacactggaatattattcaaccttaaaaagaaggaaatcctaacatttgtgacaacatggatggacctggagggaattatgctgagtgaaataagacagacacaaaaagacatttcttgcaggagctcacttatatgtggaatctaaaatagtcaagcttaaagaagagagtagactactggttgtcaggagcaggagaaaagtggaaatgaagaggtgatagttaaagggtacaaagtttcagttatacaagataaataagttctggaggtttactattaatatatcacatagtacctataagtaacaatactgtattgtatacttaaaattgctaagagggtatatcttatatgttcttaccaataataataataatggtaataattaaggggcaggaggacacttcaaaaggtgatggatatgtttatggccttgatggtggtgatggtttcatgagagtatacttatccccaaactaattgagatgtaaatattaaatatgtacagctttttgtatgtcaatcgtacctcagtaaagtagtttaaaaatggttggactgagaaaaggaggagctgctcagcaacatgaggctgggtgctgggcagacaaaacctcacacatgcattactgaacccacggactcatgtctgtgagctttgtggctgtggatgcaccatgccaatgtagtcaaggattcttcaatgtgtaccttactggattagttccctctgtttatttgtattgactcctcagttcccttatgggttgcttgttggctcttgtggaattatttaagtaaggctttggttgcaggagtcagacatgcttaagctggttttggctgtaatggggaatttgtgagatgtgacacagtgacaggaagtgcagccagacgttgtgagaggcagtaactgggaataggaaagttatgagaagttaaggcagtagtaattgtttctctatcgaaggccataatgttatcattcctgcctctatctgtttgcttgttcttctctctcagcagaatggtcttctctgcttctctgtgcacctgcagaaggtgaccaccctaaggcttatggattcaggagtcctcagtttgagagcagttaccaaatgcctcgcccctgtgaactctagtcccagtttcttattcctggtactggcttagtttgaggcagctgcccaccgttaagtccagtgagccatggcctggggacaaggtcaggtacatgtgtaatcagctgggtctgtgagttgtggtggtggcgatggaggtagctcccagaaaaatggattacaggttaagaagagtctatatatcttgaccacatttgatgtgacttggaagtttcaatgtgtgaccacttacaaagttatgatcaggtgtctcatttatttatggtgatgtatgagtccgtgtgtgagtgtgtgtgtgtctgtgtttgtgtgtgtgtgtgagagagagagagagaatgagaatatgagtggtggtttcccatttatttttctatgggccaaatcctgggctatgagctggagatataagagtaaacaaaatagacacggtgcttgctgtgacctgccctgggagacagatattacacggaggatcacataaatgcagagtctgagctgaggtcttaagggtgaggataagtaactagtcagtggagcaggggagggaagggtgttccatgcctgtgtcaaggtcctgaggtgagagaaagaggtgaatttgaggaaccaaaaggagttcagggaacaagaggggctgtttcacaagctgagtctggaagctgagcaggctgagcagattcttaggaactctgcaaaaacttaagggcttgtctatgaccgtaaacaactacggtcgtagataaacaagttggcaaacaactcaaggatttacagtcatcagaattgtaaaactgatcccatgtattttcaaacctcaaatcccttgctctgaccttgtgtgtctaagctagaaggaagcaggatgcacctctcccctggctggaatgaagggattcacccaagctctcagtcttctcacggcatccagggcccccctgcttgtgtgtggtctagatttccattcccatagtaggaattcctttgggagccttggggtctctcctgctagagggcttcacctgtgactgtctcaattcaagggaggggttctaataacattaaacctcaatatcttgctcctccattcctgatgcctccctttccttctacccttcccctacctccttctttcctatgccacaggctggcaggttagtgccagagcaagtggccagtcaccatcattggggttgtggatcctcagggttcttcagaagccccttcaccatgatcaagagtctccagtcactcagaattccacggttcccaaatgccagctctcccactactcccagcgttctccatctctgggatgttgggctccaggcttctcagatgcactgagtaccctaggctaagccactgatccatcagaacttcatagcctgagaggagagggagacaggcttgcaaaggagagttctgactagacactggggtgctacagatgcccgtaagttgtccctttaccctcgatgtccccagtgttgggaccacctgggaatgcccagccttgtgtgctgattgacttgtagtcccctggccctgactggaagcctagttttctctctcatgaccagctaggcccctagctccccagggaagaaatccaatctatttcctctgatagtaatggctaatacttaaataatgccaaccacatgccaagcactttacagttgttagcccagtcttcacaagcaccttgtgaggtgggcaagagtcttatcttactctcattttatagatgtggcccaggatcccacagctatagttcatggtgctgggatttgaacctctggccaccagagcccaccttaatgtgtcctcctcctgttgtcataacagaaaagtacaacaccatgatgacacatcaggctatcctggcaggttcccaggctgccccaatgcccaactttctaggtttacaaagttgacatttacgaagtttccaggtttacaaatctagtttctgattctttagtcagcaggaatttctctacaaaagctgcttcgaaaatttccagccaaaccttacacaccttggcattacatcttggtgagccaaggcggaagagaacaggaagtgaaggccccatgggaagtccctgcggtcgggagcacccaggcggggcggggggtggggggctttcctgtggccggctccctgcccctcccacccccattcaggccctgtgagttgaatgaagagaccctgggaatgagtccaggtctgcagggttagaggaaattgaaggcccttaccagatccctgttgagaagtttatgaattatgagcccttctgcaaatgagagggttcttccctgtcaggagggacagattgtaggtggcaagattggtggcagccagtaggctggtctgctccttcctctctatttcatatgtgtatgaaggcattacctgcagcaagggcctgtgtaaatgcatgtgatttacagagcattttatgtactgcgtgtcattcatgcttccggtgagccctaagtctaagatagggcagatagcatcaggtccattttgcagctgtcaaaatgaggtctgaagggcagaagtggtgtgcccacacacacacaactggttggctgcagacctggggactagacccgggacttcgtcctgcccaggggtctcttgccactgctccccatcaacttggatggctttaagcatttgtgagttgtctgctccctgatggcagaatgcagagacatgaagctacaagcaggttcgctcccaacggcaaaaaggaggaggggtgttcagaacatcaggtgcttctagagaaagcagggagagagtatctggccttgtggacaatgtcacggcagaggccaggtatagggcatgggggtaactggaagcgggatggaccctgttattccctaagacatggcttccacgtagtgctcaaacaaggcctttgcccttgctgttccctccacctggaatattcttccccttccttgacattgctcaggtctccactcttatgtcaccctctcagagagggcttccctggccactttccctaaaatagccacccactcctaggtccctcaaaagcatatcctgctttggattttccctatagcaatatgccctatgaagttattttatttgctaacttgtttcttgtctgttttcctttgttagagcgttggggaccttgtctggcttgttcccaatgcctggaagagtgcctggcacacaggattaagccaacacatatgttttgaatgaatgtgtgcacacatgcatgagctggcggcagtcggggttggggtaagcacgaaggcccagctcagttctctgcatgtgacctcccatcttacgcagataagaaccagtttggtttctgctagcctgagtcaccctcctggaaactgggcctgcttggcatcaagtcagccatcagccggcccatctcctcatgctggccaaccctctgtgagtgtgtgggaggggaggctgggctcctccttgtactctctgaggtgctctggaaggaggggcagctccaccctgggagggactgtggcccaggtactgcccgggtgctactttatgggcagcagctcagttgagttagagtctggaagacctcagaagacctcctgtcctatgaggccctccccatggctttaggtaagctccttccactctcattttttcacctgagaaatgagagaggaaaatgtctacaattggtgtttatcaaatgctttcaggctctggtgagcaagcgtccaggaaaatgtcaagcgcatggagctccaggcctgtctgggggatctgggcacggggaggcatccatgggagaccatgcaggcactctgaggcaggggctgcaagcctagtgcctgctggggcagcaggtgaacagagaggtgtaactgctgtgacagaagtcatggagtccttggagtgtgagggtcattttccactgttgatagaatagggaaattggtgaaatagccctgttaaatgagagaaagaacagtgtgagctcaatgagaaatactaatagaatgtggcactgagccacaaggtctgagggttgattgataaggaagggtggggactgtggagaattaagggcttggcacagtcagttccaccagttgtcacaagagaatgcaggctcaggtggccagaacttctcgcttttccagaagagtccgatattctgatttcattatatatagtattctgattaaaccagacaataaagcaagcagataaaatatttaaattataagctgccagtttgcaacctccggttaggatttgtgtggggcaaagaaaaaaactctcaggatcattggtatgtagactctaattttaagtttctaatttaaaattggcccctgaggctgggcgtggtggctcacacctgtaatcccagcattttgggaggccaaggtgggtggatctcttgaggtcaagagttcaaggcctgcctggccaacatggtgaaaccctgtctctattaaaaatacaaaaattagctgggcatggtggtgcatgtctgcaatcttagctacttgggtagctaaggcaggagaattgctggaacccgggaggtagaggttgcagtgaatggagatcacaccactgcactccagtctgggcaatagagagagacgctctctctaaaaaaaaatatgtaaagataaataaaatgaaataaaataggcctctaatgagcaggccattctcctttctgggtcttactttccttgcactcctttctgggtgttaagaggaggtctagaggaagctggacaactcttagcttgtagtaagcacagtggaagtgtcagctcttaatgggtcatggacacgttacaagctaggcgccttgctgagcactttacatggtttatcccactgaaccctctcaataaccctatgaggaagggctattattgctcacattttcagaagaggaaatggatatagagagattagataatttgcccatggccagacagctagtataagaggaggaggtggattgactgcagacattctgtcttcaaaccactacactatgctatgggggcacagagacttaatgaaatcatggagaggggaattgctttgtcaaccacaagcagttattccgggggcagcagatcctcccctgtcccccagtgggtacaatggtccctggtgggttgtgctacaatgttagcccatggtcttatgtgtttttcaaatgtgtaaagtaggatgctggaaccactcttagaaccagataccaatacattgtgaagaaataaatctctgtgcttaaaactggttcatcccaaaatattttgaactgacacacaataggtgctaaataaatgtgtgttaacttgaattggattgaattcgggaaaaaagtgcaataagcttagtgaagacaccatgttccctgggtagaggaaccacattctccatctaaggccaggagtatgggaggtatcaatgtttgcccagcacagaacagggtgccaagaagagaaaagttgacggggtgcatactcggactggaaactggaagggtgagaacagagggtaaaggatagagatggaaccatgtgcatacactttgtgttaccttggacaagtcattcatttctctggacctctgctttctctctacacaatggggtcccaccacttcccttacagctgacttgtatgaagaaggaggtggaggaggaggagaaggtgaagacaatgctgactcaaagggtaaattatttttaggatccaagtttgaaaacaattttaggctactagatatgaacaacatcttgattatgtagttgaaggaaattaaagatgaatggtttaattaaaaattaatcagaatgaaaacgattgattactaatatatctgcaatggtttattttcctgagtggcagactcactaaggtttttgaatactcctgtgtgattgctctatgtatgtatgtatgtatgtatgtatgcatgtatctatctatctgttgtctaataaaatggatcacatctctgctaataaaaacactacactggcagggtacaattataatcattaactgtgcctggaatttgcagcagcagccaccagaggtaccagtgccctttaagggttcataatttagaataatccaattatctgagtttttcagggactgaggggtttggcaaggtgtagaactttcagtaataaagtcaagaaagtcctggacaaaccaaggtagttggtcactctagtccataaccaggtaaagagctttccctgtaacctgtgtaaggttttagaatcatttctttccttattaccaaaaatcctccccaaattttcaagaaattatgaactaaatagttactctatgagataggagttcagcccaaaagaaacaccataagaacaaatataattcttgcttatgttaaccatgcaatgaagcagagagaaaaagtcagtggcctctttaggaggactgtagtgtgggaagaaataactaaactgggtttcaatcctggcctggccaggatctggagcaagtgagttaatctttctaagccttgagtagtttcttcttcttcttcttcttcttcctcccccttctcctcttcttcttcctcctccttctcctcttcttcttcttcttcttcttcctcttcttcttcttcctcctcctcctcctcctcttcttcttcttcttcttcctcttcctcttcctcttcttcctcttcctcttattcttcttcatcgtcttcgtcttcgtcttctttttattttcaaagtgaaagcaagtttattaagaaagtaaaggaataaaagaatggccactccatagacagagtagcctgaaccttgagttcttctataaagtcactatgaatttatactcattttgaaagtgggtgtcaatatgtctgtccactttgcacagctgttatgtggacaaaaggagatctgtgtgaaagtgtaacacagagcctaaactataacaggtaagcaacacagttgtccccttccccatggtgtctgttcttctccatttcctcctgtctgcagggggattataaaactaatcatcaaagccaagaaggcaagagcaagcatgtaccgctgaaaacacaagataactgcataagtaatgactttcagtgcagattcatagctaacccataaactgctggggcaaaaatcatcttggaaggctctgaacctcagaaaggattcacagtaagttaaccatgtagatctgagaggagagtagcttcttgtagataacagttggattatataccatgtcctgatccccttcatcatccaggagagcagaggtggtcaccctgatagcagcaagcctgggggctgcagcttggtgggtagaggtactcaggggtacagatgtctccaaacctgtcctgctgccttagggagcttctaataagttgatggatttggttaaaattaacttggctacttggcaggactgggtcagtgaggaccaacaaaaagaagacatcagattataccctgggggtttgtatttcttgtgtttctttctcttctttgtactaaaatatttacccatgactgggaaagagcaactggagtctttgtagcattatcttagcaaaaatttacaaagtttggaaaacaatattgcccatattgtgtggtgtgtcctgtgacactcaggattcaagtgttggccgaagccactaaatgtgagatgaagccattacaaggcagtgtgcacatctgtccacccaagctggatgccaacatttcacaaatagtgcttgcgtgacacaaatgcagttccaggaggcccaaatgaaaatgtttgtactgaaatttgttaaagcttcccgacaaactagatttatcagtaaggattgttttctgcaagggggatgaaacttgtggggtgagccatttgggctgaggaggagggaggttggagctgagaaatgtggagacaatttccctttagaaggactgaatctccctgcctctctggggtgcggcagccagcaggatccaatggtgtatatgtctccccagctccccattcagtgatatcatgtcagtagcttgaaattatccgtggtgggagtattatgtcatggaaattggcaaatggaaacttttattggagattcaattgttaaacttttaccagcacaacactgccctgccttcagagtcaatgaccctatccaagtttaatccatctgtccactgtctccaacacgatctttataaaacacacctgacaacattacccttttattcagttttttaaaagataagtttccagctcatcgggctggctttaaaggccatttctcctctggacctcacccaacttttcaaatcacttttcctacccctacctctaaatgctactcaaactccagccatcctgaataataagacttttgaaaagtagattatgggctgggcacagtggctcacacctgtaatcccagcactttgggaggccaagatgggtggatcacctgaggtcgggagttcgagaccagcctgactaacatagtgaaaccctgtctctactaaaaatacaaaattagttgggggtggtggcacaagcctgtaatcccagctactcaggaggttgaggcaggggaattgcttgaacctgggaggcggaggttgcggtgagcctagattgctccactgcactccagcctgggcaacaagagcgaaactccatctcaaaaaaataaataaataaataaagtagattacatcagatacctctggcctaggttgtttatgaccaactctcctgctgagaataactagaaaagctagacaaaacatatttccaaaagatctctttggaggcatcagagaatggccaaggctgtaaggaactgcctgagcccagagaggtggagcccagcactggtgccctttactcctggggacatgtgctggtttcaaaaacttcagctgagtttttgagcattcatggaacttggtgggggagatgaaatttgtaccttaaatcctgcctacagggagggtccctgataatccccacccaatttggaaatctgggtcagccttcacaggtactgaagccctcctctgaatgatctcaagtcctgctagggtagaggttacctgcttttgaaaggctcctggcctacctgtgcagcaggagcaaaagtgaaccatctcagggtacagataacaatcatccagagccttgaatgacctctactgtgcttaatatatagtattcagcagtcagtaaaaaggatttaggcacatgcaagatgacctgtgtatcagggagaaataggcaataaattgagatccagcagggatttgaatcatggatttgaatcaggggcagccttcgaaagaactgtggagaatatactcagatttaaaacataagattggaatttttggcagagaactaacaactgtacaaaaaaggaaccaaatggaaatcctagaactgaaagatgcaattaaccgatgttgagaaatagccaacatctattgaacacttcccatgtggacagctgtgctaaacactttacaggcatcaacataagatgtgtccccttacagcagtgcagtgtccctcctaagacatggacagcctggtttccctatctctctgcttcatcaaaacccctttacgtggggcttagacactcctgttgtctctagtgtctagtagcacagggctcagcacatggaagccactagatacaatttgatgaccaggacctccgatgaaagccatgggtgctgattgggaaggcattgtcttttatgtgctatggtcttaaagcttcatccaggaagcagaactcggggggtgctgaggacccagaaccgagaataagattagtcagagatttcctgtgggcagaaatcataaggacgccaactgtttgggtgagataagacgaaaccaagagtggacttgtggccagaagcgtgaggaagagggagagagcttcccttgtcccctttcttcctctccctaagccacagtgattgacagccccccccctttggagtcagagcaggcttgagactggactgggaaaggagggtgggtcaggatacagagcaggaaggctgggagtgcagggcaggagcaaggggctggggcattcattgtgcctgatctctcccactttacctggggtaaagaagcatatgcaaaagccacggtgtgagtatttcccaagtgccagggtcagggcatgattcatcacgtgcagcatttcattcaatccttatagtaaccgatgatgtggcttctattattagctctatcagataatgaaactgagaccaagacaggctctgcacattgtgtggggtaatgacacagggggattcagacctagactccataactcctgccccagggaccacccccaccctcaccctgtgcatgtcgacaaaggacagactgggccacttctcaggacacagcggggaaatgacacagagcagggaggttccaggagccccgagcgtcttttctccaggagaatactctctgaattcagactggggtcagagaaacatttacccaggagccgcagtgtgggtggggctttttacttgaaacgctgtctgaaggcagtggccaggatggaactctccaccctaccttggcaagccacttctcttctgcaatctgtaaggacattgttgagagaattatggtcttccaattccggagggttgaagaaagacaaataggagagaacctatcatagtcaggtgctagctgccttctctttcagagagtgtgagaataaagtgatacacttgattattagcaaatactttggaaattttaaacgctaatattcaacacactctggaagaggcaaataagtagacaggttcatatacatcatctccttcagctagtcctcacaaaaacaaacaaatgaataaacaaaattcttctttggccctcataggaagacactgtttcttgaacgtgtttcaaaaaggatgggtgactcactcaaggtcacactgtttatgaggacagtacaggaatacagacatgccattttgcctgaaaaaatccatcacccagggaggtgacacaattttgcagaaatgttctatttcctctgaaggatacattctttaaacctttgggaaattcattcatagtcttcctcctttgaaggattaactctctggacacaaagtgtttgattctgatttgttggttggaagatgtgttggttgagagaaagattctgatttgttggttgaaaatagactcatcaagatcaactgctgtagtagtaaatattttgacattttgtctgtattcctgtgctgccctcacaagctgcatcaccttgagtgagtcattcatacttttttgtttgtttttgttttggagatggagtcttactctgttgcctaggctggagtgcggtggcgtgatcttggctcactgcgacctccatctcctgggttcaagtgatcctcctgcctcagcctcccgagtagctgggattacaggcacatgccaccatccctgctaatttttgcattttcagtagagacggagtttcaccatgttggtcaggttggtcttgaactcctgacctcaggtgatccgcccacctcagcctccccaagtgctgggattacaggtgtgagccaccgtgcccagcccagccatcatttttgaaacacgtttgagaaatagtgtcttcctttgagggccaaggagacattttttttgtttatttgtttgtttttgtgaggactagctgaagggggtgatgtatattaacctgcctacttatttgcctcttcccagagtgtgatgaatattagggtttaaagtttctgaagcatttgttaataaagcccggggctggaggtcagaagacctggatttctctgcatacttttgccatcagcaagctgtgtgaccttggacagatcccttttttgtctaaatctttctgagtcttcttgaaaacaatgccaggttgggacaggatgattgccaagctcccgtccagctctaaaacactgcaacgtatgcttctgcaccagcactgtccatcctgtagatcatgcagaaattctcttcaactttttcctacccataaaataggagcatgcttacctttttcctaatgttccaggccccgggtctagaatattgtaagtaaggaagttaatgtgtatcagagcccattatgggccagaagttctcctcttccttcctacacctgcttcctccctccctccctccctctttcccttccttccttccatccatttgtgaagaagacatgatcaccctcattctgagagtgaagagacagaggctcaactaatgaaatgatttgttcaaggtcacacgggtggcacaaggcaagtggcagaggttgaatttagacccattcctgtccaaatgctgagtttatgtcatcgtcccgagaccataactttaaagatgtaagatagtgggaaaagagttgatttcaaagcacctctcagaaggactcactttacatcaggggtcagcagactcaggccaaatccggtccattccccgcttttgcaaagaaagttgtagtggaacacagctaggcttattgatttatggattgccaacgtccttttgtgaaacagacagctgagctgagtaatcgtggcgcacaaaacctaaaatatttactatctcgtcctttacagaatgtttgccaatctatggtccggagtccaaggctgtccatttttcaaagaacacaaagtgacatgagactgtcccatgtgcagggagccctatcattttattatgaaaaaacggcctttctgctcaaatctgttttttaaaaagtcaacaaacagactctgggtacctgtcaggaacagtagggagtttggtttccattgtgctcttcttcccaggaactcaatgaaggggaaatagaaatcttaattttggggaaattgcacaggggaaaaaggggagggaatcagttacaacactccattgcgacacttagtggggttgaaagtgacaacagcaagggtttctctttttggaaatgcgaggagggtatttccgcttctcgcagtggggcagggtggcagacgcctagcttgggtgagtgactatttctttataaaccacaactctgggcccgcaatggcagtccactgccttgctgcagtcacagaatggaaatctgcagaggcctccgcagtcacctaatcactctcctcctcttcctgttccattcagagacgatctgccgaccctctgggagaaaatccagcaagatgcaagccttcaggtaaggctaccccaaggaggagaaggtgagggtggatcagctggagactggaaacatatcacagctgccaggggctgccaggccccagagggcctgagaactgggtttgggctggagaggatgtccattattcaagaaagaggctgttacatgcatgggcttcaggacttgtgtttcaaaatatcccagatgtggatagtgcgaccggagggctgtcttactttcccagagactcaggaacccagtgagtaatagatgcatgccaaggagtgggactgcgattcaggcctagttgaatgtgctgacagagaagcagagaggggcaccaggggcacagcccgaaggcccagactgatatgggcaaggcctgtctgtgctgacatgtcggagggtcccactctccagggaccttggtttccccgtctgtgacatctgtgacatgagagtcacgataactccttgtgtgccttacagggttgttgtgaaaattaaatgcacagataatagcgtaacagtattccgtgcattgtaaagagcctgaaaaccattatgatttgaaaatggaatcggctttgtgagaccatcactattgtaaagatgtgatgctgatagaaatgacaggactgcttgtgcatgccctctgcagtgtgacattccagcagtgaaatcatgttggggtgacttctcccccactctgacctttatgtttgtctgggccgaggctgcaagtcgggctctgtgggtgtatgagtgacaagtctctcccttccagatatggggactgtctgcttccctaggttgcctctccctgctctgatcagctagaagctccaggagatcctcctggaggccccagcaggtgatgtttatccctccagactgaggctaaatctagaaactaggataatcacaaacaggccaatgctgccatatgcaaagcactttggtttgcctggccacccctcgtcgagcatgtgggctcttcagagccacctgatgaggtgggtacagttagccacacttcacaggtgaagaggtgaggcacaggtcccaggtcaggctggccagagctctgtttattacgtctcacagctttgagtcctgctctcaaccagagaggccctttaccaagaagaaaggattgggacccagaatcaggtcactggctgaggtagagaggaagccgggttgttcccaagggtagctgctcctgcaggactctgagcaggtcaccagctaatggaggaaaggctctagggaaagacccttctggtctcagactcagagcgagttagctgcaaggtgttccgtctcttgaaacttctacctaggtgctatggtagccactagtctcaggtggctatttaaatttatacttaaatgaatgaaaatagaagaaaatttaaaatccagacccttggtcacactatccacatttaaagaggtcaatagccacatgtggttagtggccaccctattgggcagtgcagctacagaacatttttgcatcccagaaagttcttttggatgttgctgctctacagcatgctttgctgaaacagaagtgccttccctgggaatctcagatgggaagcaagtaaggaggggagtcaaatgtgggctcactgctcaccagctgtgagggttgggcctgcctcttaaccattgtcagcctcagtcttctcatccatgcatgccgtgggtatactaaaatactatacccctggaagagctggatgcaaatttgacaagttctgggggacacaggaaggtgccaagcacaaggctgggcacatggtggctgtgcactacagctgagtccttttccttttcagaatctgggatgttaaccagaagaccttctatctgaggaacaaccaactagttgctggatacttgcaaggaccaaatgtcaatttagaaggtgagtggttgccaggaaagccaatgtatgtgggcatcacgtcactttgcccgtctgtctgcagcagcatggcctgcctgcacaaaccctaggtgcaatgtcctaatccttgttgggtctttgtattcaagtttgaagctgggagggcctggctactgaagggcacatatgagggcagcctgaagagggtgtggagaggtagagtctaggtcagaggtcagtgcctataggcacagtggtcccagggccacagctgggaagggcaaataccagaaggcaaggttgaccattcccttcctcaagtgcctattaaggctccatgttcctatgttgttcaaaccctaactcaatcccaaattaatccaccatgtataaggttgagctatgtctcttattcctggacaccatactcagccatattctggtccacacattaaacaagctggatgaccttgaagaagcttcacccactctgttcctcagctttcccttcagtgggatgatatcaactggacaacaggatgtgcgattcttttagttccagccttccaggatgttttcactcccctgtttgttgttgtaggatggtattacctccaccttcccaccttccctatgccctggttctgtctcctgtgcctcgctctgaaagtggatgagacctacaattcctgtcctggtagttctcctaatgaacacactgaagcacgaggaagctgagatttttgttgctacatgagagcatggaggcctcttagggagagaggaggttcagagactcctaggctcctgtggagccccactcatggccttgttcattttccctgcccctcagcaacactcctattgacctggagcacaggtatcctggggaaagtgagggaaatatggacatcacatggaacaacatccaggagactcaggcctctaggagtaactgggtagtgtgcatcctggggaaagtgagggaaatatggacatcacatggaacaacatccaggagactcaggcctctaggagtaactgggtagtgtgcatcctggggaaagtgagggaaatatggacatcacatggaacaacatccaggagactcaggcctctaggagtaactgggtagtgtgcatcctggggaaagtgagggaaatatggacatcacatggaacaacatccaggagactcaggcctctaggagtaactgggtagtgtgcttggtttaatcttctatttacctgcagaccaggaagatgagacctctctgcccttctgacctcgggattttagttttgtggggaccaggggagatagaaaaatacccggggtctcttcattattgctgcttcctcttctattaacctgaccctcccctctgttcttccccagaaaagatagatgtggtacccattgagcctcatgctctgttcttgggaatccatggagggaagatgtgcctgtcctgtgtcaagtctggtgatgagaccagactccagctggaggtaaaaacatgctttggatctcaaatcaccccaaaacccagtggcttgaaacaaccaaaattttttcttatgattctgtgggttgaccaggattagctgggtagttctgttccatgtggtggaacatgctggggtcactttggaagctgcattcagcagagtgcctggcttgcgctgggcatccaaggtggtccctcatcctccaggctctctttccatgtgatctctcagtgtttaagagttagttggagcttccttacagcatggcggctgacttccaaaagggattattccaaaaagagcctcaacatgcaggcgcttattatgacttctgcttgcatcatcctattggccaaagccagtcacgtggctaagtctagccccctgtgagaggagactgcataagagtgtgaacaccaggagacacggtcactgggggccaccactgtaaccatctaccacaggacctgaatctctgtgtgctactcccttgctcaagggcccccctacccacgcagacctgctgtcttctagcaaagcccatcctcaggacctttctcttccaatccttattgactcaaattgattagttggtgctccacccagagccctgtgctcctttatctcatgtaatgttaatgggtttcccagccctgggaaaacatggctttgtctcaggggcttgctggatgcaagcttaacctcaatgtgagtggccatactgtggcactgtcccatccctcaccagggacactgttctggagggtgactgcctgttctgtgaggagtggggatggctaggacattgcatggaacacaccaccaccccatcttctcagagctcaaaccctgacagaacaccagctccacaggccttggcttctgctgatggtgccgtgtatttaccagacttagtggtccaaggccagagtggccagatttcccaaagtcaaggtgtgacagtgggacagcctctttgtgtctttgctgtcctaagaaacctgggccaggccaggcgcagtggctcacgcctgtaatcccagcactttgagaagccaaggtgggcagatcacgaggtcaggagtttgagaccagcctggccaacatggtgaaaccctgtctctattaaaaatagaaaacattagacaggtgtggtggtgcatgcctgtaatcccagctactcaggaggctgaggcaggagaatcgcttgaacccaggaggtggaggttgcagtgagccgagattgtgccactgcactccagcctaggcgacagagcaagactccgtctcgggaaaattaattaataaataaataaacctaggtcccagagtcccacagaatggcagacaggagcacctgggggcttttagggtatggcatttcccctgtactaactctgggctgtccagagggccatttcatggcgtggagtggagagggaggcagcacaggacttcctaggcctcagctctcacctgcccatcttttgatttccaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaacaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatccatcaggccacttgatgacccccaaccaagtggctcccacaccctgttttacaaaaaagaaaagaccagtccatgagggaggtttttaagggtttgtggaaaatgaaaattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctcccccaccgggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgcctatattgttgactatgtccttggccttgacaggctttgggtatagagtgctgaggaaactgaaagaccaatgtgtctttcttaccccagaggctggcgcctggcctcttctctgagagttcttttcttccttcagcctcactctccctggataacatgagagcaaatctctctgcaaaaaagatatggggcagcactgtccacaacagcctctgctggaaacaacccaagcacccatcacagaatgaattagtacatcatgtatctgcacacaacacagtgctccttggcaaagaaaatgaatgaattacagccagctgcacctcacaagacagataatcttacaaacatcatgtgggtacaagaatcacaaaagcatgcatagagtgtgattccatttctacaaagttcaaaacgggcaacatggggctgttgtgtggagggctgtggaaacaggtggtgcagagagaagcaaggccatgctctgtctgaaggcccagtgacacttggcccctgcgagagagagtggcaggcaggagcacctgggggattttagggtccagacaacattctatttcttggcatggatgcagttaggcggttttctgtatgtatattttatttcacaatacctagaaaataaaaagacttgccccccaacaaccagggtgtaaagaaaggagcatctgggaaattatgagattagtgaatggagtattgaatttgtccccataggtgggagtgcaagaccatgagtgaccacttatcaaacacacctcagtaaacagtcaggagagtgaggagtgtggtcctttcacctgcccaggttgtttctgttagattctgggcttggtctctggagtccaagaggcaggccccagtgaggtcatgcacttgctgtctgattttggccaagtcacctccctttgtggggactgttcctcatttgtttgaggggaatgttgatttggatggggattttccaaa

SEQ ID NO: 114 - DNA, 23124 bp, Homo sapiens
aacacacaggaaggactcttttgttatggttgtttgttgttgttgcaagtaacataaccccaccttgatctagctgactgacggctctaccaccttgtcttttgcagcacaagaggaataggaactgcacctcttccttcagtttcagcttgaataatatcaggaagattcgtatcggtctgagttgggtcacgtacccgacgtgctatagctgaggatggggtaagctgattggagtttgcaacactgttcacatagccaagatatggaaagaacctaaatgtcaactggtggatgaatggataaagaaattgtggtatatacatacactggaatattattcaaccttaaaaagaaggaaatcctaacatttgtgacaacatggatggacctggagggaattatgctgagtgaaataagacagacacaaaaagacatttcttgcaggagctcacttatatgtggaatctaaaatagtcaagcttaaagaagagagtagactactggttgtcaggagcaggagaaaagtggaaatgaagaggtgatagttaaagggtacaaagtttcagttatacaagataaataagttctggaggtttactattaatatatcacatagtacctataagtaacaatactgtattgtatacttaaaattgctaagagggtatatcttatatgttcttaccaataataataataatggtaataattaaggggcaggaggacacttcaaaaggtgatggatatgtttatggccttgatggtggtgatggtttcatgagagtatacttatccccaaactaattgagatgtaaatattaaatatgtacagctttttgtatgtcaatcgtacctcagtaaagtagtttaaaaatggttggactgagaaaaggaggagctgctcagcaacatgaggctgggtgctgggcagacaaaacctcacacatgcattactgaacccacggactcatgtctgtgagctttgtggctgtggatgcaccatgccaatgtagtcaaggattcttcaatgtgtaccttactggattagttccctctgtttatttgtattgactcctcagttcccttatgggttgcttgttggctcttgtggaattatttaagtaaggctttggttgcaggagtcagacatgcttaagctggttttggctgtaatggggaatttgtgagatgtgacacagtgacaggaagtgcagccagacgttgtgagaggcagtaactgggaataggaaagttatgagaagttaaggcagtagtaattgtttctctatcgaaggccataatgttatcattcctgcctctatctgtttgcttgttcttctctctcagcagaatggtcttctctgcttctctgtgcacctgcagaaggtgaccaccctaaggcttatggattcaggagtcctcagtttgagagcagttaccaaatgcctcgcccctgtgaactctagtcccagtttcttattcctggtactggcttagtttgaggcagctgcccaccgttaagtccagtgagccatggcctggggacaaggtcaggtacatgtgtaatcagctgggtctgtgagttgtggtggtggcgatggaggtagctcccagaaaaatggattacaggttaagaagagtctatatatcttgaccacatttgatgtgacttggaagtttcaatgtgtgaccacttacaaagttatgatcaggtgtctcatttatttatggtgatgtatgagtccgtgtgtgagtgtgtgtgtgtctgtgtttgtgtgtgtgtgtgagagagagagagagaatgagaatatgagtggtggtttcccatttatttttctatgggccaaatcctgggctatgagctggagatataagagtaaacaaaatagacacggtgcttgctgtgacctgccctgggagacagatattacacggaggatcacataaatgcagagtctgagctgaggtcttaagggtgaggataagtaactagtcagtggagcaggggagggaagggtgttccatgcctgtgtcaaggtcctgaggtgagagaaagaggtgaatttgaggaaccaaaaggagttcagggaacaagaggggctgtttcacaagctgagtctggaagctgagcaggctgagcagattcttaggaactctgcaaaaacttaagggcttgtctatgaccgtaaacaactacggtcgtagataaacaagttggcaaacaactcaaggatttacagtcatcagaattgtaaaactgatcccatgtattttcaaacctcaaatcccttgctctgaccttgtgtgtctaagctagaaggaagcaggatgcacctctcccctggctggaatgaagggattcacccaagctctcagtcttctcacggcatccagggcccccctgcttgtgtgtggtctagatttccattcccatagtaggaattcctttgggagccttggggtctctcctgctagagggcttcacctgtgactgtctcaattcaagggaggggttctaataacattaaacctcaatatcttgctcctccattcctgatgcctccctttccttctacccttcccctacctccttctttcctatgccacaggctggcaggttagtgccagagcaagtggccagtcaccatcattggggttgtggatcctcagggttcttcagaagccccttcaccatgatcaagagtctccagtcactcagaattccacggttcccaaatgccagctctcccactactcccagcgttctccatctctgggatgttgggctccaggcttctcagatgcactgagtaccctaggctaagccactgatccatcagaacttcatagcctgagaggagagggagacaggcttgcaaaggagagttctgactagacactggggtgctacagatgcccgtaagttgtccctttaccctcgatgtccccagtgttgggaccacctgggaatgcccagccttgtgtgctgattgacttgtagtcccctggccctgactggaagcctagttttctctctcatgaccagctaggcccctagctccccagggaagaaatccaatctatttcctctgatagtaatggctaatacttaaataatgccaaccacatgccaagcactttacagttgttagcccagtcttcacaagcaccttgtgaggtgggcaagagtcttatcttactctcattttatagatgtggcccaggatcccacagctatagttcatggtgctgggatttgaacctctggccaccagagcccaccttaatgtgtcctcctcctgttgtcataacagaaaagtacaacaccatgatgacacatcaggctatcctggcaggttcccaggctgccccaatgcccaactttctaggtttacaaagttgacatttacgaagtttccaggtttacaaatctagtttctgattctttagtcagcaggaatttctctacaaaagctgcttcgaaaatttccagccaaaccttacacaccttggcattacatcttggtgagccaaggcggaagagaacaggaagtgaaggccccatgggaagtccctgcggtcgggagcacccaggcggggcggggggtggggggctttcctgtggccggctccctgcccctcccacccccattcaggccctgtgagttgaatgaagagaccctgggaatgagtccaggtctgcagggttagaggaaattgaaggcccttaccagatccctgttgagaagtttatgaattatgagcccttctgcaaatgagagggttcttccctgtcaggagggacagattgtaggtggcaagattggtggcagccagtaggctggtctgctccttcctctctatttcatatgtgtatgaaggcattacctgcagcaagggcctgtgtaaatgcatgtgatttacagagcattttatgtactgcgtgtcattcatgcttccggtgagccctaagtctaagatagggcagatagcatcaggtccattttgcagctgtcaaaatgaggtctgaagggcagaagtggtgtgcccacacacacacaactggttggctgcagacctggggactagacccgggacttcgtcctgcccaggggtctcttgccactgctccccatcaacttggatggctttaagcatttgtgagttgtctgctccctgatggcagaatgcagagacatgaagctacaagcaggttcgctcccaacggcaaaaaggaggaggggtgttcagaacatcaggtgcttctagagaaagcagggagagagtatctggccttgtggacaatgtcacggcagaggccaggtatagggcatgggggtaactggaagcgggatggaccctgttattccctaagacatggcttccacgtagtgctcaaacaaggcctttgcccttgctgttccctccacctggaatattcttccccttccttgacattgctcaggtctccactcttatgtcaccctctcagagagggcttccctggccactttccctaaaatagccacccactcctaggtccctcaaaagcatatcctgctttggattttccctatagcaatatgccctatgaagttattttatttgctaacttgtttcttgtctgttttcctttgttagagcgttggggaccttgtctggcttgttcccaatgcctggaagagtgcctggcacacaggattaagccaacacatatgttttgaatgaatgtgtgcacacatgcatgagctggcggcagtcggggttggggtaagcacgaaggcccagctcagttctctgcatgtgacctcccatcttacgcagataagaaccagtttggtttctgctagcctgagtcaccctcctggaaactgggcctgcttggcatcaagtcagccatcagccggcccatctcctcatgctggccaaccctctgtgagtgtgtgggaggggaggctgggctcctccttgtactctctgaggtgctctggaaggaggggcagctccaccctgggagggactgtggcccaggtactgcccgggtgctactttatgggcagcagctcagttgagttagagtctggaagacctcagaagacctcctgtcctatgaggccctccccatggctttaggtaagctccttccactctcattttttcacctgagaaatgagagaggaaaatgtctacaattggtgtttatcaaatgctttcaggctctggtgagcaagcgtccaggaaaatgtcaagcgcatggagctccaggcctgtctgggggatctgggcacggggaggcatccatgggagaccatgcaggcactctgaggcaggggctgcaagcctagtgcctgctggggcagcaggtgaacagagaggtgtaactgctgtgacagaagtcatggagtccttggagtgtgagggtcattttccactgttgatagaatagggaaattggtgaaatagccctgttaaatgagagaaagaacagtgtgagctcaatgagaaatactaatagaatgtggcactgagccacaaggtctgagggttgattgataaggaagggtggggactgtggagaattaagggcttggcacagtcagttccaccagttgtcacaagagaatgcaggctcaggtggccagaacttctcgcttttccagaagagtccgatattctgatttcattatatatagtattctgattaaaccagacaataaagcaagcagataaaatatttaaattataagctgccagtttgcaacctccggttaggatttgtgtggggcaaagaaaaaaactctcaggatcattggtatgtagactctaattttaagtttctaatttaaaattggcccctgaggctgggcgtggtggctcacacctgtaatcccagcattttgggaggccaaggtgggtggatctcttgaggtcaagagttcaaggcctgcctggccaacatggtgaaaccctgtctctattaaaaatacaaaaattagctgggcatggtggtgcatgtctgcaatcttagctacttgggtagctaaggcaggagaattgctggaacccgggaggtagaggttgcagtgaatggagatcacaccactgcactccagtctgggcaatagagagagacgctctctctaaaaaaaaatatgtaaagataaataaaatgaaataaaataggcctctaatgagcaggccattctcctttctgggtcttactttccttgcactcctttctgggtgttaagaggaggtctagaggaagctggacaactcttagcttgtagtaagcacagtggaagtgtcagctcttaatgggtcatggacacgttacaagctaggcgccttgctgagcactttacatggtttatcccactgaaccctctcaataaccctatgaggaagggctattattgctcacattttcagaagaggaaatggatatagagagattagataatttgcccatggccagacagctagtataagaggaggaggtggattgactgcagacattctgtcttcaaaccactacactatgctatgggggcacagagacttaatgaaatcatggagaggggaattgctttgtcaaccacaagcagttattccgggggcagcagatcctcccctgtcccccagtgggtacaatggtccctggtgggttgtgctacaatgttagcccatggtcttatgtgtttttcaaatgtgtaaagtaggatgctggaaccactcttagaaccagataccaatacattgtgaagaaataaatctctgtgcttaaaactggttcatcccaaaatattttgaactgacacacaataggtgctaaataaatgtgtgttaacttgaattggattgaattcgggaaaaaagtgcaataagcttagtgaagacaccatgttccctgggtagaggaaccacattctccatctaaggccaggagtatgggaggtatcaatgtttgcccagcacagaacagggtgccaagaagagaaaagttgacggggtgcatactcggactggaaactggaagggtgagaacagagggtaaaggatagagatggaaccatgtgcatacactttgtgttaccttggacaagtcattcatttctctggacctctgctttctctctacacaatggggtcccaccacttcccttacagctgacttgtatgaagaaggaggtggaggaggaggagaaggtgaagacaatgctgactcaaagggtaaattatttttaggatccaagtttgaaaacaattttaggctactagatatgaacaacatcttgattatgtagttgaaggaaattaaagatgaatggtttaattaaaaattaatcagaatgaaaacgattgattactaatatatctgcaatggtttattttcctgagtggcagactcactaaggtttttgaatactcctgtgtgattgctctatgtatgtatgtatgtatgtatgtatgcatgtatctatctatctgttgtctaataaaatggatcacatctctgctaataaaaacactacactggcagggtacaattataatcattaactgtgcctggaatttgcagcagcagccaccagaggtaccagtgccctttaagggttcataatttagaataatccaattatctgagtttttcagggactgaggggtttggcaaggtgtagaactttcagtaataaagtcaagaaagtcctggacaaaccaaggtagttggtcactctagtccataaccaggtaaagagctttccctgtaacctgtgtaaggttttagaatcatttctttccttattaccaaaaatcctccccaaattttcaagaaattatgaactaaatagttactctatgagataggagttcagcccaaaagaaacaccataagaacaaatataattcttgcttatgttaaccatgcaatgaagcagagagaaaaagtcagtggcctctttaggaggactgtagtgtgggaagaaataactaaactgggtttcaatcctggcctggccaggatctggagcaagtgagttaatctttctaagccttgagtagtttcttcttcttcttcttcttcttcctcccccttctcctcttcttcttcctcctccttctcctcttcttcttcttcttcttcttcctcttcttcttcttcctcctcctcctcctcctcttcttcttcttcttcttcctcttcctcttcctcttcttcctcttcctcttattcttcttcatcgtcttcgtcttcgtcttctttttattttcaaagtgaaagcaagtttattaagaaagtaaaggaataaaagaatggccactccatagacagagtagcctgaaccttgagttcttctataaagtcactatgaatttatactcattttgaaagtgggtgtcaatatgtctgtccactttgcacagctgttatgtggacaaaaggagatctgtgtgaaagtgtaacacagagcctaaactataacaggtaagcaacacagttgtccccttccccatggtgtctgttcttctccatttcctcctgtctgcagggggattataaaactaatcatcaaagccaagaaggcaagagcaagcatgtaccgctgaaaacacaagataactgcataagtaatgactttcagtgcagattcatagctaacccataaactgctggggcaaaaatcatcttggaaggctctgaacctcagaaaggattcacagtaagttaaccatgtagatctgagaggagagtagcttcttgtagataacagttggattatataccatgtcctgatccccttcatcatccaggagagcagaggtggtcaccctgatagcagcaagcctgggggctgcagcttggtgggtagaggtactcaggggtacagatgtctccaaacctgtcctgctgccttagggagcttctaataagttgatggatttggttaaaattaacttggctacttggcaggactgggtcagtgaggaccaacaaaaagaagacatcagattataccctgggggtttgtatttcttgtgtttctttctcttctttgtactaaaatatttacccatgactgggaaagagcaactggagtctttgtagcattatcttagcaaaaatttacaaagtttggaaaacaatattgcccatattgtgtggtgtgtcctgtgacactcaggattcaagtgttggccgaagccactaaatgtgagatgaagccattacaaggcagtgtgcacatctgtccacccaagctggatgccaacatttcacaaatagtgcttgcgtgacacaaatgcagttccaggaggcccaaatgaaaatgtttgtactgaaatttgttaaagcttcccgacaaactagatttatcagtaaggattgttttctgcaagggggatgaaacttgtggggtgagccatttgggctgaggaggagggaggttggagctgagaaatgtggagacaatttccctttagaaggactgaatctccctgcctctctggggtgcggcagccagcaggatccaatggtgtatatgtctccccagctccccattcagtgatatcatgtcagtagcttgaaattatccgtggtgggagtattatgtcatggaaattggcaaatggaaacttttattggagattcaattgttaaacttttaccagcacaacactgccctgccttcagagtcaatgaccctatccaagtttaatccatctgtccactgtctccaacacgatctttataaaacacacctgacaacattacccttttattcagttttttaaaagataagtttccagctcatcgggctggctttaaaggccatttctcctctggacctcacccaacttttcaaatcacttttcctacccctacctctaaatgctactcaaactccagccatcctgaataataagacttttgaaaagtagattatgggctgggcacagtggctcacacctgtaatcccagcactttgggaggccaagatgggtggatcacctgaggtcgggagttcgagaccagcctgactaacatagtgaaaccctgtctctactaaaaatacaaaattagttgggggtggtggcacaagcctgtaatcccagctactcaggaggttgaggcaggggaattgcttgaacctgggaggcggaggttgcggtgagcctagattgctccactgcactccagcctgggcaacaagagcgaaactccatctcaaaaaaataaataaataaataaagtagattacatcagatacctctggcctaggttgtttatgaccaactctcctgctgagaataactagaaaagctagacaaaacatatttccaaaagatctctttggaggcatcagagaatggccaaggctgtaaggaactgcctgagcccagagaggtggagcccagcactggtgccctttactcctggggacatgtgctggtttcaaaaacttcagctgagtttttgagcattcatggaacttggtgggggagatgaaatttgtaccttaaatcctgcctacagggagggtccctgataatccccacccaatttggaaatctgggtcagccttcacaggtactgaagccctcctctgaatgatctcaagtcctgctagggtagaggttacctgcttttgaaaggctcctggcctacctgtgcagcaggagcaaaagtgaaccatctcagggtacagataacaatcatccagagccttgaatgacctctactgtgcttaatatatagtattcagcagtcagtaaaaaggatttaggcacatgcaagatgacctgtgtatcagggagaaataggcaataaattgagatccagcagggatttgaatcatggatttgaatcaggggcagccttcgaaagaactgtggagaatatactcagatttaaaacataagattggaatttttggcagagaactaacaactgtacaaaaaaggaaccaaatggaaatcctagaactgaaagatgcaattaaccgatgttgagaaatagccaacatctattgaacacttcccatgtggacagctgtgctaaacactttacaggcatcaacataagatgtgtccccttacagcagtgcagtgtccctcctaagacatggacagcctggtttccctatctctctgcttcatcaaaacccctttacgtggggcttagacactcctgttgtctctagtgtctagtagcacagggctcagcacatggaagccactagatacaatttgatgaccaggacctccgatgaaagccatgggtgctgattgggaaggcattgtcttttatgtgctatggtcttaaagcttcatccaggaagcagaactcggggggtgctgaggacccagaaccgagaataagattagtcagagatttcctgtgggcagaaatcataaggacgccaactgtttgggtgagataagacgaaaccaagagtggacttgtggccagaagcgtgaggaagagggagagagcttcccttgtcccctttcttcctctccctaagccacagtgattgacagccccccccctttggagtcagagcaggcttgagactggactgggaaaggagggtgggtcaggatacagagcaggaaggctgggagtgcagggcaggagcaaggggctggggcattcattgtgcctgatctctcccactttacctggggtaaagaagcatatgcaaaagccacggtgtgagtatttcccaagtgccagggtcagggcatgattcatcacgtgcagcatttcattcaatccttatagtaaccgatgatgtggcttctattattagctctatcagataatgaaactgagaccaagacaggctctgcacattgtgtggggtaatgacacagggggattcagacctagactccataactcctgccccagggaccacccccaccctcaccctgtgcatgtcgacaaaggacagactgggccacttctcaggacacagcggggaaatgacacagagcagggaggttccaggagccccgagcgtcttttctccaggagaatactctctgaattcagactggggtcagagaaacatttacccaggagccgcagtgtgggtggggctttttacttgaaacgctgtctgaaggcagtggccaggatggaactctccaccctaccttggcaagccacttctcttctgcaatctgtaaggacattgttgagagaattatggtcttccaattccggagggttgaagaaagacaaataggagagaacctatcatagtcaggtgctagctgccttctctttcagagagtgtgagaataaagtgatacacttgattattagcaaatactttggaaattttaaacgctaatattcaacacactctggaagaggcaaataagtagacaggttcatatacatcatctccttcagctagtcctcacaaaaacaaacaaatgaataaacaaaattcttctttggccctcataggaagacactgtttcttgaacgtgtttcaaaaaggatgggtgactcactcaaggtcacactgtttatgaggacagtacaggaatacagacatgccattttgcctgaaaaaatccatcacccagggaggtgacacaattttgcagaaatgttctatttcctctgaaggatacattctttaaacctttgggaaattcattcatagtcttcctcctttgaaggattaactctctggacacaaagtgtttgattctgatttgttggttggaagatgtgttggttgagagaaagattctgatttgttggttgaaaatagactcatcaagatcaactgctgtagtagtaaatattttgacattttgtctgtattcctgtgctgccctcacaagctgcatcaccttgagtgagtcattcatacttttttgtttgtttttgttttggagatggagtcttactctgttgcctaggctggagtgcggtggcgtgatcttggctcactgcgacctccatctcctgggttcaagtgatcctcctgcctcagcctcccgagtagctgggattacaggcacatgccaccatccctgctaatttttgcattttcagtagagacggagtttcaccatgttggtcaggttggtcttgaactcctgacctcaggtgatccgcccacctcagcctccccaagtgctgggattacaggtgtgagccaccgtgcccagcccagccatcatttttgaaacacgtttgagaaatagtgtcttcctttgagggccaaggagacattttttttgtttatttgtttgtttttgtgaggactagctgaagggggtgatgtatattaacctgcctacttatttgcctcttcccagagtgtgatgaatattagggtttaaagtttctgaagcatttgttaataaagcccggggctggaggtcagaagacctggatttctctgcatacttttgccatcagcaagctgtgtgaccttggacagatcccttttttgtctaaatctttctgagtcttcttgaaaacaatgccaggttgggacaggatgattgccaagctcccgtccagctctaaaacactgcaacgtatgcttctgcaccagcactgtccatcctgtagatcatgcagaaattctcttcaactttttcctacccataaaataggagcatgcttacctttttcctaatgttccaggccccgggtctagaatattgtaagtaaggaagttaatgtgtatcagagcccattatgggccagaagttctcctcttccttcctacacctgcttcctccctccctccctccctctttcccttccttccttccatccatttgtgaagaagacatgatcaccctcattctgagagtgaagagacagaggctcaactaatgaaatgatttgttcaaggtcacacgggtggcacaaggcaagtggcagaggttgaatttagacccattcctgtccaaatgctgagtttatgtcatcgtcccgagaccataactttaaagatgtaagatagtgggaaaagagttgatttcaaagcacctctcagaaggactcactttacatcaggggtcagcagactcaggccaaatccggtccattccccgcttttgcaaagaaagttgtagtggaacacagctaggcttattgatttatggattgccaacgtccttttgtgaaacagacagctgagctgagtaatcgtggcgcacaaaacctaaaatatttactatctcgtcctttacagaatgtttgccaatctatggtccggagtccaaggctgtccatttttcaaagaacacaaagtgacatgagactgtcccatgtgcagggagccctatcattttattatgaaaaaacggcctttctgctcaaatctgttttttaaaaagtcaacaaacagactctgggtacctgtcaggaacagtagggagtttggtttccattgtgctcttcttcccaggaactcaatgaaggggaaatagaaatcttaattttggggaaattgcacaggggaaaaaggggagggaatcagttacaacactccattgcgacacttagtggggttgaaagtgacaacagcaagggtttctctttttggaaatgcgaggagggtatttccgcttctcgcagtggggcagggtggcagacgcctagcttgggtgagtgactatttctttataaaccacaactctgggcccgcaatggcagtccactgccttgctgcagtcacagaatggaaatctgcagaggcctccgcagtcacctaatcactctcctcctcttcctgttccattcagagacgatctgccgaccctctgggagaaaatccagcaagatgcaagccttcaggtaaggctaccccaaggaggagaaggtgagggtggatcagctggagactggaaacatatcacagctgccaggggctgccaggccccagagggcctgagaactgggtttgggctggagaggatgtccattattcaagaaagaggctgttacatgcatgggcttcaggacttgtgtttcaaaatatcccagatgtggatagtgcgaccggagggctgtcttactttcccagagactcaggaacccagtgagtaatagatgcatgccaaggagtgggactgcgattcaggcctagttgaatgtgctgacagagaagcagagaggggcaccaggggcacagcccgaaggcccagactgatatgggcaaggcctgtctgtgctgacatgtcggagggtcccactctccagggaccttggtttccccgtctgtgacatctgtgacatgagagtcacgataactccttgtgtgccttacagggttgttgtgaaaattaaatgcacagataatagcgtaacagtattccgtgcattgtaaagagcctgaaaaccattatgatttgaaaatggaatcggctttgtgagaccatcactattgtaaagatgtgatgctgatagaaatgacaggactgcttgtgcatgccctctgcagtgtgacattccagcagtgaaatcatgttggggtgacttctcccccactctgacctttatgtttgtctgggccgaggctgcaagtcgggctctgtgggtgtatgagtgacaagtctctcccttccagatatggggactgtctgcttccctaggttgcctctccctgctctgatcagctagaagctccaggagatcctcctggaggccccagcaggtgatgtttatccctccagactgaggctaaatctagaaactaggataatcacaaacaggccaatgctgccatatgcaaagcactttggtttgcctggccacccctcgtcgagcatgtgggctcttcagagccacctgatgaggtgggtacagttagccacacttcacaggtgaagaggtgaggcacaggtcccaggtcaggctggccagagctctgtttattacgtctcacagctttgagtcctgctctcaaccagagaggccctttaccaagaagaaaggattgggacccagaatcaggtcactggctgaggtagagaggaagccgggttgttcccaagggtagctgctcctgcaggactctgagcaggtcaccagctaatggaggaaaggctctagggaaagacccttctggtctcagactcagagcgagttagctgcaaggtgttccgtctcttgaaacttctacctaggtgctatggtagccactagtctcaggtggctatttaaatttatacttaaatgaatgaaaatagaagaaaatttaaaatccagacccttggtcacactatccacatttaaagaggtcaatagccacatgtggttagtggccaccctattgggcagtgcagctacagaacatttttgcatcccagaaagttcttttggatgttgctgctctacagcatgctttgctgaaacagaagtgccttccctgggaatctcagatgggaagcaagtaaggaggggagtcaaatgtgggctcactgctcaccagctgtgagggttgggcctgcctcttaaccattgtcagcctcagtcttctcatccatgcatgccgtgggtatactaaaatactatacccctggaagagctggatgcaaatttgacaagttctgggggacacaggaaggtgccaagcacaaggctgggcacatggtggctgtgcactacagctgagtccttttccttttcagaatctgggatgttaaccagaagaccttctatctgaggaacaaccaactagttgctggatacttgcaaggaccaaatgtcaatttagaaggtgagtggttgccaggaaagccaatgtatgtgggcatcacgtcactttgcccgtctgtctgcagcagcatggcctgcctgcacaaaccctaggtgcaatgtcctaatccttgttgggtctttgtattcaagtttgaagctgggagggcctggctactgaagggcacatatgagggcagcctgaagagggtgtggagaggtagagtctaggtcagaggtcagtgcctataggcacagtggtcccagggccacagctgggaagggcaaataccagaaggcaaggttgaccattcccttcctcaagtgcctattaaggctccatgttcctatgttgttcaaaccctaactcaatcccaaattaatccaccatgtataaggttgagctatgtctcttattcctggacaccatactcagccatattctggtccacacattaaacaagctggatgaccttgaagaagcttcacccactctgttcctcagctttcccttcagtgggatgatatcaactggacaacaggatgtgcgattcttttagttccagccttccaggatgttttcactcccctgtttgttgttgtaggatggtattacctccaccttcccaccttccctatgccctggttctgtctcctgtgcctcgctctgaaagtggatgagacctacaattcctgtcctggtagttctcctaatgaacacactgaagcacgaggaagctgagatttttgttgctacatgagagcatggaggcctcttagggagagaggaggttcagagactcctaggctcctgtggagccccactcatggccttgttcattttccctgcccctcagcaacactcctattgacctggagcacaggtatcctggggaaagtgagggaaatatggacatcacatggaacaacatccaggagactcaggcctctaggagtaactgggtagtgtgcatcctggggaaagtgagggaaatatggacatcacatggaacaacatccaggagactcaggcctctaggagtaactgggtagtgtgcatcctggggaaagtgagggaaatatggacatcacatggaacaacatccaggagactcaggcctctaggagtaactgggtagtgtgcatcctggggaaagtgagggaaatatggacatcacatggaacaacatccaggagactcaggcctctaggagtaactgggtagtgtgcttggtttaatcttctatttacctgcagaccaggaagatgagacctctctgcccttctgacctcgggattttagttttgtggggaccaggggagatagaaaaatacccggggtctcttcattattgctgcttcctcttctattaacctgaccctcccctctgttcttccccagaaaagatagatgtggtacccattgagcctcatgctctgttcttgggaatccatggagggaagatgtgcctgtcctgtgtcaagtctggtgatgagaccagactccagctggaggtaaaaacatgctttggatctcaaatcaccccaaaacccagtggcttgaaacaaccaaaattttttcttatgattctgtgggttgaccaggattagctgggtagttctgttccatgtggtggaacatgctggggtcactttggaagctgcattcagcagagtgcctggcttgcgctgggcatccaaggtggtccctcatcctccaggctctctttccatgtgatctctcagtgtttaagagttagttggagcttccttacagcatggcggctgacttccaaaagggattattccaaaaagagcctcaacatgcaggcgcttattatgacttctgcttgcatcatcctattggccaaagccagtcacgtggctaagtctagccccctgtgagaggagactgcataagagtgtgaacaccaggagacacggtcactgggggccaccactgtaaccatctaccacaggacctgaatctctgtgtgctactcccttgctcaagggcccccctacccacgcagacctgctgtcttctagcaaagcccatcctcaggacctttctcttccaatccttattgactcaaattgattagttggtgctccacccagagccctgtgctcctttatctcatgtaatgttaatgggtttcccagccctgggaaaacatggctttgtctcaggggcttgctggatgcaagcttaacctcaatgtgagtggccatactgtggcactgtcccatccctcaccagggacactgttctggagggtgactgcctgttctgtgaggagtggggatggctaggacattgcatggaacacaccaccaccccatcttctcagagctcaaaccctgacagaacaccagctccacaggccttggcttctgctgatggtgccgtgtatttaccagacttagtggtccaaggccagagtggccagatttcccaaagtcaaggtgtgacagtgggacagcctctttgtgtctttgctgtcctaagaaacctgggccaggccaggcgcagtggctcacgcctgtaatcccagcactttgagaagccaaggtgggcagatcacgaggtcaggagtttgagaccagcctggccaacatggtgaaaccctgtctctattaaaaatagaaaacattagacaggtgtggtggtgcatgcctgtaatcccagctactcaggaggctgaggcaggagaatcgcttgaacccaggaggtggaggttgcagtgagccgagattgtgccactgcactccagcctaggcgacagagcaagactccgtctcgggaaaattaattaataaataaataaacctaggtcccagagtcccacagaatggcagacaggagcacctgggggcttttagggtatggcatttcccctgtactaactctgggctgtccagagggccatttcatggcgtggagtggagagggaggcagcacaggacttcctaggcctcagctctcacctgcccatcttttgatttccaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaacaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatccatcaggccacttgatgacccccaaccaagtggctcccacaccctgttttacaaaaaagaaaagaccagtccatgagggaggtttttaagggtttgtggaaaatgaaaattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctcccccaccgggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgcctatattgttgactatgtccttggccttgacaggctttgggtatagagtgctgaggaaactgaaagaccaatgtgtctttcttaccccagaggctggcgcctggcctcttctctgagagttcttttcttccttcagcctcactctccctggataacatgagagcaaatctctctgcaaaaaagatatggggcagcactgtccacaacagcctctgctggaaacaacccaagcacccatcacagaatgaattagtacatcatgtatctgcacacaacacagtgctccttggcaaagaaaatgaatgaattacagccagctgcacctcacaagacagataatcttacaaacatcatgtgggtacaagaatcacaaaagcatgcatagagtgtgattccatttctacaaagttcaaaacgggcaacatggggctgttgtgtggagggctgtggaaacaggtggtgcagagagaagcaaggccatgctctgtctgaaggcccagtgacacttggcccctgcgagagagagtggcaggcaggagcacctgggggattttagggtccagacaacattctatttcttggcatggatgcagttaggcggttttctgtatgtatattttatttcacaatacctagaaaataaaaagacttgccccccaacaaccagggtgtaaagaaaggagcatctgggaaattatgagattagtgaatggagtattgaatttgtccccataggtgggagtgcaagaccatgagtgaccacttatcaaacacacctcagtaaacagtcaggagagtgaggagtgtggtcctttcacctgcccaggttgtttctgttagattctgggcttggtctctggagtccaagaggcaggccccagtgaggtcatgcacttgctgtctgattttggccaagtcacctccctttgtggggactgttcctcatttgtttgaggggaatgttgatttggatggggattttccaaacagagctccttggacccttccctctaaggtctctggaaatgggaaaccctgcagaggggaggataaatgggccaggattcatcccttgaaccctgttcccaacagagccctctgtttttctcttctttacaacttggacctcccatgggactttgtttgaaagggagacttcattgctaaaagcaaactaaactaaataggctaaaaggcaacaaactggagaaaatgtttaaggtcttctctagatctaagatcctatgacgttgaggaggtcatccaacatctctgagttttctctttcttgtatataaaatggaagtagctggcagggcagggcatgtgtcgttgatatatttttaatcctggtaaaaggaggaggttcaaggcagaaaactcatcaaagcttaatgacaacaatagcagctaacatttgtgaggctctaaggtaaaacccaaggctaagactcttcatgcaaactaggtcaagcagcaagctacaggttagaaacaggcaaggcagcaaaacagtcacatgggtgaactctgggactctgcagtcacactctgactctcctgcttgcactgggtgctcataagcaagctagttaacacctcagagccttggttttcccaccactaaatgcaaataataggcaatgctgagttattgcaaagttacttaatctttgcaatagccttctgtggtgagtggcatattcttattattattattagtcctagaagagtcagtgctcaaaggaatctgctttagagccttattgctccaagtgtggttcatgcatcagcaacatcagcatacttctggagttaaaatgcagattctcagatctcaggcttgacttactgacttagggtcatcatcttaaccagatctcccatgcacattaaatttgagaagcactgttttaaaatgcattagccgggtgcagtagttcatgcttgtaatctcagcactttgggaggccgaggcaggtggatcacatgaggtcaggagt

SEQ ID NO: 115 - DNA, 15996 bp, Homo sapiens
ttcctgactaaagcacccacagctgtgtctgttccctgtaatgaatccagatactaaagtaggcgggctgcagtgtggagaccgtgacccaccagaaacaaggacggcaactcaaagacggaggaggcacatccaggaggaacctgtggggagggcccgtctggccagatctccactgccctgtccagacttgggcttgcctaatagatgaagcatcagtcatttcagcaactcaagataggagtcatcattatcatcatcacactcactgtgtgccaggcactattctaaatacttgaaaactttaaatgtattcattcctcagagcaacttcatgagacagggacagctatgacccctatttcacagatgaggctgagtagcgtgcccaaggtcacacagccaggaggcacagcagccaggcctgacagaccacctgggcccagcgtccgctctcttagccaccgtgtactatagcagcctctgttaacagaccccctttctggatgacacatgccaagtactttccatggaaccactcacttgctcctcacaaggaagagccacattattcccatttcacaggtgagaaaatcgagacccagagagagttaatgatctactcatggtcacagagttgataagggctcatttgctggactcccaaacgcagtgctcataactgctacgttccagggcctgaaggaaaaactctgcatccatggaggggccggcgctggttctcagctctcacacaggggaggggaaggggcctgtgaccgacacagccagagacagcagtattcaacctccctcctgaactttggtgtcaggcccaccacaccccgccaaggcactgcccatggccctgcaggctcggagactccttcgcagtggtggtagtggtggtgatcactgccctcctctttgtccctgacaatgcaggcacccaccttccccatctctacccacctgccgcacctgcagctgccatggtgctgtccctgcaggcgaggatggcccatcccccacttctgccctctggggagactcctggtcactctcgaatgttctggacagtttatcctttcatctttggcctcatttcaccattgaaacaaacaaaacaagctggattctgcttctgagctgaaggtgcccacctaatattcccttttcactcaccagctctgccctcagagcctcaagcccagggtctgccctttagtgggtgcttagaaaaacaccagatggacacataaatggctgttccactgcccccacagacgccccagaaccccgccctccccaccagctccccttctgcatccccgactctccttgagaacctatttggcagaagctctccacccagcaagtccgcagcttgatgagctccctcctgtgttaactggaaccgctgctgtacttcattccacataatagttcatcggatccaaagtccccacctgctttggaagcaaccacctgctcttctcataactctcctccagtttgtgcagtgaagaatcaacctttatccaagaagtctggcctttgtcctggctcttgggaggtcctaccagctacaaacccttggagtaaacaacgtggctagtccttgtcaccagttcccaggaggtagccccaaattcctagggatttcccaagtgataggagtatcttattattcatggtggtctctgagagtttatgcgagtgaagtgactcatggtgggccctaggtagtttttgctgacaatacgaccatggaggggctggccacgccactgaggttctgtgatatcagcctggcctcccggaaggagaccaggaagatgagttcaacccagtggccaatgagtccatcaaccacacctatatgataagactcaaataaaaactctggaccccaaagctcaagtgagcctccctgcttagaaatagtcagcattttgtcacacgtcaaagtgctgagaaggtgatgcctctgacgccacacggggaagacaatgagagctttgtgtttgggcccctcctccatctcgcccctgtgtctcctcttttggctggttctgatttgaatgcttttgttatgataaaactgtggccttacgtatagcactctcctgagttctgtcagtcatcctagtgcattctggaacctgaggggtagtggaaactcccagatttgcagccagtcagcagtgagggtgggctgggaacccctgaatgtgcagctggcgtctgaagcaagggcagtgttgtgggggaccatacccctcacctgtgaggtgtggctcatctcaggtggtttggcatctgaagccactgcatttagtttggtaaccttgctgcccagtcccaatggaaggatcctaaatatggtctaaggacctcctgatacaattatccagattctctccttcacagaacttgaggcactgcgataagatccaaaactatatatagacagtggaatactatacagccttaaaaaagaaggaaacgctgtcattcaccacaacatggatgaacctggaggacattatgctaagtgaaaaaatccaggcacagacagacaaataccacatgatctcacgcatatgtgaaatataaaaaagccaaactcagggaggcagagagtaggatgatggtcaccaggggctgggagggtggtgatcaggaagatgttggtcaaaggatataaaatttcaactgggaggagttaagttaaagagagccattgtacgacatggtgacaacagttgatatcaatgtcttgtatacttaaaaatcatgaagacaggccaggcgcagtggctcacacctgtaatcccagcactttgcaggggctgaggtgggtagatcacctgaggtcaggagttcgagaccagcctggccaacatggtaaaaccctgtctctattaaaaatacaaaaattagctgggcgtggtggcaggcacctgtaatcccagctactcgggaggctgaggcaggagaattgcttgagctcaggaggcagaggttgcagtgagctgagactgcgccattgcactccagtctgggcaacagagcaagactctatctcaaaataaataaataaatcacagagtagattttaaatgttcttaccaaaaataaatatgtgaagtattgtataagtagcttgatgtagcaattccataacatgcacatttcaaaacattatatcatacagcacaaatatgtgcaatacttatttgtcaatttaataataataataataagggaagaaaagatccaaaacagaggcaaaaccttggccgggcatggtggttcacgcctataatcccagcactttgggaggctgaggtgggtggatcattttgaggccaggagttcgacaccagcctagccaacatggtgaaaccccatctctactaaaaaaaaaaaaaaaatacagaaattagccaggcgtggtggcatgtgcctgtaatcccagctactcgggaagctgaggctggagaatgccttgagcccaggagatcaaggctacagtaagctatgatcaccactgcactccagcctgggtgacagagtatgggggcagggggtggtgaggggggggcggggaagtggaacagaggcaaaaccttagcaacacacatttttagatgatcttccagaataaattcatagggaggcccaggcacagtggctcacgcctgtaatcccagcactttgggaggccgaggcaggcggatcacgaggtcaggagatggagaccatcctggctaacacggtgaaaccccgtctctactaaaaatacaaaaaattagccgggcgtggtggcaggtgcctgtagtcccagctactcggggaggctgaggcaggagaacggcatgaacccaggaggcggagcttgcagtgaactaagatcacgccactgcactccagcctgggtgacagagcaagattccatctcaaaaaaaaaaaaaaaaaaaagaaattcatagggaaaagaaggtcagagaccaagggaagggaaggttctgggagaaaagcagggggcaggcagggcccaagaatcctgctgcccatgagcccttactgggaggtggggtggccttgcacagggcccaggcacctgagtgagtggtggggtccttacgttcactgctggggtgaggcaatgagcaccttattgtgtccacatgaattcaataaaaaacaagcagggcgggtggtggggcactgactaggagggctgatttgtaagttggtaagactgtagctctttttcctaattagctgaggatgtgtttaggttccattcaaaaagtgggcattcctggccaggcatggtggctcacacctgtaatctcagagctttgggagactgaggtaggaggatcacttgagcccaggaatttgagatgagcctaggcaacatagtgagactcttatctctatcaaaaaataaaaataaaaatgagccaggcatggtgcggtggcacgcacctactgctaggggggctgaggtgggaggatcacttgagcctgggaggttgaggctgcagtgatccctgatcacaacattgcatttcagcctgggtgacagagtgagaccctgtctcagaaaaaaaaaaaaaaaagtcattcctgaaacctcagaatagacctaccttgccaagggcttccttatgggtaaggaccttatggacctgctgggacccaaactaggcctcacctgatacgacctgtccttctcaaaacacctaaacttgggagaacattgtcccccagtgctggggtaggagagtctgcctgttattctgcctctatgcagagaaggagccccagatcagcttttccatgacaggacagtttccaagatgccacctgtacttggaagaagccaggttaaaatacttttcaagtaaaactttcttgatattactctatctttccccaggaggactgcattacaacaaattcggacacctgtggcctctcccttctatgcaaagcaaaaagccagcagcagccccaagctgataagattaatctaaagagcaaattatggtgtaatttcctatgctgaaactttgtagttaattttttaaaaaggtttcattttcctattggtctgatttcacaggaacattttacctgtttgtgaggcattttttctcctggaagagaggtgctgattggccccaagtgactgacaatctggtgtaacgaaaatttccaatgtaaactcattttccctcggtttcagcaattttaaatctatatatagagatatctttgtcagcattgcatcgttagcttctcctgataaactaattgcctcacattgtcactgcaaatcgacacctattaatgggtctcacctcccaactgcttccccctctgttcttcctgctagcatgtgccggcaactttgtccacggacacaagtgcgatatcaccttacaggagatcatcaaaactttgaacagcctcacagagcagaaggtgagtacctatctggcaccatctctccagatgttctggtgatgctctcagtatttctaggcatgaaaacgttaacagctgctagagaagttggaactggtggttggtggcagtccagggcacacagcgaggcttctcccctgccactcttttttctgagggtttgtaggaagtttcctcagttggagggagtgagagctgctcatcaaggacttctctgtccggttggaggttaactctgtctcttgctctctcatttctgcctggaccaagactctgtgcaccgagttgaccgtaacagacatctttgctgcctccaaggtaagaagccgtcccacggtctgttttagcaaatggggagatccatccccaaatgtctgaacaagaaacttgtctaatggaaaacgagcgggcccaaattaactctaaggtgttagatgttttcaaagaacgagaagtctgatctttactcttaagcatgttttggtctttctggtttcacttgatttagaagacatgtaatagaaagcttacatgctgtagtcctgactcagatcctggtcaaagaaaagccctcttgggttttacttagctttggcatagtgcctggaacgtaggaggcactcaataaatgcctgttgaatgagagaatttttctggcccatacatttctgaaaaaccaaatactctcacagaaacagatattgagatgacaggttgagggagctttcattttgtctaagagacttcctatggcaacagaaaaggtatcgccagagcccctcctcttccacagcctggccacctaacagccctctgggttccggggctggccgtccagagctcctcagcttgctctggccggccgaactcccctccagctcggtctggaaccatcctgctgggcagcgtccagcacatccctgcttcgggctgcctgggcacctcgcctctctgcctcctgtgctgcctcacccccacccctctatctgtagtgggaggagatagatttgacagctgatagtgcattttctctgacaaacacatgactacagccgtatcaatagttttgtgcatttcagttcctgttttcatggaaacacacggctgagaatgaaagccccaaagcctcaatttcacagtggtctcctaactacctgctttccatgcaaactagggagatgatatggccaggagtgaagccctgtgtgttgggcagggtcacactccagcacccagaccatagaacagggcccatcctgcttcatgagggaaactgctcttcgggcctttagctggactatctcatttcattagttatcccgggagtccgatacaggatgagattctgaagggcaaatacacacttttttttttttttgagatagggtcttgttctgtcacccaggctggagtgcagtggtgcgatttcagctcatagcagcctccacctcccaggctcaagctatcttcctacctcagcctcccaagtagccgggacgacaggtgtgcaccaccacgcctggctaatttttgtatttttttgtagagatggagtcttgccattttgcccaggcttgtctcgaacttctgggctcaagcaatccgtccacctcggcctctcaaagtgctgggattagccactgcacctgggcaacagtttatgtgtgtgtgtgtgtgtgtgtgtgtgtgtatatatgtgtgtgtgtatatatatgtgtgtatgtatatatgtgtatgtatatgtgtgtgtgtgtgtgtgtgtgtgtgtataaaatctccaagtccatccaaccgagatggctcctactagaagccaagagtccaccgggttgagcactgggtctctggaggcctgtggcactgctgagaaggctctaacaaagccaagggaagggccacctcactagaagccaggcctggaggaagggtgagggctgagggcctggaggtaagactgcctgtggttttagacccagctctgccactgactagctgtgtggctggccttcagcacatcttcacacctctctgcacctcagtttccacatgtgaagatatgaaagtgattctgaaggtgattgcaaggttgattggaatccagctcttgagttagtgcaaagtgttattgtgagatgatataaccacgattaaaagcaagaacaggtgcagagaagcgatgattctaagaaggaggggaccgggttggaaaggatcaaaccatccaggatgccgagtctggggcaatccatctgggctgtttctggaagacccccgggtgcaggccaggacactgctgccctcccgtccttaactcccctcttcactcagtcctcactcacctccctctcacacacacaaacatctcctagaataatccccactgcctgccttcactcttacccgtctcatttgcctcccctgaacttcatcctcctggagttcacgatctcactcttcactcttttcttcccctcgaagattcagcactgcttacttacatgttaagatatttcagaacagtgaaatgttgctattttcaaaaacctacaaaggtggtatgcagaggaaaaggtacttctttgtgttcccaaagaaaacatctttccaaaatccagcctattgattttatttcttcgggggaacaagaattttagtatctctaagttgggtagcattctactcttggcagttgctggaaagaaggcactggtctaggtcctgggcttcacaggtaacacctgtcagggtgtctatgaagtcaaggctgtctgaggaacagcaaagtgggaagaagcaagctggctggctgatgaagggtttcttgggtggacaagtagttggagctatttcctatttaccaaagagagctaaagttcataattctacagagagttccataatgaacctcaaatacctctgttttttgaaggagtttctcatatacagcactagctgactatcctgggcaggatgggagataatgaatgcagtgccaatcgggctggatttatatggtcctcagtgaggctggtcaagaaccgagttagaactctcacagagtcactgccacagaagaaatctcccaagtggctgtttcctgacattcccgggagggacaggcctccttctgagtcactccctaagcagttctgaactgtgaggtcagccaggctgtccaagtgcactccctgagccactggcagacacactcagcagccagagctagacaggcaggtggtaggagtccagggccacggcagggatggagtgtcgccccctcgctgcgataccagagcaactaaaacgttaaggccttgcactaaagctgcccttaggatgcattcttttaaagtttttccatttaatgcagactcttttcaattcttattttatccttgtttcctttagaaagtcctttcaaaaatatctttagagggttttttcctatactatgtggccatatacgggtcaaaattaagtttaatttccaggctccaagccagcgtttcagaaaaatctcaccaaggtttgtggtaaaagaagcaaagggctgactttttggttttcttgaatctcactgttccctctgcagcagcatgcatgtctgcccacctccagacacacaggcaccatctgccgccccccatcagcccgtgtcccttccacctcgactcgcctacaaagcccagagaggtctgtttcttggcccccagagcccaaagatactgacacactcttacatttccaactagaatcaggaacgaggagtgactctcagtcagttcattaagtaaatgtctttctaaccgctctgcccatgggacatcacgccccacaggggaaaggggaagcttctgtagcctgggattctggtgcctcagtctgggtctagactttcctgaaaaaacgttaaaatatgaactgcattcctagaatttagcctacataaataagagatgaacacaaagatttctatagtttactcactgccgcttatttacagaagcaaaaatctgccacgataggggcctgacaaatgacagtaccactgtgcaatgcgtttctacgcagctctcaatcccatgttctctaataccaccgaagggcttaggaaatgcttatggtatatgtaaagagtaaagaagttacaaaacagtatcaacagttgacccctattttaaaaagtatttttaaaagtgtgacgatatttaccaaaatattaacagcaatagttacctctggctggtgggatgagtgaatgtatttttgttgaatatatgttacctttatagtaaatatatgttatcttgatcatcagaaaaaaaaatatgtaagaacttgaaagctgcttggacagcgctgctgatagaaacccctgagcatcttgtcactgttcttctgattcagagggtctgggtggggcaggggtggtctgagattctgtatttctaagaagctcccagtgatgtccatgctgctggtccatggaccacactttgagtatcaagggaccagagcatgtcgggggagaggctggggatagctttctttatctgaactggataaaggaactgggctcaagctaagaaccctctccaggttctgcatctttgttcttcagtgaaaaatgagaggacacaccaggccaggttcagactgagacacaatccctctcctgggttcccaatgacttgtctcttgtccattcccttctctaaggctaagggcccccaggaagagccatgtggccagaccctcacagttgctggcattccaaggagattctcactccgcatcatttggggccaaaaggccccttacagaagctctgcccaaggctcagatcaatggcacctgctcccagagcctcctctgatctcccaggacacctttccctgatctgtgcacttatctcttgctgcctggcaaaatgtcttagctcctcacttgggccatgtgctgctctcctctcccatggggagagccacacggagagtgctggccaaagcagcagagttcaggccaaaggatgtgcactcatttattcaacaggcatgcaggatttccagggaaagctggattttaaaacctctgggaacaagagcagaacctgactgagagctcatgtgggcacttttcatagcagaatagctcatgaggtatagagacacggacgcagaacgtgggctgtagcgacagatggtcctgcattctagtccccactgtgccttttcctcatgggatgactttattcaggtaccctttcggcaaaatcctccaagagaaaggaaactgggaggttctggggagaaggctgctgcgtttgcaattgggagaggttgttgacagaggtttatgtctgtggcaagcagccttccttcagtggaatacttgaagacaggtctgtagttgagcaaactcacctccatttgtcctcctggaaagaagaaatcaagaggaaaaatctctctcccatcctccaaatggagctggcacattgctatctgtggcatttgtctttccagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttggtaagctgcactgtattcctggcaagccggccgcgtggctcctggtggacagcagcctcacttctaaacactccttaggagctgcagcacccttggtcaacccattcattcattcactcattcaataagtatttgctgaagttccacaagtgctgggtgtggttctaggtgctgaggacgtgtcactaaagacacgcaggccgagtccctgttctcatggaatgttctaatgggagagttagaaaaacaaacatgtaaaatgatggccagcagtgatacgtgctacaaagaaaaacatagaaataaagaacataagagtcatgggggagggggctgacttaggagctggtgacattatctgagcagatatttgaattgagggagcaggccacatgactaactagggagaccattccaggcagaaggaggaggtatgcaaaggccttaggatggaaatgaactaacttcctgtatttaaagaccagtaggaaggccagtgtggctggatcagagtgagtgaggggtagtttccaggacagcagatcacacaaggcctttagattccaccacgagtatggcagggaacacctgcagagctttgggcaggacaaagactgtacaatctgatttacgtgatttaaaagggtcagtctggctactgtgtggtaaataggctgaaagggggaaagcatagaagcaagatggcctgttgggaggctaccacagtaaaccaggctagagatgatggtggcgtggacagaatgaagcaagatggcctgttgggaggctaccacagtaaaccaggctagagacgatggtggcgtggacagaatgaagcaagatggcctgttgggaggctaccacagtaaaccaggctagagatgatggtggcgtggacagaatgggagcagttgaggtgaacagatttgggatatgactaaaaataaaaccagaaggatttgctgacagatcggttgtagggggtaagatacaggggaggaaaagatgacctctttgttcctgcccaaacccctctggcgatggtcagtactgtttacagagagatgaaagactggcggcaaggcagggctggaggttcagcagaagatcaagagttcaattttgtacatcgtacatgtaaggtggctcttggatagccaagtgaaggtgttgagaagatggttagaaaagtctggaacttaggggagaggtcagaacttgcaatacaaaaaggagagtccttagatagatactgctgaaaatctgaatgacagaaagggagagatcaaaggactgagcctgagatcaacacatggaggtcaggagaggaggatccagccaaggggcctgaggaggagtgaccagtgaggcaggagaacactggagagtgggcggtaccccaggaagccgttgaggacactcaaggagggagggttgactgtgtcaaatgtactgaaaggacaggtcaggtgaggaccaagaaaggcccctgggtttggctgatggaggccatgggtgaggctgatgtaaatggagaggcaggaaggaaagcccagctggagtgggctcaccgaggatagggtggcgagaggagacaaagaaggaacagtgagggcagaacactctttgaagatgtttagctataaggctgcagagaaactgacccacagctgcagggtggttatggagtgagggaagctcttttaaggttgggggtatacccagcatgttaatgcacctgggggaatggtccagtggagcaggaagaactgaagagagcagaaagaggaagaatcattagggggcagaagtccttgtagcccagagtggatgttatctaatatcgagtggaggaattaattggctttagaggagaacaaggacatgtatcccctctctgggcctatcaccttgtagacaatgggataggtcatgggataggaacttggcacaacacatgttctctcttttaattctctccattatcttatgaagcaggcaagtaggcaaacaattgtcccaactttacaaaagaaactgaagcttttataaattaagtagtacatcctaagcaatacaattaataaatggtagagctgagattcaaactgaagcagtggcctgggggtagcatctggaatccttcccacctttagggctgctgtgctgcggtgctgctgtttaatggcacaggagggccacatgactgaatctctctcagcagtccaggcagtcatgcagaaggcccagtagagcaccgggcaggtctgagccagcatcttcaagttccaccctctgagcaagcacctagctgtgacacacctctccagagactgcactcccccccgcgccacccaccccaaaagcagataggtaatggtatacagtaaccatttctagaagtgtaagtagtatgcacccaaaataggcaaaacctgctggcctagtgatagagacaactcccagtcaggctagactggaggccttggttttataagtgttcaggtgacaagtgccacagtaggcttgatcaagtagacaggcaggcaagacaaatgcttaccaatgcaagctaatgaaatgtttcttttgcagaattcctgtcctgtgaaggaagccaaccagagtacgttggaaaacttcttggaaaggctaaagacgatcatgagagagaaatattcaaagtgttcgagctgaatattttaatttatgagtttttgatagctttattttttaagtatttatatatttataactcatcataaaataaagtatatatagaatctaacagcaatggcatttaatgtattggctatgtttacttgacaaatgaaattatggtttgcaacttttagggaaatcaatttagtttaccaagagactataaatgctatgggagcaaaacaggaaagaccacttccccctcgaggggttccctctcgagttagggacataacacacaagataattaaagaacacaaggccatacaagatgtaaataagacaccttgggtccaagagtgctatggattaccatatatttctgaacagaaaaggtatcaactggatgtctaaaacgttcttcccccattggcacaaggattgttatactgagtctacagaaaaaagaagtaatcctcagtacaccatatggctctacagtgaacgatatttaaagtcataataatgtaaattctcttgattctcaagtcttagaaccaaccaatagacaaagcacagaagacttgatgatggttggagaacagactataaattgctatccactgaataatataagagctatagctggaaatggctgggaggtgaaagcagaagaaaagagaggtgggcattgctatctccagcttgcatggacaagagggcagttaaataacactgtgcacagtggatggaacaagaacacaaggtttagggatttatcaaaatcacaaaggtaatagaagaactaaaaataatttactataaaaatttggaagtgatagaattaggtaaattctctttcttcaatagggaaatcaatgtatgacatctaacactgataaagcaaaaaacagtgggtaaaagtggtcatgggttgacagttgttgcagctggggatgtgtacagggggttggttatactattctccttttatacataattgaaagttttcataataaaaagagaagaaaaaataatgaatcaagcaatctttggaattataatgatagtcactggaaaaactaaaaataaaaatggaagatggtttgctgggaatgggacttggactaaggaggggtgaaacagggctggagactttcggctttccattataagtctgtgtgtgtgtgtgtgtgtgtgtgtgtgtgtgtgtgtgtgtgtgtgtgtgtgtgtgtgtgtgtgtgtgtgtgtgtgtgtgttatgtgcataaatgaagttttttcattaaagaaataacatttaaaagacaataactgttggtgaagatgtagaaaaattggaaccctcctacattgctggagtgattgaaaaatggtgtagtcactttggaaaacaattggcagttcctcaaaaggataaaatagtttacgatccagcaattccaatcctaggtatatacccaagggaactaggaacacatgtccacacaaaaacctctacacaaatgttcatagcaacattattcataatagcaaaaaagtagaagtggctgggagcagtggttcacacctgtaatcccagcactttgggaggccgaggcaggaggatcacttgaggccaggagttcaagaccagcctgggcaacaaggcaaaaccctgtctctacaaaaaactagctgagcatggtggcacatgcctgtagtcccagctactggagaggctgaggtgggaggatggctcaagcctgggaggttgaggctacaatgagctatgactgcgctgctgtactccagcctgagcgacagaataagaccctgtctcaaagaaaaaaaaaaaaaagtagaagcaaccaaaatgtcaatcaactgatggataaacaaaaagtggcatatccatacaatggcatattatttgtcaataaaaagaaatgaagtactgacagaggccacaacccggatgaaccttggaaacagtatgtgacgggaaagaagccaaatgcagaagaccacatatggtatgattctactgatatgaattattcagaacaggagaatccacagaggcaaaaatagaatattgattgctacaggctgtgggtgggaggcgggtgtggaggcatggggag

SEQ ID NO: 116 - DNA, 11856 bp, Homo sapiens
ttgcctatgattcttcaacttgcttacctggatctgaattcttccataacattttaaatcaatccaggtgtacccatcttcagaaataatggaccccaatgtctcagaattacagagtcagagtcctcaacatgtccaaagcatttcgtggaaattattccaccatgaagactagaatggcctctcttcatggattcagcttgctaaggaggcttagttcagagggactaagacactctccttttagcaaataactaaccaaaagggcaaggaagggaggtaaaagacgtcataaccaggccagtagaatcccctggtctggccattacatgtgatgatttcaaatgggttcttagtaccttaattttgagggaaaagagtagaggaaaccagccaatgctttgcatgcttagtttctgctgatgacactgaactcagagaactgagatctttggagaaaccaaagatggctcaaatgactcgtgataataaatgaacgtattactgctcgctgacaaattgaagtttcctaattataaagcagaaccagtggccaccaccatagaaagcctaccccacagatggagaagaggcaaagcactgaaactcagtccgagggacctccccagccatgggggcagggccaaatggggcttcttcaggaccagcaaagccatttttctcatcagcaaactagcttcagagaagtttgcaatcagggcactctcttccaagcctagagacccagggaaaggggtacgggggtgtcccaaggcaaagagaatctacactttttgcccccggagaggctacttccctcccaagatgcctgggattttccacttcagcagggggaaggtaagtcacatagcaaaataatgagggcacagaacagatgacctccctatagagttttgaatgagaaacacagcagggcagatgtgccccttctctagtctaggaggagctaggtccagcccctgaacatcctccccctcagaaaagctgaggccagactaagaattcaccagaccaaggagctacaacaggacatcagagctgaggctgcaaagccaggactgagaccagaccaggcaggaaactgtcaagagctttggtcaccaggcctggctgccctccaacatcagctggctctttctaaattgacacaccacatgtccctaaaattctctcttcaagtaataccaccatcaaagcaggacatttcccagagccttagagcctggtgtctgctcagtgggactcaaccccagaagaagctgttaaatcacccactgtttcagtttacaaacttcttacgacttggcaacaagtgaaactacattctggcagcaactgcaagttccctagtacccaggacttcccgttttttcttgctgtactccctcctgttaaatcacagactcatccatctccaacccccagaatatagagaaagagcacaacactacatcttaactcctgagacgtggagaacacttctcctcctgagagcttaagtaccaaatggaagctacttttcccccttggtctcaaatgtattactagattctgaactggactccaccatcacgtaagaaagcagtcatgggcagtaattctgggagatccagataggacatgccagccccacactggtggcataggaagccaagttgctgcttcctccctgtgcactcccatttgtctggcctctcttgatctcagctggcgctcacttcacatcagctatgatgcaatccagcaactaaagtattagttaataaatgctgacagcacagccttttctggtcacgtattcatactaaaatacgggggagagttggggggagagggggatatatgggaaatctctgtaccttcctctccattttgctatgacctaaagctgccctttaaaaaatacaaggggctgggcacagtggttcacgcctgtaaacccagcactttgggaggccgaggcgcgtggatcacctgaggtcaggagttcaagacccgcctggccaacatggcaaaaccccgtttctactaaaaatacaaaaagtagctgggcgtggtcgcatgcatctgtagtcccagctactcaggaggctgaggcaagagaattgcttgaacctgggaggcggcggttgaagtgagccaagatcatgccattgccctccagcctgggcaacagagcaagactccttctcaagagaaaaaacaaaacaaaacaagaaaaaacaaagaatgagctctccacgcgaaaaatccattgagatgcaaaggaaggaagctatcattgtggaattgcacatgtcagttacattaacgtttttggagcaaggtagagctcatctctcccacaagcaaattccagcccaaagcattgatactaataaagtgccatgctgcgatgtgcagggggcagacagtgtctccaagctccctacacacatgccttcccacagtttgccctttcttgaccccagaagcatcaggccccttcaccctcgagggccactatcaggagtttgaattaatggcaatcaccatgcacagggaaggctgtggaattctgacataaaaacacttagtggagggcttggaaaaagtctagtaggagcaagacgcaagctggactaattatctaaaacaagagacctggtttggggatcttaatgttctcaaaaaagaaaattattattatttttcattttgcactttgtgccataaaacattttcaacaaaacatagaatctcatttcttttgagggaaaatgattgggagaccagctcattgctggcacagaggcctggttcattcataattccttcataggcaagacaccaggtgaaccgatatagccgagctggaagagctctccaaggcagagactctgagccaaggaatgttcaaagagctagcatgtattgtgggattactatgcgccaggaattttttacactgcatcacgttccatcttcacaacagccctagaaaggaagaactattattacccccgttttataggtgaataaacaagggcacaggtccttgatgtaacagccaggatcaaacagctgggaagacgagaaaacctttcccaggctaggataacagaggatttggttgaaaatacaggcaattaggtgctacctctgggaaaaggggccaggagaggaaggagacacttttccctgcatgccctgatgtcctatttgaacattttatcatgaacacgaacttcctatttaaaaaacactttttattgaaaagataaatctgtgtgttgtattgtgtcactcagttcaagtacttgaaatttattgaattgtattttctaaaaaatagatagttgagtaaaagcaagctcacattacatagacggatcacagtgcacggctgcggagctgggagcagtggcttcgtttcatgcaggaaagagaacttggttcaggagtgtctacgttgcttaagacaggagagcactaaaaatgaaaccatccagccatcctcccccattttcattttcacaccaaagaatcccaccgcggcagaggaccaccgtctctgtttagacaatcggtgaagaatggatgacctcactttccccaacaggcgggtcctgaaatgttatgcacgaaacaaaacttgagtaaatgcccaacagaggtcactgttttatcgatcttgaagagatctcttcttagcaaagcaaagaaaccgattgtgaaggtaacaccatgtttggtaaataagtgttttggtgttgtgcaagggtctggtttcagcctgaagccatctcagagctgtctgggtctctggagactggagggacaacctagtctagagcccatttgcatgagaccaaggatcctcctgcaagagacaccatcctgagggaagagggcttctgaaccagcttgacccaataagaaattcttgggtgccgacgcggaagcagattcagagcctagagccgtgcctgcgtccgtagtttccttctagcttcttttgatttcaaatcaagacttacagggagagggagcgataaacacaaactctgcaagatgccacaaggtcctcctttgacatccccaacaaagaggtgagtagtattctccccctttctgccctgaaccaagtgggcttcagtaatttcagggctccaggagacctggggcccatgcaggtgccccagtgaaacagtggtgaagagactcagtggcaatggggagagcactggcagcacaaggcaaacctctggcacagagagcaaagtcctcactgggaggattcccaaggggtcacttgggagagggcagggcagcagccaacctcctctaagtgggctgaagcaggtgaagaaagtggcagaagccacgcggtggcaaaaaggagtcacacactccacctggagacgccttgaagtaactgcacgaaatttgaggatggccaggcagttctacaacagccgctcacagggagagccagaacacagaagaactcagatgactggtagtattaccttcttcataatcccaggcttggggggctgcgatggagtcagaggaaactcagttcagaacatctttggtttttacaaatacaaattaactggaacgctaaattctagcctgttaatctggtcactgaaaaaaaattttttttttttcaaaaaacatagctttagcttattttttttctctttgtaaaacttcgtgcatgacttcagctttactctttgtcaagacatgccaaagtgctgagtcactaataaaagaaaaaaagaaagtaaaggaagagtggttctgcttcttagcgctagcctcaatgacgacctaagctgcacttttccccctagttgtgtcttgccatgctaaaggacgtcacattgcacaatcttaataaggtttccaatcagccccacccgctctggccccaccctcaccctccaacaaagatttatcaaatgtgggattttcccatgagtctcaatattagagtctcaacccccaataaatataggactggagatgtctgaggctcattctgccctcgagcccaccgggaacgaaagagaagctctatctcccctccaggagcccagctatgaactccttctccacaagtaagtgcaggaaatccttagccctggaactgccagcggcggtcgagccctgtgtgagggaggggtgtgtggcccagggagggctggcgggcggccagcagcagaggcaggctcccagctgtgctgtcagctcacccctgcgctcgctcccctccggcacaggcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggaggtgggtaggcttggcgatggggttgaagggcccggtgcgcatgcgttccccttgcccctgcgtgtggccgggggctgcctgcattaggaggtctttgctgggttctagagcactgtagatttgaggccaacggggccgactagactgacttctgtatttatcctttgctggtgtcaggaagttcctttcctttctggaaaatgcagaatgggtctgaaatccatgcccacctttggcatgagctgagggttattgcttctcagggcttccttttccctttccaaaaaattaggtctgtgaagctcctttttgtcccccgggctttggaaggactagaaaagtgccacctgaaaggcatgttcagcttctcagagcagttgcagtactttttggttatgtaaactcaatggctaggattcctcaaagccattccagctaagattcatacctcagagcccaccaaagtggcaaatcataaataggttaaagcatctccccactttcaatgcaaggtattttggtcctgtttggtagaaagaaaagaacacaggaggggagattgggagcccacactcgaattctggttctgccaaaccagccttgtgatcttgggtaaattccctaccacctctggactccatcagtaaaattgggcgtggactaggtgatctcatagatccttcctgctggaacattctatggcttgaattatattctcctaattattgtcaaaattgctgttattaagtatctactgtgtgccaggcactttaaataaatattgtgtctaatcttcaaaacaaatttgcaaggaaggtttttggagataaggaaactgagactcaggattaagtaacacacctaaagtcacaggtgagcttggaactgaacccaagtgtgcccccactccactggaatttgcttgccaggatgccaatgagttgtagcttcatttttcttagagactttcctggctgtggttgaacaatgaaaaggccctctagtggtgtttgttttagggacacttaggtgataacaattctggtattctttcccagacatgtaacaagagtaacatgtgtgaaagcagcaaagaggcactggcagaaaacaacctgaaccttccaaagatggctgaaaaagatggatgcttccaatctggattcaatgaggtaccaacttgtcgcactcacttttcactattccttaggcaaaacttctccctcttgcatgcagtgcctgtatacatatagatccaggcagcaacaaaaagtgggtaaatgtaaagaatgttatgtaaatttcatgaggaggccaacttcaagcttttttaaaggcagtttattcttggacaggtatggccagagatggtgccactgtggtgagattttaacaactgtcaaatgtttaaaactcccacaggtttaattagttcatcctgggaaaggtactctcagggccttttccctctctggctgcccctggcagggtccaggtctgccctccctccctgcccagctcattctccacagtgagataacctgcactgtcttctgattattttataaaaggaggttccagcccagcattaacaagggcaagagtgcaggaagaacatcaagggggacaatcagagaaggatccccattgccacattctagcatctgttgggctttggataaaactaattacatggggcctctgattgtccagttatttaaaatggtgctgtccaatgtcccaaaacatgctgcctaagaggtacttgaagttctctagaggagcagagggaaaagatgtcgaactgtggcaattttaacttttcaaattgattctatctcctggcgataaccaattttcccaccatctttcctcttaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggtgggtgtgtcctcattccctcaacttggtgtgggggaagacaggctcaaagacagtgtcctggacaactcagggatgcaatgccacttccaaaagagaaggctacacgtaaacaaaagagtctgagaaatagtttctgattgttattgttaaatctttttttgtttgtttggttggttggctctcttctgcaaaggacatcaataactgtattttaaactatatattaactgaggtggattttaacatcaatttttaatagtgcaagagatttaaaaccaaaggcgggggggcgggcagaaaaaagtgcatccaactccagccagtgatccacagaaacaaagaccaaggagcacaaaatgattttaagattttagtcattgccaagtgacattcttctcactgtggttgtttcaattctttttcctaccttttaccagagagttagttcagagaaatggtcagagactcaagggtggaaagaggtaccaaaggctttggccaccagtagctggctattcagacagcagggagtagacttgctggctagcatgtggaggagccaaagctcaataagaaggggcctagaatgaaacccttggtgctgatcctgcctctgccatttctacttaagccagggtttctcatatgttaacatgcatgggaattccctgggcatcttcttgtggtgtggagtctgacttagcaagcctcgggtgggtttgagggtcaaatttctaccaggcttatatccctggtgatgctgcagaattccaggaccacacttggaggtttaaggccttccacaagttacttatcccatatggtgggtctatggaaaggtgtttcccagtcctctttacaccaccggatcagtggtctttcaacagatcctaaagggatggtgagagggaaactggagaaaagtatcagatttagaggccactgaagaacccatattaaaatgcctttaagtatgggctcttcattcatatactaaatatgaactatgtgccaggcattatttcatatgacagaatacaaacaaataagatagtgatgctggtcaggcttggtggctcatgcctgtattccctaaactttgggagcctaaggtgagaactccttgaactcctaaggccaggagttcaagaccagcctggataacatagcaagaccccatctctacaaaaaaccaaaaccaaacaaacaaaaatgatagtggtgcttccctcaggatgcttgtggtctaatgggagacagaacagcaaagggatgattagaagttggttgctgtgagccaggcacagtgctgatataatcccagcgctatgggaggctgaggtgggtggatcatttgaggccaggagtttaagaccagcctggtcaacatggtaaaaccccatctctacttaaaaatacaaaaaagttagccaggcatggtggcatacacctgtaacccagctactcaggaggctgaggcacatgaatcacttgaacccaggaggcagaggttgctgtgcaccactgcactccagcctgggtgacagaacgagaccttgactcaaaaaaaaaaaaaagaagtttgttgctatggaagggtcctactcagagcaggcaccccagttaatctcattcaccccacatttcacatttgaacatcatcccatagcccagagcatccctccactgcaaaggatttattcaacatttaaacaatcctttttactttcattttccttcaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattcagcaactttgagtgtgtcacgtgaagcttaatataaacaagtttcttgtcactgccaccaccacgaccaaaaaaagctaatcaatcactatatataatacatatatatactatatataataaatatatatactatatataatacatatatacactatatataatacatatatactatatatacacatatatactatatatacacatatatattatgaatgtatatatatagtatatatagtatatatactatgtatgtatatatatagtatatatagtatatatactatgtatgtgtatatatagtatatatagtatatatagtatatatactatgtatgtatatatatagtatatatagtatatatactgtgtatgtatatatatagtatatatatactatatatgcatacatagtatatatgcatatatactatatatactatatatttatatatactatatactatatatactatatactgtatatatactatatatgtatgtatacgatatatatatatactatatatgtacacacacacatatatatatacatataagcacctactacatgccaggcatcattaaatgtgttgcatccatcacgtcatttaaccccagcacttgcacactcctttctggttgtggaagactaagtaatttatctaagtcacccagctggaaggtcaggcagggacccagatttgaaatccaagtctacctacctacaggtcccctactcttaacctgtaggtcccactgcctacccaggaactgagggatgatgtagaaaatcccaaaacatgttaatatagggaatacctataaacatgcaatcaaagtctttgggactatacaaccactgtataaagcataacaatgtacaagcttccaaacaataactagaagttctgcctccctcttctgggttcctaaagcactgcacctatctacctgtcaaagcatctaccacattgtaccacaccttaaaatcaatggtttttttcttctcagccagcatgtggatgcctcaataaagcagactcctttcatgacctaaaactaatttcaggggggaaaaaaagacgagctgggcgcagtggctcacgcctataatcccagcactttgggaggctgaggcgggaggatcacttgaggtcagaagaccagcctggccaacatggcaaaaccccgtctctactaaaaatacaaaaattagctgggcgtggtggcgcacctataatcccagctactcaggaagctgagacatgataatcgcttgagcctgggaggtagagcctggggctgcactccatcctgggcaacagagggagattctgtctcaaaaaataataataataatataaataaataaataatttttttaaaaaaagactctttcctatattaatctttgcatcctgtgcccatggccccaaacctgaccaatgaaggccccagtaaatattttttgaacaaaagaaaatatagatgatcaaagataagaaagataagaagacacctcaattcttgtaggcatatagtggtaggggaaatcactaattctgtgtgtttatgtatcttcatcttcaggaaaaatagcaagaattagaaagaaacactcaaaggacttccagagaaggcaatgaatagccagtgcatgtacacctacctccatttcctttgccactggcaataatcttcatctgccctctcccctccccgacctaaagtggtagtgagagaggcaatctggacaacttaacatggcctcgtttatccatctcaacacgacactgggaatttggtgcagatttcccctttctaacccctgcaatataaaagttctgtctccttataaagcttttccccattctacccgctacagacttcttcctccaggggaggagttcttctgggtaagggaaagtccacagaggtcaaaaaccgagaggagctgagccagaaaagagccacatactctgatttctagaaaccacgca

SEQ ID NO: 117 - DNA, 10211 bp, Homo sapiens
ttttttttgtaaacattggagtaaataatgcctaagtgttttagaataaatctaataaagaaattaggtctacacataattttgttttacttttttttttttttcttgcgacaggctctctgtcacccaggctggagtacagtggcatgataacagctccctgcagcctcgacctccctggctcaagtgatcttcccacctcagcctcctgagtagctgggactacaggcatgcaccaccacgcccggctaatatttgtatatttttgtagagacagagtttcatcatgttgcccaggctagtcttgaactcctgggctcaagtgctccacctgaattggccttccaaattacaggcatagccaccacagtgagcccttttttttttttaataaatttaacaaagaaaagaaatagctacaagtaaatatttacttttttttttcatattgaagattggctgatcttagcctttctaagtccttaataaatgcctgtttacacaactggtgtcttcccctaaacctaaacttcaaacttcaaactccaaactccaccgatttggatagtattagaaatggcattcatattaatggttatagtacatttttttacatgtgaatgcaaatggaattgttaggcattaaaaaagaaagcttatatagtggaagaaaataaagcatctagacataagctttaagagatctattgtgttaatacagctttactttttgagtggtaagcttttaaaaagaaatgtggtgctctaactccaggaaaagataagggtgactgaagtgatagtctagaggaaaaagatgcagacatttactgagtacctccaatgtgccaggtgccattctgggcattttcattatgtttcctcatttaattctcatggtgatcctttggaactgtgttattctcatttttacagatgaggtaactgagagacagtcagattaaagaactgcctatgattgtttggctaataataagtggaggggtgaggcttgaaggcaggtttgtcttattccaacacccatacatacccttaaatttaagttattctgacttgtgttgctcaaatccaatgtgttcagctgtttgcttctccaattaccaagatttttctttaaaaggtaggacacttttggcaacacgaaccaactttgctcagtattgttataaactgttaactggagacatttgaatttggagatggaactgaaatggtcttgcggtactagagaagatcaagttatcacataaacaaagtacagagctgagaacatattttaaatctttccactaactctgacttttattgactaaaattttagtgggcagtatgtttatgtttatgactcttaacattaacaacatcgtaagtcaaactcactaatatatgttaagcattctgtttatgattcttttaacctagaggattgttgagctgggactaatttcctcaaatgggaaaaaaacccaggtgagagctgagactgctcctgagactgagaaaggcagctctgacgggatctcagattttagcagcaggagttgaacaatgggcatagaatcagcttgcccaagatctcctgattaataaaccatggaacaagatttaaacccaagttcatttcatttcaaagctcataccacattttgcccaccatattttgctttgttatatgactacaacttagttcaggcttacaaaaaagtcctaattctaaaattcctatggcgtgggtgggaggggatttagatgattttgcataggcaagaaacacccagtttcatggagtttgatggaagagttatgtactaatatgggaaaagtagaggccatctttgtctttgttctttcttttttagacgggagtctcgctctttcacccaggctggagtgcagtggcgctatctcggctcactgcaagctccgcctcctgggttcacgccattctcctgcctcagcctcctgagtagctgggactacaggcgcccgccaccgctcccggctaattttttatattttcagtagagacggggtttcaccgtgttagctaagatggtttggatctcctgaactcgtgatccgcccgcctcggactcccaaagtgctgggattacaggcttgagccaccgcgcccggccgtctttgttctttcttgaactcttccttttcttgggtgatagacttcgtcaacgtctaatgaggatatctaggtgctagtctctgctcatcaaatgattcttatggctcaggagccgaatgggacgtaaataaacagttaagtctcatgaactcactttgcattcatctctagaagatgacaaaacatttgtatttatgtgtagcgtggcactttagttaaactttgtaccccactttgctctattttaaagcagaatatccttaaaaaggatacttagtcctgcttttttttttccgcctaagcccatttagtccttctactcattatgcaaggactcaaatggttatctttacagaagtgagacaagatagaatcaatgctcttgtagtcacttcatctttgtccattcccacttctgatggagagggttctaggacataatgcactgaaggttacattgtgagagatgaacaacatttgcaaaagaggtctttttgccttggaaaggcttcattcttaaaaaaaaatgtgagcatcaaggttaagtagacctcattagctcaaactttaaggatgatatcaggataaagttgggcccatgagaagagaatgagagggagatatagtgacatgaaaataaggaggaaaacgaggtgtctatgtaagttgggctcaccataaatacaaaggcaaccgttagggaaaagcaaagaagtctttgcacatcctcagaactctgaatgtcttagtgatgctgtatgagtgagtcttaatgatagtgaactgaatcagtcaagccaggttgtgtccatatgagaatgtgtctttgctaaacatgccaacatcactgaagcaaagaaacttggagttttctttaagatataggtcttttttacctatccggcccaagctttctcttcttgtcactccatgcactgtgttccgtatgctaaatagtttgagaaacccaaatgggccatgttcgcctacatttcattgtcctgtacttcctgtcctgtactagcaaagcagtcccattggtctttcttctcctcattaacaataaaggtaacacttttgatgttgtttcttcagaaaaccttcattcatcaaaactgcctcaaagatcatgtttgtttgattccagaacttcctgtaattacctgttattgtaacactcatcactgtattttacttacttgtgtaactaattttccatattctgcactagacaacaaagtcctttaagtcaggtactatatctatttacatagcattcacatctcctacaataagggacattagcagataaacaacacatattaaatgaataatgaagtttctgaaatactacagttgaaaactataggagctacattatatagaataaacatttactttgctatagaattcagtgtaacccaggcattattttatcctcaagtcttaggttggttggagaaagataacaaaaagaaacatgattgtgcagaaacagacaaacctttttggaaagcatttgaaaatggcattccccctccacagtgtgttcacagtgtgggcaaattcactgctctgtcgtactttctgaaaatgaagaactgttacaccaaggtgaattatttataaattatgtacttgcccagaagcgaacagacttttactatcataagaacccttccttggtgctctttatctacagaatccaagacctttcaagaaaggtcttggattcttttcttcaggacactaggacataaagccacctttttatgatttgttgaaatttctcactccatcccttttgctagtgatcatgggtcctcagaggtcagacttggtgtccttggataaagagcatgaagcaacagtggctgaaccagagttggaacccagatgctctttccactaagcatacaactttccattagataacacctccctcccaccccaaccaagcagctccagtgcaccactttctggagcataaacataccttaactttacaacttgagtggccttgaatactgttcctatctggaatgtgctgttctctttcatcttcctctattgaagccctcctattcctcaatgccttgctccaactgcctttggaagattctgctcttatgcctccactggaattaatgtcttagtaccacttgtctattctgctatatagtcagtccttacattgctttcttcttctgatagaccaaactctttaaggacaagtacctagtcttatctatttctagatcccccacattactcagaaagttactccataaatgtttgtggaactgatttctatgtgaagcacatgtgccccttcactctgttaacatgcattagaaaactaaatcttttgaaaagttgtagtatgccccctaagagcagtaacagttcctagaaactctctaaaatgcttagaaaaagatttattttaaattacctccccaataaaatgattggctggcttatcttcaccatcatgatagcatctgtaattaactgaaaaaaaataattatgccattaaaagaaaatcatccatgatcttgttctaacacctgccactctagtactatatctgtcacatggtactatgataaagttatctagaaataaaaaagcatacaattgataattcaccaaattgtggagcttcagtattttaaatgtatattaaaattaaattattttaaagatcaaagaaaactttcgtcatactccgtatttgataaggaacaaataggaagtgtgatgactcaggtttgccctgaggggatgggccatcagttgcaaatcgtggaatttcctctgacataatgaaaagatgagggtgcataagttctctagtagggtgatgatataaaaagccaccggagcactccataaggcacaaactttcagagacagcagagcacacaagcttctaggacaagagccaggaagaaaccaccggaaggaaccatctcactgtgtgtaaacatgacttccaagctggccgtggctctcttggcagccttcctgatttctgcagctctgtgtgaaggtaagcacatctttctgacctacagcgttttcctatgtctaaatgtgatccttagatagcaaagctattcttgatgctttggtaacaaacatcctttttattcagaaacagaatataatcttagcagtcaattaatgttaaattgaagatttagaaaaaactatatataacacttaggaaagtataaagtttgatcaatatagatattctgcttttataatttataccatgtagcatgcatatatttaacgtaaataagtaatttatagtatgtcctattgagaaccacggttacctatattatgtattaatattgagttgagcaaggtaactcagacaattccactccttgtagtatttcattgacaagcctcagatttgtcattaattcctgtctggtttaaagataccctgattatagaccaggcatgtataacttatttatatatttctgttaattctttctgaaggcaatttctatgctggagagtcttagcttgcctactataaataacactgtggtatcacagaggattatgcaatattgaccagataaaaataccatgaagatgttgatattgtacaaaaagaactctaactctttatataggaagtcgttcaatgttgtcagttatgactgttttttaaaacaaagaactaactgaggtcaagggctaggagaatattcaggaatgagttcactagaaacatgatgccttccatagtctccaaataatcatattggaattagaaaggaagtagctggcagagctgtgcctgttgataaaatcaatccttaatcactttttcccccaacaggtgcagttttgccaaggagtgctaaagaacttagatgtcagtgcataaagacatactccaaacctttccaccccaaatttatcaaagaactgagagtgattgagagtggaccacactgcgccaacacagaaattatgtaagtactttaaaaaagattagatattttgttttagcaaacttaaaattaaggaaggtggaaatatttaggaaagttccaggtgttaggattacagtagtaaatgaaacaaaacaaaataaaaatatttgtctacatgacatttaaatatggtagcttccacaactactataaatgttattttggacttagactttatgcctgacttaaggaatcatgatttgaatgcaaaaactaaatattaatctgaaccatttctttcttatttcagtgtaaagctttctgatggaagagagctctgtctggaccccaaggaaaactgggtgcagagggttgtggagaagtttttgaagaggtaagttatatattttttaatttaaatttttcatttatcctgagacatataatccaaagtcagcctataaatttctttctgttgctaaaaatcgtcattaggtatctgcctttttggttaaaaaaaaaaggaatagcatcaatagtgagtttgttgtactcatgaccagaaagaccatacatagtttgcccaggaaattctgggtttaagcttgtgtcctatactcttagtaaagttctttgtcactcccagtagtgtcctattttagatgataatttctttgatctccctatttatagttgagaatatagagcatttctaacacatgaatgtcaaagactatattgacttttcaagaaccctactttccttcttattaaacatagctcatctttatatttttaattttattttagggctgagaattcataaaaaaattcattctctgtggtatccaagaatcagtgaagatgccagtgaaacttcaagcaaatctacttcaacacttcatgtattgtgtgggtctgttgtagggttgccagatgcaatacaagattcctggttaaatttgaatttcagtaaacaatgaatagtttttcattgtaccatgaaatatccagaacatacttatatgtaaagtattatttatttgaatctacaaaaaacaacaaataatttttaaatataaggattttcctagatattgcacgggagaatatacaaatagcaaaattgaggccaagggccaagagaatatccgaactttaatttcaggaattgaatgggtttgctagaatgtgatatttgaagcatcacataaaaatgatgggacaataaattttgccataaagtcaaatttagctggaaatcctggatttttttctgttaaatctggcaaccctagtctgctagccaggatccacaagtccttgttccactgtgccttggtttctcctttatttctaagtggaaaaagtattagccaccatcttacctcacagtgatgttgtgaggacatgtggaagcactttaagttttttcatcataacataaattattttcaagtgtaacttattaacctatttattatttatgtatttatttaagcatcaaatatttgtgcaagaatttggaaaaatagaagatgaatcattgattgaatagttataaagatgttatagtaaatttattttattttagatattaaatgatgttttattagataaatttcaatcagggtttttagattaaacaaacaaacaattgggtacccagttaaattttcatttcagataaacaacaaataattttttagtataagtacattattgtttatctgaaattttaattgaactaacaatcctagtttgatactcccagtcttgtcattgccagctgtgttggtagtgctgtgttgaattacggaataatgagttagaactattaaaacagccaaaactccacagtcaatattagtaatttcttgctggttgaaacttgtttattatgtacaaatagattcttataatattatttaaatgactgcatttttaaatacaaggctttatatttttaactttaagatgtttttatgtgctctccaaattttttttactgtttctgattgtatggaaatataaaagtaaatatgaaacatttaaaatataatttgttgtcaaagtaatcaagtgtttgtcttttttttagttttagcttattgggattctctttgtttatatttaaaattatactttgatttagaaaacataaatgcttccccttagcattttgtttatggaaaattacaaacttttatttttagaaaacagaactcctttccagaaataggttacaaacagtagtgtcctccacagaatgttggaaatgttttcaactccccactgtatactatcttgctaataagtctgtcttcagatttcgattaaccggtttgtatgtctgtgcgctttagcatagctggacatttaaagaggaaagagagtacatattataagttgcttatcagtaactgaggagtaaaactgataaatgtgaggcaaagaagtttaaaatatggttaaagcctaagcatatttgcaaacaaatcaaacaatactctgagaagtaaaaacataattatttaattaacaaatttcagtggataaattttataacaaattagacacagttgaaaataaaattagaaaactagaaaatagaaccaaagaacttctggaattcttataagagagataaagaggtagaaatttggagaaagaagtcgaaaggcgtagaggatacaggtgtaagctctaatagatgtctaattggattgtcacaagcagaagagagaggaaagggtcagagacaacatttgaagtgacaatggcaaaaatctttcggaaatgatgaaaaataacaatcgatgtttcaacaaatccaatgtatcaacatttacctttatattcctactcaaaaaacaaggattttggaacttttacgtttaaccaccttctagctcataggctattgttgctgggtattttaagttcacttctttttttttttcttttattttattattattatattttaagttttagggtacatgtgcacaatgtgcaggttagttacatatgtatacatgtgccatgctggtgtgctgcacccattaacttgtcatttagcattaggtatatctcctaatgctatccctcccccctccccccagcccacaacagtccccagagtgtgatgttccccttcctgtgtccatgtgttctccttgttcaattcccacctatgagtgagaacatgcggtgtttggttttttgtccttgcgatagtttactgagaatgatgatttccaatttcatccatgtccctacaaaggacatgaactcatcactttttatggctgcaaagtattccatggtgtatatgtgccacattttcttaatccagtctatcattgttggacatttgggttggttccaagtctttgctattgtgaatagtgctgcaataaacattcgtgtgcatgtgtctttatagcagcatgacttattgtcctttgggtatatacccagtaatcggatggctgggtcaaatggtatttccagttctagatccctgaggaatcgccacactgacttccacactggttgaactagtttacagtcccaccaacagtgtaaaagtgttcctatttctccacatcctctccagcacctgttgtttcctgactttttaatgattgccattctaactggtgtgagatggtatctcattgtggttttgatttgcatttctctgatggccagtgatggtgagcattgtttcatgtgttttttggttgcataaatgtcttcttttgagaagtgtctgttcatgtccttcatccactttttgatggggtttttgtttttttcttgtaaatttgtttgagttcagtgtagattctggatattagccctttgtcagatgagtaggttgcgaaaattttctcccattctgtagttgcctgttcactctgatggtagtttcttttgctgtgcagaaggtctttagtttaatgagatcccatttgtcaattttggcttctgttgccattgcttttggtgttttagacatgaagtccttgcccatgcctatgtcctgaatggtaatgcctcggttttcttc

SEQ ID NO: 118 - DNA, 13175 bp, Homo sapiens
tgtggaaatagaatttggaggaagacagacagaattacacatgattgactataataaatacaaacctatatgatacaacatgttattgctgtacataagataacgggaaaaatcagtactgaatgagaatgagaagacgaagatttcaatctagtttttgccacataccaaatgtagcactagatcaatgagggtctcaacaggcaacagggggttcaaataaaagattcaatgaaagaactactttgagaaatattaagaagggttaggggtggaggttcgtaatgcacccagacactagcaacagtggaagctgatagcacccctaggatggaaagattcttactctttttttaagattctttaagattctttaagaatctagggtaagattcttactcttaccctaggggaggtaagagtaagaatgggcatcatagaacaagggccaccaaacaagagctgtacttgtggagaggtactgttactaccagacacatggccctgaacctgggagagggcagggaagaaagacctggacttccaccttgtgctttctgatgtgctgggaacttccaatgctaagccctcagaagccaccctgacagggaactaatgagacattttctacaggggtctgtcccaggtggagcttggatctgaggaaaggggatggagaatcaccttcggcaagtcacttttcctctctaaatttcagtttgctcatccctcgtttattgcctaccatattatcaataatataaatagttggccttataatttttacttcgaatttttttgaaaaacctcaaatttacaaagaatttgtaagaatagtaccatgaatacacacatattcttcacctagattcaccaattgttaaaattccatcactgctgtttaattctttttatctttctctgctaacacagacacatatttttgctgaaccttctgagagtacttgctgacattctgacacttaaacctcgaatacctaagtattatctataaacaaggatggttctctacataaccccagtacgatgatcacattcaggaaattaaacattaaggcaatgctattatgaaatataggctatactcaaatttcccccagtttctcagtaatctgctttgtattatttttttcaatcaataataacacattaaattctgtcatcatgtctctattttctcctttaatctagaatggttttccaggcttttccctttttaatcttttgtgatgttaacatttttgaagaatctaggccagttattttgcagaatatggatttgtctgattatttcttcatgcttagattcagttaaaacaattttggcaataactacacaaagttgttgtgtccttctcagtgtattgtatctggaggtaccctatgtcagaacactccatcacagtaatcttcagttgggatcacttggttaaagcagtatctgctagatttatcaattatcttttccctattgtaattatcaagtaatctgtggggtggtatatgtgaatatatcttttctcaccaagattcttttacccagcattttttttacaaccatttgaattttcttgctatgacagaattagtttcataagagcataattaaggaatctggtaagacgtctttgaaagttctaattacaaccctcatctcttcttatgttaaactgtcttcattcatttacctacctatttaacaaatgtttaaagaatgctcgttttgtggcttggcctgtgacattttcatcagtggcttcatagttgcctaaactaatgtctcttcctgtgctccagaatcctattggcaactattttccatggatgccataaactcaacatactcagatgaaagctgtcatttcactttctcacctgctcttcctgttatttattccagtaaattgcacctctgtcttctcattgaccctataataagaaaggcctatttattgagtggctatctgttacaataggccttttatacatgttacatgcattatctcatttaatcttttcaataatcttgagaattagatgccattatctggaggacattgggctttgagccccaggtcaacagtaagcaagtccccatgctaggatacaaatccagttatcttgggcttcaaaatctttgctttagcatagtgataaaggtatcactggcttcaaatcctggctctgccacttcctacgggtgattgggtaagtcaccttaactttcaatgccttgatttccttctttataaaatgggaaaaatggtaactcttgtcttgtagggttgttatggacttgaaagaaacaaaacttgctgggcgcggtggctcacgcctgtaatcccagcgctttgggaggccgaggcgggtagatcacctgaggtcaggaatttgagactagcctggtcaacatggcgaaaccccatctctactaaaataatacaaaaaattagctgggcgtggtggtgggtgcctgcaatcccagctacttgggaggctgaggcaggagaatcacttgaacccaggaggcagaggttgcagtgagccgagatcgcgccactgcactccaacctgggaaacaagagtgaaactctgtctcaaaaaaataaaaaagaaaagaaagaaaacttgacatttgaataatttctgcagcaccacagtacttctcaaaccataattctggtgtcattcattctaaacttctccttccactcactgattgtggactagtcccatagggacctcgtcttcacagctgtcattaccttagtttagaccaggggttggcaaaccatggcccacaggccaaatccagacttcacctattttcgtaaatgaagttttattggaatgcacacacacccatttgtttacatataatatatggcttctttcattctaaaatgacagagttgagtagtggcaacagagaccccaccgtttgcaaatcataacatatttactattttgcccccttcagaaagctttccaaatcctggtgcctggcgtattccaatagtcttcttcccagtcttcctggttacatttctccctgaacccatcctccccatccctaaaatttcccccaaatgtgaactcagtcataccagtttgctccttatgtttcattggcccttgctgctaagagcatccgcttgcaccttctgctcatccccagacaagctttgtcctgtgaccataatgaactcttcatgccgtttccaactttagcccatgttattcttcttgtctgaatatccacccttttctctgttctcaataataagttcaggcttttcgtcttctgagaagccctttctgacttccacaggctgaaccactggcttctgctcctctacataatacttcaattccagcattgatctcactctatcatgatcatgggtttagctgtctgtccctgccactgctgtgtgttcctcttgagggcaggaacatttgtttttcactttttaaaaaacctctgttgcccagtctggcattaggaagtgcccattaggttgttattgcttgttggcgcttgagctggggcttgaaggtttctataatgtgtagcagtgtatagaaaacaggcaggtcagaaaaggcttctgtgcatcacaccaacatggcacatgtatacatatgtaacaaatctgcatgttgtgcacatgtaccctaaaacttaaagtataataataataaaattttaaaaaaaaaaagaagaggcttcctggaggagatgacagctgagctaagtcctggaggatgagaaggagtataaaataagataataggagaaaaaaggcagtaggaacagcatgggtaaaggtgatgaggcctgaaagaggcacgtggaaggaaagacaaatgcaggaagggggaatgggagggaatgctggggtacaggccaaagagggaggcatttggtgagtattctgcagagtctcctctgctgtgctgaggtgtggacaatgggaaaccatggacggactggagtaggcaaatgtcatattccctgttacaactgtctgtttgcatgtcagccttctagaagccccttaaggtatcaactatgtttttgttttgtcatcattcaatcctaagtgcacagaattccgggcatattacaggttccccatgaatgtttctttctttattaaaatgtatgaaaactctccagatttaaggaaggtcctcaatgtttcaaattctttttgttagatcattggtcctgtctacagctgtcacaaatttaaggactctggttatatttaatcttcacttttgaattttctgcttgaaaaatttgtattagaaaaaaaagtctatccttttatggacggctctaatctcttgaatcatttgggttggcttttctttggaccttcttcaactctgttttgtctctgttgagttaaggcttttaagaacacctgaattctttccttctgcaaaaccagaggcagcttcttttccgcctattttcagtttatttcttgtgattttagtttttttctcttaaccaaatgctaaatggatttaggagaaataaacttatttgtaaagctgtcaagggaccattagaaggatggtgcttcacagatagaatacagtttttattaatgatgcctagacaaatcctgccattagcccaagggctcagaaagttagcagcctagtagttttggagttgtcaatgaaatgaattggactggatggttaaggatgcccagaagattgaataaaattgggatttaggaggacccttgtactccaggaaattctccaagtctccacttagttatccagatcctcaaagtgaacatgaagcttcagtttcaaattgaatacattttccatccatggattggcttgttttgttcagttgagtgcttgaggttgtcttttcgacgtaacagctaaacccacggcttcctttctcgtaaaaccaaaacaaaaaggctttctattcaagtgccttctgtgtgtgcacatgtgtaatacatatctgggatcaaagctatctatataaagtccttgattctgtgtgggttcaaacacatttcaaagcttcaggatcctgaaaggttttgctctacttcctgaagacctgaacaccgctcccataaagccatggcttgccttggatttcagcggcacaaggctcagctgaacctggctaccaggacctggccctgcactctcctgttttttcttctcttcatccctgtcttctgcaaaggtgagtgagacttttggagcatgaagatggaggaggtgtttctcctacctgggtttcatttgtttcagcagtcaaaggcagtgatttatagcaaagccagaagttaaaggtaaaactccaatctggcttggctggctctgtattccagggccagcagggagcagttgggcggcagcaaataaggcaaagagatagctcagaacagagcgccaggtatttagtaggggcttcatgaatgcatgtgagttggtttagtagagagacacaggcaatttcagacccttctatgagactggaagtgatttaagagggaaaggatagccatagtcctgaatacatttgagctgggtttcaggatgagctcacaagttcctttaaaaaaaattgacttaagcaaatcctgggaagagtttttttgctatacaattcaaggttttaaggtcctcggattcatatactttataaatgaattagccagcttgtttaaaatgtagggaaattgtgggaagaatgccttctttacttaattcaaggttttaaggttctcttaatcaattctactagctaattagccaattatttaaaaataaaagtttgaaattgccaaaaaaaaaagacaaggaaaaggaaagaaagaaagccaccagtctgtttggcatacaatacttaattgttgcctgacctacgtgtgggtttcagatgcagatcctcagttttcagctcttcagagactgacaccaggtttgttacacggcttaaaatgatgagtatatccattgaatctcaaccttatctctctctagaccttcttggttaagaaaccatgtagtttgtatgaagtaggtactcaaaagatatttgatgatttaatttttactggagaagaaatattcatatatgttttcttatttttacatgttttaaatatgtaaagattaaataaacactcttagaagtatttaaatttcctaaagtaaatttatctcaaccagtaacaggaccctcccaatactggaaagttgagtgtgaccgcatttagtggtgatgagtgtgagcttgcttggggagagggcaggacatttaggatttcttaagcttagagtcaatacaataaagattattgagtgctcacttgggtgggctataatcactgctcacaggagttcatgaaccacaagtaaaagagtgaggagatatgattagctcacaaataactttaatacagagcagaaagtaatgaactactgcaatggagttatcacagtgctaaggatgctcagagggcatctctgataggcagaggtgagggttagggaaggaagctgtagtctagctagctagagctgctggaatagacatgacaatggctgctgccaaactgttttctcttctgaggacagatgtcccgtgcaagtggcttggtggaagggactagtgtctctaatatagggtgatttataagcaggaaagtgtgtcctagaaattcagaccagagtgatagattggaattggatcatgggggactcattgaatgttatttattgtatttgtttttgcgatcagtgttagtaaagtgtcaaagggattgagcagatgagtgacatcatgcaacacaagttttgagtttcacttgtcagactgactggagaggggcctggttagttacaggaaggtaatttggcatgcagccactatttttgagttgatgcaagcctctctgtatggagagctggtctcctttatcctgtgggaaaagagaacaaaggagcatgggagtgttcaagggaaggagaaataaagggcagagaggcagcggtggtgtcaggggaagcccacaggagttaacagcagggttgcctcaacctagagaggaagcgacctggtgccctcggctctgtggcttccttcatctaacaacatcttccactctacaacaatgccagggaaggcggaggctggtacagtgcatcaagacacagctactcctgggtgacagaggttcagggccagctcactaagtaggcagaagtttttgacatatactttgagagataaagcaagattctgtacctcaaccttcagaatttcccctaccactcattatagttccggagctatatagctcctatcattctatcataaccttagaataccagagaacatatcatctcatctaattatctcttactatatgtgaaaaaaatgaaggacatgggggaagtgtgacttgccccaaatcacatatttcatggtagagccaggtcttctgtttgtcatatcagtgttcttcctgccacaaccatcttgaagaatctatttctcagtaagaaaatatctttatggagagtagctggaaaacagttgagagatggaggggaggctgggggtgtggagaggggaaggggtaagtgatagattcgttgaaggggggagaaaaggccgtggggatgaagctagaaggcagaagggcttgcctgggcttggccatgaaggagcatgagttcactgagttccctttggcttttccatgctagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattggtgagcaaagccatttcactgagttgacacctgttgcattgcagtcttctatgcacaaaaacagttttgttccttaatttcaggaggtttacttttaggactgtggacattctctttaagagttctgtaccacatggtagccttgcttattgtgggtggcaaccttaatagcattctgactgtaaaataaaatgatttggggaagttggggctctcgctctggagtgctaaccatcatgacgtttgatctgtacttttgatatgatatgatgctcctggggaagtagtcccaaatagccaaacctattggtgggctacccatgcaatttaggggtggacctcaaggcctggaagctctaatgtccttttttcaccaatgttggggagtagagccctagagtttaaaactgtctcagggaggctctgctttgttttctgttgcagatccagaaccgtgcccagattctgacttcctcctctggatccttgcagcagttagttcggggttgtttttttatagctttctcctcacagctgtttctttgagcaaaatggtgagtgtggtgctgatggtgcaccatgtctgatggggatacctttagtggtatcaactggccaaaagatgatgttgagtttagtgttcttgagatgagatgaggcaataaatgaagaggaaggacagtggtaaagaacgcactagaaccgtaggcattggcatttgaggtttcagaatgactaatattttagatgaatttgtttgacattgaatgttcatgtgcttctgagcagggtttcaatttgagtaaccgttgcaataacatggggcagctgttttgctctttgtcttcatgacaactgtacttaagctaacagccctgaaacatgagattaggctgggcagaatgctgctagagaggaccacttggatggtctttattctccttctccatgtccctctccatcacctggaagtcacctctgggtgccactctggtgccttccttgtcgaagctgtagctgctcacatgacacctatccctgttatccagtttgcttgactgggacgttttgccttccccttcagccaggaagtgaaagtcccagtttttatttatcacaggtgttggtattggtggtagaagaggtagaattatggaatcaggcctcctgtcaggatttctttttgacagtccctctcagacacctctgcctaaggccagctttgccattacaaactctcccttctccctctctcccttcttctcttcctcttccttcttctcgctctttctctctctctctttctccctctctgtctcttatacacatacacaaagatatactctattccaacatcctctacccaacctgacagagatgtcctttgctgtaggttcagcagtggggatgagaaatacagctctcaaacaggataactaaagcttattatcttatcaagcttgttcccttgcagacaagattgatcaattatcataggctttctgggtgttctttctgaagctttctcaaagtctctttctcctatcttccattcaaggcaaatgattgccatttaacatcaaaatcacagttatttatctaaaataaattttaatagctgaatcaagaaaatctcctgaggtttataattctgtatgctgtgaacattcatttttaaccagctagggacccaatatgtgttgagttctattatggttagaagtggcttccgtattcctcagtagtaattactgtttctttttgtgtttgacagctaaagaaaagaagccctcttacaacaggggtctatgtgaaaatgcccccaacagagccagaatgtgaaaagcaatttcagccttattttattcccatcaattgagaaaccattatgaagaagagagtccatatttcaatttccaagagctgaggcaattctaacttttttgctatccagctatttttatttgtttgtgcatttggggggaattcatctctctttaatataaagttggatgcggaacccaaattacgtgtactacaatttaaagcaaaggagtagaaagacagagctgggatgtttctgtcacatcagctccactttcagtgaaagcatcacttgggattaatatggggatgcagcattatgatgtgggtcaaggaattaagttagggaatggcacagcccaaagaaggaaaaggcagggagcgagggagaagactatattgtacacaccttatatttacgtatgagacgtttatagccgaaatgatcttttcaagttaaattttatgccttttatttcttaaacaaatgtatgattacatcaaggcttcaaaaatactcacatggctatgttttagccagtgatgctaaaggttgtattgcatatatacatatatatatatatatatatatatatatatatatatatatatatatatatatatattttaatttgatagtattgtgcatagagccacgtatgtttttgtgtatttgttaatggtttgaatataaacactatatggcagtgtctttccaccttgggtcccagggaagttttgtggaggagctcaggacactaatacaccaggtagaacacaaggtcatttgctaactagcttggaaactggatgaggtcatagcagtgcttgattgcgtggaattgtgctgagttggtgttgacatgtgctttggggcttttacaccagttcctttcaatggtttgcaaggaagccacagctggtggtatctgagttgacttgacagaacactgtcttgaagacaatggcttactccaggagacccacaggtatgaccttctaggaagctccagttcgatgggcccaattcttacaaacatgtggttaatgccatggacagaagaaggcagcaggtggcagaatggggtgcatgaaggtttctgaaaattaacactgcttgtgtttttaactcaatattttccatgaaaatgcaacaacatgtataatatttttaattaaataaaaatctgtggtggtcgttttccggagttgtctttatcatccttgcatttgaatattgtgttcaaatttttgattgattcattcagtatctggtggagtctccaatattagaaatactggaaacaaactgaaaaaccacaaaaggacaaataatgcttcatgagtcagctttgcaccagccattacctgcaagtcattcttggaaggtatccatcctctttccttttgatttcttcaccactatttgggatataacgtgggttaacacagacatagcagtcctttataaatcaattggcatgctgtttaacacaggttcttcacctcccctttcttaccgcctgctttctcagctcaactatcacaggcattacagttgtcatggcaaccccaatgttggcaaccacgtcccttgcagccattttgatctgccttcctgaaatatagagcttttccctgtggcttccaaatgaactattttgcaaatgtggggaaaacacacacctgtggtcctatgttgctatcagctggcacacctaggcctggcacactaagccctctgtgattcttgcttaaccaatgtatagtctcagcacatttggtttccacttaaggtttcctatttttgacctccacatcctgttattgtcttttgcagtgattctcagtgtggtcagggaccacctgtaactgaatccactgggtcacttactaaaaacaccaattcacacttattcccagatgcgttgaatcagaatctttgagtggtggggccaggagtttgtatttccaaaaatcttcccagtgcttcttatacccatcaagttttgagaatcactgcttaggcaactctgtctgtgtctcttcctctgtctctcccccttgctcattctcttgcttgctcttgctctctttctctctttcttgccccagcaagtgctaacaaacacacaagacagcacatagtaagcatttcaaaaggggttgttaaaaaaaaacgaataggtctttcaaagtaaggtaatgattattacctagaatgagctccttttctgtggaagactgtgcttcttgctctctaggcctttggatggtagccatgaagaaaaacaccaatcaggagcctcagtggatagtatatcatttccactcctctaaacatctttagagagattactctttttcatagttgatgggcagatctgagtgacagagagtgccttacacaagtgatgccctccaggtacctttcagacacccttagataatggactactttatataagcttaatatttcctctgctattgcctaaaattctaccatttgttgttcactgagataagacttgccctcttcattcagtctcaatcttataaaaataaaataccctttagctcaaaaaagcttcccctatggcagaagaattgtacaagaaagactatgacccggggttccgctgaaaggggagaaagcaaatatgagggtaagggagtgcatctgggcaccaaatgaaagcctcttctaattcccatgtcatttatgagagaagattctatgtctgtagtactcagagaggtgaactgcccccaaatcagaggaccaactgtcacatgggattccaaccttcatcatcatctccatttctgggatcagggtcctggggcagctccaagcccaaataatccatgaacccaaaaggggcttggcctaccatgcagggggagcctgggcgttgcaaccctcctccttggaaccacttgtcacaggcaagactgtggatgacaagggagccggatagagtggccaggggctccagagtcagttcctcctccaacatggctcctagcacacgcctctgctcatgtctcttcttcagcatggtactcgcattcctgctacttcccatttcttcatttctcacctccaaatattataatccttcattgtcacacccatcaagactaatcccaaattttgcctccaccgtcagatttgctgacactttaagctcgtggatttc

SEQ ID NO: 119 - DNA, 10228 bp, Homo sapiens
aaaagaggttgtaaactcagagcttataggctctgtttagccaatagatgcagaaatgcattgtgtttagttttcataatgtttttatgtttgtttgaggcaaggtctcactcttttgctgaggctggagctcagtgacacagtcatggcccactgtagcctcaacgtcccaggttcgagcaatcctcccacctcagtctcccaagtaggtgcgactacaggtgtatgccaccacacctggttaatttttttatttttatttttatagcgatggggtctcactatgttgcccaggctggtctcaaactcctaaacgcaagtgatcctcctgcctcaccctctcaaagtgttgggattgcaggcatgagccacggagctgggccaattttttttatttctttaacaatagaaggtcactactgaactgaaggtgctacatgccaaaagttgtctgagctaagtatgagggaagtacacaccctgacctaagctgttggagatcccactcctttcagactcaaatggtctgtttcactcatctgtgaaatctgcctgctccctatgcgtaagtattttgagtttgagatcctttaaagctttgatctccattaaattgcagaatttgtagcaaaggaaacacattaaggatttgacggtcttattttcacttcactacaaatgaaggttctgacagctgtttttccttttttttttttttaattgagacagagtctctctctgctgcccaggcaggagtgcaatggcacgatctcagctcactgcaaccttcacctcccagggtcaagtgattctcctgcctcagcctcccgagtagctgggattacaggcatgtgccatcacgcctggctaatttttgtattttgagtagagatggggtttcaccatgttggccgggctggtcttgaactcctgacctcaggtgatcagctcgcctcagcctcccaaagtgctgggattataggtcggagccaccgcccccagccttgtttttctatgggaaaagatccatagcttgggggctccctgttctgaagaggatgcagacagttgctaccacgatagcaagaaagaagacaacctgagactgaaagcagaatatttagaatttcacagtggggctgccagttgggagtgcacctgccttgctcttcaggaactctgtcatggctcgtctttcttccagcctagctgcactgagcaataggtagcctgattccagagttgaatttgctgtagccaaaacaatttagttttaattggccaggccagttgacacaggccaccctgccctgaggattttggaccacctggctgaacttgtagactctctccatggcacttcaggcaataggttttgtccatactgtgcttgttccatgcaggaggagtgcctgaagtgcctgcagccaaaccaggccctggcgatgctccttttcccatgggggtgctcctggagcatcactggccgtcctgtcatttcctacggctgcgtcctgggccaaaatacaaagcagctttgaggtgtaacttattattttgccttatgcattaaaaaggccaaataacattaaaaatcagttatctaaagtattttgttttgttttgttttgttttttgagacagagtctcaccctgttgcccaggctggcgtgcaatggcgcgatctcggctcactgcaacctctgcctcctcggttcaagtgattctcctgcctcagcctcctaagcagcatctgcaccaccacacccgactaattttttgtatctttagtagagacggggtttcatcatgttggccaggctggtctcgaactcctgacctcatgatccacctgcctcagcctcccaaagtgctgagattataggcgtgagccactgcgcacagcctacagttttaacattttttttaacggttatgttagctagaatgtttgacaatgaggaggagaaaaatctgagtgctgaagggtccataaaggtctttctttttcttttttttctttttgagacagggtctcagtctgttgctcaagctggagtgcagttgtgccatcatggctcactacaaactccacctcccaggctcaagcaattctcccacttctgcctctggagtagctgggactacaggcacctgccaccacactctgctaattatattttttgtagagacaagagtttagccttgttgcccaggctggtctccaactcctgggtggcaagcaatcctcctgcctcagcaacccaaagtgctgaaattacagcagtgagccacaactcctggcccataaaggtctttctaaatcaaaaccagaccctcttgttgtcttctgcccagtctgctcatggttctaatttccaagtgtatattctttttcacccacgttgtaatgtttgttgacttaaaccatatagcgctatagatgatggggagaaaacctgttggatcacattattcaaaactgtttcccagagtcgcaggaaaacaataggtacctccttggaaatctccactgtagggacaagtcatcaaggctcagcacctcccaatttttgcaaaaggcactatgacaaatcaaggaacgcctgtgccttactcttaccactaggtgtcacacaaaacaatcaggaataaacaccaagatcctgaaggctaccagaaatataaaaccatagaaggtggtgcaaaaaaaccttattcctcagatatttttattttttataaccatttttatacctgaagccaaatatcttcttctgatgtatcctctataattgggacatttagagtttgatgtttctgtacttgtttaaaagaagtgactatccccagatgggggtcaggggacacccaccctttattttccctaacagttggagtagaaaatacttttgagtacattccaaattatagtgtagtcagtctgattttcacagcgtgttttcaaaagtgttaatactaagactgctttggccaaatgattaatatactgccaaggggttcctgggtaaatttctatgcatttcagtttctttttagggaagtaggaacaatatctatcttacaagattagttctgagaacaatgtggtacataaggcgctggaattctttcaaaaatttataccctgacatttccaagagccatggtcacccttacaaaggagagttaggcatgcatttgtagacaagcaatatgacttttaagctagaaaagaccttaagtaacattcaatctaatcttattataaaagttaagtataccacaaaaaagttaactgacctctccaactaatgtgcagtcaagtaaagatagaacctggaattgagatgtcccattgcagtcactgtttcaccttgcctcaatcattgtagatttgcatatcataaacctgcagagacaatggtgtctatccctgggaagttttcattgtaacatgtgatcattattaccctggagggtctatctgattttaatttttttcccttcatacttggtctctctctatcgagtcagatttctttcattctaatattaggggaggggtggagatacgaagagaggggaaggattggttagtacaaggccagtcaaggtaaagatgattgcaataatcaccagaaatctctgcagaactacagaggcctcctgggactaattctgaagagctctgaccgaagtttgctttattttgcctgtaccaattctaccaagtagatgaattcagtccatggcttgcaggcttggaggtggaggtggagtggagagcataaattatatttctgcaggtgaaaccagagcatagtgagccgaaacccctttctacccaggtggcgtgaatgggtccaactgctactgtcccccaggacaagagagcaaggaggggcgggcaggatgggactaccttgagcctcccgcaggatgcctggcaactactttcttagtggtccttaaggtccaatcgcggttaaggcactaactaaactcattcttcagcctccttctctaagttctcctcgaatttgggtgcccagaagtaggctcacgatcctttttctgcgggagcacaatgtaggtcagatagcataaacgaatagctacttatgaacacaatagctactctgctattgcaaggtatccacaaccaccactacaaacattaacaccttgctttacttgggaaacaaaaaaaatcatggtcctattcagcagtttccccatacagggagcgtttgcccctcccccagtcaacagggctgttcatccctaggaagtgatttgagagttctccaaggatttaggcttttcactcctccaaagctttcacaacttctacctggcgggggtgcgtgggggggtgggggcgaagctaaaagttcctttgctgcaaagagcctggtataggcagaaacaccggcgcggcctgcagccccctaaccacagtgcgtccttccccttagaagtctggggaaagcaaatccctacgcccagccatcatttccactcttgcgttttcaaaagatcaaaaaacggaaaggaccggcaggttggcaaaccccaaagagggaccgcccatcaggtcggcgtccttgggatctcagcagccgacgaccccaattcaaatcgatcgtgggaaaccccagggaaagaaggctcacttgcagagggacaggattacagggtgcaggctgcagggaagtaccggggggagggggcctggtcggaaggactttccagccactcggcgctcatcaaaaagttccctgtccgtgaccctagtggctcatcgcagggagtttctccgatgaaccccagctcagggtttaggcttctttttccccctagcagaggacgaagccagttctctttttctggtctgactggcttggaaattccccgagcctgaccccgccccagagaaatccccagccagcgtttatagggcgccgcggcggcgctgcagagcccacagcagtccgtgccgccgtcccgcccgccagcgccccagcgaggaagcagcgcgcagcccgcggcccagcgcacccgcagcagcgcccgcagctcgtccgcgccatgttccaggcggccgagcgcccccaggagtgggccatggagggcccccgcgacgggctgaagaaggagcggctactggacgaccgccacgacagcggcctggactccatgaaagacgaggagtacgagcagatggtcaaggagctgcaggagatccgcctcgagccgcaggaggtgccgcgcggctcggagccctggaagcagcagctcaccgaggacggggactcgtaagtggcggaggcccggggctgggggtcgtcgggagggcgggacgcgcgcggcctgggggtgggaggcgccgggcctggagcccgagccgggggaccagcgatgcagccgggcgggcgccgcggcaccccggggctcctgcaccgccccgaacgctgcagggctgcagggccgcgcggggcaccggcccaactcggaggggcggcggctgggccctggtggatggcagcgtcgccacacgccccctccccccagacccgtcctctaggccgggggcggggcggcagcggggacgcgaagtccccggttgcataaggcggggcgggagtttctggccgctggcgggcgccgagcagccgggaggggaagtacagggcgttccgagctggcgggagggcggggcggtctggccggcgcccgccggggccgcggggggaggggggcggaaagtccctgggcgcctgccaggaacactcagctcataataacctcgcggaaaacaccgcggcctcggcctccagaaaccccggccttgcgcaatcccccgcagcccgcgcctccctgggccccacgcggtgcactcaccacccctggggtttttccctctcttccccacaggttcctgcacttggccatcatccatgaagaaaaggcactgaccatggaagtgatccgccaggtgaagggagacctggccttcctcaacttccagaacaacctgcagcaggtgcgccgcttgcctggcccgggttctctctgaccctgggacgtagctgatgtagcagagtcaccccagatcctttctgaattcagggccactgagcactatttaccctcaccttttacttcacatcagcccacatcctagagagtgaaggaaattccactgatttggtgaggtctttatgacccacctggagcctctgctatttgccagccctccccacccccctgtctaggaggagcagcacccaaccaggagacacgggttgaggggaactcggggtgtgggtttggtccatggcttactttctctggtctctcttgcattcgtagactccactccacttggctgtgatcaccaaccagccagaaattgctgaggcacttctgggagctggctgtgatcctgagctccgagactttcgaggaaatacccccctacaccttgcctgtgagcagggctgcctggccagcgtgggagtcctgactcagtcctgcaccaccccgcacctccactccatcctgaaggctaccaactacaatggtatgtctgcctccctgccctgccccaccccctcggagggcaggtgacgtggagaaggggcaggtgggccaacttaaggagtccaggcaagagcttaaactcctaacatttggaaggttgagaaaatatgtgtgcaaagtgcctattggatgcctttataaagttctttcagaacccagactgtgggttcttaaaattcagaagataagactacttgttccataaaagaataggtgaaaggagtgagggttgaaacaggtggttatacttttttccttttgttcttccaggccacacgtgtctacacttagcctctatccatggctacctgggcatcgtggagcttttggtgtccttgggtgctgatgtcaatgctcaggttggtgcttcctgcccccgacgcactgagtcaggctcctcgtgcttatgttgtgagcagaaattccaaatgcagccataagcatctcaaattccttttggtttcaggagccctgtaatggccggactgcccttcacctcgcagtggacctgcaaaatcctgacctggtgtcactcctgttgaagtgtggggctgatgtcaacagagttacctaccagggctattctccctaccagctcacctggggccgcccaagcacccggatacagcagcagctgggccagctgacactagaaaaccttcagatgctgccagagagtgaggatgaggagagctatgacacagagtcagagttcacggagttcacagaggacgaggtgagtctgtgaactccttcggcgctctaactaatgaggtgccattcccttcaccctcccaggcccctagagctgctccttatcagaggggtatctacataatgagtctctcaaatttctgtgcataaccagtatcccaagaatgtacctgccccccctctttttttaaagagaagcattaaaggcaaactctctcagcctgtaaagttcattatatttgggctatggagaatggagtccaagagttatttccagtagtggcctccccatcccggtagcttggcagagctccgctccggaagcttaacgtgtcttttttccccttgttttcagctgccctatgatgactgtgtgtttggaggccagcgtctgacgttatgagcgcaaaggggctgaaagaacatggacttgtatatttgtacaaaaaaaaagttttatttttctaaaaaaagaaaaaagaagaaaaaatttaaagggtgtacttatatccacactgcacactgcctggcccaaaacgtcttattgtggtaggatcagccctcattttgttgcttttgtgaactttttgtaggggacgagaaagatcattgaaattctgagaaaacttcttttaaacctcacctttgtggggtttttggagaaggttatcaaaaatttcatggaaggaccacattttatatttattgtgcttcgagtgactgaccccagtggtatcctgtgacatgtaacagccaggagtgttaagcgttcagtgatgtggggtgaaaagttactacctgtcaaggtttgtgttaccctcctgtaaatggtgtacataatgtattgttggtaattattttggtacttttatgatgtatatttattaaacagatttttacaaatgagttatcctgatcattttcttcaggctgccgtgggcctccggttctgaagttccatcctccggcagaggtggaggtggagcagtgtgagggaggaggtaactcccagcagcttctcacacctgacacccagcctccagcaaagctttgcaggcaccctgggcccagtgtgtgggagggaggctgttgcaaggttataggaaattaccaatctcaggcttggggtggaagcatgctgtttggcctgtggttggagacctctccgttgtcattctgtcatcccgttgatttaaacttcctggaccctaattgccagttgaggaaattattttggccaagaagaatatgctgaattcatatagagctgggaaagatctcactaatacaggaagttcctaaggacataggctcagaaatgggactgcactgacatttgttagcagatgctcacctctgagcctcagttttctcatttattagctgggctgttgtgaaaattacataagacaatccacgtaaaatgcatagcagagcggcaggcacgtagtgctaaagaaaaacaagctgttacgcaacgttctttatgtcataccaggagaaacaagtccagagagaatgtcctgtctagggtcaccttgcaggagaaataggtggttaacatatttcatacgtgctcgttttggggtaaacatcttttaagagcattccatctcttaaatctatgtgttattggctgtagtagaaaagtaataaaggcttgaagggtcttatcagttcagtctactgattgtactggacgattctacaggagcaaatgtcacaagaaaccagtaatgtgaaacttggcttagatgctctatcttgaggttttcatccctaaaatggctctcttgcctaggtagagagaagaataaagatgggtatactgaaagcccagactttaccgctatacaatataaaaaatctgcatttgtactctgtaaacatttttattaaatttttgggggatagggtctcactctgttgcccaggctggggtgcaattgtgcaatcacagctcactgcagccccaaactcctgggctcaagcgatcctcccaactcagcctcctgagtagctgggactacaagcatgcaccaccatgcctgactaattttatttgtagagacagggtttctctatgttgcccaggctggtctggaactcctgggctcaagagatccacccactatggcctcccaaagcactgggattacaggtctgagccaccacacccagccaaaacatttctgaggaaaaagtaaagttattgcattctagaaaatgcagagtagtgtataaatgcaattcatgtctttgacttatacctaagaaaaaattattacattatccctttcctgatccacaaacgatgtcaaagtcaaaagaatatggtcaccttggaccaaaaaggagccagtatttagtggtcttccacatataaggagtgttactacagactggttaaagccaattctgccccaattctttctctttctttttttttttttttttttttttttgagatggagtcttgctctgtcgcccaggctggagtacagtggcatgatctcagctcactgcaacctccacctcccgggttctagcgattctctgtgcctcagcctcccgagtagctgggattacaggtgcctgccaccacgcctggctaatttttatatttttagtagagacagggtttcaccatgttgcccaggctggtcttgaactcctgacctcaggtgatctgcccacctcagcctcccaaagtgctggaatttcaggcgtgagtcaccgcgcccagctctgctccatttctgtagccttagtccatccggaacccagcactggcggtccctctaccagtcttattctctgtcaaagtgcccgggttcttccctgaaattccttgtccatccccaagcacctcaacacaggtgttaaatgacctaccacaggaatt

SEQ ID NO: 120 - DNA, 16026 bp, Homo sapiens
cctcagaaattgctgtgaatcaggcgctgcctccccaccgcggtgcccttggcccggcggctgcagcgtggtttccacccggccagccgggcgcccggcgcctcccactgcagaagcagtttcgggatggtcctgctgtttccagtgcttccccggaccgtccctgcgcggatggaaacttcccactggcccgagcgagcacggggattcccctcccctcgccactttccatcgctgctggctcatgttccagcacacggggcccatctcaccactgccgtgcgccgggcgccatgcgtcatgacgtgcaggctccttggaaactgtagggtttctcttctcagtccaggagcatgggagggcccccagcgaggggaaccttctccaggcccctgagagcaaggccccagtctcggaaagcagtcctagatcccctggcctcctgggcgctctggcccacagagggcttccggggcctttagtcatagtcaccaagaactggagtccacccagactcacttaaactgaagaatggctgactaaattgtggtgtggtacgtacatccacaccacagcctggatggctcggcacagagaggaacgggctgctgaaacacacgcgctggaggcatctccagggaatcccggaaggatgcgtactgcaccttcccacggctgtcgctggaatgaaacagtgatgggggtggagggcaggctcgtggccaccagcagggaggtgggtgtattgccaagggcacccggaggaccccgtggtgatggggacgttctgcagcttcgctgtcaggggatgcaggagccttcgcttgtgcccacattgtgcagcctttgataggcacacattagccagaaacggggactcaggatgggatcgaggtgtcacatcaaagtcattaacctggttgtgacgttgtcctgtggttttccaaactgttatcattcagagagactgagcagagtgtatgaggaaacatctgtgtaatttcttacaactgcaagtaaatctacaattatctcaattgtaaatgatacaatactcaaccaaaacatacaaccatcagccaggtgtggtggctcacgcctgtaatcccaactctttgggagtccaaggtgggagaattgctggaggcccggagtttgagaccagcctgggtaacataaagagacctcctctgcccccaccccaaattctacaaaaaaaaaaaatgttagatatgagctggccacggggtgcacgcctgtggtcccagctactcaggaggctgaggtcggaggatcgcttgagcccaggaggtcgaggctgcagtgagccaagatcacaccactgcactccagcctgggtgcagagcaagaccctgtctctaaaagaaaataaacagacaaaaaccacatacaactttgcttgttgtaaattatcttttaactgaatgccctggattgaatctggctgctgccatcccagggccagtgatttggatggggtatgaccctctgtgaggaaggagcaggcggtgggggaagggcctgggtgtccaggttccctgggaaggaaggctgagaaaaggagatgggggaggggtgcgcagggccggccagccaagggccccttagccccatctaccctgctccccggactccgcctgcctttcctcctcgtgacagaagacagtggaagcctactgggtggaaggcacgggcttaggatgtgtgtgggaggaaagtgtgtgtgctggggagcatgtatgtttgggagttgtgtgtgttggaaatcgtgtgttggggattgtgtgtatattgcagattttgtatgtgtgttggggattgtggtgtgtgggtgttgtagattgcgtgttggggattgtgttggggattgtgtatgtgttgggggttgtgtgtgtgttggggattgtgtgtgggggagattgtgtgtgtgtgctggggattgggtgtgttggggattgtgtgtgtgttgaggattgtgtgtgggggagattctgtgtgtgtgctggggattgggtgtgttggggattgtgtgtgtattgggaattgtgtgtgtgttgaggattgtgtgtgttggggatttgtgtgtgtgttggggattctgtgcatgttgggagttgtgtgtgagttggggacaatgtgtacagaggattgtgtgttggaaattttgtgtgtgcgttgggaattttgtgtatgtgttgtggattgcgtgtacgttggggattgtgcatgttgggaattttgtgtgtgtgttgagaattgtgtgtgagggaattgtgtgtgtttgagattgtgtgtgtattgggaattgtgtgtgtgttgaggattgtgtgtgttctgaggattgtatgtgttgggaattttgtgtgtgtgttgaggattgtgtgtgttggggattctacgtatgttgaaagttgtgtgtgtgttgggattgtgtgtgtgttgtggattgtgtgtgttgggaattgtgtgtgtgtgttgaggattgtgtgcagggggattgtgtgtgttggagattgtatgtgttgggaattttgtgtgtgtgttggggactgtgtatgttttggggattgtgtgtgttgggaattttgtgtgtgtgttgaggattgtgtgtggggggattgtgtgtgttggagattgtgtgtgtgttggggactgtgtgtgtgttggggactgtgtgtgttggggtgtggtgtgttggaaatcgtgtgttggggacaccgtatgtgtttgggggagggtgtcaataagtggtctggagtgtgatattggggtgcaggctccatgagtccccaccccacacctgagccctgggaccgcctatctgcttcggggtggggtcccaggaccctgtaggttcagcctactagtccaggcccaatgcccaatgcctgcatccctgcaggccctgtgctctccaggctcagacccctcgcagccctgcagaccctccctgggtccatgtgtctctttgcaggtgctccagcgagtagcaatgtggagagaccatcaggcagccctggcctcagtggccgcagtcccctggctccacgctgggcccaccccaccaggtctcctctcccatggcccaggggccttcagtgggactgagaggaggagggaaggagagtgggtgacagggaagaactgcagggagagaggagaggggtgggagaaggagaaggaaggaaggggtaggatggaagctgggtttctccctgtgcccgccccctactccaggacatgtgtccaagccctggcaggtggaattttgggggcagggccttggtggtgaggagaccttccaggggtctgatagcatctcccatctcagagcccacctcctgggcccagcctcccctccagcccacacagtggcattcccagtcctcagaggacagcttcgtcccacaaagctcagagccttgaggaaggcccactgctgccctggaacagagacagcattcaacagaggttggaacaaggctctacagggctgggggcagagggaggttctgtccagaatctgccttcaggacaagtacagccagcaggggcagcttagccacttatccactgcctgggcgaggcacagggctatggaggcacctaccaaccaacagttctccagccccagagccccagcccctgaggcacaagggtgggtgtgccaggagacagttgctgcgggccaccttagctgtctggcagcacagtgggtgctgccaggctccctgggggccccccgccaagcccacctggccagctgggccccccccacctccccaccaagccacccacacagcctcacatctctgagacccgggagtggccctttgttcataaacgagagcttcctcgccgtggccgcgcctcgcagacatcatctttgatgctctttttccactgtttcggtgctttaatgttttcccttcagagccgggccgagtgtctctcggagccaggcagccgcgccagctgtcaggcggtttctagcctcgcttcggttattttaagctgatgagcctgacgcatctcatcactaatatcagcagtttcatttctcctgttttccattcgctgtaataaaatgctcagcacagaatacaaggagataagcaagccatttcacaaacgccgggccgccagccaggcccaggcactggaccccctgaaccaccccaccctggcacgagtgggctggagggcagggccccggggaagaaggtcaaggctggaaggggaggtcagcctcacagccagcccctgccaccgccccagcccccccgtcaggctgttgcaggcatcacacggtggaaagatctggaactgtggccatggtgtgaggccatccacaaggtggaagctttgagggggagccgattagccatggacagttgtcattcagtagggtcacctgtgccccagcgaagggggatgggccgggaaggcagaggccaggcacctgcccccagcaggggcagaggctgtgggcagccgggaggctcccagaggctccgacagaatgggagtggggttgagcccacccctcactgcagcccaggaacctgagcccagagggggccacccaccttccccaggcagggaggcccggcccccagggagatgggggggatgggggaggagaagggcctgcccccacccggcagcctcaggaggggcagctcgggcgggatatggaaagaggccacagcagtgagcagagacacagaggaggaaggggccctgagctggggagacccccacggggtagggcgtgggggccacgggcccacctcctccccatctcctctgtctccctgtctctgtctctctctccctcccccaccctctccccagtcctaccccctcctcacccctcctcccccagcactgcctctgtcactctcgcccacgtggatgtggaggaagagggggcgggagcaaggggcgggcaccctcccttcaacctgacctgggacagtttcccttccgctcacctccgcctgagcagtggagaaggcggcactctggtggggctgctccaggcatgcagatcccacaggcgccctggccagtcgtctgggcggtgctacaactgggctggcggccaggatggttcttaggtaggtggggtcggcggtcaggtgtcccagagccaggggtctggagggaccttccaccctcagtccctggcaggtcggggggtgctgaggcgggcctggccctggcagcccaggggtcccggagcgaggggtctggagggacctttcactctcagtccctggcaggtcggggggtgctgtggcaggcccagccttggcccccagctctgccccttaccctgagctgtgtggctttgggcagctcgaactcctgggttcctctctgggccccaactcctcccctggcccaagtcccctctttgctcctgggcaggcaggacctctgtcccctctcagccggtccttggggctgcgtgtttctgtagaatgacgggtcaggctggccagaaccccaaaccttggccgtggggagtctgcgtggcggctctgccttgcccaggcatccttggtcctcactcgagttttcctaaggatgggatgagccccatgtgggactaaccttggctttacgacgtcaaagtttagatgagctggtgatatttttctcattatatccaaagtgtacctgttcgagtgaggacagttcttctgtctccaggatccctcctgggtggggattgtgcccgcctgggtctctgcccagattccagggctctccccgagccctgttcagaccatccgtgggggaggccttggcctcactctcccggatcgaggagagagggagcctcttcctgggctgcccgtgaccctgggccctctgtgtacactgtgaccacagcccgctcctggaccctctgtgcccggctggccctctgtgcccagccagcctgcacctggggatgccaaggcctggggagggtggtttcacccaggccaagcctaagacagtccctctgggccctgctgggtaccggggtgtgacaccactgggaggacaagatgaggggcacccctggggccgccctgacaccccctcgaggctcctgccccgggggtcctggtgccccttcactgtggcaggcgactgggggttccccacctcggcccctctcccggggcctgctccccggcacctgaggcagcatccttgtcagggccgtgccttcctgcctcagcgccacctcttaaggttggcccgtgggtcactcaggactcagaactggagattctgggcaaaaggcaaagagcaaagggccaaaaggcatcccagggagacgactgcgggggaaccagagggcagaggggcgctcgtcacaggggagggggagctgagcgaggcaggaggggagccgagcctctccccccgtgtcccggctcttcaggcacgccctcgggacgccaccctccccgacccaggcgggaaagataagagcaaggtgtccgcagcctgacactcgtgcctcaggtgcccgcgcttgtgccggacaagactctcacaggtggcatgcctcggtttccccactggtaacagcacagggcactcagcaaggcgcagtgggcatgactggggtcctgtgggtcctgacccagatgtggccaccccggccgcagtggtcttcattccaggatgcctcttttccctcctgatctattcactgcgttcgccattcggtcattcccggggccaccactcccacctcaggtgtgtgcttcccttgtgttttatgagatatccccaacccggctgcttattggccccgtccgagggcaggagcataaataagagcctctgctttggcgtgggaccactgtgagctccagtcagcgctgccactgctgagctctgggccttcgacaggacttggccccttactgacttctccgtgtgctttgggtcatgggtgaggacgcctcctggcaaggctgcgtcctgaggattaaatcgggtcatctgtgaaaactacccagcccagcccctgacacttttttgtttgtttcttttagtgacagggtcttgctctgtcacccaggctggagtgcagtggtgtgatctcggctcactcgacctccggggctcaagcaattctcccacctctgcctccagagtagctgggactataggcacgtgccaccctgccaggctaatttcttccattttttttagagacagggtctcgctatgttccccaggctgggctcaaatggtcctcccacctcagcctccccaagtactgggattacaggcataagccactgcatctggcctccatgacacatatttttaaagtctgatttttaaagtcaaacttttgaagtcagattttaaacggactattttgaaaaatatacaaaaacgtttaaaaacaatgaatatccctcacctagaatcaataactaagaatattgacacatttgctttggggactgggcggctggagctgccatgacaaagctccgccgaccgagtggcttttaaacagagcctgccctctcgccgactgagggctggacgtgcaggatggagctccgcagggtcggctcccctgtgctctgaggggctctgctcagcctctcccggctgtggcttaaaaacagagcctgtcctcccgccgtggggggctggacatgcaggaccgaggggccacagggtcggctccctgtgctccgagagggctctgctcagcttctcctggctggggggttttgtggccaccctctgtgttcctgggttcagaagcatcccccaggctctgccttcatctctgcacgggtgactctgtacaggaagccaggcctgctggtcaatggccacccagccctgtgccctcatcttacctagtcccagctgccgtcaccctattcctaataaggccgccttctgaggtcatggggttaggacttccacataggaatctgtggggacacggttcggcccacagcccttcccacctccacacacacacacgactgtgaggagttggaagacctcactcctcacccctgccaggtcctctagggacaagctcgctgtcctcatcccagcacagcccgtgggacggtttccttgtccctaatgggaccacggtcagagatgccgggtctggtctgggccagcaggttcctccgcccggggcaggcagccttcttctgtgcgcttctggaaagcaatgtcctgtaatgcggtctctctgcgggagcacccccaccgccacctcacaggcctgttccacagccccgggatgggctctgtctccctcctgaccctgcatagggcacagccctctctcatcaacccacgatcctacgtggatccgagagggagcacctggggaaacaatggaatcccatagaaacaccccaaatctaacttgatccaggaccagccagtggtcacttctgaatattcaccttcctagtagacactaccagccaagggaggccaggaagccttcctggaggaggtggcctgaggactggggtgaggcaggccctgcgtgggggtcgccacccagcacccccacactgggtgggagccagtctctgagactggctgggggaggtgggagagggggctgcttgaactgcagacaccgaggtctagcccccaccccacccagccagttggtggaggcaggggaggccgaggggcccagctggacctgctccccggggtggattccaaaataggggggttggggggggcggaacaggagcccagggtcctggcttgaggcccagtggctgagggctggtgcaagccagacaggaaaagggttgagcctgtcagcgccagcacagatcaagtcaggagcaggtccctccaccaatgtgtgcaaataaatagcagctaagtttccagttacaagaacaatgcacagatggtcccagggacattgcggtgtggacacacagcggccattgtcctgtcgccagcacctcgccctacagctggggggtcccttagcacttcctagccatgcagggtccctgctcacagtacccgtgatgacttctgttcctcacctgcctgtctgtcccgacagctgcatggcagccctggcctgggagatggagaccccgaggggctgcctgcggtggtggggcccctgggtccccactgcattcccagaaacccagagggcagggcatttcccctgctctgtgccgagtccacccagccccagcctaggcccagtaagggctgcagcccaccctgtcccaggctgcctcccaggagccctcttggccctgatgccagaagcccatcttcctccattcaggcaggtctctgagtgccctggcctggctgcctgctggccctgagagtcacactaccccacagccctccttggtcaaaatccactctggagtggctggaagattccccgggcccacgccgcacacgcctatgcagggagcttcccctggccggccggcagacaagggcggtctcagagagggggctcacctcagcagccccttgtgtagctggccctcgcccctgccacctctgggaacaccaccaggaagctgggggacaggcacgcaggtgaaggaggcgagcgcttgtcagccgggaggccatgggcacagagggaacagggacaccctgggtggcctcaaggtcacttcaaacccctcactcgtcccctgggagggtgcccagtgaggttggcactaggagttggtcctggtcacatgacagacccacccacctctggtgtccagccagcacgccgtgggccagcctggctgcagggacacgagggcagcagccccctcctcctctgagctggttgctccttgagtcatcaccaccgcctgccacggaggccgcctgtcccaggaagcagagggaccgcagctgtggcaaccagggcctggtctctgtgtcacctcgctggggggccgtgcccaggcctgagacggaactgagtgacagtgcactgggtctgacagtgtggggctggcgccatgtttggggaaccctgtggcatgggacctgtgggtgagccgggaaaatcaccccgttgcatggcatctcgggcctggatcttaagcgcctgtgttggtgcctccgcctggcggaagagccgcgacccccacgttgccatgcgggtatcccaagccctgaccctggcaggcatatgtttcaggaggtccttgtcttgggagcccagggtcgggggccccgtgtctgtccacatccgagtcaatggcccatctcgtctctgaagcatctttgctgtgagctctagtccccactgtcttgctggaaaatgtggaggccccactgcccactgcccagggcagcaatgcccataccacgtggtcccagctccgagcttgtcctgaaaagggggcaaagactggaccctgagcctgccaaggggccacactcctcccagggctggggtctccatgggcagccccccacccacccagaccagttacactcccctgtgccagagcagtgcagacaggaccaggccaggatgcccaagggtcaggggctggggatgggtagcccccaaacagccctttctgggggaactggcctcaacggggaagggggtgaaggctcttagtaggaaatcagggagacccaagtcagagccaggtgctgtgcagaagctgcagcctcacgtagaaggaagaggctctgcagtggaggccagtgcccatccccgggtggcagaggccccagcagagacttctcaatgacattccagctggggtggcccttccagagcccttgctgcccgagggatgtgagcaggtggccggggaggctttgtggggccacccagccccttcctcacctctctccatctctcagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacaggtgcggcctcggaggccccggggcaggggtgagctgagccggtcctggggtgggtgtcccctcctgcacaggatcaggagctccagggtcgtagggcagggaccccccagctccagtccagggctctgtcctgcacctggggaatggtgaccggcatctctgtcctctagctctggaagcaccccagcccctctagtctgccctcacccctgaccctgaccctccaccctgaccccgtcctaacccctgacctttgtgcccttccagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgaggtaacgtcatcccagcccctcggcctgccctgccctaaccctgctggcggccctcactcccgcctccccttcctccacccttccctcaccccaccccacctccccccatctccccgccaggctaagtccctgatgaaggcccctggactaagaccccccacctaggagcacggctcagggtcggcctggtgaccccaagtgtgtttctctgcagggacaataggagccaggcgcaccggccagcccctggtgagtctcactcttttcctgcatgatccactgtgccttccttcctgggtgggcagaggtggaaggacaggctgggaccacacggcctgcaggactcacattctattatagccaggaccccacctccccagcccccaggcagcaacctcaatccctaaagccatgatctggggccccagcccacctgcggtctccgggggtgcccggcccatgtgtgtgcctgcctgcggtctccaggggtgcctggcccacgcgtgtgcccgcctgcggtctctgggggtgcccggcccacatatgtgcctgcctgcggtctccaggtgtgcccggcccatgcgtgtgcccacctgcgagggcgtggggtgggcttggtcatttcttatcttacattggagacaggagagcttgaaaagtcacattttggaatcctaaatctgcaagaatgccagggacatttcagagggggacattgagccagagaggaggggtggtgtccccagatcacacagagggcagtggtgggacagctcagggtaagcagctcatagtggggggcccaggttcggtgccggtactgcagccaggctgtggagccgcgggcctccttcctgcggtgggccgtggggctgactccctctccctttctcctcaaagaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctgaccggcttccttggccaccagtgttctgcagaccctccaccatgagcccgggtcagcgcatttcctcaggagaagcaggcagggtgcaggccattgcaggccgtccaggggctgagctgcctgggggcgaccggggctccagcctgcacctgcaccaggcacagccccaccacaggactcatgtctcaatgcccacagtgagcccaggcagcaggtgtcaccgtcccctacagggagggccagatgcagtcactgcttcaggtcctgccagcacagagctgcctgcgtccagctccctgaatctctgctgctgctgctgctgctgctgctgctgcctgcggcccggggctgaaggcgccgtggccctgcctgacgccccggagcctcctgcctgaacttgggggctggttggagatggccttggagcagccaaggtgcccctggcagtggcatcccgaaacgccctggacgcagggcccaagactgggcacaggagtgggaggtacatggggctggggactccccaggagttatctgctccctgcaggcctagagaagtttcagggaaggtcagaagagctcctggctgtggtgggcagggcaggaaacccctccacctttacacatgcccaggcagcacctcaggccctttgtggggcagggaagctgaggcagtaagcgggcaggcagagctggaggcctttcaggcccagccagcactctggcctcctgccgccgcattccaccccagcccctcacaccactcgggagagggacatcctacggtcccaaggtcaggagggcagggctggggttgactcaggcccctcccagctgtggccacctgggtgttgggagggcagaagtgcaggcacctagggccccccatgtgcccaccctgggagctctccttggaacccattcctgaaattatttaaaggggttggccgggctcccaccagggcctgggtgggaaggtacaggcgttcccccggggcctagtacccccgccgtggcctatccactcctcacatccacacactgcacccccactcctggggcagggccaccagcatccaggcggccagcaggcacctgagtggctgggacaagggatcccccttccctgtggttctattatattataattataattaaatatgagagcatgctaaggagttctgtcctgtctggtggctgtgggggcagtgggcaggagggagggaggcctccagggagggacaaggcggcagccaggctgaggctgtgtgtggagggtgctggtggctcccctggatcctggcctgagtcaggccttcccctcgctgtcccagaaacacacaggccctcctcgccctgtttgccccactcacatgtagagccctccggtgaaaaattcctccaacatcacggggctggggacaccatcttccccaagcccaggccttttaggggggtcccacggccccctggccccaggcttcctggccaggcatgggtgtccctgagtgactgtgcctgctccatagcccccgtgtccccatgcctaccagtccctccgggcaggggtggtacctggtgccagggcgtccacagcggggtgggagggctgccctgggctatgagggacacagcatctgcgggtccctccctaagtacacagggtgggggcagcgggggaggcggggtcacgtctgctggagccctatcaccctgctgcggtccttggcagctagcagcagggacggtgcagggaaaggagccctgccaggccctcgggcaccgcagccaaacacccactgacaccccggggctcattctctgcttgccatcctggagcccagtcctggaccccatgcctgtgtcagggacaggcacagagacctcgcagctctgggaactcccggtgagatccggcagatcaggcctggcagggaacgctgcccgggggctctgcacatgatgtgggtttccatccgcaaaacccgataggtcccatggaaaatgaccacaggcaatgagaaagccatagagtgctgcctgtgagatcagcgtccagaactcacagctctcaaacacccactcacagcgcagcgcgccagcgtccagaattcacggctctcattcatccattgacagcgcagcgcgccagcatccagaactcaaggctctcaaacacccattcacagcgcagcgcgccagcatccagaactcacggctctcattcatccactgacagcgcagcgcgccagcatccagaactcaaggctctcaaacacccattcacagcacagcgcgccagcatccagaactcacggctctcattcatccattgacagcgcagcgcaccagcatccagaactcaaggctctcatacacccattcacagcgcagcgcgccagcatccagaactcaaggctttcattcatccattgacagcgcagcgcgccagcatccagaactcacgtctctcattcatccactgacagcgcagcgcgccagcatccagaactcaaggctctcaaacacccattcacagcgcagcgcgccagcatccagaactcaaggctctcattcatccattgacagcgcagcgcgccagcgtccagaactcacgtctctcattcatccactgacagcgcggcgcgccagcatccagaactcaaggctctcaaacacccattcacagcgcagcgcgccagcatccagaactcaaggctttcattcatccattgacagcgcagcgcgccagcatccagaactcaaggctctcaaacacccattcacagcgcagcgcgccagcatccagaactcaaggctctcaaacacccattcacagcgcagcgcgccagcatccagaactcaaggctctcattcatccattgtcagcgcagcgcgccagcatccagaactcaaggctctcaaacacccattcacagcgcagcgcgccagcatccagaactcaaggctctcatacacccattcacagcgcagcgcgccagcatccagaactcacgtctctcattcatccactgacagcgcagcgcgccagcatccagaactcaaggctctcattcatccattgacagcgcagcgcgccagcatccagaactcaaggctctcaaacacccattcacagcgcagcgcgccagcatccagaactcacggctctcattca

SEQ ID NO: 121 - DNA, 20013 bp, Homo sapiens
agttctgcgcagcttcccgaggctccgcaccagccgcgcttctgtccgcctgcaggtagggagcgttgttcctccgcgggtgcccacggcccagtatctctggctagctcgctgggcactttaggacggagggtctctacaccctttctttgggatggagagaggagaagggaaagggaacgcgatggtctagggggcagtagagccaattacctgttggggttaataagaacaggcaatgcatctggccttcctccaggcgcgattcagttttgctctaaaaataatttatacctctaaaaataaataagataggtagtataggataggtagtcattcttatgcgactgtgtgttcagaatatagctctgatgctaggctggaggtctggacacgggtccaagtccaccgccagctgcttgctagtaacatgacttgtgtaagttatcccagctgcagcatctaagtaagtctcttcctgcgctaagcaggtccaggatccctgaacggaatttatttgctctgtccattctgagaacccaaaggagtcctaaaagaggaatggaggagcctaagaataaaaatagtataataaaacatttcttagacacattgaccttggcctatgtcaaagttcagtctgggtttgtcttataacacaaggagtaaaagtaccattgttctacctctttttttaatacttgaaaaaaatttactgtggatgcttttctatgaattaaataaccttctaaaaaatgttttcattgctgcattcgattagattgggtaactaaatgaaattaattcctcactgttgggtataaaggttatttacagtggttctgtcttagccattcactgaactcattgcatatatatctctggaatattgctgattgtttccttcaagtaaacttagaagtgtaactacttagtcaaagagcctgaatattttaaaggccttttgaagaaaactgaaaatgctttccagaaaggatgtatcagttgacaatgacagtcgtcaacagtatttaaggagaactatgatactctgaagaaaaacttagcctttctcagtaaaagtaggtaggcagaggccacatgacagcagttagagtgtggtcttcaaggaagtcacagaaatactgtggggaattgaaaccccatgtggaaaatgtacaagagtgtctcagtgtgactgagaaggaggttgggcatggggtttcatggagtttaataaagtttggtcacttagtagaggtttaataaatcaactgtcttaatctttgatcctacttaagaattttttttttgtttttgtagagatggggctcttgttatgttgcccaggctgttctcgaactcctagcctcaggcgatcctccctcctcaggctccagaagtcctgggattactggcgggagccaccatgcaggcctcttgctcctacttttgagaaaggaagtttaaccggttttttttgtctttttttttttttttttgagacagagtctcactctgttgcccatgctggagtgcagtggtgcaatctcagctcactgcctcccgggttcaagtgattctcctgcctcagcctcccgagtagctgggactacaggcacctgccaccacgcccagctaatttttgtatttttagtagaaatggggtttcaccatattggccaggctgatctcgaactcctgacctcaggtgatccgcctgcctcggcctcccaaagtgctgggattacaggcatgagccactgctcctggctgcttaactttttctctatctcatcctcctacccatcctacccttggaagatagagaagtagtattagttccatagtgttatactgggcttcccccagggacaaacccacttccccaacctgaatgagccatcacttcttccccagtttacatttcattgctctttaaatgtctccattcggatatgggaattcacatatggtcataattcttacctgaagaagatgtcagtcttcttctcttagaccaactgccctgatatgaggtttagaggttaaagaacatgtgtgtatttacatgatctttgtattctgccttttcgtccctcactaatgacagctgcaccccaaggaaatggagctgtggaagagagggtttgataagaaattaagtaaatattggatctaatccatcaccctccaggaagcctttattactcctaaaaatttcaaccaaattcattaaaggacaagaactccaccagagtaggccataaacattggcaaaattagttgtaatccatgactagatttaatgtccctttgttttattcccatatggttataatgctttgcttggcattaggggtattttaagttttcttctgcctagtaagtgaatttgtgtttataatacaataatcataaaatatcacattaatattttataactgtacagttataaaatattttataagtaatatttatattttataagtaatattttataactgtacagttaactctggcccaaggaaaagatagtctgatagatgctgcagccccattttagcaaatgtgacctcacaggcctgaatgccatcgctattccacatctacaggatagacggaaaggaaagaaataaaaaaataggtacctaacactggcaagaggatgatgactcatgttatttcacttaacctttttatcttttaacatgaaggactcatacaggttgataagaaaccagtgacataaacagaccaaaaaatgatcagatctttcaaattagcaaaaaaataatattttttaaacaatgggtgaaaatacagtgtaacagtaccaattatcaacatgtgttgagaaccagaaaaatgttctttttctttgatcagcaacactatttgggaaaatctatcctcagggcctagcctggggccctggcacacagtaggcactcaacgaatatttgctgaacacacaaatacttatgatattttaaaaaattggcaacaatctgatacctaacaatagagggattaaatattatggaactgttaaataagatgcttatgaataccatgcagtaagatgggcaatatttatgccataagctttaatgaaacaaatgggtattaaatgtatgataaggttataaattactttttaaaagattacagggaaaaaaattgaaagatatacactgaaatgttttttgctcacagtggtgacaaggtttctcagcactggcactgttgacgttttaggctgtatgtctttgctgtgggaggctggcctgtgcactgcagggtgtttggcagcactcttggcctctgcccctagatagcaatagcagtcctccctcaaccagcccaattttgacaaccaaaaatgtttccaggcatcaccagatgctccctgggtgagagtgatgaaatagtaggggattttccccttcttttcttattttctgtaattccattatattactttaataataaagaaaaaaacataaaaaataaacgaatgttattattctacgtcagtttggatgtttggactccattttggggttctttccattatatcacttggtctgctaaacattctacggtttggtaaggtgaagtgattcatgaaattttggttttatttttttcctgatactaaaaataaaacattctttcacttggaaatttggacacagaacaccaaaaaaaatccataatctcatctctctttttctgtcttttccttccttttttccctttaaaaacaataaagagtgaaacctacctgttctccctctaatttaattcctaaatataatcactgtcaatatcttggacatttcctgtgtctaaacacacacacacacttttttttttcagcaaaagtggatttctgctacatgtagtgttctgcaacttactttctatgtgtttacaaaatcagtacatgtacatatgctgaattcagtccttaatggtattatattttgtgaatataccaaaatttgtttaaccacttagacaatctaggatattctcagtttgctgttatgagcaatgctcttcctttacatatacagacatatatatatatatgtgtgtgtgtgtgtttttgttttagtaggatagatttctaggagagggtgaaaggtcttatgacatccgcatttacgattgtaataggaagtatcaaagtgccccctaaagaaaaaaatcctcccattagtgggtaagaaagcctatttgttcatatcttcacaaacactaaatattagaaatatttacaattgtggtcaagctcataagtgaaaatggtatttcatatcttatattttttattgtgagattgaacatctttcatatgtttacatgtcacctgtatttcttattctctgaactatatgttatgacctttcactttttttcctcatgggttatgtgtagtttgtatagttgtcttattgattgttaggagctatttatatattaggaacattaatctcctgtcttatatatacgtggcatcgattagttgatcatttgtgagttcatgtctgtatacaaagattggagaggcactaagagggaaaacttacctctttcttatcaaagtttgtaaatatatgtataacagaagagggagaaaatattaataaatgcacagattggctgaaatagagtataaatcttttactcccctacttcaacataaactgcaaaaggagagtgacttttctttcactctgacttccgtattcctcatgcttaaaatagtgcctagcacagaagaggtgctcaatcagtgtttgctaaacgaaataattagtcacatttcaagcaggatgactaaatgaagaatagaatctaggcagatactctggaagagtggctgtgagtcattcatatcttagtatgaattagtcaaatccaactctctccccttcccactccccactgttagtagaagaatctgtttattgagagaatagatttataatttagaataagtgagaggggcagaagaggagattttgaaggatggcacctgaaggaggactagcatggctgagacagtgaagtggaagccttgaatagctaaagggtaagatgaaagtatttagctgtagggggaaaaagcattgacaggttggaaaagtaaaagtcagattctccttgctctgaaattttgtacagggcaggttctactaggtatgttacaatgcagaaaaaacatgaaataattgagaggaatttggtgcaatattatcttcttggcttcttttgagtgggcagatttttttcacggcctgtaactataataaatttgaaacttctcatcttttagtaacttttttcacttaagtttatgtggctgtgggcaatggaatgaagatattgaacttccaattccctgttgggtttccacaattacaagtcaatcatgactggttattagaagactatttcagttagaaccaccaagtcccatattgtcatattgtatgtttaattattaagtgaagcagtcttcttttcgtgttttccataattagggcattccagaaagatgaggatatttgctgtctttatattcatgacctactggcatttgctgaacggtaagacaccaaatccttccattaggttctatattttaaatattttaaccatgagtttaaaactaaaatgatcatttaaaatgcatgcaattttcttatagagagaacattctattctttcttctactttacacaatggcaaagtcttctttctactttacgcaatgataaagttacctgtgtcattttgtaaaaatatagagaatatagacaaattgaaagacacaaaataatctattacccatttcccagggttaactactgaaaatatctggggaaatggcctgtatgtatacatttatttgtttgctttcaacaaggccaagatcctttgatctttcagtcttggttgctctgtgacatgcctttcctgatgaggatactttaaggaagaattgtaagatacatggaaaatgtcaggctaacacagtactggcatcaccctgtgctctttcctgaactccataccaatgtacttcttgccagaaaactgatcaaaagtttagggaagtaaaaagagatgactgttagaatctaccattccctctatgtaggaagcaaataggtgtcctgtcaaaggacattctggggatgtctacatgaaaccaagtctccctggttgtaaggactccatctccatataatatttatacagtaatatatgtttataaattgtgggggcaacttgtttagctaattttattattctgctattgggacactgtgtctcagcatgagatatagtgtcccaaaacatatttcaagcccattggataaaatatgtgtttagcaagttcttaaatataatgataacataaccgaccagataaagtgatttataaacgctgtgccaattttgtaaatgtttcgaggaattttcccttttctgaagattgtccttctttctttttagcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatggtaagaattattatagatgagaggcctgatctttattgaaaacatattccaagtgttgaagacttttcattcttgtaagtccatacttattttcaaacagaacagcatagtctgttcattcattcattcaattcatgaattcattcacataattatccaatttcttgagcacctatttgatagtcactggaaatccagagacaaacaacacagagccatgttctacagtatgtacagttttccaaaaagaatttctagtctttacttttttattacaaatggaatacgtatacttgcaaataattcagatactgtggaagagatcaaatgaattgcaaaagtgtccctcctcccttcaccactatctcccatggcatgcagagagagtaaccattatttgtgtgtccctccagaaatttttttattcaactactatttttttattttattaggtccgtcagttttccttttttgagcctctctatatcaaatgcaaataaatatattcagaacaaaccccactgtaaggttcacattaaaaaagacttgaagtcaccctatgaagacaaaaaataatcacattaagtgtgaaagaacctattcttccagtacaggataagccatacttactgggcatatattcatcttgaaaatctatactgatgttgtcttggggaattgaaaaggaactaggagtgttagttcctcggtattgacccacagttatgttatcaggtcacttgagttcaaagttttgtgttggcactagctaagtaaaggaaaacacctctgctttcattgttgagtttcacagaattgagagctgaaaggatcccaggcaggagcagctaatccaaactcccacaaagaacaaaaatcccccagaggatcttctgttcttatatttcctgcaatggcgtccctgtcatatcccacaatggcctccctgccatttggatatcccttccatatcctgttgaaattactccctaatagtaagctgaaatctgcccctctagttgtagtcttgggattatttcatttacatgatgaccttttaatatttgactagaattaaatcatctccccttggtctttccattcctgggctaactaccatcaatctgagggctaacaatacaagtagaaaaagtatacatttgtcactgatcactgatcaattattaatcaatgatcactgataactataaactcaaaaacaaaatcatgtggggattaagagaaatgtatcagttttatgttgtatttctggtccctgatactggctcaggtaatgccactattgtcaagaagataccacttgtaaagtagatttaattttcattatattttaccatatgcttctccattcatgacatctcttgagatgttgtggtttatactttcagtttttctccagtccatccgcaaatatcaggcatctactgtgttccaagatattaaagaaatcatcatgacttagcctcatcaacagcattgctagatctgggatggaaaggaagagtataatcctggcagtcaggaagaaggcagcataaagtataagtttctgcttccaaaaaaggtctctcatcagcctgtagggagtgtgtagggaagggacagctgtccttgtagtagggaagggttttattcaggtcgtctgggctccataatatcccttgtgtatctgcagtctcctttgccatggatcaacacaataggaaatcttccggcactgatggtttttccaagggggagttcttcctggagcaaagcaaatgaccaaccaggtttgaggacctgatttgtttgacaattccattttgtattgtaaattacttaattggcattctactcccaatccatcttgtcatttgcatacagtggttttgggattgagttcagctataccaaaagtctgaaccttctgcacttagaacaaggcaaccaccaagcttcacttgcactgaggccgtgtctccaatggaaatgaggcagctggcttgcaggagcttcccaactcagggaagtagaactcctgagtcacctccatatgcaaatgatttcacagtaatgctgttgaacttcacttcccatcacagcaaatgtgtggtaacatagcttccccacaggagtttactcaccatggtattttaaaggtgaaacatttcaaaactgaaatttgaaagaatttagttttggattcactcaattatcactatcacttcgggtgttattgcacctttcttgtttgtgagtttaaatgccagactctcaggccactaactttcaattaaaagtgtttttctttaatcgctgaacctaacagcagggaaaacgaaatgttcattcagactttcagaaccttcaatgagattaggcagctgaaagatcaaagtgttgcatagttgtcccgataaagctatttggatcatatggaccaaatcgactgctgtcattccccaccaaccccatctctccccaaaattcccagccctgtttaagtgttctctgtagcatttatctctatctagtatattgtgtagcatatcatatcatacttttctgttttgtttattgtctctctcctcctagaatataaactccacaagcacaaagatttgggcctgttttataatattgttgcatccccagggcctgatatacagcagagtggtggtacgaaaagagcacacaaaaaaatatttgttgagtcaatgaatgaatgatttcctcaaataggattagcctaaaattttggaaacatgaacagatttggatatgtgaaaatttatttccagactgttcatcaggaactgttagcagcttctaaagggtacactggagcagcagtagtaaaaggaggaagaggagcagctctgctactgctactatcgagtactactacaattagcacttgcttattctgtgtgttaggccctgtactgaacactctgtctaaattagttcatttcctcctggaaatgactctagggggtaagtgcttcatcatgtaagatgagtatttttcacattttgttgtgtctgaaatctgagtgtgtctttcaatgatggaatctttgattccatgataagtggtattattcccattttaaggatgaggaaactgaggtccaaagaaattaagtaatttgcccaaattcacccagcctagaaaatgataaagctagttctaaacccaagcagattagctctgaagtctgggcccttaataaccactttttattgcctatatttgtacctctggtgtacgtatcaagttatatgttgacttcaaaactatcatgaccttttcttggttttgattgtccaacattagtatagtgttctgggtctgcaaaaattttgattactcatctcatctgtaaaacattttgaactcgtgtgtttgtgcatgcacatttgtgtgtaattataaaaattttactttctgttaatatataagttgtatcataagaaactgccgtttttgaagagcaaaaaaaggttgaatgttaccagttacatctggttcaacctaatagacatttgtacaaaaacagacattttaagaggttgaaataaaaatttaataaacaatattttcagtttttactaattgtgatgcttcactatcattagctaatatgtcaaggcataatataccttagggtgaactttatcattaacaaaggtggatggtgtcaataatcttgaggtttgtgtttttttatataacactgcgaggtctaattaagtacttactgtttaccacctcatacagtggccgataaaaagtgtcacttctgctgtttcctctgggttgtgcttgaattattagtattatcttcagtcctcagtttctttgtgggaaactttttaattagttgtttaattttgtaagatggttagtttagtcaaaattagataagagaatttgaaaatccgtagctaccccaaagcaacctacacataagaactattatttttgtgttttgaaatcataattttattgatttccagtgtttccactggtagtggtttcattgatataggagtatcaaaacatcactcattatttatttcagtttcatttgatcctagccgttttgtattaactctctgtgaagaaattacctcacaaatctattgctgtccttggtaaaggaatggagaattaaggctctagatcattagtggttacactatagtattagaagtaaaaaaaagattataccaacaaaataagaacatgttaatgtacttgtaatgaataaacatgaataaagctcttatgctatataggtgcactaaacaatctactagaattgtcagcaaactacgtatcttaatcctgaaagggtcccaaaccaatgatctaaaattgaatcaaactttcttccttgagcataattacttaaatgatttattaaaatagccagcatttaaaagcttaaaatgtaaatatcataatgtggtatcctagatagcatcccagaacagaaaaaggatattagggaaaaactggaggaatggaataaattatgcagtttagttattaataatgtactaacgtccttagttatgacgattgtaccatggtaatgtaagatactaacaatagaggaaaccgggtaaggagtatacagtaactctatactatctttgcaacttttttgtaaatttaaaacttctaaaataaagaacaaatttaaacattaaaaagtatcaccaggaacatatatcactgtttacagatgaaatactatgtattttcatatctaatttctgatcattgacttcaaatcagaaaagtgaatgacacctcaaaatcaggttttctgtttactgaagtctaagaaaagaaagcataccagctggagagattcatgtttataaagacagatttataacaacaaaaataaaatatccaagaataaatttaagaagaagcactttactgagaaacatatgaaaacctgaacaaatggagagggatattttgtatttgaatagaaagacttctggtttaaagataattctctttaaattattttttgtagaaatttaaggggtacaagagcagtgttgtcacatggatatattacatagtggtgaagtctggggttttagtgtaaattaatctttacattttgtttgagcccaataaatgtaccaacatgatttttatagaaagatagtcattcctattaatccaaacttgtcccaactttgaattgaattgaggcagagctagcaggtgttccccacggctgaggcatctgaacattaagcatatccctctgagaaccagcctgcattgatactctttctaatgtggacagcatcaagctatgtacgtagttctgtgctcagcaaaagccctgacttctttttgtttatgtcctagccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccaggtaatattctgaatgtgtccattaaaatatgtctaacactgtcccctagcacctagcatgatgtctgcctatcatagtcattcagtgattgttgaataaatgaatgaatgaataacactatgtttacaaaatatatcctaattcctcacctccattcatccaaaccatattgttacttaataaacattcagcagatatttatggaatataccttttgttccatgcattgtagtactcattggatacacatagaataataagactcagttcacactcttcaggaaacagataaaaaactaagaaacaaacaaaaaacaggcaatccaacaccatgtgggaaatgctttcatagccgggaaacctggggaatacctgagaggaatactcaattcaggccttgtttcaggaatccaaatcctggcacatcagagctgcttccctctttccagggtggcaggaaataaatggaacatatttttctatcttatgccaaacatgagggaccctttctccccggtgcctctcccaaggtagtctacaatatttcaactctagcagtctgcttagtgcatagaacatgaggctgtgtgtccctgggcaaattactagacttctgtgtgcttcactttccctgtaggattataatctactgagcaagcttattgtaagggtcagattagcaacagtgtatgaaaatgatttgagaccattgcctgcacaaattcaactatttttttttatctcactactctacagaagtaggtagggtgggagacagagtctgatgagaggctcagaatgtgaaagaaagtgaggcgagtgagcatgatatttaatataaacacaaagatattctgagaagagctgctcactgccccctcccccaatacatgttgataggaaaatgccacgtacttcagcaaaaacaactgaaaaattagatagaaaagtcaatcaataggaaaagataatccaggacggtgttgtgaacagaaagagggggaaaaaactttagaaaatgatggggatgctcttactggggtacgagtcctcaggtattgaactggctttcagtaaaagctagattagtgggttcctgccatttacaagctgttttatgacaacttacttgttgggtggcctacagtaactcacctaactgcactgagtctgtttcctcatctgtaaattggggatttttttttaaatacctggcatgcctaactcataaagttgttctgaaactgaaataaaacatacgtgaacaggcattgtaaactgtaagttacggaaaaagctggctgttgttgtgtctttaaagtttcacctgggtagtcaaagatggatcatgggtctcagtggagagctgagccaggcaggagctgactaagggtgagaggtgggagttagcagcctctgaacatctgtgtaccatgggaccccctttcctcctgcatggtaccccagacaaggagcctagtaagagatactaatggcttgttgtccagagatgttcaaactgcagagaaagataagacaacaagcattggcctccaatcatgatgacagataggaggaggtgggagctccttagcagtgctggttggccttccatgttctactgtgggccatctctgccatgtactgtaggctactagcttctatattaaagaatgcaagaggggccaggagcggaggctcatgcctgtaatctcagcactttgggaggccaaggtgggcagatcacttgaggtcaggagtttgtgaccagcctggccaacatggtgaaactctgcctttactaaaaatataaaaattagctgggtgtggtggtgtgcacctgtaatcccagctactcgggagactgaggcacaagaattgcttgaacctgggaggcggaagttgcagtgagcccagattgcgccactgcactccaccctgggcaacagagaaagactctgcctcaaaaaaaaaaaaaaaaagcaagaggaagtgaaataatcaaggccgccatttaatagtgagcagccactccatgtggtactgtgcaagcacattataaatattagcctcacaagaaatgtattagcatttgtattttgtacactggttaagtatcttgcccaagacctcaaaactggttaagggcagcagaatttagccccagcaccaccttttcaaagcctgggcttctcacacttctccatgctgttcccattttaacacaggtatctcgccattccagccactcaaactttggcatttaagaaaattatcctaaagctaaactaaacttcaaggatgaccattctcctgaccccttcccatcaaaattttatctttagtcagtttgttttcgttttgttttgtttttcagaactacctctggcacatcctccaaatgaaaggactcacttggtaattctgggagccatcttattatgccttggtgtagcactgacattcatcttccgtttaagaaaaggtagtatttccttaattgcagtggtctccactgggggtgaggaaggggtgagaattggatcatggctgcaaggaaacccgacttaacctctgcaaggtggtgcaaaggcattccactgttcaacagcaattatattgaagctgagtgggatcactgggtgaagatgaagcgtaaggggtgaggggcaggagaatgggtatggatggaggtagaagatgcagtgtcatacagtttttttctatcatgaaaataaccacagacttacagaagagaaagagctaaaatgcccgtcattttcagttgcattttagtcttgcattagttgcaaccagctggtttctgggtaccctaagtaataaaaatagttcctctgtagaactgtagtatgtttaccatagagtattttgcaaaatttttggtagaggatgttacataatttgcatgtgttcatttctccatttacctgtgggaacaattaaaatccaggaaaatgagtatattcaaataatttcctcccatttaagatgagtcagagtaaataattcctccaatacttagagaagtataccaagagatccagtgatggtatagagttgtctgatgttaaatagggaagtagaatatggaaggggattccaatagtcgttgaaaaattccccataaccccttacatgggggaaagtagtgttaactgagagagtagagataagctgtttccaaaaattatattcttaacaggactgagatagccagaatataaggatcaagtttcaatgacagtaagatcctgagatggagttgatttgcacaaagaaataattgttgccagcatgcattttgaatatttctctggaaaaaaagattagttggcagtagaaatggatagaaatcaatagatattaaaatacctcagaatttggttcatctctgggaaaagatgaaaaataaaagtgtatactcctcaagaacatctaggatcaaaagcatgtgccctacactattgaattaattaacctcataagttgggacctgtggaataaggatgtccaccagacttcctagggattacaaatgtttcacagaacttgaaatttaaacttgggtcactgtatgggatgtagagctgtgctatatggaaataaaaatgatttctttttctcaagggagaatgatggatgtgaaaaaatgtggcatccaagatacaaactcaaagaagcaaagtggtaagaatatcagaaggaattgggaagtaaaagtcaaaggaaacaaaaagctaaagcaataacaaagagaaatccatcagtcataatctcctctccttttaaagaatgctggttcccctttgcctcacagctaacacaagaactcctccaccgtctgaggaggtttaggagcagggaaggggaaggagtcagcttcatttgctaatcttctgttgccctgcaccctagcagctccttgcagcaggggacaaggatgacttaggtggatggataattaattgattctaaaatattgtgtgtcagtattgtaatactatgttaattgcaccatgcacggtatctcatttaatcccccaccccttgccattaccaaagagagagagagagagagagagagaaatactagaatttatcctcattttacagtagagaaaacagagggtcaagaagataatgtaaagtgcccaagaacacacagctgatcacaaaaatcaagcttgggggccattagcctaaccacagacccttactcttaacccatctgcttcaatccattttgctacaaatgtttacatttataagcagggcagaaaaacctcatccaggttattgaactaagaagaaagttatattaaggtttctaatttttttaatgtagttagaaaccaaacttaacaatgagcccaagtttaaagcagtctaattaacctggacaagctcaggcaagtttcattctgtggcccatagcatcatctgtgttgtaaagctaagtagcaaatgttgtttgggtcatgctgggggacaagccatcccaatttgctcaggactgaggggttttccaggatatcatgtaaggataattgggtacaaatataacctgctgctttctctcatttcaaatttatcatttatcatatcagcaactatgagttatgttttttattagatttcttgttactttttccccagaccacttcccatgaaattaatatactattatcactctccagatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatccca

SEQ ID NO: 122 - DNA, 22093 bp, Homo sapiens
gcctcctatcatatgtctctctactggtgtgaataccagaatacacagaactttagggtggacttttcagaacactgaaaacaaaacaaaataaacatgtggattccgtgtttctttttcttccttttcaagtatagtaatcacatgcctgacgtaactcctattctgggtgctctccatgaggcagcaggatacattggaatagcctgcattcaaatcctggctctgccctgagctaggtattaacctatgttagccactgtggttttctcatcttaaaacaagagtgccagtttacccactttccagggttcttaaaaaatgccatattccctaatctccaagtaaacacttccttctgtctggaataccctccctatccactcttcattagctacctcctattcctccctcaagtctcaacttagatgtcacctccttcaggaagttttccttatcttctatcccaagtgtggtttagtgcctctgtcttttgtccagtcagcctgtacttgccttcccagagcacttaccactgtgtattaaaatctctgttcactttctgcctctgcccagattgcttcttggggaaagggacttagttttgatcaccattttattcctagcatcattatagcatctggcccacaacgagtgtgcagtaaatattgtcatatgaacgaattaaaaattttgatcctccttcaaaatggtaacgcgtaagactcttatacgtcaacaaaaaataaaagtaaagacaaggtcaggaactggccaggtgcgttggctcacgcctgtaatcccagcactttgggaggctgaggcagatggatcacttgaagtcaggagtttgagaccagcctggccaacatggtgaaaccccgtatctacctaaaaatacaaaaatttcgccaggcgtggtggtgggtgcctgtaatcccagctacttggaaggctaagacatgagaatcacttgaacctgagagatggaggttgcagtgagccgagattgtgccgctgcactccagcctgggcaacagagagagacgctgtcttaaaaaaaaaaaaaaaaaaaaaaaattgccgggcgcggtggctcacccctgtaatcccagcactttgggaggccgatgcggccatatcacgaggtcaggagattgagaccatcctggctaacacggtaaaaccccgtctctactaaaaaaatacaaaaaattagccgggcgtggtggcggatgcctgtagtcccagctactcgggaggtgaggcagcagaatggcatgaacccggaaggcagagcttgcagtgagccaagatcgggccactgcactccagcctgggtgacagagcaagactctgtctcaaaaaaaaaaaaaaaaagttctggaagaaacatcacagtgtctttctcttttaaaaggtcggggtaactaagtagttgtttcagaaggcagctacaccccttgggtcatgacagggtactcctgagcaatttaccatcttaattcctcacttgagcaacagagagtataaaaaaaatcttcacacattaaaaaagcctggggcttgccctctccactcatctgggggtgtggcaacaagagctgtctcctgagggtggcccaggctgatgatgtgtcctggtaggcctgcagatatcactctcccccatccatgtagctgaggcacccctgccttcgctgactatcgtcaagagtgaggtctttctagtcccaatgtagactcttagatacaggatgggatctcccaactcccacccccagccaacctggcacactttcagtccaggccactgggcagagagatttccgctctgaaaatattgaaatagggtttatatctccacaaaccatggggataatgcctcactaactcctagaccacgcacagtgctggtgtcacacacctaagttaggggccctttctcaaaagctgcagtaaataaggccctccatgtgggcattgggcactgccagggaaacctcccaggagcaggtgatagttacaggaggcagtcaaatgcctaggcagatagggcgggtccctggtgaaaccccacctccaagttgaagacaacttaaaacctgaaagccaagctgcaagttaaatcttcagaccagattgagaacctgtcttcccatttggcacgctttcctctgattgatccccacccttcacctactttacatacacctaccctttcctaattggttttctacactgtcgtacccacctttgagtggtattttcactttaaccttttttgcatactcacaaaccaatcggcatgcactccctattctgagtccagaaaaagccctggatccagccacactgagaaaaaaaaacacccaactgtgggagcggggaaccacccccctccccatcccctctctgctgagagctggtccatcactcaataaaattattctctgcccatcctcacccttcaactgtcagcgaattctcattcttcttgaacacaggacaagagctcaggaacaaatgaaggtacaaactacaatacaggcagggcggggtatggccgggtgggtggggcagcaggcctggagtggggccttgctggccagaaatgtcctgagcccctcttctttggtttgtctgagaacaagggcagggcttgagggatgcacacacagtcccaaacacacatatgcacttggcaaacagacaacgttctccacaccacaatgaaatccaaatccacagactggccactgcaaatcatatatgactgcaagtgcacacacacacacacacacacacacacacacacacacacactttgaagcaggtgtcagcctccaagaaaaagtgatggacagtctgtctctcatatcctcacatatttggttcacctggagatggcctcagatgagccaagtgacacagaggtgcgagagactgaggatgtgctgaggtgttacccagcactccagtagtgctattacccaacacaaaaattggtctgaatatcagatttcaagcaagcaagggaaacccagaagagactgggagaagtccttatggaggctaaagcaactccatgttggatgctaatccacagttttggccgtgttggctttggagcctcccagaagacctctgagattcccaatttatcaattgctccttgtgtaagtgcaaacacttactgtaaatccggcccttaggtcaaacaaccttgatgttatcatacttcaactgtcctacacatcttttctgaatcacccttcccctgtggtctataagccctggtcctgggggtgaatcatggagacacaaacgtagcttctgtttataagtccttattaaatgtttttttcgaagaaactggatgtgtcagcctgtaacctgcaaaaggatctggctgctctctgcttgtagaaagaatggataataaaggaaaatctgctagctgccatgctagcaggggaagagcagcaaacaattccactcttcaattcatggggggaacgcaggggttttttaagaaaggatctgaaatgcagaaaaggcaagggggaggttaggaggtaccaggtggtgtgacttgctccagtggtcctctttaattatcgtcttatttggtggaggggctggtgcaatcgtggatcctgccaggttataaattagttactcttcagcagggtatatgttcaactcctgaagtaagcttttgttggagagagaattccggaggtgcctggtccctatcaggacgcagctcctgaggcatctaaggaaatatatgaccaggtaagagcatagcgtgtgcttaacaagcacgtaggtaaataaacgtgcgtaaggcatgggagtacagtatgggacaggaaagggagtggaggtcacagcacgtcctgaggctgtgttttaagatgagaggtaacatacatgcagtttgtctcaaaattatatcttgaggcccagaggaggaagagaaaaaaggaaagaagagaaactaagtttaaaacacggtttaaggctgggcgcggtggctcacccctgtaatcccagcactttgggaggctgaggcgggtggatcacctgaggtcaggagttcgagaccagcctggccaacatggtgaaaccccgtctctactaaaaatacaaaaattagcccaggcgtggtggcacatgcctgtaatgccagctactcggaaggctgaggcaggagaatcgcttgaacctgggagacgaaggttgcagtgatccgagatcacgccactgcattccagcttgggcgacagagcgagactgtaaaaaaaaaaaatagataaaaaaattttaaaaatcgcggtttgaggctcagctgctgagctgctggttaaaagcctctttcttgggtctctcagcttcctgggactttgggggcaggtctgcataaactgcttagttgtggacccccctccaacaattaggtaggggactactcagtattcatccatcggtgtcctcagacccccagacggggaggccctggggcaacgccaagactcacaccggcgtcctggcgcctttgccctcaagggcctgcaactacctcctccccattcggagcggccgcggccacatccttaacgccaagccggcacgccaggcaaaagcggctcggacgccctcaccccaccttccgcactcagggccgaggctgtgcctggccacctgcttcccagcacatgcacacacgcgcgcgcgcacacacccacacacacgtacccacggccggccgggctacccggaccactttgatccgaaggcactggaggcccagagggaaggaaagggaggggtggccggagcccgctagcgccccaggacagcctcgggttgccccaggcggtagccctgtgggacagtggttccccgtccgcggcctcgggggcggggcgtctgtaggcctcgccccgctgacgtcagccctgcggggcgctggcggagcgggcggcggagctgtcagtcccagcccaagggtagctggaggcgcgcaggccggctccgctccggccccggacgatgcggcgcgcccaggatgctgccgtgcctcgtagtgctgctggcggcgctcctcagcctccgtcttggctcagacgctcatggtaaggctccgggacgcggcccttccctgccctgccctctccgcgcccgctccattaaagttctccatccagtggagagccctggctggctggccctctggcagagcggtgcccgggtgctcccttactctggcaaacctggatctcaggctcacctgttgtggaagtggaagaggctgaaattgacaggaactgacggattgggaaggatagagaagtatgcgcaaggccaaacccccaacccgcaaacctcatcatccacccacttctagatgagccgtaagtttagctctaggttttttgctgtttaggttcaaatgatgggacttcttgtccccttggggaataattttgatctcctgactctcccttctcttagtcgactcttcaactaacctctgtctgtccttctcccatctcaccttcatccctcactgatcaccccgatctgatcccccttgctccacctgtagcccccaactcactcccactgccccctgcctctctcctcctaacccagccagttcaggcttctaacccagttcaggctcctaacccagacagttctccggcaaggtttctgcaactaagaaaagtaaaccccagagacagaatgaaagagccgggagttggaaggaccaagagcagagacagagagcaaggaccgaaactgcttcttcatgacaccaaaatacaaagtaaaacatgcagtcagccattcacgtgatcgagagctgagtcggagatgtgcagcccaaataccacaacgactcgctgcatttttcactttccgtctaatgtttcgaaatcaaggcactgacgtgaagatcaaagacggtataagaaagaaagcaagagacagatgacagagtcctcccccccaaaaaataaataaaaaacaggtgcatgattagagagatgtagagaagttagaacactagggagcgggagcagagtcgctgtcaaatggagagaacttagaggatcaaggaggagagaaagggaaccaaccagccgttggccttgcacatacttcagtctgcttgatcaacaagactcttaaagtagaaatgtttgttgcattcttacaaagaagacaatgccttcagcagtgtttcccatggtgaatggtgtgtactgggggtaaagggagagtagagctaggaattggtattgagtatctctcctcggagctacaggtgccagctcttgtggggatgtcctcagccatgtgtagagccagagctgatttattccatctgtccttgcccagccagaggttggacatctggctataggcaaagccctcccagaaactagggaatgaggcaagcacttagcttggggagcccaggagacctatgaagagaaggaggcagatgtcagcaagcaggctgtctgtgtacttagtgaggtggctttgagctggagcctgggccaagccagggtggggatcaggtgtgtggatggggccaagcctaggaatagtgctagagcagggacagggattgggcagagccagggactaacacatctgagagctcattgccaacttgcttagccctcggcccctgccttcttccctggtaggcatcagactcagcttggctcttctcagcccccactgcagcttctgatcacagagagcacagggcagtgatttagggagacacactagctctagaatcagacacatgtggattcatgtgcccactctgccccttacgggctccgctggcctcgggcgagtcatagcctctctgaacctccctttcttctttggtaaaattggggtcatcaatgcctgccccccctcccagctgtggtactgacactcttctccccagggacagagctgcccagccctccgtctgtgtggtttgaagcagaatttttccaccacatcctccactggacacccatcccaaatcagtctgaaagtacctgctatgaagtggcgctcctgaggtgaggaaaagggaagagggagggggagggaggagtgaatccccgccttgtcctctactctcctagcatgggaagatacctgccttgttaatgaagtgagtgcctgagggcccctaacacatagccagcagttgacaagtactgactgagactcccttgctgagcaatgtgccgggagctctgggtgaaaaggccagagcctttgctcactccagagtaagaaaagccttgtgccaaggcttttctcttttttgttttgttttgttttgtttcgttgagacagcttctcgctctgtcacccaggctggagtgcagtggcgcaatctcagctcactgcagcctctgcctcctgggttcaagcaattctcccatctcagcctcccaagtagctgggattacaggggtccaccaccatgcctggctaatgtttgcatttttagtagagacgggttttcaccatgttggccaggctggtctcgaactcctgacttcaggtgatccacccgcctcagcctcccaaagggctgggattactggcgtgagccaccacctccggcctccaaggcttttctatcttactggaaaaggagaagcatccacagtacagccagacaacaggggaacactaatgactgtaaaggcagtggggagaagggagagaccaggctgtgctgtaggagccagaaagaatttctggaagagctgggcctactcgtattcctatctaaaaatctgctagaccacatggaaattggtacggggtcccaagaccaagggagacccctcacaatgagctgccgtggactagaggatatagcctgagggctgtcccagtttctcccaatgtgggagctctcttcctggcctcttgcgtctcccttaaaggaggtaggattgagcacaagctcgtttccagtgcctaacctggtatctcctcaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaaggtgcttttcctcccttgacttagaacatggctctgaagtcccttccagccaggaactctagtctagagcttttctgtctattaccatagctcaccatgtctgccagcctccctggccggagaactagttgcccaaacagggcaggactttggagaatgttagataaaaatagcccaagttgtttgagatttaaaagggaactggagttgtttatcctacagaagagaggtcttggggtgggttgggacagctgagtcaccaggtccaggcctgtgaacttgttttttcagggagggttcagggagaggaagcgaatccatgaacactcagcatagggcagcaagcagtaggcagaatttgggtgaacgccccgcttctttaggggtgaacacccaggggtcatccctttgaaatgggaagtgctactaaagagaagttatggatccctctctggagaggtaagaggaaaggataagcccccaactcagtgggatttttcatcccaccagttgggattgagaaagttttgtgtgcatggagacctcgctctgtaggattcagagttagagccgatggtctaggaggtcattggcttccaaatgcctgcccacaggccactgctcagctggtgcagaaacgtttcccagcaagtgtgtttaaaatatctatttccagggcccagcctcagaaattcagaccaactgtgtcttgaatgggcccagggcatctgtattttttttttttttaacattttgttcttctttgtattttgatttgagcaaggcaattataaaaagacatttttgaggaaatcaccatatttgtatatgaaatggtttttagggcatattactaggtatttgtttgttacatgtgataacagcatggtagttatatttacaaaggtgtgatatgtgagagatacatacttacgtaattaggaattaagtaatacagtgcttgggatttttctttaaaatgctaaagtaaatttcatggggggatattgatgaccctcccctgccaccaattacttcaaaattggcctagtgtttgcaattactaaagctgggactttggaaatcattatattctcttctacttttctgtctattggaaatttctacataaaaatataatcaatgcaatttatcacattaaccatttaaaaaagaaaacaacatttaaaaagaaactaagcaaagttaaagtagaaagaaaatgtcctaacctgtagaaaggtatctatctacacatatctaccataaatattatgcttaacagtaaagtgttgggctgggctcggtggctcacggctgtaatcccagcactttgggaggccaaggtgggtggatcacctgaggtcaggagtttgagaccagcctggctgacaaggtgaaaccccatctcttctaaaaatacaaaaattagccgggcgtggtggcgtgcacctgtagtctcagctactcagggaggctgaggcaggagaatcacttgaactcaggaggcagaggttgcagtgagccaagatcaagccattgcactccagtctgggtgacagagcgagactctgtctcaaaaaaacaaacaaataaacaaaaaacagtaaagtgttgaaaacgttttataaaaaagtttttcttttaattgacaagtaataattctacatgtttatgaggtacagtgttatgtatacataatacatgtataccttgtggaatgatcaaatcaaggtaattagaatatctatcacctcaaatatttagcatttctttgtgccaaaaacatttaaaatattatctttcagctattttgaaatatacagtccattattattactacagtcaccttgccgtgcaataggcaccagaacttattcctcctgtctcactgtaacttggtacccctctccttgttccctatccacttctcccacctgcctccttccagcttctggtaaccacttctactctctacttctatgaattgaattctaaaagaattctacttagattctatacataagtgagatcttatggcatttatctctttgcacctggcttatttcgctcatgatgtcctctaggtccatctgtgttgtcccaaatgagagtttccttttttttcaatttgagacaaagtcttgctctgtcacccaggctgcagtgcaatgtcttgatctcatctcacttcaacctccgcctcccgagttcaagagattctcttgcttcagcctccttagtagctgggattacaggcacgtgccaccatgcccggctaatttttgtatttttaatagagatggggtttcaccatgttggccaggctggtctcggactcctgacctcaaatgatccacccgcctaagcctcccaaagtgctaggattacaggcgtgagccactgggccgggcctcagtttcctatttttcaaggctgaataggattctgttgtatatacataccacatttaaaaaacccattcatccattggtgggcatttccatatcttggctgttatgaataatactgcaatgaacgggggatacaggtatctcttcaatacactaatttcaatatctttcgatatatacccagtagtgggaattgctggatcacatggtagctctatttttagattttcgaggaacatccatactgttttccataatggctgagaaattcaccaccagcgacatgcaagggtttcccttttcaccacatccttgccaatgcttgttacttccatcttttggataatagccaatctaacaggtgtggagtggtatctcattgtggttttaatttgcatttctctgatgattagagatgttgaccattttttcatgtatctgttggtcatttgtatgtcttcttttaactgtctattcaagtcctttacccattttaaaaacaggtttatttgttttcttgttattaagtagtatgagttccttgtatattttggatatgaaccccttattcaatgaacatttgtaaatattttccacccattcatagattgtgtcttcactctagtggttgtttcttttgctgtgcagaagctctttagtgtgatgcaatcccatttgtctatttttgcttttgttgcctgtgctttagggatcgtatccaagaaatctttgcccagaccaaagtcttagaactttgccccaggttttcttctggtggtttttatagtttcaggtgttacatttgtctttaatccattttgtgttgattgttgcgtaagaggtgagacaaagatctattttcattgttctgtttgtgaatatccagttttcccaacaccatttgttgaagagactgttcttttcccattgtgtgttcttggcccctttgttgaaaatcaattgtgggtttatttctgggctgtccatcatattccattggttgatgcatctgattttagggtatatgtatttttaatgtgctccccaagaagtccttacattctgctgcattgacaaacctgtggccaagttttaggcctaggttctaattaagcttaattctggaggcaaagtctcggcggggacacccaggccctcctcagccctcaagtctcatggtattcccccccaccccaactccatttagtgactctgacagttggcagtgtgaacctagagatccacaatggcttcatcctcgggaagattcagctacccaggcccaagatggcccccgcaaatgacacatatgaaagcatcttcagtcacttccgagagtatgagattgccattcgcaaggtgccgggaaacttcacggtatggggttccccaaggccccagggccagaactcccttggcttccctgtcccctgggctggaagcacccttgtgtgccattgggaactttgcttatggactaaagggagggtctgggatggtgggtgggcagaggagggagtagaaaccacctcagccctcagagctatgctcttgtgagatgctcctcaaaggaattgcctactagggcaattgggcaccccagagcccttgtagatgaagcaataaggctctgagaggctgtgtaacctaccagagggcgcccctgttatgggagagctggcatctgcaaaatccaccctctctcctctccaccactgttgattccagccccttctgggaagatcttcagcatctataaaacaccgctttttcttttaaaatacaaaataaaattgtggtcattacagagatcatgtgctgcattggtaattgtggggtttgtcatcttagacaaaggatactgaagggggttggctgaaatcacctctaaaggcccaccagctctcagtgtccgtgtgccatgaaataaaaggattttgttaattgctatcatttttgtttcagttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcagtgtgagtcagctgggctgctctcagcatggggagggagactgggagggcctggtgcaatccaaaacgcgtgcacctgggatgctggtgccagccactgtgttaggtgctgcttcacatagctctgcatccttaataggagagaggtcctactgtcctcattttagagatgagggaactgaggctcagtattagcaacttcctagggttgcacagctaggacttggtggcagaaccaggtctcttggatctagggtcctcccactggagcacagcggtgtttcccaacaacatccccctcccccggggccatcttcagtgagctgtgctagttcctgttgttacagtcttctgctgttcttcctcagagccagggatacaaagggcttctccttacttcccctactctgctctgctcctcgccagagctgctaaagtcagcaaatactgctccaggctgtcagtcaggtgccggggctgtagggggagggagataatagaggacatggaggccacggtcttgcctaggaggatcttgcatcctggctggggagatagagtgtgcacagaggagactgtaacaagcaactcttattggacagcagatgagatgtccagtgcccaatgaaacatgccaacagtaagtgctctgggagggcccagtccagtgtgggcagagtctggcaaaggtgaaagtgactcctgctagatctgcaacaaatttcagtttgtgaaggacactcaacagctcagtgtcatttaagtctcaatgcagaggaaggcactaagtgggtatcaggcagtcttaagagtttttcatacattcattcactgatacatgaaatagttccaaatctcagctttacaacttttgaggtatatgactttgggcaaattacttaacttctatgtgcttcaggatcctaatatgtaaaaatggggctgatgatagtatttacctcctataagtataaaagttaaaagaatctgatggtattggtacatggtcagtgctgtgtacttcacaactaccttatgaggtcagtggctctgacagagtaattaatttgtccacaattgcacagctagtaagtggtgaagctgggacttgaacccagatctagcagcccccggagcctcgattcttacctgctgtaccatcctgcctcactgttctgtttgctgtgatgggagtcatgtgcagagagctggaagcagttagcgcatctcctgagggaatccttcagggcatggtttggaaggccaggtagggattcgcagaaacctagacacatctagagttgtggcctgtagtttgccttgggccactcactgaatggttcctgacagtgggcaatggatttcatgggaccagagtcctatgtccagaatcaccgtgccccatggtgacaggccacaaacacatctctctgggcctgcagatttcaccgtgaccaacgtcatcatcttctttgcctttgtcctgctgctctccggagccctcgcctactgcctggccctccagctgtatgtgcggcgccgaaagaagctacccagtgtcctggtgagtcttgcaaggaggtcactgccccgtccttcccagccacacccgagcttccaggagggcagggagctcttttcctcccagccagtccattgccttagcttgcctctgctctcagccctgatgtgattgggagataccttcatcaagcgctgtggggacagagggaggaagtgagcatgaagcccagtaccgagaatgttgccatccagtagctcagatgagacacatggaatcacaaatctcaatgaccacgagccattttgatatgatctactagtagttggccatggactagcccctgtgcattgcatctctgattatgaggcacagttattatccccatttcataagtgaggaaactgaggcacagagagattgttacacacagagggccacacagccaggaagagtcagggagggaacccactttcctactgctattagccatgggtgttccacaagagagaaagaaggaaaggccagaagtgctggtcctcgggctgctgccagagtgggccagaggagagaggacactggggggcggtggggatgctgcgccactggagaggggatgctggcaggggacagggaggggctcatggaggaggccaagccctcctcccctcccagggtgtgtcctcttgctacattgtactgacaaatgatacatttgtggcaagccagtctccaaaactcagcttgcaaagattgggcagcattttccaaacctcggcctggtctctggcctggagttcaggccgcattgaagattgcccaccatagcctccatagctggcactattgcatgatggctgagggatcagctgggaaggaaccagctccgtctgcaaggcaggggccccacaactggaaggggaatctccagagcgatcctgggatctctagggaaaacaagacgagagacaagtgcagtggcacaatctcagctcacttgcagcctcaccctctcaggttccagtgattctcctgccttagcctcccgagtagctgggattacaggcatctgccaccatgcctggctaatttttgtatttttagtagagacggggtgttgaccaggctggtctcgaactcctgacctccggtgatatgcccacctcggcctcccaaagtgctaggattataggcatgagccaccgcaccctgccccttctgtacttttggatcacagaactgacttgtccttgagggttttttctcttcaggataaatatagctcaatcccttcatctgatgatcgtagtcattaccacatgaataaattataatgcacagagcactgacacattcattagtttgttctcattcattagcttgtttgatttgatctttcaacagttcatcaaggtaagtattattggacaaggaagggatttcaaatcaggacagttgaggatgaattccagatgtgttctttactgttgtgccactttgtgcaagtcatttaacttctctgagcttcagttttctcctctaggaaatagaggtaataatacttctcttacagaattattggagggtaaagttcaattcaacatatatttattgagcaccttctatgtaccacactttgttctaagcactgtagacacattacaacataaggaggcaggtactgtcattatttccatgttacagatgaggaactaaagcacagagagggtaagtaatttggtcagggtgacaaagctgataagagtcagagctgggatttgaacccaggaagtctggctctagaggacacatcctaaatcaccaagcaatgtgataggaatagggactcaaccaataagagctgcatttgaatcatcatgtaccaaggaaatgagggcagatgtgcaacagtgagtcagtgagtcagtcggcagaccttaggagggcctactgtgtgccagctcagtttagactctggagacagggtagtaaacaacactagacagttcctgccctcaaggagtttgtgttcttttgggaatgtggagtaagtgagtgtgtgtatggtgagagacgaaaacaaacaagtaaaataattctaggaacaaagtgctaggaagaagtaacagggttaatgtggagagagtggcaggagtactgctttaggcaaggtattcaggaaggctttgctgaagaagtgacatctgagctgagacctaagccatgtacaggacacagccatataaagtgccgagagcatttcaagcaggagggagagtgtgtgcaaaggccctgaggcagcacggtgcttgatgtgttctgggacagaaagaacaccagtgtggctagggcagaggggttatgtggcttccttgaaacccttggcaattaacagagagcagccccttcactgtgagttccacacttgtgccactgccccacgctgcagccaccatgttcgcgtgagtcactactcggggaatagaaaacttaatttggtgtcttacatccagaattttagaaaatgaagtaggcaggagggcatagactgggatacaatagaataagaactgcagaaatatcgtggcacagctcataatgatgagtattattttgtgaaactggtttcagttatttacatacgggttatatatacaagtcattatgcaacttatttctgattttgagtcatagtcaaaaaagtttgaaacatgcttctagatgggtctagcctgctttcaggtttgaaaagcactgtggatggagccacctgagagttttcaccatgctcagcagaccttggttctgcctgttactccatctgggcctgagaatacaagtttcttgtgtatctggcctgagccaaactgggaattgcattaggaggaaagctggattcgaaaacaaaacagaatacattccagtgtaatttagactgtagtctcctccatcgagctctcctcctgggcctggccttccccggcagcactgggatgggtgctggaaggtgactcctgcttctctcactctgccctctcttccccagctcttcaagaagcccagccccttcatcttcatcagccagcgtccctccccagagacccaagacaccatccacccgcttgatgaggaggcctttttgaaggtgtccccagagctgaagaacttggacctgcacggcagcacagacagtggctttggcagcaccaagccatccctgcagactgaagagccccagttcctcctccctgaccctcacccccaggctgacagaacgctgggaaacagggagccccctgtgctgggggacagctgcagtagtggcagcagcaatagcacagacagcgggatctgcctgcaggagcccagcctgagccccagcacagggcccacctgggagcaacaggtggggagcaacagcaggggccaggatgacagtggcattgacttagttcaaaactctgagggccgggctggggacacacagggtggctcggccttgggccaccacagtcccccggagcctgaggtgcctggggaagaagacccagctgctgtggcattccagggttacctgaggcagaccagatgtgctgaagagaaggcaaccaagacaggctgcctggaggaagaatcgcccttgacagatggccttggccccaaattcgggagatgcctggttgatgaggcaggcttgcatccaccagccctggccaagggctatttgaaacaggatcctctagaaatgactctggcttcctcaggggccccaacgggacagtggaaccagcccactgaggaatggtcactcctggccttgagcagctgcagtgacctgggaatatctgactggagctttgcccatgaccttgcccctctaggctgtgtggcagccccaggtggtctcctgggcagctttaactcagacctggtcaccctgcccctcatctctagcctgcagtcaagtgagtgactcgggctgagaggctgcttttgattttagccatgcctgctcctctgcctggaccaggaggagggcccctggggcagaagttaggcacgaggcagtctgggcacttttctgcaagtccactggggctggccccagccaggccctgcagggctggtcagggtgtctggggcaggaggaggccaactcactgaactagtgcagggtatgtgggtggcactgacctgttctgttgactggggccctgcagactctggcagagctgagaagggcagggaccttctccctcctaggaactctttcctgtatcataaaggattatttgctcaggggaaccatggggctttctggagttgtggtgaggccaccaggctgaagtcagctcagacccagacctccctgcttaggccactcgagcatcagagcttccagcaggaggaagggctgtaggaatggaagcttcagggccttgctgctggggtcatttttaggggaaaaaggaggatatgatggtcacatggggaacctcccctcatcgggcctctggggcaggaagcttgtcactggaagatcttaaggtatatattttctggacactcaaacacatcataatggattcactgaggggagacaaagggagccgagaccctggatggggcttccagctcagaacccatccctctggtgggtacctctggcacccatctgcaaatatctccctctctccaacaaatggagtagcatccccctggggcacttgctgaggccaagccactcacatcctcactttgctgccccaccatcttgctgacaacttccagagaagccatggttttttgtattggtcataactcagccctttgggcggcctctgggcttgggcaccagctcatgccagccccagagggtcagggttggaggcctgtgcttgtgtttgctgctaatgtccagctacagacccagaggataagccactgggcactgggctggggtccctgccttgttggtgttcagctgtgtgattttggactagccacttgtcagagggcctcaatctcccatctgtgaaataaggactccacctttaggggaccctccatgtttgctgggtattagccaagctggtcctgggagaatgcagatactgtccgtggactaccaagctggcttgtttcttatgccagaggctaacagatccaatgggagtccatggtgtcatgccaagacagtatcagacacagccccagaagggggcattatgggccctgcctccccataggccatttggactctgccttcaaacaaaggcagttcagtccacaggcatggaagctgtgaggggacaggcctgtgcgtgccatccagagtcatctcagccctgcctttctctggagcattctgaaaacagatattctggcccagggaatccagccatgacccccacccctctgccaaagtactcttaggtgccagtctggtaactgaactccctctggaggcaggcttgagggaggattcctcagggttcccttgaaagctttatttatttattttgttcatttatttattggagaggcagcattgcacagtgaaagaattctggatatctcaggagccccgaaattctagctctgactttgctgtttccagtggtatgaccttggagaagtcacttatcctcttggagcctcagtttcctcatctgcagaataatgactgacttgtctaattcgtagggatgtgaggttctgctgaggaaatgggtatgaatgtgccttgaacacaaagctctgtcaataagtgatacatgttttttattccaataaattgtcaagaccacaggaatcgtgtgaaactagtcagattgctgatggcacattggctcatggaggactcggttgttgataatgaggagggtaataatgtgtgggatgtgtgatgtcactttaaggaaacagggctgaggactcaggcttaaatctgacagacccagggagtcctcatgctacctgtattgtgaccttgggcccattacttatcctctccaagttttaatttccccacctgtcacgtggagttgacaactccccatgaggatgttgtgaggtgtaaatgaaattgcacataaaaagtttaacatggtgccaggcatgtggtactatgtgccactagctgtcattggctgttgagtctgtaaagaaaaatggacagcttttcttggaaacaagtgaaaatgaaagcgataaggagtgtgcagcgggactctttcagaagacagaaaaggaagaggtggaaagcacaatgcaacgttttggatgtgagttctgacaatgtggagagaagaacctggaaacccctcctttccaggaagacagcaaactgggcctccctggagctctgttcccagagccctgtccctggctccccttgcccaggaggcatgtcccagtggtggttctcctaagtgcggaagccatctgcactctattctagaagcagcctgctccctccatgaaaaaccccatgtgggtttgaatttctccccatgctcagcccagtgggtagaacaaacccaggcccgcggcctgagcttcctgggtgtgcttttctcttcgtcctacctgaatcccctagaaggtgactggggacacctcacctcttttcccctgtgtcatctccaatcttgcctcacccagttcattctgagaaaataactagttctgaggaagtgctgatcctatcatcttcagagaacttgactaatatcacgccatttcagacctgctcaggcttttgccctagagccctccttgtcaatattggaaagaaaagtagtgttggtcactttctcctggaaatgaggtgttagcgatggcttattccccatggaaaaggcccctaaggtctaaatataaagattcagagagtaaaaggaattacattcaaggaactgcagccagtttggttggatacgacagatcaaagagcagcttgcaggacagaaggaagtcagaagacacagcccttggtgcaggccctgcctgctccgtttctgtggaatgagtaggagtttgcgctggttcatctctgctttgaggctgctctgtaaccactcaccacagacaggcccttgggagaaactgagcgagtccttccaagcagccggggctgatgagacacttgcagatttcttgggaatttggattacagcccccaacagaaggccccagctgttcttcaactgagcaatctccttcaagggcttttccaagctgaggaatgcgtaatgctgtctttgtgtgagctaatggaacttcctcttagtacagagagggggtaaatgggccaccattgagtgaagaggaaattcaataaactgtgttttcccaactttgtaaatagctatagtcagggtgtgccttgtctacccagcatatattcatcccccggcctcagtgttcacactttcacagaagacacacagtccctaggaagctgggtcccacccccagagcccatgctgctctgctgggtggaaggtctctgagccccacatgggaacagcacagcaagggaagctgtaggctgtgaccacctgggaccactgacaggtctgctgcctaagtggcaggtctgggcagtcagggctgatttgatcaagcctcaacctagaggaccctagatactaggaagcttgactcattcatttcctgttgtgctcatgggttaaaaaagcaaacccacagttttgggtttgaagtaaaccccaatttaaaattaaatgcattgttattaatgacacacagcacattttcccactatcatctcgatcctaacttcactta

SEQ ID NO: 123 - DNA, 37849 bp, Homo sapiens
atcacataatttataatgatcaggtagaatttcattattattaacataccatttttcatttaaccatttctcagctaaagggcatttggatttcttttccttttcttttctttggtgatgatgaatagtacaataaacatttttatatggatcgtctttttgttttggattatgtcagtggaacatatacacaaagtggatttatcctataaaaagatttaaaaaggactaggcacagtggctcacgcctgtaatctcagcactttgggaggctgaggcgggtggatcatttgagcccaggagtttgagaccagcctgggcaatataaggaaaccttgtcttagagaaaataaaaataaattagccaggcatgctggcacatgcctgtggtctcagctactcaggaggatgtggtgagaggattgcttgagcccaggaggtggaggctgcagtgagctgtgattgcgccactgcactccagcctcggttacagagtgagaccctagctctaaaaatatataaatatcttaaaaatataaagaagcagccaaaggagaggaagttaagaggagtaagtcagaacatagtctatgtggccagcagcctgcctggtttgaaaggcagctgctcccactcactagtcacaaaccttgagcaagtttcttaacttctgcacctcattttctcatctgtaaagtgggcataagaatagtaagcctggccaggcgcggtggccacgcctataaacccagcacttcgggaggccaaggcaggtagatcacgaggtcgggagttcaagaccagcctggccaacatggtgaaaccccttctctactaaaaatacaaaaatttgccgggcatagtggcgggcgcctgtaatcccagctactcaggaggctgaggcagagaattgcttgaacctgggaggtggaggttgcagtgagccaagaccgcactactgcactccagcctgggcaacagagcgagactcccgtctcaaaaaaaaaaaaaaaaaaaaatagtaagcatacctcatagagcagttgcaagggaaaggtaaattatatataaagcaccaagagtcatgcccagcatatagtaagcatggtgtaactgttagctgttattattgctgtcatcatcatcaactgcatcatcattcaatctcattaagtttattttttatttttttagaggcaaggtctcgctaagggctggaatgcagtggctattcacaggtgcagtcataatgcactacagtctgaaactcctgagctcaaacagtcgtcctgcctaagcttccccagtagctgggattacaagcgtgcatccctgtgccccagtgattaagttttattatgtagaaaataaagagcaaacagtacagctgatacggactctctctctctttttttttttttttaagaattttcataactttttagcctggccatttcctaacctgccaccgttggaagccatggatatggtggaggtcatttacatcaacagaaagaagaaagtgtgggattataattatgatgatgaaagtgatagcgatactgaggcagcgcccaggacaagtggcggtggctataccatgcatggactgactgtcaggcctctgggtcaggcctctgccacctctacagaatcccagttgatagacccggagtccgaggaggagcctgacctgcctgaggttgatgtggagctccccacgatgccaaaggacagccctcagcagttggaactcttgagtgggccctgtgagaggagaaagagtccactccaggacccttttcccgaagaggactacagctccacggaggggtctgggggcagaattaccttcaatgtggacttaaactctgtgtttttgagagttcttgatgacgaggacagtgacgacttagaagcccctctgatgctatcgtctcatctggaagagatggttgacccagaggatcctgataatgtgcaatcaaaccatttgctggccagcggggaagggacacagccaacctttcccagcccctcttcagagggcctgtggtccgaagatgctccatctgatcaaagtgacacttctgagtcagatgttgaccttggggatggttatataatgagatgactccaaaactattgaatgaacttggacagacaagcacctacagggttctttgtctctgcatcctaacttgctgccttatcgtctgcaagtgttctccaagggaaggaggaggaaactgtggtgttcctttcttccaggtgacatcacctatgcacattcccagtatggggaccatagtatcattcagtgcattgtttacatattcaaagtggtgcactttgaaggaagcacatgtgcacctttcctttacactaatgcacttaggatgtttctgcatcatgtctaccagggagcagggttccccacagtttcagaggtggtccaggaccctatgatatttctcttctttcgttcttttttttttttttttttgagacagagtctcgttctgtcgcccaagctggagcgcaatggtgtgatcttggctcactgcaacatccgcctcccaggttcaagtgattctcctgcctcagcctccctcgcaagtagctgggattacaggcgcctgccaccatgcctagcaaatttttgtatttttagtagagacaggattttaccatgttggccaggctggtctcaaactcctgacctcaagtgatctgccctcctcagcctcgtaaagtgctgggattacaggggtgagccgctgtgcctggctggccctgtgatatttctgtgaaataaattgggccagggtgggagcagggaaagaaaaggaaaatagtagcaagagctgcaaagcaggcaggaagggaggaggagagccaggtgagcagtggagagaaggggggccctgcacaaggaaacagggaagagccatcgaagtttcagtcggtgagccttgggcacctcacccatgtcacatcctgtctcctgcaattggaattccaccttgtccagccctccccagttaaagtggggaagacagactttaggatcacgtgtgtgactaatacagaaaggaaacatggcgtcggggagagggataaaacctgaatgccatattttaagttaaaaaaaaaaaaagcaaacacaaagatgcttcaagatcttcaggagaagtatggtatacaagtttcagggaccctatttgacaattttcagagtgctctctatgctgattccgagtcgagtgtgtcagctgtgattacagtgcctgtggatctaggccgggttgggggggtgtgggcgggggaagggaagtctggcccggagcaattgctcctgccggtaaccccagcactttgggatgcctaaacaggcgtatcgcttgaggccagtaattcgagaccagcctgggcaacatggcaaatctgtctctacaaaacaaaattagaaaaattaactgggcgtagtggcatgtgcctgttgtcccagctacttgggaggctgaggtgggagaatggcttgagcccaggaagcggaggttgcagtgagccaagatcatgtcactgcacttcagcctgggtgacagaaccagaccctgtctttaaaaagggagttggtggggagaggttctagaatgtcatgtagcaaccagtttaaggactgggactcagggatccaactcccacagtttccctgtgtgaccctaggcatttgacttagcctttctgagcctcaggttttttgtttctgaagtaaaaggattggactaggtaatctccaagatcctaggaacccaggagaaagatgagaaaatgtacaagaatgaacactcaggtggaaatgctgcaatcctgagaagctcccaggatgaatgaaaggcacaggacctcttacccctcacccctgcccccctcaagagctggtttctcaaacctttctcttaggccccttcaaagggggaaaactaaaaattatacaagttatagttcaaggactttaaatagattatttatatgattgctcataaggatgaggctgtgaggagggaacaccttatttaatctaatgaaattccataggaaagaggccttttgtatattgaatcaatttatctgccttctcagtgcatctgtcatattctgaaagattctgggttgatcttttgcgataacctctatggctgtgagtgtgtgtgtgtgtttgtgtattttttaacattttgtataatgattggaggttggtaaaaagtaacacaacagtacttttttaatacaaacttggtgtggtcttgagttgtcttccattggagagcgtacgtgtgccacagattggtgcacctgccggcatctctaggattgcaccaactccctctagctcttcttggtggcctgcaggggcctagggcgcattcacttccctctgcttgactttacgtggtgttctagcgtcagacatcaggcgatgccgtgcattacccaagacactggacagctccggatcttcccgtctaaccactaatcaagttaaaaagaggaaattaacgctttcttagtagtctctgaaagaaactagttatttgcgtaaagatgacttggttttgtgttgtttatccctggggagaagggtaaagaagaccctcaaactggatttgctgctgaagtgcttttaactcacctgtgcggagagcggacttcaagagagggcgtgccggtgctccggacaaaacctggggaatactgaaagccaccaccaccaccgtcattccagcatgcagatctcagtcctgggttggtgttgtagggcatttatttatttgacttgacggcgcctttcaaagcttgcgaggatcccttcctcttcctcgtgtgcatctgccaagcctgtgccccgcgcctcgcgggtggacccgggctggtgggcggggcctgcagcggggtgggagcccggtcgcccggcccctccccaccccgccccgcccatctccgctggttcccggaagccgccgcggacaagctctcccgggcgcgggcgggggtcgtgtgcttggaggaagccgcggaacccccagcgtccgtccatggcgtggagccttgggagctggctgggtggctgcctgctggtgtcaggtgaggggtccgcggggagggggcgcgcttgggaaccgggaggccccgcgagatgccgcccctgatcccatccctgggcgccctgcaagtgacttaagagccttcggggcctcgggagagaagatgcaaacgccacggccggctgctgagacgcaactaggctgctgctgccgtcgattctgtaacaggagaaagccccatccgcgcccggattacagaccgtctgggccctaaactcagggggagactcccggcgtcttttgattccctgtcctcctgcagctctctgctggcgggcgtctaaataaagtagcttccccaaaagacccaagatcccaccgtggctgtcatctgacccttctggagactatcagttcaagtggaggcccccaccagtccccgagtgccctgacagccctccctgaccccaagtctgccccctcagtgccagccctccctacagatgtgcctcccatcacccgctgcacctcccctccttccggttcaggcccgagcaccctgaaatctggattctgtcccccagcgccccagctccatggcttctaaacctaaatccattgaaaccttttctgtcctctaaattgactgcctaggttccaataacaccatggtcacctccttcctttctcatgaactcttccctggccttccgggatacccctccggtttcccgccctgtcaggctgtctctgcttgggaccctcctgctctgtcaggtttctatgtagctaattgtgatgcccaaggccttttctctccttccctggcactgttcctttgggggatttcagccaaaccctggcattaaacacgggctataggcccatgactcgcacatttatatctctcttggtaccagttgggtgatgggtggtggtttattatagtcttctacttttctacttgcatgtaggtttcaaattttcttttctttctttcttcttccgtttttttttttgtttgtttgtttttttgtttttttgtttttttttgagacagggttttgctctgttgctgtggcccaggctggagtgcagtggcttgatcttgcctcactgccacctccgcctcccgggttcaagcgattttcctccctcagcctcccgagtagctggaattacaggtgtgtgccactacgcctggctaatttttgtatttttattggagatggggtttcgccatgttggccaggctggtctcgaactcctgacctcaggtgattcaccttcctcagcctcccaaagagctgggattacaggcgcgagccaccatgcctggccaggtttcgaattttctattaaaaggtttgaaaataatttctgtctctagcccacatctcttcttggtgtgacctctgggctcattcatctggctgtctccctgacattcctacttcagcctctctccagcatctccaacctaatgcagctcaaatgggactcgtgagttccccagctgagctccaatcggggcaccagctgcttaagcccaaaatggacattgacctcagcttttatgcatcaaatgtatcaggaagtctccagtttgtttttacgtctggaaatatatctgaaatccatgtgcccaccctaccccctcatagctttctgccaccagacaaatccaaggctcctttgtctgtccccattttactcctgcccctccagaaatttctcctcacggctgttcaaagaaaatctagactcctcagcacagccaacctgtctctccctccctcacccacgtggcctttgaagacatggagccatagaggagaaccaagtgctggatgtgggctttttcatgggcatctgttttgaggagaaaagttgtaaatgtttttgtcttattttcatagcattgggaatggtaccacctcccgaaaatgtcagaatgaattctgttaatttcaagaacattctacagtgggagtcacctgcttttgccaaagggaacctgactttcacagctcagtacctaaggtgggtctggcctcactattggcaggaacgcaccggaggagccagccctgggctggtcactgggttgggccacaggaggaagggagtctgactacggtcaccgtgtgactgtgacaaccctcagggagcctccctgccagccttttgctgtctgggaaattctcgtggccaccttttctcttttaggttttctagttgtgatggttgacgatttgactgttaatcacagggctgctggcagatacagacattttatcatgcgtatgtagatataatcagccatagtggtaacctcatgctgtgcccatcccagcttgtttgtcacagttgatatattttaaaagaaaaataccactgctgtcatctctaggtatgagtagtattgctggaagtcagcaatgttgagtggatgctacccaagaaactgagatatgcaaagcggtcaggaagccccttgctaccatatatcacctgttcccctccaatggattataacatcatgccccagtgccatgcgcgatgcacataatttgcataatgagtgctcagtttctccgtggttccatcctgaaattaaaagtaagacagctactgactgttaaaaaccccattctcggacaacagcaggggctgtgaaggaagagtgagtgtcaccctagacggtcatctcagtggtgatctgaacgccctctacctctcctgcctgtcagtgtttccctacgcctggtgaaggaagtcagtgccagcactcccttggcagcctgccaccatacgccacatgctggactgcaggtgacacacctttaggccaggcgcaaacagcagcaggaggcccaaggctgacaagccaacacacctgctttctgtgtgactcagaaacatacgaagatagacaagtccttcagtgaggcaggaggaaatttcttcccagatccagctgtcatttcacaaaaatccccaaggcctgctacatcagagaagaagtagaaaggtgaataagatagtcctcgtctgtgcattgctggcttttgttttttaacttttagttcaaaataattccagactaacagaacatttcaaaattttaagtagaatacaaagaatttccaaatagcctacaatcagattcaccaatttttttttttttttttttttttatgagacggagtctcgctctgtcacccaggctggagtgcagtggcgcgatctcggctcactgcaacctctgcctctcgggttcacgccattctcctgcctcagcctcccgagtagatgggagtacaggctcccaccaccatgcccagctaattttttgtatttttagtagagacggggtttcaccgtgttagccaggatggtcactatctcctgacctcatgatccacccacctcggcttcccgaagtgctgggattacaggcgtgagccaccacgcccggcccaggttcaccaatttttaatgttggtcacattctctcccttcctctctctctctcatgccctttgtgctgtaatatttcactgtgtatttcacaagaacaaggataatctcttacatcacatagtacagtgagaagctcaggaacttttacattcataaaatacttgtatctaatttacagtccatagtccagtactgtcagctgtcccaaggattgaaggagggcatactctataattaatttttttcctgatgtgcattttctagtactgattggctttatcatttgctttctgtgctgtctctactcagtctgaatagccacggagattttactgtggaaaattaaatttcttctctgtctgtctgtctccctgtagtttttcttgttctgtgctgtgcttttgggtttgtttgtttgtttttcttttttttttttttgagacggagtctcgctctgttgcccaggctggagtgcagtagcgcgatctcatctcactgcaacctccgcctcccgggttcacgccattctcctgcctcagcctcccgagtagctgggactacaggcgcccgccaccacgcccggctaattttttttagtagagacactgtttcaccgtgttacccaggatggtcttgatctcctgacctcgtgatccacccgccttggcttcccaaagtgctgggattacaggcgtgagccaccgcgcccagcctttttttttttttttctttgagacagggtctcactctgtcaccaaggctggagtgcagtggtacgacctctgctcactgcagcctccacctcctgggttcaagcgattctcctgcctcagcctcctgagtagctgggactacaagcacatgccaccgtgcccaactgatttttgtacttctagcagagatggggtttcaccatgttggccgagctggtcttgaactcctggcctcaagtgatccacctgcctcagtctcccaaagagctgggattacaggcgtgagctaccacacccagcccatgccatgcctttttgccattatagtcgaggcacttggctttgcatgtttatcttttatgttagcgatcccaatgacttcatgaagtgacttcatgagggtatccacacctgggttccaggtctcccccgcgtggaactacagagtgagtatggcatgtgctggagaaaacaaaatatgtgctggccgtgtccttggggaagatatttgatccctaaagatgtgattgcctcatgtggctctacaaaagagttgtttagagtttagcatgagcaattgcacagcacaaactctctgttgtttgcgtgcattggggcagggcgtccagagccgggggcacttcagaggatgtactggtgattttaatataaaaacatccaaaacaattagatgtaaatatgaggaatgggtcatattacctatttaatgcggggagaagtatttgtacatgcaataaaaataacctagtgaaggccaggcgcggtggctcatgcccgtaatcccagcactttgggaggccgaggtgggtggatcacctgagatcaggagttcgagaccagccttaccaacatggcaaaaccccatctttactaaaaatacaaaattagccgggtatggtggtgcatacctgtagtcccagctactcagaaggctgaggcaggagaatcacttgaacccaggaggcagagattgcagcaagctgagattgcaccattgcactccagcctgggtgacagagtgagactccatctcaaaaaaaaaaaaaacctagtgaaaaagatgttattgcaaaacaacttttgaaatactgcacatgctggacaattgatgcattagtttaaaagaaggaaatgggccagtaatcccagtactttggggggccaaggtaggataatcatttgaggccaggagttcaagaccagcctgggcaacatagcagcctcttgtctctacaatttttttttttaattagctggacttgagttggaaagaaggaaggcggaagggaagaaagaaggtagggaggaaaaaaaatatggccacatgtgagaaaacccccttaatgaaatgaccgctggattttccaaatgatgggatgttaagcagccgtgaagaaatggttgattgacaaatatttgtggtatattaagtgaaaaaatagcatgatcccaatgatctctatctactttttgcaagggatttttctaggtatcactaagaatgctgtgagatgtgatgtgctaccttagactatgaagtcaaacagatctgagttcagttgggcagatttcttaggctctccacgcctgacccctgtgtacttaataaatgttggctgttgttatttttatcaccatcactttcatcatcacttctgggagcaaccaaattggttttggcaccagaagatgtcttgggggaccaatcagatagctcctcacttttttattttattttataaatttttaaattaaattaaattaaattttttgagacagtgtcactctgttgcccaggctggagtgcagtggtgcaatcacgtctcactgcagcctccacctcccaggctcaagcgagcttccctccttagcctactgagtagctgggactacaggcgcatgccaccatacccaaataatttttttattttttgtagagacaaggactcaccacgttgtccaggctgttctcaaactcctgggctcaagtgatcctctgcctcggcctcccagagtgctgggattataggcgtgagccactgagccgggccagctcctcactttttgcaggatgagagcctgggcttcctttgatcacgtggagccaattacacctccttggctgttgcgagcttagattcaggagattgtatttccctagcatccaacaggcatgtccaaatgtcatctcttttcttccacttcactttctgtcatgttccctttagcggttgctgtcggggctggtgttctgactgagactgatgaattgttttataatttttccagagtaattgcaatgatagtcatttataggacaggacagtcacgggtcacttaacaactggggtagattctgagaaatgcatcattagacagttttgctgtgtgaacgtcatgcagtgtacttgcacaaacctagatggtgtagcctcgtatacacctaggctatgtggtttagcctattgccccaggctgtaaacctgtacagcatgttactgtactgagtgctgtaggcaatcgtaacacaatggtatttgtatacaggcatatcacagagataaggcagttttggttccagactaacacaataaagctaaaaccacaatagagtgagtcacatgaatttttttgtttcccggtgcatctaaaaattatgtttatactgtagtccattaagtgtgtgatagcgttatgtctaaaaaaatgtgcacagcttaattttaaaatatttacttgcggaaaaatggtagcaatcatcagagccttcagcaagtcataatccttttgctcgtggagcatcttgccttgatatggatggctgctgactgatcagggtagtggttgctgagttgcagtgactgtgacaatttcttgaaataagatgacaatgaagtctgctgcatcaactaactcttcctttcatgacagatttctctgtagcatgagatgctgttcggtagcattttactcacagaagaacttctttcaaaattggaatcctttcaaaccctgctatcaactaagtttatgtaatattctaaatcctttgttgtcatttcaacaatgttcatagcatcttcaccaggaataaattccatctcaagaaaccactttctttgctcactcattagaaacaactcttcatctgttcaacttttatcatgagattggagcaattcagtcgcatcttcgggttccgcttctaattctagttatcttgctgttttcaccacatctgcagttacttcctccactgaagtcttgaaccccttaaagtcatctatgagggttggaatcagcttcttccaaacccctgttaatgttatattttgacctcctcctatgaatcaccagtgttcttaatggcattttgaatggtgaatcctttctgggaggttttcaatttaccttgcccagatccatcagagaaatcactatctatggcagctataactttacaaaatatatttctttattttattttatttcattttattttattttttaagatggagtctcactctgttgcccaggctgaagtgcagtggtatgatctcagatcactgctgcctccacctcccaggttcaagtgattctcctgcctcagcttcccaagtagtgacaccacaccaggctaatttttgtattttttttttagtaaagtcagggtttcaccatgttggccaggctggtctcgaactcctgacctcaagtgatttgcccgccttggcctcccaaagtgctcagattacaggcatgagccactgtgcccagcttcaaaatatatttcttaaataataagatttgaaagtcgaaataactccttgacccatgggctacagaatggatgttgtgttagcaggcatgaaaacagcattaatttccttttaaatctccatcagagttcttggggaaccaggtacattgtcaatgagaggtaatattttgagagaaaactttttatcctgagcagtaggtcttaatactgggcttaaaatattcagtaaaccatgctgtaaacagatgtgccgtcctccaggctttgttgctccatttgtagagcacaggcagagtagatttggcataattcttaagagctctctgattttcaaaatggtaatccagtgttggcttcaacttaaagtcaccagctacattagcccttaacaagagtcagcctgttctttgaagctttgaagccaggcattgacttctcctctgtaactatgaaagtcctagatgacatcttaatagaaggttgtttcatctacattaaaaatctgttgtttagagtatccaccttcatcattttagctagattttagctagatcttctggataacttgctgcagcttctacatgggcacttgctgcttcaccttgcacttttatattatggagatggcttctgtccttaaaccttatgaaccaacctctgctaacttccaacttttctttttttttttttttttttttgagatggagtctcgctctgttgcccaagctggaatacagtgacacaatctcggcttattgcaacctctgcctcccgggttcaagtgattctcctgcctcaacctcctgagtagctgagattacaggtgcatgccaccatgcccggctaatttttttgtatttttagtagaggtggggtttcaccatgttggtcaggctggtcttgaactcctgacctgatgatccgcccacctcagactcacaaagtgctgggattacaggcgtgagccaccacacccagccccaacttttcttctgcagtttcctcacctctttaagccttcacagaattggagtgttagggtcttgctgtggattagactttggcttaagggaatggtgtggctggtttgatcttctatccagaccactgagactttctgcatattttcaataaggctgttttactttcttattattaatgtgttcactggagtagcactttaaatttccttcaacaacttttcctttgcattcacaacttggctaactgtttggcccaaggggcctgttgtggcctgcaatatgccttcctcactaagcttaatcctttgcagcttttggaatagggatgcccgaggagaaggagagaggcaggagagtggacagtgggtggagaagtcagaacactcacaacatctattgattaatttcactgtcttatataggtgtggttcatggtaccccaaaacaattacaacagtaacatcaaagatcactgatcacagatcaccataacacatataatcatgatgaaaagttttgaaatattgcgagaattaccaaaatgtgacataaagacatgaagtgagcatgtgctgttggaaaaatggcaccaatagacttgctcaatgcagggttgctgcagaccttcgatttgtaaaatgcaatatctgtgaagtacagtaaagcaaatcacaataaaatgaggtatgtctacatctaaatgtatttaaacatagaaaaggtacagtaaaaatacagtatcattatcttagggcatcaacgttgtatatgtggtctgtcatctaccgaaatgtcattatgcagcacatgactttattgaaaatcattgtaatatctttattttggtaaatttttgtaaatcaatgtctcataaatcacatgcccctgttttcaaaaatacgtaatcaaggaagcaagctttcttggtcaagtatgctgaatactcaaccccttggagaggcacgtagtcattaagatgttcaagtctctgagaagttctgttttgaagacacgtatttctatgtttaacacagtttccactcccgcgccgcccccccctccaaattaagtaccagtcagcctcagggagactgaagccagaactctcctgattgacctatctttttgattgtgtagttataggatattccaagataaatgcatgaatactaccttgacggaatgtgatttctcaagtctttccaagtatggtgaccacaccttgagagtcagggctgaatttgcagatgagcattcagactgggtaaacatcaccttctgtcctgtggatgacagtaagccattttgttttccctttttttgtaatgtcatcatcttgccttgtttttttccacattggttggtccatcaacaagaattctttgagggcccacaaatggatggccttgcacaaggagcttaaggaatataataaagagagttgtatccctaaagaattcatgggagagacagaaaaagataggcatatcatcaatgcaacaattaaatataaatctaacaccttcagtgcctcaaggatgtgactatgttggggcccgtgctgttgaggagaagttctggaaggattcttgtggtgaattttctgggagaaagtcagtatggaattatgacttaaacaaagaaacataacgtgataggcaaaagaatgaaggcagatgtagagatggaaaatatgatgcggacagccggcttgcccagagcgaaggacagagggagggatagggctgatggcaggtgggttgggtcagactggttggtcagcatcaaaaattgttcccaggttaggtggagattgcagtgagccgagatcacgccactgcactccagcctgggcaatagagtgagacttggtctcaaaaaaaaaaaaaatattgttcccaggttagatttggagggtcatggtgaccaccagagtcccctaaatatgagatgagcatatttagaatatcaggaattgaaaactaattaaaccatatattagcaattgagagagtaaagaagattagaggttaaaagaacctaattcacaaaattcacatacagcccttcaaaactcacatagagaaaagcactaaaagaattgtctctggctaggcatggtggctcacgtctatgatcccaacactttgggaggctgaggtggtggatcacttgagcccaggaattttctttttttctttctttctttctttttttttttttttttttttgagacaaagtctcactctgacacccaggctgaagtgcagtggcgcgatctcagatcactgcaaactgcctccagggttcaaatgattctcgtgcctcagcctcctgagtagctgggacttcaagtgcctgtcactatgcccagctaaatttttttgtatttttagtagagacggggtctcaccatgttggccaggctggtcttgaactcctgaccttaagtgatccacctgcctcagcctcccaaagtgctgggattacagacatgaaccaccacacttgtccttgagcccaggaatttaagaccagcctgggcaacatggtgaaaccctgtctctacaaaaaacaaaaaaaataccaaaaaaaaaaaaaaaaaaaaattagccaggcatggtggagtgtgcttgtagtcccagctacttgggaggctgaggtgggagagtcatctgagcctgggaagtcaaggctgcagtgagccattatcacaccactgcactccagcagcctgggtgaccagagtgaagtcttatctcaaaaaaagaagtgtctctatatggggagtggtctttatgccggtccctgctctggacccccatgacccctacagttacatcctccctggtagagaatgatgctaggatgcctttcttccctttcccaattccatcccaagcctgggtaaaaacgtcttacttttggaatcataaattatcaaagttagattatattttctaatggtacctttacaaaaacaaaggaaagatgaacagaaaagtaagtttcacaatagttaaaagtacaagtttttgctcctataattgctaactggcttaccaggtcctaacttgtcccaaatcctgttagctcccatccatggaagttccctttagagcctaggtacaacacggttaagcagcttgtcaaaaagtcatacaggtcatgagtggctgagcaaggattcagacccaggctcttgcttctgtgctcttagcaactactacccaatattggagatttgcctcttgaagtctatgtattttagtgataggaaagtgtttccaggcatcatgcataacctatttatacttgctttggcaaagtattccaacaagttttcttgtctggttttcagacgagcactttagaatgatggaagcaggtccattaacagccctgaaaaagtaacagcagccaaagaagaatacaggacataactcaaacaccatgattgcctggaaccatttatgaatcattctatagttattattgagtacatctgtgtaacagctgcattctgagcactgaagctacaacaatgaacgagacaaaatacctcatggagcatatatgtgctaatcaggaatacagataataaataaatagatacatattacatgacaggtagtgactatggttcttaagaagactgaagcaaggcaataggacagaaagtaatgggagctgcacttctacatagaatggtaagatacagtctctctgaggaagtagcatttgagcagagacccatatgaagtgaaggagccagccacatgaatatctagtaaaagagcattcagaagggaaacagctttagacattgttggaaatatatatggttaattatatatgttaaacatgtaaaggtggccactaaaaataatagagatgccatctgtagcttccatcctgaaaaaggaaataaaattcaagccaacaaaaagcaggaataaaagaggagaagacaccaaaaacataaaatagaaatttaaaaagaaatccttatagaaaatacaaaatagcgtagctggaataattccaaatgtatcagaaaccacaataaacataagtggactaaacttcattaagaaagacaaagattatcagattttttaaaagaaactaaatccaactacccttcttagccatgtcaatcaatgtcatgacaatagtatatcaaagcagtgtacttccgtggactaattgttctgcatggttatgaaaaattaatgttgaagtttttaaaatgtgatttttgattgtcattttgcttgttcttctgcttagccattattggaccccctggaatgcaagtagaagtacttgctgattctttacatatgcgtttcttagcccctaaaattgagaatgaatacgaaacttggactatgaagaatgtgtataactcatggacttataatgtgcaatactggaaaaacggtactgatgaaaaggtaaggttggctaattgcatttcagaggtagtaggctttcatattctttgcagaatcttgcaaggtggcagcaccttatggactggtcctctgtaagccaagagcttcccagactgaggggttactggtatctctggctcaagcttcccagactcctaagggatatgtgtagagcagactttcaggacaaagtctgcacagtgacagcagacctttccctccctgaagggcagctgcaggggaaggcacagttgagtcagagttagctaagggaacttggagctggctgaattaggtgatcagcaaattgacaactgtcccatgacatcaacccctgtgtcccacacacaccgcaaatcagggacgtccgtggtcaagacagccccttgttgctctattcttcaggggacaatttctttaaaaacatgggcttggagtcaaaagatctgatcggaatatcacccgtgccaccatgtgccattggtcactttcttgaccccagcctcctcaagtctgcaaaatgggcataataacttactttattaataaaacaaggataataacctacttcgtaagattgttggggaatttaatgaaataatagaaaatataaactcatcccattgcctggaatgatgtatatatggtcagtacatcagtacatgaggtttccttcctctttgctagacatgttaaggttatttttattcatccttggtcaaggtttattctgacacctaatgcttctgagctgaatgccattcatggtcctaaactgacatatatatgttatgtatgtcatattacatatatgttatcccttccacattaaatgttgattttaccacttaattctcaaagtgtttgggaagaagggatcacccatgtttattatcttaggaatgatgtagttaaatttatatacacacactgcatagcccaaaaaatctcaagttaatttttttccccaaaaaatgttccattaaatgatgcagtgtcttagtcctttcctgctgccacaacaaaataccacagactaggtaacttataaacactagaaatttatttctcacagttctgcaggctgggaagtccaagatcaaggcaccagcagattcagtgtctggtgagcactggcgtgctctgcctcctagatggtggaaggcaaagggcaaggatgtaaacagctcccttgctgctcttttctaaggtcaccaatggcattcaggaaggctctgccctcattatttaatcacttctgaaaacctccacctctgagtcctatcatagtgacagttaagtttcagcatatgaattctgatactcacaatcactgatcaaatgcaggcatctctgctaagccctggggtataaagataagagcagattgctcccctccgggagctagcaacagacctcaattaaggatctagttttacagtgagcagggtcaggtgcctcctgaggcaagtgtgaatgcaaactccacttactgtttagcttcttttctcacacatgcccacaacatgggcatctggttacacatttgtcctctctcttcggccagttgtgtggcaaccttctgtgtgacttaccgagtgtggaaaggctgcctgctgcactgctcctctcacaggtgcctgtgatgccaccaatacaggttttttgggggtttttttgaggcagagtcacagtctgtcgttgcccaggctggaaagcagcggtgcgatctcagcttactgcaacctccgcctcctgggttcaagcaattctcctgcctcagcctcccaagtagctgtgattacaggcatctgccaccacaccccgctaatttttctatcaatacaggcttatatggttcccctctagaatgtagagattcctgaggcggtgccagactaggtccctaaactcacagcatgaagggcttttgagaattctttcttcctgttgggagaaagtgagaagataaaaccagaagttgcccagacgtgaaagggcagaaaaagccccagcagggattgagaaggtccctctactactatgccaccaccttacctgcttgcctctgagtttaatctttgaaaaaatttgcattgtaaggttttagaggcactggacttctggaactgcaagcccgccatttttttcagttgttgctttgatttgcttttggtcttttaatttacttattagaagttaaaccattttcttcatatattccataagtctaatccaattattgacctccatgggcttagctcctcatgcagtagaggcatataggcatgctgtcaacgaccccaaccccaaaaaagaactaaccaattgcgactgggtgtggtggctcacacctgtaatcccagcactttgagaggccaaggtgggcagatcacctgaggtcaggagttcaagacaagcctggccaacatggcaaaaccccgtctctacaaaaatacaaaaaaaaattagctgagtgtggtagtgtgcacctgtattctcagctactcgggaggctgaggcaggagaattgcttgagcctggcaacggaggttgcagtgagccatgatcgtgccactgcactccagcctggacgacaagagtgaaactccatctcaaaaaaaataataattaaaagaccagtttattttatttatttatttacttttttctttatttctgagacaaagtctcgctctgtcaccgaggctggagtgcagtgtggggtgatcctggctcactgcaacttccacctcccaagttcaagcgattctcgtgcgccaccacgcccggctaattttttgtatatttagtagagatggggttttgccatgttgactaggctggtctcaaactcctcagtcccccaaagtgctgtgattacaggcacgagccaacacacccggcctgctttatcttttttaaaaaagaagtgatggcttgtgtgtcatttaagctggagaattcaggaaccaaaatggtttgtgggcaccactgtcagtccttgatgcaccccctgagtggcctcagggtccctgaactgagaggagcacctgctacctgctccttcctattttaagtctaaaacggctattatcactgataaatgtgaaaaaaaaaaaggtggtgcccttccactgcttagtcatgttcttatttttgtctccattactagtttcaaattactccccagtatgactttgaggtcctcagaaacctggagccatggacaacttattgtgttcaagttcgagggtttcttcctgatcggaacaaagctggggaatggagtgagcctgtctgtgagcaaacaacccatgacggtaagccctgagatgcacctccgctaagcatcctaaacagctttatagacacctgccctagacatgattgcatccaactcaaattccagctcagttcagaaatctatcgccctgacattatctctcagccctgactgctcagcttcatgctaggaaggagaccacacccgcttctgaggcagctcattcactcttagatcaaaggagttgctagaaaccttttctgtctggtggagcgaggtctgcctccatgagccccccttggcagtacttattctaccatcccaggctccctaaagcaaggctcatcagtcctcccaagaccaccagcagagctcaagccctgtcacataccccaaaggtctttcctccccaggcggcacatctccagccactgccgacatgtgatgggaagcaagtgtcaggaaatctgaccacggtcctcagccagtgctgcctgattcagggccccttatatgcaggaatccaggatgtcaacacagtccttcacacaccatatggatcacttctagcttggaggaaggaacgggagaaggaaagagagaaggttcccatgggcctaaacactgcatatttatgtgcagacacattgcagttgcatgttggtgaaaactgaggcccaggtgtctgcatacaaacagcaggccttcctataatttaaagatgttggtgaagggttcagcaagctcagacaatggaaggatgaaacatggcatgcggcatgcagtttgatcaaaaagaaaaacatgtctgccggacgtggtggctcacgcctgtaatcctaacactttgagaggccgaggcgggcggatcacctgaggtcaggagtttgagaccagcctggccaaaatggcaaaaccctgtctctaccaaaaatgcaaaatttagccaggtgtggtggcgcacacctgtaatcccagctacttgggaggctgaggtgggagaatcgtttgaacccaggaggcagaggctgcagtgagctgaggtcatgccactgcactccagcctaagcaataagagtgagaccctgcctcaaaaaaaaaaaaaaaagaaaagaaaacatttcaaatagcttgcaaataccagtaatcacccatgaactgacaaaatccaaacattttacaaaacaaaagggtatgagccattgaaaacctgctttcatcaaacaccattttcattttttcttaaaacacacctatcccatttaaatcccacaagttttcaatgagagcattagaaggatcttctcccattttacaaactagaacatgaaggttcagagagtgatgcactttggatgttgccctcatccagatctcacgctgaaatgtaatccccagtgctggaggtggcgcctggtgggaggtgacgggatcatgggggcccatttctcatgaatggcttatcaccatccccttggtgctgtcctcccgagagtgagtgacttcctgagagacctggcggtttaaaagtatgtggcatcttgctctctcttgcccccacttagctttccaccatgattgggagcttctagaggcttccccagaaacagataccgctatgcttcctgtacagcctgcagattcatgagctaattcatcctcttttctttataaattacccagttttagttatttgtttatttgttttttggatggagtctcactctgttgcctaggctggagtgcagtggctcgatcttggcacaacctccgcctcctgggttcaagcaattctactgcctcagcctcctgaatagctgggattacagtgcccaccaccacacccagctaatttttttatttttagtagagagtggggtttcgccatattggccaggctggtctcgaactcctgacctcaagtgatccacccacctcagcctcccaaagtgctggggttacaggtatgagccaccatgcccagccatccagttttagttatttctttatagcagtgcaaaaacagcctaaaacagagaggttaagtaacttgcccaaatgctagtaaatgaaaaggatcaaccagaacacaggctcattcatctaagcccaggttatgtttcctgtaacttactacctatagattaattccataggtaaactgaggcaccgtgtccttgtggataaaacaggaaagagtagtctgcctcatggtgtggtcagatgaatctgaaacaagttagagaactttacacggaggatgtagattagtagattctcccacagttcacaacctatgcagagcagggtcctctctcctgccccctgtggaactctccatctggggtgagtaacaaaagcattccttgaatcatgtcaccatcacccagatgtgtccttgtttaagatgctaagatgccccagtttgagacaccaggagacccaagcactgccccagaggttcattacccaaaataaaataacagggcggggcccatggatccctgtttgaatgagtctccgggcgccaaattagagaactaccgaaatattaatggaaggatggatggatgagtaggagcctggaactttctcaagacctgggatgtcaaatcccaaggagacctcacgtgtccagctgtggtgtggactgggggccgctccagccagctcactgccgtatgaccctggaatcaccaagagcagactcacatgtttgagctgaacatgctggaagtgtgggccagacggcaccgcttctgggctccggtgcatgaaacaaacaagaaccaagaactgggataaggagtgctgaaacccgcaaggacaggagctggggagatgggaggccgcctgcgggaatgaaagcacaagagaacgagggccatggggttgggggcggcacaagtgctcaggtccttccagccgcttccctcatacagatgccacttccatccctcagacgtttaaggagtgtgcactgtgagccacgcactgtgacaagcaccatgtcgtgccagcaggtgactgcacttgctgtagctccagggtcatgtgctgtgtcagggctctttgatccctgtgtccctttaaaacgacatgatcttcatgggcagtgtttgttctcttggcccctctcagagccaaatgctgtgctcgtgagggcggtgctggttggcaggggatgggcacagagaaatcaggtggccagggcagctggaggagggacctggagccgaacaaggggagctgggaccacgagcaacccaggtgacatcctggactctgtcaaagataagcaaagcaggacgctaattaaagcagtcaaaacattttattcagtaatgactggagttgggaaaagggccgagttccatttactcagaggtgttttaaagggagaacgagggccggggcaggaggatcgcttgaacccaggagttcgagaccagcctggccaaaatagtgggacccccatctctataaaatattttaaaaattagctgggcatggtgacacatgcctgtcgtcccagctacccaggaggctgaggtgggaggatcacttgagtcctggaagtcaaggctgcagtgagctgtgtttgcactactgtactccagcctggacaacagaacaagactctatctcaaaaatgaaaataaataaaaggagaatgagggagtagaaagaaagaatgcgtggggagttgagcagaatcagggaagtgaaaaaaattttaaatcagctaaggggatgggtcagtgccgtgaggccctctgtgtctgtgaaccggtgtttatcaaggctaggcttctaccctcccacagagacagggagacagaggccctgtacttcctgatgatttcaaaagaatggctcgcagatcattgagaaaaatatccctgcgttgtgggagatacatacatgtctcaaagggacagaggaaggattcaccactataagccctttttaataaatgctctccgaaagagaggtcaaggcctcgcatcaggggctgggaagaacaaatggtaagttccttaggcagccttgggtttttccagacaggaattcaagggtgtggccttggcctgctagaagctatgctggggtttggccaagtctcttagtgcaggggtttgtttgttttggctttggttttggtttttgaggcggggtttcactctgtcgcccagtttggagtgcagtggtacaatcatagctcactatcacagcctcgacctcccaggctcaagcgaccctcccacctcagcctttcaaattgctgggactacaggtgtctgccatcatacccagctaatttttaaaaaattttttttgttgtagagaaggggtctcactatgttgcccagactggtctcaaactcctgggctcaagtgatcctcccacctcggcctcccaaagtgctggggttacaggtgtgagccaccacacctggcttagtgcaggggtttggatagagttgttcacgcagagtttctgtttttctcaatatagaacgagatggtgcctgtgtttaactcaggctcaggtgtccaggggagtatcacttgctgtgtcaaggcagaagtggtatatccagacctgggaggcccattgctacactgccagtagtaggtacagtggatcagaattcttgcttgttattcttttgatgtatgtcagcaataattccagaaaccaaactttggagaagataggaaaagggtgaaactcttcaaaaacctctgggtcctccaaccagttgctgtttctgagacgtcccccaagataaacgtacaggctctgttttcagggattgtgatggttaaaatgctcttgggatcactgtgacccgacctgtgacaagaatgtaacatgcccattacccctggcagaaacggtcccctcctggatggtggccgtcatcctcatggcctcggtcttcatggtctgcctggcactcctcggctgcttcgccttgctgtggtgcgtttacaagaagacaaagtacgccttctcccctaggaattctcttccacagcacctgaaagaggtaggtaggatggagtgagatgtggatttgaaaaccttgatcggaaagagctttcctttctagttagggctgcccaattccagacaacatgttttccaggaaaacacacacgcgcgcgcgcacacacacacacacacacacacagacacgcctcttgtgtgctggtccattctcttgtaaccatgtcaggtgaaggaacagccccgaggaaaggggcgcggggttgtccagtatccctctctgcacctagggcatcgctccttctccagccttcactgcccaaagccccaggtccctgaggagcaaaggtgatggttctagggcaggaggggaaaaacagagctcagtgtggaagaaagagaaaactggaggctaaatgccaggaaatcaccagagggagagaatgggaggaaagaaaggaacatttccagttttggaatatacgagagcgattgaaattctgggtagtgtttcctggactttaggaaggctatttgtaaaagggttaggaagaagaaacaatgcacctgactaatgataacaggactgaagaagccctaagaaatgaaactctggatagtcactgacactagagaatgaaaagattccaagcccggacccaaaggaactatctttggtctcatttcccaggaagcagactctaagatagagatttgtattcaaaaggcttgatggggaacatccttgggaataataccctaaaggagtgaaggtagccagatgtggcagagagaggttgagctgtcacacagccacaacagaggcctcagccaatcccatgtggcctctcgagctggggtggccctttagagatgtcctaagctgaggcaagagggatgggcgtgtgaactccacattggccatgcgctggatgtgggctactcccaggaaggggccctaagctagggtgagaggtgagacagcagcagccaggagctaggagcacgagtgcctcattcctgaaggcaccacagcctccattacagtcacccctgggaccacttggatctactggaagcagctcctccaggatctgagttgatctcgtttcctggggagggttacaggaggaagatgagtggaatgcactccagcccccaccgctgctgctggtgtcgggaccacaaaggaatgcttgtgggctggctccttcctccgctgccctgagggcccgctctcctctcccgggaggagcctgtgctgtaggggctgctgacctgattgggtgacccagccctcatcccagaaagcactgagcctccggtccccaagcccatctcaggcaatggcctgtccctgcacttgtcaatttgctgtcaaaatcgggcaggggagtcccaggaggtgcccagtgactctggcgggtacccactgtgatacagtggtgacttctctcttgcctgccagtctcttggcctgaggaggtggcttaaagtttatcgggccttgtttcatgtcccctggtagaaatgctcccactctgggaaccttctagaccaggtgtcagcaaactttttcttaaagggccaggtaaaaaatattttagaaggccaggcacagtggcttatgcctgtaagcctagcactttgggaggctgaagtgggtggatcacctaaggtcaggagatcgagaccatcctggctaacatggtgaaaccccgtctctactaaaaatacaaaaaattagccaggcgtggtggcgggcgcctgtagtcccagctcctcgggaggctgaggcaggagaatggcgtgaacccgggaggcggagctggcagtgagccaagatcgcgccactgcactctaacctgggcaacagagcgagactccgtctcaaaaaaaaaaaatacaaaaatacaaaaattaacccggcatgctggcgcatgcctgtaatcccagctactcgggagactgaggcaggagaatcgcttgaactcgggaggtggaagttgcagtgatccctcacaccactgcactccaccctgagcaacagagcgagactccatctcaaaatttaaaaaattttaaaatttatatatatatatttgaggttctgttggcaagaaataaaaatcaaaattatgtagtcacttatataaccacttaaatataatcatttaaaaatccgaaagccatgtgaaaaaacaagtggtaggccggatctgccccgtgggcctttgtttgctggccctgccctagacccacagagcctcaaatcacaaggccagggagcagcggtttccccagtaggtcactgggcatgatgagggtgaacagggccactcctgccctgcgcttgcttcctgacgcaggtgttccacctgctggggacacagccccacatgattgttggtcttgggtgcatgccaggggctggaggacaatgccttgccctcaccaggtgtcatctccaagctggcgcctcccctgcactttcaacaggccgctccgtccttcttccagtctggcagctgctgggtggtgcagggtgggatacgaccaggggggtcccgccttcactgtcagtgggcccattggtctggcaccatgtgatacggcgtctctactagtttgtcggggctgctgtagcaaagtagcactcgttgcgcagctgtaacaacgggaattgattctctcacggttctcgaggccagacgtccaaaatcaaggtgtcactaggatggtttccttggaggctgtgagggaggatctgtcccaggcctctaccctaacttctgagggttgctggcgatctttggcgttccttggcttctagaggcatcacccagtctctgccttgatcttcacaagccttctccctatgtgagtgtctgtgtccacattgcccctcctttatacagacaccagtcacgggaggagggctcaccctgctccactataccctcattctaacttaactgatcccctctgcagcaaccctatctccaaatacagtcacattctgaggtagagggattaggacatatgaatttcttttttttttggagacggagtctcactctgtcacccaggctggagtggagtggcatgatttcggctcagtgcaagctccgcctcctgggttcaagtgattctcctgcctcagcctcccaagtagctgggattacaggtgccctccaccacgcccggctaatttttgtgtttttagtagagacagggtttccccatgttggccaggctggtctcaaactcctgacctcaagtgatctgcccgccctggcctcccaaagtgctgggattacaggcgtgagccacagcgcctggcccagaacatgaattttgtaggaacagaattcaacacctaacaggatcctctaccaggagagaaaacacctgtgaaaccttgggaatcctgacaggccctgcaggacacgaacagcaaatcactcctaaactaaaggttagctccagccaggatgcaccactgccccttccagagtggtgcaagagtcgacgtcatcagcttgctaccaggtggctggtcagtctcctcggggaatgccgtcatatcaggggctcagcattgatctctgtcgcaggcaggttggacattccacagcagtggtggctacccagccgtgggagtgggagccccttctttcaggcccacgcgcagcctccatccctgccaccacggctgctgtgttcatgagccctgtgccatgtacaggtgaccagtggcagaggctggctgacatccactggctgtgtcatcatgtctccctggctgtctgctgtctatgatacatgctctcagtgggagttaatatgggatgcagaaatctccacgctccagctactccctcaggtccatccttcttcccaaacgtccttgtccctaaccttccagtcttccttccccagggcccctgaccaccaggcttgtcactaacccctgcctgggatcccacagctggcctgatcttgggctgcttttctttccacataaaggtgatgatcagacccacggcccaaagctctgcccgtggggacactttctccctggttggggctgaagtgcagcagcgggccactttcggtgtgtgcccacataccatgctaaccttgccatgaaaccaggccggcctttcctcctacttctgctaggcataattacccatccccctttatggtcatcatgtgagcagacacagacacggtgcgacaggggtagaggggacaggagtccgagccacctgcatgtggccaacatgcaccctctgaccccatctcaggacctactgggtctcaggcctcatgatggtcacttctgggcagcaccatcacctcgcgtccctcgtcagaagctctgtctctgccgggaccccatagcgtcccaggagctgctttttcaaacagagtacaattatctgctgcagatggccccgcctgaccccagaactctcgtatctatgttgtgactttcctcttgggatttgccataaactccacccagcatcttttcccaccccagacacttcaaggatatgcgaagtcacacattccccaggttggattcacaggccgtccctggtgggtaatgtaccagtcctgaggtgtgcatagggctgcaggctgcacggcccataccttctgcagggccctttcctgcctggggcccctccgtgccagcagcctcctgtgtggtttgataaacatattggaacagtcagtattcctaggcgtggaacatgctgcctccagaaccctgaggcctccacagcacaatgcttccttcctagtggtgggaggggcaagatacaataattggtcctttaaagaggatgtcctggcatgccccactgcaagggtcccctcaaatatcacagatgtggcaggtccctgaatctctacagggtgagtttctcaccctctgcagtgtgtgtgcctcaccaatgccttgccacttcttggcgtgtctggtcctattaatgtattgttgtgatagctaatatcaggcccttgtccccgcaccaccagtcagctgatgcaggctgcctctggcaagggcacagagccttgaccgaggcaactgccttctgggaagggcacagcctagaaagggactcagctgtgagccatttcagcctccgatattcctggaggctgggggaacaagcgcctcctccacaaaagggatctgggcaacatagcactgcatctaccctcgatggcaagtggggcacaggaccataggcaagtgtttcagagcgactagaaccccctacagagagtgccaggccgagcatggtggctcacacctataatcccagcactttgggaagccaaggctggcagatcacttgaggtctgaagttcgagaccagcctggccaacatggtgaaaccctgtctctactaaaaatacaaaaattagccacgcctgtaatcccagctactcaggaggctgaggcacaagaatctcttgaacccgggaggctgaggttgcagtgagcagagatcgcgccaccgcactccagcctgggcgacagagtgagactcgatctcaaaaaaaaaaaaaaagtaccaaggagactgcagcaccagcatgaactaagaatcctctatgagcttttgagaagccacagctgcagcaggaagaacaaagaatgagggtgggccctttgcttgggtaaaaggtgttgtgttaactgagaaccaaaaggaaacaaaattattcacctgctatttttgcttctggcttcaccatcagagaaactggtcagacaggaaagaacagacggaatgaacggtagaggggcctgctggcataagctctcacctccgaggctagaagactttgcagacttgaccgtggagccagcccctgtaagatctgagaaatcagagatgggagtggagggtgctggagaagggccaggcaactacggagcagttcgaggtcctcacatcacgactttctccagcttgactcataatgatagaatccattaaccaggatccagaggatcatttcgagttcagggagatttgctaggaatggagactttgagttaaaaaagcactagggaggttcatcctacactgatctattgagccagccaccagtttgtgtgtgtgtgtgtgtgtgcgtgtgtgtgtgtgtgtgtccaaccgggagactcagctgatactgcttccttgagatttaatacaccttcctttgatctctcctgtccccattatcccaggaaaatccagagtagcttccagtccattctcattaatccactggatccaaagtttagagaggttccccttccctccagcctccttcctggcccaacagaggagcaccccaccaccctccatcagctgctcaaaacccacaagggaaaaatccctacaggtccatgccaggaggtagtggagctaccctcaggttccattaagtcataccagaaggctgagtgtagaaatgacattaagaggggttccatctgtagggaaagggttcaagatgcaaagctttacagaaggttctccgtctaattgtgaagattaagagcactggtggacctaggaagatgaagaatggagagtgggaaaccagcagagatttcaggaaatgtataggggcatcttcatcgttcaaagcaccggagagacaaagattattagactgtggtccctgcccttaaggagccctggctacaggtgacacaaactgagaagagttggtgtctattaaaagtgagaaggcagctctgtctgatagagcatattgcaagccatgtgtgttttagatttgctagtagccacatttttaaaaagtccaaagattgggccgggtgcggtggctcacgcctgtaatcccagcattttgggaggccaaggtgggcagatcacgaggtcaggagatcgagaccatcctagctaacgctgtgaaaccctgtctctactaaaaatattagccaggcaaggtggtgggcgcctatagtcccagctactcgggaggctgaggcaggggagtggcgtgaacccgagaggtggaggttccagtgagctgagatcgcaccactgcactccagcctgggtgacagagcaagactccgtctgaaaaaaaaaaaaaaaaaaaaagtccaaagattggccgggcatggtggctcacacctgtaatcctgacactttgggaggctgagacaggttgatcacctgagatcaggagttcaagaccagcctggccagcgtggtgaaaccccatctctactaaaaatacaaaaattagccaggcatggtggcgggcacctgtagtcccagctactcaggaggctgagggaggagaatctctggaacctgggaggcaaaggttgcagtgagccaaaactgtgccactgcactccagcccagacgacagagcgagactccatctcaaaaaaaaaaaaaaaaaaatcttcgtttgtatagcagtgagtatccccacttgtaaaaaaataaaataaaaataaataataaatcaaaagatgccagaggccgtggctcacgcctgtaatcccagcactttgggaggccaaggtgggtggatcagctgaggtcaggagttcaagaccagcctggccaacatggtgaaatcccatctgtactaaaaatacaaaaattagctggcgtggtagcacatgcctgtaatctcagctacttgggaggctgaggcaggagaattgcttgaacctgagaggtggaggttgcagtgagctgagatcgtgccactgcactccagcctgggtgacagagcaagactctgtctcaaaaaaataaaataaacattaaaaataaataaataaaataaaatagattttccagccaggagttctgtgaaaaaatctttgtaaacccagaaaaagaaatggagaaaattgatcattaactgtcctctttccctccacagtttttgggccatcctcatcataacacacttctgtttttctcctttccattgtcggatgagaatgatgtttttgacaagctaagtgtcattgcagaagactctgagagcggcaagcagaatcctggtgacagctgcagcctcgggaccccgcctgggcaggggccccaaagctaggctctgagaaggaaacacactcggctgggcacagtgacgtactccatctcacatctgcctcagtgagggatcagggcagcaaacaagggccaagaccatctgagccagccccacatctagaactcccagaccctggacttagccaccagagagctacattttaaaggctgtcttggcaaaaatactccatttgggaactcactgccttataaaggctttcatgatgttttcagaagttggccactgagagtgtaattttcagccttttatatcactaaaataagatcatgttttaattgtgagaaacagggccgagcacagtggctcacgcctgtaataccagcaccttagaggtcgaggcaggcggatcacttgaggtcaggagttcaagaccagcctggccaatatggtgaaacccagtctctactaaaaatacaaaaattagctaggcatgatggcgcatgcctataatcccagctactcgagtgcctgaggcaggagaattgcatgaacccgggaggaggaggaggaggttgcagtgagccgagatagcggcactgcactccagcctgggtgacaaagtgagactccatctcaaaaaaaaaaaaaaaaaaaattgtgagaaacagaaatacttaaaatgaggaataagaatggagatgttacatctggtagatgtaacattctaccagattatggatggactgatctgaaaatcgacctcaactcaagggtggtcagctcaatgctacacagagcacggacttttggattctttgcagtactttgaatttatttttctacctatatatgttttatatgctgctggtgctccattaaagttttactctgtgttgcactatatgtgttcatgatactacctgtgtgtcattgttagatactgaaaaaggctctagtttgccatcttgatgtccactcattccacaagtacttcctagaccctgcctgctgttgaccaggcactgtgttaggtactaggtctcagtcatctgggagctctcagtctaaagcttcaaaaactagaattgcagtcaagggccagatccgttattaaaaaaaaaaaaacctagaaataaaaagtatagttgtggaagtttaaaaagccaggggacaggcttaacaacagattagacacagaactgagcaaaaaagggagtctccaggtacaatggctcacttctgtaatcccagaactttgggaggctgaagtgggaggatcacttgggcccaggagttgagaccattccaggcaacatggtgagaccccttctctacaaaaagttttaaaattaggcatggtggcatgtgtctgtagtcccagctactcaggaggctgaggtgggaggatagcttgagcccaggaggtcaagactgcagtgagctatgttcatgccattgccctccggccctgcctgggtgatggaatgagaccctgtcaaaaaaaaagaaagaaagaaaggaaagaaaaaaaagaaaagaaagagaaaaggaagggtattagggttctccagagaaacagaaccaatagaatgaacatagagatttatttgaaggaactgtggaggctggcaagttcaaaaactgcagggcagaccaacaggctagagacccaggtaagagtggatgtttcagcttgagcaaaagcagtctggaggcagaattaactcctcctcaagggacctcaggcttcttttctcttaaggccttccttcaagtgatagaagtgaaactcacccacattatggagggtaatctgctttactcaaaaatctatggatttaaatgttaatctcatctttaaaaataccttcacaccaacatcaagactcgcattttacctaatcagtctagctggttgacacataaaattaaccatcatagggagggtagatctgtgattgcttttttgaaacttttttcaatgtttgctgcatgtatatagaaataaaatttggccgggtgtggtggctcacatctgtaatcccagcactttcggaggctgaggcaggtggatcacctgaggtcaggagttcgagaccagcctggacaacatggtgaaaccccgtctctactaaacatacaaaaattagccaggcgtggtggtgcacacctgtaatcccagctatttgggaagctgagacaggagaatcacttgaacccaggaggtggaggctgcaatgagccaagatcgtgccactgcactccagcctgggtgacagagcgagactccatctctaaataaataaataaataataaaataaaaaattatttttgaaattgaattttaagatcttgttacacttttttaaagaaaaaagaagttattatccagggcacagtacagagaaacaaaagaatggaaaatatgagtggttaggatacaagcctgaaaaatcaagctgcaagcgtagattagcaagctggaagcttgcacaggtgaatgcggcagctgtgccaatagaaaagggatacctggaagccaagtacatccaatatggaggttcctcctcccttctctttgtcgtcatgtgtgcaggtgtcatggtgctggccaggtagagaccccatctgcagaataaaagattagggtgggatggccagcctctttgtgggctatgtaaatggcacagctggcccaaccaatccactatgccctatataaatcaaacactgcctcttcaagctcatctgtaaaaccaaccgtatcttgccccaaacccggaaacccactcggacccactcgcaccccctccctctgcacaaggaagctctctcttctttctttcaactattgaacttccactgttaaacccagtttttgtgtgtccatgtcttc

SEQ ID NO: 124 - DNA, 47941 bp, Homo sapiens
gcagcctgggcagtgggtcctgcctgtgacgcgcggcggcggtcggtcctgcctgtaacggcggcggcggctgctgctccggacacctgcggcggcggcggcgaccccgcggcgggcgcggagatgtggcccctggtagcggcgctgttgctgggctcggcgtgctgcggtgagtggctcctcgctcccagccctgcggctgctgtcgcttcgcccccgcgggcgtgtgggctgcgccccagccagcccggcggcgccctgaagagggtggccggggcgcagaacactcgggccctgagcgcccgaagtgcagacgtgggagggccccacggggaatcgggcgccccccttcttcctcccttcctttccctggtcgtcttcttccccctgggtgagagcggggctcatctcctccacccggttctccatctccaaatgcacacacacaggaagactccaaaccgcgcacctcgcgcaaaaattaactagcaagaaagggcgcgatcagaggcaaggggctgcagctgctagggatccccttctttcgcacccctctcccttctcagtgcttagacgcatttggggttgagggagggagagtgggagggcccgggcctcttgcgatggaagtgcgcattttggcgaagtcggtgagaaggggtttctgccgtttgcctcccacatgaacataggagcgaagaggacgtaaaggacacaattaagtttcattttcaactcagcattcaatcagaagaatcttccggccgtgtaatttttgctgtcgtttttaatcctaaaaataagcttgctggaaactcttcctttctcgtgaccctcacgccccatcagccacttgcatacattctaaattgtacgttgaagtttttcccactttatttgggggaaccgttttaaaagtaatctggttttcgcctgaaataggaagacagtaacctccagtcaaaacatgtgcagcagaatgggatttggtgtttttcgacgccaaagaacctccacccccccacccccaccccgagcttttggaatccatttcgcttttgtaaaacgtgtgcttcgtctgtaaaaactgagcaaggaatagaaacattcgtagctctacagtgagtgcctgtcacacttcacatccatagagccttttgtgaacttttataagattcagaattcagcttgagcaccagtgacagcaattgttacatttattctagaatgctaaattaattcgattttaaactgattattagcctcggtgtgccgttcctaaaggcttgtgactttaggtatttccaagatgcccttaagtctctgcttaccactttcctccctccccgagcatcctggatgttgggactgtgaatccaggtctccgttatctaaatggttgatgttagtgtttcctgtcatcacgtttagtatgcttgttgcctttactatcattatagctaaaataatactgctttcagagatgtgttgtataatcgcataataatttcagaacgccttcatattgaaccagatatgaagtgatacagtatcatttattcaaactgcctaaaaagtaaaaagtataaaccatatactatttttaaaacaggtaggttagatttaaatcacgtagtttagaactgttggaatggtactttgagtgataggatttatgttaggccttctttagtaatagttaatatcatgtaattagcactcgtttacacacaattttaatgtgttcgaatccctagagtcatcgaattcagaatttatagtattttattttacttagttaatattaacctaaaaaaaaagcaaaatacaggcattatgcagttgagttgtgttaagtgtggactgtaaaacaggatagtatttgttataaaatatgtttttgtatgtgtttaatatatagcttcaaaaggacatgtatgaagaaagatgtctcagcacataggtagttataaaccaagggtttgagagactgactttagaatctagaatgagtaagaaagtgatggatctgtacattcatttgtattgaagtttgattttgctttcagtttcgttaattaagccccgggaatcagggacctttcctggcaccctctacagtgttagtggcctttgtggtaaaagaagttatctcagatactcatttcatgcaattacaggcaaactggaaggcaccttaatggtatgcaagatgtgaaatgaattactatgttgcattccactctgcccccactcatgtacaattatactttgtttaaaagtgcatagtttcagtggtatttattagctagcaaataaaactttaaataaataatgatggtgttgtgaactgtcttttcagtggttgaatgattcctctttgttcaaaatgagttgtttttttttttaaatcagggtacatatgattaaataaaatttttttccatcgtctaagtctagtagtaatttggcttgtttaaataaaaatgttttctcttaaagaatatattatttttaattacaagttgccattttaaggaagtaaatgcctatttaaaagtacgtatttatggggccgccccagcagtctagaggcagtgttttttaaagcattacactaaatgcctaccattaggacacttgctcatgctgtcatctgtgttttggcagatgttttatctttgtcactgagttgttcatggagatttgaaaggccaagttctaaagatgagagatagatgactttccccccaaattgatacatattctaaatccaaaataatcacgcatattcgcaaaactagatttgcatttcatagtatgaattcagctatagctagtatataggcattgcttttaatatagtaatgttctttttgtggcataattttcaatcactttccctgtcttaggtttctgcctacctttttatacaagataaacatttctattttcatatcttatctctcagtctgattttaataatatcgtcttgggttatcataaagttactttcactttgtatactagtgtagatatttcttctgtgataaaaagaacaagaataatagtgtaggatcagttttgttaaactcattgtagtactaaagtagaaaatacagagcacccaggaaatttgattctgatatacctagactaagacaatagaatctgggtttttcctctttacgattatacagtgttgaaacagaaatcaagctacacccacctctgaatatttcattccaaatccatggagcagttgcataagactcagaatctggtcaatctcatggtttgagattcagattaggagaattaatgtgcatatttgaatatatatcacacttgtaggttaaaaggtaaagcaaggggtttggctagttcaaggcctgtcaaaaccacttttttccatttcaagcaagtatgagcctatactacacaccaagcagtttgcaaagccctctttggcatatagacaaatttcagacagtatgtcagtatgtctcttaatgacttaagatgtagtatatagacagtagggatatcttgtttttattccctcatggtacttaggtgcttgttaaggccataattctggatgttattgactttaaactgccctgccctttttaattctaagttggcctcttccattcttataaggccacttttaaattcattagggggattgcgttgggtcagaactaagtacaacttgtggacaccccactctaccccagacctttattttagtgtccattgaggagagatctgacttcatcctttttgtacgtaacccagtgtagctagagactccagttcttcaagccagacactagttctaaggggcaaaagtaggtaggattgctttctggcatgtgtctttactgatgatctttgcttctttacctaagaggctgctgagacttcttactccattcctgaacatccattcccagcagataattaacttcctactctactgcaaaaaatagtggtgtgtggtttggactccctctgcttccctcccaccattactaaactgatctataacatcttagataaccttcgtctcattctcttccgtcatccagcttcagaagaaaacctactcacttttccaacgaaaggacaacctgtctaccaccttttcacttctaccatcttggagacctgctcagtcagcataattcttcagtcttgcatcttagatctttccctctccattgactccttccccgtagcttgcacacatgatcaaattttaccccatattaaaaaataaaacaaaaaacctcaagaaccaaaatgtccttgcccttgttcctgcctgcacattgtcagttgttgccttctctgctttcttacactgccaaatttattgtaaaagaatcactgccacttaatatattttaagtgcttgataaagccatcttgtttctctctcatttctcaaacatagtgtgcatcagatctcactagtccattgacagatggaaagatattctccccaaatatttaagcctcttttctctataattagcctaccatctaactagctacacatactattttggttcacaattttaattttcccctctttatctcccctgcttcccacaactaacttctgttgattactgtaagttttaggtcttctctccagttgttctttacctggactattgcagtagtctgttaacggatctccctttctcttttcccttgctgccctcagtccttcctttacactgctgtcaatgttttttttttaatgaaattaaatctgagtggtttacagttgtttatgttatcaagtctaaactcagcatgacattcaaggaagccctttagaatttttcttattcatcttttccaacctcatttcctagagctcacctaaataattctctgctttcagttcttcactttctttatctggctgtacacgttactctgcctagaaagccccttctttcccataacattcttactcatcctttaagacccagtttaaacatcaccctctaggaagatcattcatagacacattcttgtgatttttaaggaacttttctatccaaccatacaagtgacttgagattttccatgaaaatatgcagcttcatgatttcataatcaagtctatacaagtgagaagcagtggcaggttctcttgaaatacagaagaaaaatctatcttctcccacatgatttttagatttttcttctatggaatttatactttaaactttttacattcacaaggaaggtattgaatcctactttctggaccctgtgttagattcttaggataccaaatgaagacagtgtcttactttctgagcctggagagatcagttaagtaaaatagtaattacacagtagtaggtacagtgacaacatttgaacacaaggcaatgggagcaataaggaggggaagaatacaattgagtaatggtagggaaggggaacaagattgaacaactcaactgtgctttagtaggaagagcaaaagttagcaaactgtagctgccatttattgagtacttgctgtatatgtgaaaggtgatacaaaaacatgttatcttattaaatcttaacaatatctctatgaggtatataccatcactatgcacattttatagatgaggaaactgaggagcagaggtaagtaacttgcctaaggttatacagctaagaagtacatgaggtgaatcttaaactagggtcgaccccttaatgtgtgcacttaactattatacaccctatgtactcagtggggtaatagtgtataacagttaagaggctatttaggtttgaggaacagtatgtgctaaggcatggggatgagagtgcatttgcatagtcatggaattgcaataacgtcattatgattggagcttagtgtggaaatgagggagtacaggaggtggggctggaaaggatcttgaaggacctcgtttgtattccatgctaatgtacttagaatttagcctgaaaagggttgagggtgttgttaaagtatcttaggcaaaggagtgcacatttgcatttagaacagttattttgggaactgtagaaaatacatcagggccaggatggagaaaccttgaggatggaggcagggagctggttagaagggtatggcagtagttcgaggggggaggatgagtgtctaattcattcattcaacagacattggatttaggaactattaattaggttgaacctactaagaattctccatggctaaggagagggagtaatctaatgtgattcttaggtccaggtctggttcactggtagatgatagaaaatactttagcaaagtgatttaggctgagggtaagaagtgggggtacgtaagtttttggacatactgactttgaagtaactgtattacatccaagtggaagtgcacagcagacagttggctgtccaacatctgcagtttagaagaaaggcagtgcatgattcagttttttatttaaaagtttgcaggtgaagaaaaccatgggagtcagggaaaatactagggggcagagggtactgaatctgaagcccttagggacactaacatttagtgtgagtcaaggaatagatttcaacaaagaaagcaaactaggatggggtggacaaagcaggaggaaattcgtaatcaggagataccatggaggccaagagagaagagcaagaaagagactgcccactggctcaagaactgtggaaaagtcaagacagaaagtgaaccatgcatatttgatttttcacaagggattggtggtgactttggcaagagctgtagtaatgaagaagtaggggtgaatgggtggaggccacgtaacagatttaaggagtgaattggaagtgaggaaatgcttatagattgttaatttgggatgctagggagagagaaagtatgagggattttttttaagttggcaaatattcaagcaatttttaaagacacacacaaaaacaacaccttgtaaatggaatgggggatgtgggaggctgataatcaactaaggaaagtcgttgaggagactgatgggtatggaggcagaaatggaaactagctttcaggtgatgggattcatttcagaaaaggaagcaaaaaaaaaaaaaaggtgtggatagttggggttacaggtaggtttatagggaaggagtattggaagcttcaggattctcctctgaaggtttcagtttgtattaagaaatagggagagaggactgttggagagtatgggataaagggcagaagggagtaatttaagagttgttaaaaagaagcgaacatttacatgaaatatgtaaaatggtaaagattgaaggcccggaggagcagggactatgacattcttctgtttttgcctattagcaacatgaattttcttagaaatctgtaagaccaattactcttctgcccatccataaggacgttgtccatgcagaaaagataacacttgtaaccttgtattatatacttatcatcgcccatttgagaattgtaagctcctaaaagataataactatattttttcatcactatatccccacatcctatcacaatatttcatcacaggtaattcttgacaaatgttgattgcatttttaaaatttctaacctgaacttgtgtgctgtgaccaccatggattgagtcttctctgccactacaaagctcttttctagactatgatatgagatggtttgggctgatagtctatattcaccaatacttgtacagttccaatgaaggtttcaagtctaatacttttggcatttgatataaaatcattttcccattttatttgctaatttatctataactctggcattactcttggttacatttgtctgctgctatctgatttagactgctataagcatatcttgtttattcagagttttcttattcatgttaattatctgtgtttctttatttgctctctacattttaaatagtttcttcactttgctgttttatgagaagggagtaggcaaaaggaaaaaaccccaaatcagacagttttactacttaatagttttttaatgcatctttatagagattgaagttgtagttaacagctagagtgatatttttggcctgcccttatttattaattactagtgcaaaggttattcaaattgtggtttatccaggtcaattttactgttatttttactaatagcatttattctacttaaattgcttcagctataaaatgtttttattgtaacaaataatgcagtataattattgctttctgtattcctttgaaaattcattctctaaacatatgttatgaaatggtggattgttcaccaactacttcttacttacttaattaaagcctttgcaaaaagtttcataggatgactgagttcttcattcgtacctcttttttttaagaataatctcttgaaagtcaaaccatgattcatacaaatataaaatgaacatgtgtcaaagattttatttcactaattaattaacaagcaaactagcaagaaggcaaaacccttttaaaagaaaattggaaaaacagacacatttataggcaatcaagaatgcctaaatgaatttgctaataaatgcaaaattggtcaatatcctcagggcagtaaaatgtagtcttttggaatcctctcttccaccaggtggaaatcaattgcagaacaagatttatttttatctgtatggacagcaatccatttgaattactctcagttttctacaacttatgaaactgtaaaacagcccagtcaaagtcagtgaaaggcacaggcttctatagagtcagataattccaaagcgtctgttagacattgcccagcacttgactgaaaggatacccagtagtctttgtatccctttcaaaatcttcaccatattttctgatactttccttcctttataatttgaacatcttcacccctttattcttctctggtatactatgtcctctctccctcagtgtttctctctcaggggaaataattcacaattccaaagttttaaagaatgcaacggaagtccaggttttgccttgggcttcctagtatttgggtggctagaaatgtagaaaactgggaagaggtgagctgtggatgcccacaatataggttcagactgcaatttcccagaaatataacaatttggactagtcaaagagggcccatatcattacattaaaatgccagattatctagtttttatagtatcaccctacatttttacagcatagtattgtttagagagcacttgccgctatgttttccatgttaatgctcaacacagttctgtgaagtagcccggtgtttgtttttgatcctcctacttaagagatcctcctattttagagagtaaaccaaggcatagaaaagtgagttgcttaaggcagcatattaaaaaggggcagaaatagtatttttactcaggtgttttgaacattgtccagtacctatattccagtatgcatccttgcattcaattcatggggtatttattgagtaaacatagtgttcttacaatagaaacattctatgcctctgatttttattccagtttatgtagaagtaaatcttaagtgtgaactattaacaaagttgatattttatttatatttgttagtaatttgtgttttgtttttgtttatgttttgaggggaaggccaagtagctacttaggtaaaagagttgctgagtggctgaagaatatggaagacaactacaattcctacacattcttgtacattttagttgaacaatgagtgattacatttatttacccagtgcctcttctataaggcaacaactggtagtttatcctattgagaagcgtagataggatgcttattagcagtaactgctcttggtttcaacttgcatcttacttagctttttcaccgttttgtggtttcttggaggaagaatacccataatatacatttggagactgttgttctgtagtgtcaatgaaatgtgggggtgggaaaatgtcattcaagactcccatacaaaatgtctattgctgcctatattttgctatgggaaagtagccacagataatgttttttttttcctcattagtattttaagattttccatcctagtggaaagatatgatttgattcatcctatttactttgtatattaaagtacagtagaacctgccactttttttggaaatgcagcataaggataaagataaatttcatatcagttcagcaagttctatttagcagtgtgttgaagttgagactgaataaaatatttggtttggttttctgttcaaaattttaccttgataaggacaatatttttctacatatatcagtaggcagtaatgattacttcaaagcttccaaagccagatactacacctgcatgttccaacatagttgctgaatttattcccaagatgcatgtaatgtatactttgtattattgagaatgaataaagaaaagtcataatgatgccttccagctgtgcaagttaatattaaaatataatttgtttgcatatttcacctaataggtcttcttcattgctatactgtttacttaagtgaacaatggaaatgttgctgtttatcttaaggatttgtaacatgcctaagatcttacagtacagacttctataattaatgaaacatttttctttttcctttccaggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttggtaagacttctatgaaagcttctttttttatttgtcctggtgcaacctgatcctctttcgagaggaggccaaatggggataggtactcctttgaatcaaaaagcaggctgttattaataaatgttgatgtaatgtttagcaagtttaagattgtgatttctatcctatttgttaatctactcttcatggtaaaacacatttactatttaatttgtattacttgttaatttgtatattgtgtggttctcaaacttttggtctaaggaccattttatttggctgattggtcctggggtaatgtactcttcatttctgtttgctgccatccaagtgctttctttcctgtgactaattttcaagtctctttccaattattcagccttctgttttttgtgttttttttttttttttttttttttttttaaccttacactttgaccaagggatgacaagtcccagccatgctccctctggagagaatagaggagagtcaggagattaaatcacgttatcatggatgatttcttcagattttctgtgcttagccttactttggtcttctttttgttggccaaaatatgagagaactagagattcaaattcatttattcaaattcagacttgatgatagcaccatgattctgccctttttatcacaaacttgggttgctgaggtgtagaaggtagtaaaattagctgaagtaaatagtctcctctccttgctgtttcctgctgtgctagctgagttcttcaaccccatatgagtatgttctttctgttttgaaatctctatttagaattacctgtcatattaccgatcatggcttaaccacattttgagaaccttaactctagataggttcagttctgttcagttggatcaactatttgcctagcttttataaaataatctactaccagattaggaatggggtgtttggtttgatgaaatgtttgcacattacttaggattgagaatcaggaggtctgagttacagcccctgttcagcaacttactattcacctgtgtgatcttgagaaggtgcttaaccgagtccaagttacctcatgtccgaaatggagatgacaatagatacttgcttttatgtattctacagtctaatgagcataaaccaaagtgtgttgtaaatgttagttgttttcctgggatgtttattttatgaagtaaggtatcaagttgttgaagttagcctagagcattgtgagagggatttgtgtgcagtgaggaatgggcaagggatggctccgagtaggcaaggagaatttgaaaggtctttctgctcagaaatctctcttacattgtagcttttactattgatattaatttgggcaaaggagctttttttgttgttgttaagtatacctttaatgaagggtgtaatcagtcagtcatatccactgcaactaactggtattagatcatctaatacctagttgaaatttttctgaggtaatgtacaattttgtggaaatgaaagaataagatatgggggaacagtaacttttggggggtaatgcaggtcactgagaggtaaactggaaaactcaaaatatgagatttgaaaggccagaagatgaaatgaagatggaagagaaaggaatgccaggatctgagataaaaacagtttattattttgcgtgtgattgaaagactgttttatctttgtttgcaaaataccctataaaaataaaaactagacttattttgggagccattaaaaggagaaagtattgttttattatagagaatgtagaaaattctaccagaggtttgagacatttggctttgcatccattttaattttttgaaataattattgatttacaggacgattcaaagatagagagcttccatgtgcccttcacccagtccccccaatggtttcctcttatgtgacacaatatgaaaatcaggaagttcacaatggtgcagtgtgtgttaatggttcgctctcattttatcacgtgtttatttgcgtaactacctacacaatcaggacatagacctatccatcataacaaacatctccctcatgcttattcttactagtcataccgccacctcccactgtccctaacctggcaagcaccaatttgtcctccatatctataattttgtcatttcaacaatgctatataaatggaatgatacaagatgtgaccttttgagaagggctttttcactaagcacaatgcccttgagatctatccaagtttttgcatctatcaatagttcttttttccttacttttttttttgctgagtagtagtccatagtatgaatatatcacagtttgtttaaccacttacctatagtaggacattttggttgtttccaagtttgggctgttacaaatgaaactgctctgaacaattgtgtacaggttcttgtgtagacacagttttcatttctctgggataaatgctcagaagtatggttactgggctgtatggtaagtatatgcttagtttttaaagaaactgccagacttttccagagtggctatttcattttacatttccatcaacaatgtaagagtgatctggtttctccacatcctcaccagtatctggtgccactatgttgtaaccatttttaactgaaaaaaacaacaaacaaacaaaaaccagttaaaaagttatctgcacaaaaaggttaaatggggcaggactcttactatggagtataagttcttttctaataagaatacttacttgtcacggactttgaggatttaagcattagaaaccatttttactatgtcgtgatttttgcaaatacaggcataaatgagaaacagaatttgctcaatagagaagtgaattctacttaatagaatgcagataatagtgaccattctttgtgcctttttaaattttttggaggactaggactgaatacatggaaacactataagatatagttttatatacctctgtgcctatgtatgatatagtccattagaaggagcactggacttgaaattagaaagctgttttctagacttcactgtttaatagcttaagaaaagttggataaaaaactcaacctatctgtccctcaaattcttcttagtacagtaaagatggcatcaccatcatggaaatgtttgagatataaataatacctctcttacttaacaaaattttactaagtgcctaccatgtgccagtcaccgtactaggcatcacagattctgtgatgaataagacattattgctgatttcaaggaagtggttgcagtacaaatatgaagagtgaagtgacctaacatttatttgcacatgtgccacatactttatgtaagtcgtgtcctgttatcctctcaagaactctaggagatagatggagttcttaccatttaacaaatagggaagcaaaagagattcaaggtattgttcaaggtcagagtaatagccaagaattaaacctagatctctttggcatcaaaacccagtatttttaccaccgtaatatgtggttcatgtgaaaacaccttgtaaataactaaatactgaatgcaaatactagtaatcataaaatttatcattaatatttctgttctattagcaatacaaaaacttgaaaacttaaagtttatttttctctaagctattagagttttgtttagaaaggtcagttcaagttccgcagtgttgccaattccattattatcaacaggacatatggtctgtttataatgaagacatgaaggcattgcaagatctttgtattagttttctagtgctgcataacaactacaaatatagcagcttaaaacactacccattttttagctcaaagttttgtaggtggcaagcctggtcatggcatgactgggttctctgatcaaagtcttaaaagactaaaatcaaggtgttgcccagggcatactttatttggagtttggggtcttgttctaggctcacataattgtgacagaattcaattccttgtgattgtaagactggggtccctgtttctttgctaagtatctgccaaggaattgtagcagctcctagagattgccctcatttcttgccccatggccccttgcatatttaaagccagcaaaggagaatcttcctcttgttgaatccctctcacatattgagtttattttgccaggaagaacccagacccttttaagagaccacttggttaggtcagctctctctccctcaaaaataatctctctttcttaaagtcagctgatttgggatcttaattacatcagcaaactcccttttgctgggtgatataatcatgaaaatgaaatccaatacattcacagtcctagtctcccacacacaaggggagggaattataaaagatgcatggctagggcaggggttttgggaccaccttagaattctgggtttgattaaataaataatgggactgtagcctagcaagtcgacacatcagaaaagccatcacaatcctgaaacatcaagtaaaaatttggtttgcattttaggattgtaaatgttactgtggtgtatgcgtgtgtgtgtgtgtgtaacctggtgttccctatgtttgtgggagtttgaaggagacactttgttgataggaatggtgtcttcacttttttaggttgtctcatttttgtacagtgataagacaaatgaggtcctgggttttaataacacttcagcttgaaagcaaaaattaaacacttattcattgactacacccttgtaatatcacttctttgcctttccactagcaagaagttcattttcgtggaagccattctgcctgtccaggaattggaggagagtgatagacacagttgtcagccatagcttgggtagaataaggatgtgaatgtccttggcttatctttattaatcttgtgatggaaaaatatctgacattgttcttagtccattttaagcttaatttatgttcttagtggcatagaaattcagagctgaaagaagtatcatgtctcactctccccttgaggaacaagagttaacgtcatctgacagtactaccataacaaatcaatagcttaataggcatataagtggctttatataaaatgttggtttttttcccccagcatctcagttggttcttaaatatctaattccatgatcctcaaacttttcccactgtaacaaattagagagaggaggaacatgttcatcagtgactgtagttcaaatacagcagaaatgtgcagcagtgatttcaaactgagagaatcctggatgcccttctccattgtgtgccccccccccccacccgccccacatatcagggaacaatttaaaatcctggcacaataatgagaagggagagtgacaaactgataagttccagttaagaatcacttagacaggccaggtgtggcagcccacgcctgtaataccagcactttgggaggctgaggcaggtggatcacctgaggtcaggaatttgagaccagcctggccaacatgataaaaccctgtctctaccaaaattacaaaatttagctgggcatggtggcgcctgcctataatcccagctacttgggaggctgagacaggagaattgcttgaacccgggaggtggaggttgcagtgagctgagaccacaccactgcactccagcctgggcaacagagtgagactccatctcaaaaataaaaaaaaaagaatcacttagacagtattttttgttctgtaatcttcctctctgtcattgaaatgtctcatatttcttgtttcacatagaatctgggagtcatcaagtagtcatctagtctatctcatcatctaatttacgaattatttctatcacaccccaagtagctggcgattcagattttttactgaaagcttgacatgatgagacattattacttcccagaggatcttggtctgttatcaggcaatttcagttcttccttaaatcaagctaaaacttgctactattctcactcactgggagggctgtagtatttctctcaagtttatacctctctgtgatgaaaatctctgttcttttagccattgctcatggtttcgggatccccattctagtcatcttcctttggagcaattccctgttagatcttcaaatgtgatgagtaaaactgagctcaggactgcgtttgttggtcacacccttctcccttccaaatgactgctgctgtaactttctgacaggtcctgcttccctgtaatccattctccatgtggctgccaaagaagatttgaaataagtcaggttatttttcttccctatttaaagccttccagccgcttgtcattgctcttagaataaaatccaaactctcccaacaagcttttaagactctacatggtcttgccccagcaaactcttcccatttgatctcatcactgaattccagtcacaatggccatcctttggtttctcaaagttgccgttcctttggcatctcttcctccctgtcttcacaatgtaggctccttcccatcctaggcttagtttcgttcctctccctagaggcctttccttactgcctcagcttctcccactctgcacactcagggtcctctgcattgttcactgctggccttcaagagcctagaggagttcctccccatggtgggctttcaataagtgtttgtggaataagtgaaaaatgagtggtcacaccaggatcgaatgcacctcttacttttgttaatatattcaaattaatattaatatattaatattctattaatatatcctaaaattttgttgtgtataggggcagggaccaatgaaagaacatttcattgtcagctcatcttgactatgagatcagatattggctatattttgaaggtagagtcagcaggatttgctgtttaactagagtgggatgtgggagaaatcagggagtcaggtataactccaaggttttcagcctcaagcaactgaagaaatggggtcaccacgactgaaatgaggaaaatgagagggggagtttagtgggggatggggtgatgaaaatatcaggattcattgtggaacatgaacagttaagattaactgaagagcctgaattcactgttcctcagtttctacaactataaggaggggttaataatttctcacatcataattgtgaggatttgaggagttgaggtacacaattaaaaacaaaagcacaggggaagtaaggaaaaatacatcaattaagcatggctattagaccatacagccttaccaaatccattggggattagatacaaaaatccaagcatgatctccaaaattttttcctggtagggttttaggctatactgaagtgacttttctttggcataagaagatattcagttatacagttggaaataaaggtattgatttggagtatccaaaaacatctctcagtagagatccacaaccaaagaagcataaaaaaaagtcttccattcactcgcaaaactgtcttatgaccattgcaaccctcaacagcaaacatatggagtctctactacatatagaggaaaatgctagattctgaaaagctaatatctataaaacagagtttatgatgttgttatattctggggattgatgtaagcattttaccaggacttattatagcattataagcttaacacaagaaatatgtggctatcattatggagtgaaaggctggaaaattcctcacctgtgacctgagagatagtgcgtggtagaatttgaaagaagtgttgatttcagaggaatgctagtgcttgcttagggcagtattcagaagaactcttccaatcacacagccccttgtacaggcaaactccggaatactttcagtacatttacttggctttattgttagagcagaaacttcacagctaaaattatggtcttaggggtttataagatgcaattaaacttaatttttaaaagttctcacaaatcttatttgaagtcttaatatcttaatttcctttatataggatatgtggatatatttttatgataaaagtaacatgtgaccaacttaataacagcttcgttaatatcctaaagccactcatcttactttacatataaacagtccttctgaagcctaattatgcatggataattgcagagtgagtttgggaaaagaccaaccgggcacatctctctccattttaaccacaatgagaagagagtacctagtggtgcatcttcctcgcttaggttccctgagtctgtctttacaggaagacttcattgttacttgaaggtacattcttggaactttacacacccagccactcaacacatgaatacatacttattttagttaactgagtacttagtgagtgctaagctttgttttaagccctttgcatgtagtttattaactttgttaatagttacaacagtccttcaagatacatgtactgtgttatactagtgtaacagttgaggacaccaaagaacaaagaggtgttgacacgtggtcacggttccatagagtgttaagttagagttgggttcaaaccctggcagtgtggccacagagccttgttctcagccactgcactgcactacctcctcccgtgaaacataagaaaaatgtgagaaatgcttaagtaagtgtagtttttattcataaataaaatttacataagtacattatgtgtaatttgttttatgtatatgtgtgtatgtataaataaatacatgtaaaaataaggccacagttttaatttttttccatctctataataaagcatgtattatagaccattagcagaatttaaagtgttatagtaaatattaattgtgacttttgttttcttcttccccagtttcatggttttctccaaatgaaaatattcttattgttattttcccaatttttgctatactcctgttctggggacagtttggtattaaaagtaagtattatttctacttttcatttatgtttcagtgatgatatagttatttctacgagacattgtcagcgaaatatttaaagttgtactaggaaaagtgctattatgataaatatgagtatgtaatttgaatactactagtctccttgaagtatatgttgtcgcccacattttgctgcagttcacttttaattcctaagaaggttgttttcacttggtgtttttttaatctcttaagaatgaatagtaggaatattagtaccaacaccttaaactcatgtcacattttaatattcacagaacatctacacacacattatgttattaggtaaacaggtggtgacagcctgcattagttttaaggtaggacgttatactttggagcatttagattcccctctttttattttcccagtttgattttctctgtgtacacgtgttcacccttggaaaagtccagtcggaactatgttttgtcatcctctgcgtgcagttctgcagcctctaaagaagcagccaccagagagttaggttctttgatcttgctttcctataatagtaacgtaaccagacttctgaaggcagatcttgatgctgcattagatttagcttcaacaacacagaattgtcattactaggcaaataggtaatatgcattacggttaatgtttaatcaaccatattttcatattttggtaaagaaaatttacaaaattaatgaagttctgaggtgacagtctaaacttttaagctttttaaatacaagatttattccttctttccttgtagcatcctgaagaccagtaaacatttatataagcaaggaataaatactgcttatttaatttattctgcaacctttaaacacacaaaagctagtaaactattgcagtggattgccctgttgtatattttatgaattttacttttactcagcagtttaagctgtctatatctatggtggtgtataaacatggaagggagatgactgattgattatatgttttaagcgcttttctcagtgtatggcattctggaaatgcttagtgatttcagaaatgttctcaacttttgtctgaaaggaaaaaagggggaagaaaggggtggcagtggcaactgtcaagacattttataacttttactttcaagatagtgtctagacttcttttggaaattttcttataatctcttagttttttatgtcaagaaaaggactggtgtagcattaagacctattctggcatcaatgatattagggaaagcttttaaccatattggcagagccagactttaagggctaagtcatatgacttgggcagggaacagcctttttcaaagatcatgaaattatttcacatagtgcgattttttgagtttggcttgatggatgtttgctgatccagacgttatcattggtgacattatttttagtgaaatgaaccaggagtgagtttgttcactgttggctgtatttttacaaaatgagctttacaatattttttctgacttaaaaaagtcatacatatcactttagaacacctcgtacaaagtagggagttatttaaaaaaaaaaaaaaaaaactcatctgtagtccccaaacctagaaataaactattggtatattctggtgaatttcctgtttccctttctgtctttttctattcatttttgttatcttttttttaaaaaaaggtaaccataaatggactcgtacagcttatatgcttgtacgttctgatgttcccccatgtcttcagaaatcttgttatagctgcctgtgttctacttttgtggatgcactataatttacttaactctcatcttgatggattttaaggatgtttccaatttttttgatactatgaaaataactctgctaaagatatgaagcctggaattggcagagatattttaagactctagatacatactggcaaaatgttttccagaaacgtttgtatcaactactattgcagatgagaatacctgtctttcacctcagttataattctgatattataacgctaaaatctctgcaaatttgatgggtgaaaaagcatcttattgtaattgtaatttttgtgttagtaaggtggaatgttcttttttatattttttgtactggtcatattttaaggaacgtgcttataaacctaaagaaatattttggtgggaattttttgttttggctcatcttgaaacaggtagatgtgtgtgtatgtgcatggaagaggtatgtctatacatgattcacccagcctgcgctcacatttaaaggtgttgatgataatagtagctaaccatttgtggagctcttgctctgcttgacaggttctgtgcaaagtactctatatctgaaatgacatttatttctcccagaaactctatgggcatagacactgttgttattcccgttttgtagataagaaacaggcacagatagattaggcaatttgcccgcaatcacccagccgtttcctgatagtactgggatttgaactagtactgtttaccactgcactgtactgcctccccgttttgcatttattttgaggattttatttccatgaagggtgaacctatatctaagcacataatactgagtagctaaaacttattaggagagcagaatgttgacctgatttgtttacttattctgcaaatacctagtgacagggtgcctactgattgctggacaccaagctacatgcctgaaatgtgggtgtgatgtgcatattgccctggttttgtagcacccacatttaagccagggagacacgaatgtatgtgcatcccatgccttgtcacatctttgagatagtcatggatccaaatctaacttttctgttagtgcctctgttgatggcctgagcattccaccaaatagaaatagaaagcacctctattctccacctgcttagatgtatattttttagaatccagtatggtaaatcttctaaagcattcataataactcagacccatgacttttattttatagatttctagttctgcttaacttttcctgattaccatttatggtctgtacttggctacaggggatttctctcagtggtttttccagctctgcatgtgagtctttgtggtccaagacaaacctggacctttaccaggctaaactttcagtaaagacagcaggttatgccctcatttgctcactctgaggagaaagagtttctttataccagagctgtatcttgaaagatgtctcaggatgccattggtcctactgaggaagaagcctgagaagactcttaaactcccagagcccagccagcacttggtgagccctggaccacgtttcaaagataaaggcctcttacagggaaatgttcctaaataccttcacctttagcttagctttaacttaggaacttttaagcagaatctctatgcttagcaaacagctcagagattgctagaatcaaacaccaaggcttaactgatagtattgaatttcagacgctttgtttgtgttctgaaaactacaccaactcacagtttgccactctactgacaattaagttcctggctgattttcaggattcttctttctcactctgatatcattttaagtgctgtccacctagtcctccagttcctgcaggattaaggtccaactgtatgtaaagaactggctaacattttgaaattctttgagataggcctatctgttctttctttgcctttgtaactttgttttatacaggcaatactggcttcgcacaaaaccgcaagaccataaacatgaccatgtaagctgaaattctgcaaaacaatcttcatgatgaatgggaaaaactattattatattgttccatgacctttaaaattttttttgttgttaaaaccttaaaaactctgttattatcaataacaacggcattgggaaatgaaaaatagtaaagctagtatttagtatgtggtaaattaaatcattagaaacatcgagaatgaaagtgtgttatcaagagtagtttgaacaacacttgtgtgttcttcttgcataactttggatacagagcaagcatcttctctatggcttggtgaattgtcatacccctttctaagtttggatcggtttctaccgttttatcctttgcactctcaatgtcatgaaagaattcctttaatgtttttttgaatgtttaaagtttattttattgccagtcacttcctctgggacatctttattctttttgttacatcctcctcactttcctcatttatatcagtaagtccaccttcgctgagttcttctggcttcatctctagagtttcttgcattgcagtaatgtcgacattcgcacaatcagctatttcttctcttactccatttatggtctattcaaatttcactcctagcatttgtcagtttttatttctttactctttcatctttcttgcctagtttcctcttttgatttatccattataaaatgtcacatgggtttatcactgggaaacaaggaggtaacaaaactccatactttgctgtctgtgcatgacctaaatatcagatgtacagtgactagtcaccaacagtcttcaaaagaagtgacatggttgatcactgatcatgatgggacatctgctatttacatagttatttttgaactgaagaaatagcagtgaagttgtactttatgcagttactcagttaatatattgtggtaattgaaatttggactgtttatgagggatttatttgattaaaccatggtaactggaattcatatcagaatagtgcaaagtgaggactgctgtacttgattccaaatttaaaactgtattctaggcatctttaatttttttttttcaactttcccctccctgctcaaaattactccacagagattttggttacggctcagatgtctttgagtagatgttcctgtagaaatcattgttagaaaattcaaggaaaaaagttctcattacactaccttgagatcctaccaagtccttgagttttgacttgaggacagcttaataatgaaagtaattcagccatgaaagcatatttaaaataagtaatggaacaggaaatttcttcatagattagaaaaattattctgaaaaagtgaagacatagcctatcttggactaggaaatttccatccaagaaagtaaaatatacaataaatattatcacaaagaatcattctgtgacctggttgatggcccctggtaataagactttgttattaagtttactgtgaattctttttttttttttttttttttttttttttttttgagacggagtctcgctctgttgcccaggctggagtgcagtggcgtgatctcggctcactgcaacctccgcctcccaggttcaagcgattcttctgtctcagcctcccgagtagctacaggctactggcgtgcaccaccatgcctgggtaatttttgtatttttagtagtgatggggtttcaccatattggccaggctggtctcgaactcctgacctcgtgatccgtctgtctcggcctcccaaagtgctgagattacaggcattagccaccatgcatggccccttacagtgaattctgttcaataatcttgaactcccagtgctttcccttggtcctgtccatataatgatcacattctgttattaaataatgtgcttatgtgctgattttattgtaggaggaattgaacaaatctatagctcagcattttactcttttgtagaaatcccttgaccctttccaaatccagacatcacaaccttctccatatactcaaaaattgtgaaatccacaaggcagggaaagttgataatgaaaaaaaggaagtttggtctgttggacatcctcttctttcatcttttatgttcaaagtacttgatgcacaaagcctggactcttttatctctgtcctatagactgatgtctagtcagtattcattgtgacaaaattgtttcattagagcaagttggatagtgtatggagattattataaaatgatttaacttgtcttctgtcaagtaattttataattggtattaatgttcatttttttccagtaccagtttcagaagctttcatttgtgtatgcatgtgtgtgttaaataagtgtattagatacttgaaaaatagtaatttaaatttaaacaatttaaaaaatgaaattgtaattttatgtgtaaaggtgtctgatgtgctttattctgcactgaaaacaatattcatttacagctcaacagacaccaaatatattctaaaattactttcctagagttatcagaaaacagcactgtaataatgaaatcaaacccatctttctttatgatttattcttagtctgatacgcatcagcctggtaagccttcctgtctctctgcttacttaccaatcactccaaatgtcatgtctttgggcaggcattaaattcttgggttttgggttttgttggatggactgcagtctgtgtgagcctatatgggtgtgtcaaatccagtctttggggtgtcatggaaacttagcatgatagacttgattttatccccaagttgacttggtaatttcattagatttcatcagtcacaacctgcattttatcttgtatgtgctgtctattggtcacaaaatcagaaaaccttcttgtccattcataacattagctgttttttcaggtggctagagggacatgtcattgcttcatctgcatgaatttgaaagattaaatgcataaaggaattttcttagagtagagtaggccttcaccatctctttaactgggaaagaagttttgggagtaacatactcatcactcacacccccctcccctcaaacacacacactcacccattagaatgtaagggccttgaggcaggccttgtgtttgcttttcgtgttcactgtcagcatttagaatagtgatagtcatatagatggatagtgctcaatcaattattgtcaaataaagtaatctacttgttccttgatttagactagcaaaaggggctggtacattgtaggtacacagtaaatatttgtagaacagatgaatgaaccaacccaacagatttcttagaagtagcctgcttttggttactaattattttatagaacatataaaagaaaattttagaatacctaatttgttcacaaaaatgttatattgtctcccctgatacttggtattcctgtgtcaggctgagcactaaggttatacattttttgttaagcatgtaattactgttatttctcatgtactttattttgtgtattgatttcagtagttgtagctgagggtaatccttaagagcatgcagattttaaaaattacatagattgccttgccaagccgtctgaaagtcatactagaaaatttgataagagcctgctgttaacaacacattgagttatttttatcttaatgctaagtggagaattacttgaatttattttttttgctgcatttaatgttttgatacattcaataaataaggataaatccacttactgaaggacaagaaaagatcaataagagatccttgaggttattgggttctatggcatctattatatcaaattccttgccctttcactatactgagaaatttgacgactgggctagcaacaagatacctaccccacattagctgtggtacctgctgaaatgtgaccatgtgagcaaagaaaatgagccaaaagaaactctttctgaagattcactgaagaaaacctggagatactctttcattagtagaccccacagagacaggtggagtgtccgttatccagaatgcctgggaccagaagtgttttggatttcagattttttcacacttgggatcctcaacatgtgtatttgttggaaatggctacttaatttaaggaaaagtttaaggtggcctggaaaataagtagtagttgaactaatcaacaggaaccaaaactactttcaatatatagtgtacttatactcaaagagagacggaccattttatggtggaaccctgtctatggtagtattttgtgatgttttatttttgttgctattgtttgcactgttttctcctataacagctcttctaagccttaagaggataaattttatatgagattcaaactcttatttttgttaagaaggatgtaagtcaccaggcatggtggctcacacctgtaatcccagcactttgggaggccgtggcaggtggatcatgaggtaaggagctcgagatcagcctggccaacaatgtgaaaccccgtctctactaaaaaaaaaaaaaaaagaattcaaaaattagccaggcctggtggcgcatgcctgtagtcccagctactcgggaggctgaggcaggagaatcattgaacctgggaggcagagtttgcagtgagccgagattgtgccactgcactccagcctgggtaacggagtgagagactctgtctccaaaaaaaaaaaaaaagaaaagaaaaagaaaaatatagaagaagaacataagtcaacctttttccatgaaattatactgtactctaaggcaaattcttgttgcttgatcttagtagcttttttatgcattgacttaaacctggaagagtttcttatgaatgtttgattgactgtgaggtgcatttgaaacagcttctactttataaccctttgcagaactttcaagctctatttagataacaatatatatgggattaaaatggaaaatgtgacatatgtctagagaaagttcctttttctgtgtatgtgccttttgtgacttatacactcagtttcattctagccagcatttgagcctagaaaatagggaagttatgaaaactctgtgccttataagaaggcaaggctatgactgagtattagacacataagtccagggctgggcggaagtaatgaatcaaataaaaacttggggaacttgccataggatgttttctgattaatatgggtttctcttctctgattaatgtggatttcagttactcaactgtgtaactgaaatccacattaataaattggttatttattgcttaggacttatctccccataaagagactaaaattgaggttataaattatgggtagatagaaattccagaaatatttaggtacctgtcatataccttaaacatagatttttatcacaatcatttaggggcttatttattgcttttactttatttaatgtttgtcactgtagaagaaaaaaaactaaatgctaaatataacatttaaaatattttccccttcatgacagccaccaattgattactgtcatggagactatctctatgtggatgacacacagcagcatttcaatcacacgctgtttctttccgctactcagtttgcttttaggttgccttaaacaacttccttggtgaaaaaatcaacttcaatattaaccaaaatttaaaagattcatatatagtaaaagaactaatattcgcagattgacccattccattctttcagagaaggtatgagatacttgacagtggagctagcagcagaaacaataacatgtatgatgaatcccattgagtctttgcagtttatttttattaaaatattttaattgaacaatttaagctttttttcttcatcaggatttcacaagggtgtaatctgcccagttttattctgtcatttttttcaaaacaaaataactagatttccagattgtttactaattttttataaggtaggacaaaattctctttttctcaatattgttaattaacccattatttcccattagtattatgtacatcagtgttgtgttgtcacttggagtggttttgctcatcgggggcattgacaatgtctgaaaacagtttgattaacataactggcatctagttactagcatctagtatgtagaggccagggatgctcctcaacattctatattatgcagagcagtgtcccccctccccccaaaaacttatctagctcaatttattagtaatacatcaaccgagaaagactgatgtatagaacctcatttgttagtgtaggaaaataaggtgtcacctataaattcaccatccatatttatactgccgtgacgttatccatttgcttatgaaagagatgtgaggtgacttgatgatattaaggagttgttcctcataagttattacattatagtacttctgtcagtttgtctctgtaccttacaagttattaaaatggcttcacttgtgattgagttcatataattctttgtttttcttttttaagcacttaaatatagatccggtggtatggatgagaaaacaattgctttacttgttgctggactagtgatcactgtcattgtcattgttggagccattcttttcgtcccaggtaagatgtgcagttcctaggcaggaacgcaggaggtagatgagtgcattccaaggtgaggagggctgcattagtttcctagggctgttggaacagatgaccacaaactgggtggcttaaacaatagcaattccctttccatgctggaggccataagtctgaaatcaggacgtcatcagggccacactccctctcaaggctgtatggaagaatccatttcttggctcttctagcttctggaatttgctgaagaatgacagtgtttatcattccttagtttgcggcagcataactccaccctctgcctctctggtcacattgcctcctccttcctctatgtctttgcctctgtgtttttgttaaatctccctcagcctctctcttatgagcacatttgtcattggaattagagcccactcatataatccagggtaagctcctcctttcagatccttaacttcatcatatcttttgccatataatgcagtattcactcttttgctgtattaggtaatattcacaggttttagggattaggaggtagaaataattttagggccatcattcaactcactacagaggcaaacttcattggccaagacatgaaagtagaatgaatgtgggaaccaagtctggaaattggcagttgcatttggaggatgggaatgtgagtgggaaatgaggatatgtagcacatgtaaagaaaagcaaaggaaggctgggactcaacctaataactatagcacaccatcgtttgtggagaagaatgcagtgcaggtgtaatatttaggaacatgggcttggcagctctattggtatctgaagttagtgaagacagcaggaagaaggaggtgaattcagaagacaacttgaaggaacaattgatttgactttggggcatactgaatacagcagatgaagggaacagtaaaagatgaatacacagtttctcactggaattctgtaaactagttaagaaggaggcagagccaattcaaggtttcagcatgttgaaacttaaatgttgagatttagatgtcttaagtttggtcttaatcaaaaaatagtcactaattttgtgtgaggttttcagggaaacgtatatttttaaaattttcacagttgtcaagcacagaatttagattagtacatttaaaaagtatgtaggaaaatattttaaatgtttttatttattgacaggtgaatattcattaaagaatgctactggccttggtttaattgtgacttctacagggatattaatattacttcactactatgtgtttagtacaggtgagttttcattgttacagtatgattttgtctacctttttcatttacaaaacagcagttttggtgaaaatgctcatataaattttttacaactcaataagagtaggtttattaaaagatttttcatgctattcttagtgacattttcccattcatattaattttaaggttattctaggttagctgttttgtaaaaagtgactttcatatgtatttgatgccaatcagcataattttaaattatgccatataacttcttaatgattattttcatattctaatttcagttttttgaagttataagtggatgttaaacgcagtctttctcttcttttgctaagcttctgcttctgcattaagaagactggttctataaaatggaattatgaatcacaaatagtagaaacatatttgttttaattattagcaaaccttaatactgtagttttaagagatggtatggaaatccaaactataatcagtatcattttcactgcattttgaaagtagatcactaccatatttagttattactattaaagagtcacatttaacacttggtcttagagcctatctctggctcgttgtacattggaaaggtattagtttgttataaaatgacatctagagatagagagcatgatgtttacagtcaggtatttgcataatggttccctcagcaagagcaattccatgtgcatgtggagaagacattcataatagccattcatattgtaaaatgcacaagtgtggtaaaagcagcattgttctaagatttagggtaaaaacttccagtccagcttagctgactgtgaaaattaaaggctgccgaatgtgtgaatttggagctgactacttgtgtggaagggttagatcactgagttaacatctaccgtcaaacaatgaactccagagtcagtctttggtcttaggagatccctgattataccaaatgcaagtggaagttattgctttttaaatactcttgacatgcttctgttacatccttttccttcctccagcgattggattaacctccttcgtcattgccatattggttattcaggtgatagcctatatcctcgctgtggttggactgagtctctgtattgcgggtaagagtcatctttctgtagacctaatttgggttacttttggacagagctcttcttcctttttctttttctttctctctttttaaaaatatatagacttgatttttttttttagtgcagtttttggttcatggcaaaattgagtggaaagtacattcacatggatatttttatgtggacataagttttcaattccatttgggcaaataccaaggagtgcaattacgggactgtaaagagtatgttgaaataaactgccaaactctctgtatcaaaatagctgtaccattttgtattatcaccaccaataaataagagtttttgttcattcatatacttgacagcatttagcattctgagcttttctttttaaacttccaatttaccccagtggtaatagtgctttcctcctcgctacaaagatattagctgtatatatggcttggtggctgattgttctagcacccaaactgatatgcctgtatttgtggaagagcttttaaaataactgggctcaaattggttggagccttagacttgaaacaccagttcccatttcttgatatgataaggtatgtgttatgcaaaggagggctttgttcttctaataattttgagtcattttactggttaagttaataaacatatatggataatttttgttttttgatcgttagaataactctcttaaaacttgggattattactgttttttagtaagttatttcatatgcttttctaatacagaattttatttgtttttacagcgtgtataccaatgcatggccctcttctgatttcaggtttgagtatcttagctctagcacaattacttggactagtttatatgaaatttgtgggtaagtcaatcttattttcattaacctatgccaataatttcagatatatcacttagaaaatgctttttagtttgttcttccagtttaggaccaaaaatgagaaaatacaattggagtgattcgaggataattaaagagggtagaagacatataggatttttagttggttcttccagtttaagaccagaagtcagaaaatacaattggaatgatttgagggtaattaaagagggtagaagacatataggattaatgaaaatttggtttccaaagtagtttaaaggaaaatggctttatctattagaatgtgtacctttttatactaagtaaaaggggagagatctttgaggatccattttaagtaatagaataggatttttaattgttccagtgtttctgtgatagagctgtcctgcacagacctgtttgtttgtcacttgctctttttcttgcagacatagacaccccagacaggaattaaaattcacaatctatcaattttgttcatttaaagagcagtgacctctaatgcatgactttaaaacagttctagttaaaaccaatataatgaaaacattgagtttcaaaatttaggcttttactccttttaaaatcaattattagtaagtatggaatttacttcattgtttctaacttgtatatttaatctgccaattttcaagtaacatttctgcataaattcttattttttattgagatatatgtacacagagagatattttcaattgtgcctgaaactaatgttatcttacctaagctcaagatgttcccaataatgtaatttatattagtttccgttttttaaaaaaattatatttttatgaaataaaacatactcttaaccacctatcaaaataatcaaaagttataaattaatggagtaaaaaaatagtgtttctgctttgctttaggtaaactttgctgtatgtgtttctaaaacttaatacgaaacttgaattgttatagtcaaataatttctcatatgactcacataatagtttcaaaaaacttttacctttatttctgaactttggttcttgatgattgttaattgaattcaattctgtcatatattctgtgtctttctttaattaatgcttattagataaataattaaaatacttaactaaaatctgcgtatccttagcatatgagttcataagtcttagttgttgctcaatgaaattttctaattttataccacataatgccataaaatacaatggagacatctaaagcagaatggaattcatgtggtagctacagtgaacatcttgaatgttggtgcatattctatttttgttacatcttccaatcaccatgtgtctggttctggaagatgacactcctggttttgttgctccccacaaatgcctgagaatagtgtgtgatttgcagtatccatacaactctggtgaagtagtatgagatacctttggctgacgggcagcacgctcttattttttcctcactatctgggttgtcctcccttttactcccatgccaccccatgccttccatatctagcatagaatgatcttcagtacagttgccagcaggtctggtgacaatgtctcaagtggaactaagcattgtctatctgccacctccttaacttcactctcctgccttctccatcacttacgttcctccaagcctgtgaaaccactgtactgtacctgtcactgttctgacattaaaattaaaatgacttaaatcttgacaagtaccccaaattattttttctttgtcataggttagcatataagtatactatatgctaaaatttatgctatatgtttaaaatttagtgcaattttattgatagtgtcctaattttattgatagtatcctaaattaactttttaaatcaacttgtctgatgccagggttcagagggacacctacagtcagttgaaaggcaagaagagacaaggtacaggaaagttgctctttagataacatggtagactaggaggaacattaatatggtttgcttatataatctgactgtgtaaatctgaatctatgtaacattaaggttgcaaatttgagcgtttatattaggaagttaaaattttaagtgtctccaaaataatttttactcattgcacgtgttctgttttagaaaagcctaatgattgtgttttgatttaaatgcaataaaatcctcaaatagttaaaaatccaagctttctcttcaagaagaagttaatgtcgctatgagatttttaacttttataatttttattatttccaactttaaatttgtagccttaatttgctattttaaagagtaggcctttcactttctacaactttctgtgaaagtgactttcactttctattttttaactttttaaactgtgttgtattttttttcttttattggaagcattttaattttataagatgagaaaaaggactgggcacaataacttaatgtgaaagcatagaaaagattacaagaacctaaccaaactcactaaagttgggcttgttgtttgtagagaacgtttatataattataaggatcaatactttctcatttttaaagccattaccagttagttcaatataagggcatatagtgttttgatacaaatcaatctggtagcagtaagtaccatatttaccacaacatcccagatattttagaatgatgcagatgcagaatatatacgtagaatttatatctatgtatagatacaaattcagatattttcttgttcaatttaaggagaggtaaatttggtatcaatagaaaaaatgtttctgaaaaatttaaaccctggaaatgtatttatggcatggagtcagatgtttcagggagagaagaacaaatcaagaagcattgcaagtatgctcatatggaatgcttaaggcttgtggttaaaaaatatatatatatggctgtcaatgtcttaggctcatggtagcagcagaaatcgtaataattcttttgtcacatgggttatatccatattggagagaattaactcaggtgaaattaacttgtacactgtttggttttataatatttagagggatcacaactgactgatgtccctttgaagtaccattcttcataaatctttttttttcagaatgggccagccaactgtgacatcccttggatcggagatttagaactagaaagtattctttctacattattagggaagaaaaggagttacttggcggttagcaatattctattttgttttgttttgtttttagagacagggtctcattatgttgaccaggctggcctcgagctcctgggctcaagcaatgctcccacctcagcctcccaagtagctgggactacaggcatgtgccactacacctggcagtgtttattctgataaatacatttatgagctcaaaaatgtaactctaaaaccttatctctgaacttccatattaccatcagaaatttagatagttgtttagttctctttttctttgtagaacatagatataaggcatggtttcattgaagtcagttgtatatacatgtaactatcctgatgttcccaaataaagctctgtatttctgcttagtttattggggaggctgctaaatgtagtgcatcccaacccattttaccctgttctactttaaaaagaggttggcttcttgtttggatacaaggaccaagtcactcccccaggttcctccacagtaagggaggcctatttaaagccgcccatggcactaacagaaactggactcctatgagctcagatacataactgggcctcacaggggtgggacagtatgtagtctaggaattggaaggatccattccatatcaaagaactgaagcatcgtgttgccctctcagcagcaagagtaaggtgatgcccctgtcagttatagttcctgagttcctctgtctttgattctttgcctattagccagctagctcaccctcttgtttatgccactgttttttatcctattcatgccttctcacagacaacttttcttacctacagctttggactcatccttgtctcctttctgtttctttttcactttcccttcccatcaccaactttctgggtttttttctgtttcttcttagagtccagtggcagggagaaacttgtcagtccagtctgttgccatttttcctgtttgagaaagactcaccagcttttggctggctcacagattggctttccttgggtcaggacccacccttttccctgccagctttggaagcttgacagaattcgagtgtgcagtggtggtaaataaatagtaaggaacacagagcagtcctggaggcgtgcctccatctgctgatgagaaaatccagtgctgtcatccagcccaggtcccagcggaatgggcctctctgttcagtaggatccccctcctgctgagtggttcatggcatgtttctgttcaacgcttttccatctgtaggattcttattctgtatttatttgtttttttgggtttttttattttttgagatggagtctcgctctgtcgcccaggctggagtgcagtggcacgaccccagctcgctgcagcctctgcctcccaggacgagggagatcctcccacctcagccttccacgtagctgggactacaggcatgcaccacaggcatgcaccaccacgccagctaatttttgtatttttggtagagacagggttgcatcatgttgcccaggctggtcttgaatgcctgagctcaagcaatctatttgccttggcctcccaaagtgctgggattacaggcatgagccaccacggccagccttctcatttgttttttttataaggaagctatctcttcttccctccccaactagggtattctttttccctttcgtcactttgctcatgtactgtattccttcaacttcattaatgaatccatttggaagcagtgaaaaaggcaactcagaaagctaagaagaaatagatagaggaatactcagagctatctgagtattttctttagtttgttagctctttggagctttgaaactggaaagacccagggagtgatgtggagaaagagactgagcttgtaagacacaggagcagtgagctaagggagatggagtagtggggacaaattctggcacattctgtctacactctgggtagatagaggagggaggatggagcacccatggtgggggtatgttggtgacagcattttcccaccagccagtgtaacaagtggctgatttgggggaaagatggcataaacaaatgagagaatgtgtttactatttgatgtagatgggttatttgcttcatttttcaaatcagtgtatataatcaagaatattcagcatgtttgaatagactgtcagagctggaactctttcattaacatctctggcacctttagttttagccctgaacattttatcttaaaattaaacattaccaaatgccttagtttatttcatttattaaatttatattcttatttgttatttatatcagcttccaatcagaagactatacaacctcctagggtaagttaaagtttattaaatgaattgtgaatgatcatttgagggattagactgaggaacttggtaattgagatattttgctatctgttttgtctcacgtcaaattaagagaatgttgaagtcattgcatgacctttgcatgaatgggtccagttctattttaaaacctgtgtttggtcattttagtgtcaatgggatggaataaatgatttcttaagattgtactgacttctcacacccaaaactggaaagtaggaataatggctatattatctctgcaatcagaaggaagctgattccaatatatcacctcacctgttggattcattggatgtgcatacacagaatgacaatttcaggcttaaaaatgaggagaaatctatactaagttgacatcactgataattataatctataaaataaatgtaaatattgctgaaaacatctgttcggagttatgattcgatcctctcccatacaaatgttttataaacattttttcccttaaaactgtgcttaaggtttcattgtaccttagataccttattaagccatctgaggaaattgcaagaaaggagtaattttaggagggcataaatgaagagaaaagcatatttataaacatagacttatcacagtgacaggcccaagaggtatgttgtggacataagctctggaaaggattatattgtacttagcttcctataacggagtgatgatagatgtatctggaatgccaaagagagtcttctgtctgtggccggaggtagaggtcacatatgcttctaagtctgacaagcctcattgtgcctaaagggcaggtggggcgggtatgtgtgtctaccacaggggtatattctagaaagattggtgccatagctatgttggtcacaagaggccagcaacttaactggacctgtgaatcctaacacattttctttcccagttactgagttcaatttgcgatacttaaagatgattctgttttgcttccacctcttcactgtgttatttattctgttgttgctatttatgcttgcactttcatatttttagaagttagaatttcttgagctgagagtggtgaagtgggaaattctgtttagaaaaataatattaagagaataatacagttattattaaactattaacccgacttggcaagctttgctttaacatttacaggcttatttcgttgttttgttttttcttttttttttgactgtaggatttactgcttactacgtaatttaaatattagcatatataagttttactataaaatgacatgacataaattatatttttatgtaaatatattttaaatatttttcagaaagctgtagaggaaccccttaatggtatgtggttatttcactcttaatcctttaccagatcataatttgatctggcccgcaaaacagttagaatgccctgtctatgccttaggaagaatctaggtttttttcctctttttttctttccttggcatctctactcttgattattcatcaagaattatgggctgggtgcggtggctcacgcttgcgatcccggcaccttgggaggccaaggcgggcagatcacgaggtcaagagattgagaccatcctggccaacatgttgaaaccctgtctctactgaaaatacaaaaattacctgggcatggtggtgtgtatctgcagtcccagctactcgggaggctgaggcaggagaattgcttgaacccggaaggcagatgttgcagtgagttgagatcatgccaccgcactccagcctggtgacagagtgagactctgtctcaaaaaaaataaaaaagaattatgaatattacttttataatattctcaccactgggaaaaatgcactattctgtgtctaagtagctgcttaccttaaccaagtgatatttgggcaaggggatcgttgccttttgctactggttgagacgaagcatggcaccccctagtagagaaggatcccaattacttccaatttgtgatgtacacattttagaaagatacaggctattgccacagagatagaccaaaacatctcattttctttctttgtaaacctagatctgatttcccaactaagtctgtttcctttgtgaatgctgtgggtatgatccacagaaaggctacataatgaaatgatagctttacaattaatttggctgtagagttgtagactagttagcatatcattgcatatttgtttatttagaaatgatttccaattgtggaacctcactaactgcctgcttggcttgttactaatcctagcattcaaagaatcaaaaggaatgatgaatgatggtaagtataaaatgcacttaataattatagatcagttaaaacatggacattggaaaacaaaaaagcttcttgaaaatgtggctcttttttagtaaaagggacactgtcagatgataaaggttcacatttcttgatgtatacaacttaaatctactttgctaaaaattgcaaaactactactgtaaaaactgtagggtgtcacgaatcagactccagtcatatggctcccagcaaagagaaattaccactttttgtaaaatgtttttcagattccgtgtctggtgactgtaactttaagatgccttttataaggcacataaataatctggcacaaatctttatcattttgacagagtttcttttatgcttgtgttggtgattttgttgcatttaacccatggggacttaacatctctgcttttccaatcaggagcttggttctaaccttttgggagatgattaaagagagaggttgagagagagagagagagagagagagagagagagagagagagagagagagatgtttcgtcctcagcttttgcttccattttttttttaagaactctgggctaataacttctaatctttatagaatatttcaaagaaatatatttgttcttaaagatacataggtttgagatattgagtgctacaagcatttattttggttttaccttaacatattatgattcctcagttttgttggcatttagtaattatgtttatgtttttatcttatcaaaaaatgtcttcttacctttgatatttataatcactcctcggtcatgtaaatagtttgctttatattttactgttttaaagtctgtgaccttacctgccctcttctgtagcaaagtgcagcatttaactcaggaagctatattccccccaagtgtcattaatatttgcataagattaaaaacactccagtcggccgggcgcggtggctcacgcctgtaatcccagcactttgggaggccgaggcgggcggatcacaaggtcaggagatcgagaccatcctggctaacacggtgaaaccccgtctctactaaaactacaaaaaattagccgggcatggtggcgggcacctgtggtcccagctacttgggaggctgaggcaggagaatggcgtgaacccgggaggcagagcttgcagtgagccaagatcgtgccactacactccagcctgggtgacagagcaagactccgtctcaagaaaaaaaaaaaaaaaaaaaaaaaaaaaacactccagtcatgcattggtgaacaaagtttaaaacaacgtgtattcagcatggagtcacagaatgatcctacttttgtatgtttgtgtcacagcctttaaaagcatgtcttgttatataagccattaccctcctaaaaaagactatagttcacaggaataagttaaaagacataacaaaacataaaatgactagtaccaggaattgtgaccatggtttgttctggtaactgtggcatggcatggtttgttccaggaattgtggcatggtttgttcatgtttacattctgatgtcctattttttttttttaatttctatgtcctttccttttccttggtggtgtcattgttctgtagctgtatgaagaaactaaacttttctccattttcaggaaagcaatctaagaatcttgagtgcctcttcctttgttaatttctcttaagatgtgacttttttaaactactgcatcaggaaatattgtaaaacagttttgccttgaatatttgtgatgaaatctacgatgatcttcaagattctcttaattttgctaatattcagctgatcagaatttgtttttaaaatgtctggctggtgggtacttcccactgacaactgcttattgcttacagtatgtctgccttgtcaatgaatgaggttcagggtgcttcctagggatcagagtcagtaccatttttctctttcatctacagctgatcagatgtttattttacttacattaaatgaatgatggagatccaaagtgaatattatagaatattattctaggatcaacatcttttgctttgaaaaatcaacatctcttggcttttcctcagccaacccagcaaacagagattatcagactctgttgattttttactttcatttggcattggcctttttcttactgaaattaaaaaggctaatgatttgcctggtttctgtctctgacctttgcaggtctattttcttaatttttagatactatatatctgaaactttttttaatgtgtcaactttttaatggatagaaaatagacacgaatagtgattatgtgttcatttttcaattttccagaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtccaa

SEQ ID NO: 125 - DNA, 30227 bp, Homo sapiens
acagagcgagactccgtctcaagaagaagaagaaggagaagaaggagaaggagaagggaaaaaagaatcctcatcattaatgcaagtggaaggaaactcttcaccaaagaattgatcacatcatgaaaggtgaaatcattacggaattgcttaaatatataatttgaatctggatttaaaaataataaatgtaagttttttctttaaaaaaaaaaaaagtccattggttaaattcagcttgaatcctttgccactacatcaagagctaacagttcaacaactgatctccacaacctcccacctcatttcctttctcagtccaggtagcaggaaaatggaaaaaaaaaaatgattttttttttccaaacactttgtcagtttccccagcggtcacctttgaaaagggaaaaatgtctgaaaatagacaaagctgaatataaacatcatttaattccccccacacagacagcctccgctcctgtgagggcgtggggaaaacggagtgggagaagggggctagcgaggaggaagaggcgggaggtgcggcaggggcacaggtgacgctcctcccgcctgcctagcagagctccaggcgcacatccgcagtcagccacctcgcgcgcgcctccaggagcaaggatggagaggctggtgagcggggccggggcttgggaaggagggcgcagggcaggcgatgttgagccggtggccctggggcgggcggcgagtggaaggcgcggagcttcggctctcgccacaatctggtccggggactagatcagcggtgttgatggcagcgcttttctctaagcgctttctcttgctgagaaacggattttgccttccaaagggtggtttacaggcagctgctggcgtccagattctccattccaattgcctgaaaaaaagatgcaccaggccggggctccgggggctcttgatccgcgctgacgaagctggcgcatcctccgggcaggctcggctcggctcactggctccagctcccaaaactggggaggcacagcctattttaaactgcccccaaaagggaaagagaaatgctgtattggttccatattggcaccaaaagctgcggcggagatacagatttccaggctcgctttctccgggagagctcctgcgtctcctctttgttatgcagcccctttctccctcctagcccttgcctgggggaagccatgaaggatcagttaaagctgaagctacacttgagagagctgaaatgctctgtgatcataatgctttgaagggttaaccccgggtatctagagaaacagacattttgtttattcggaaccagcttacaagacaaaaaacggagaaggttttcatatatcccattggtatatgaaatccaaatacaatggtattggtataagaatccaaatacagtgaatgcctcaagataattttttcatgtatggtttgttggcaagaccacatagccaatgatttcactgtctttcttaacaaaggagaatggtgttctgtgagctacggtctactgcaagctagatgctgttaatcttctttgtaactatccttctaaaaatgaattattacctcgttttacacataaacaggcggaaaggctgatatttccaagtccttgtgactacatgtggggtaatgtggatttggacatagaaacttgctgaggaagaggcatgaataggggcaatatagctaccaggtgggcagctgtctggtaagacccaggtttttgtgaaccctggactgcatggcaagggttataagtgggcttctttgttttcaggactgtagttccccctatatgatttgatcgattaatttttttagctacctaacacgtattggcatttactataacctaggctctatactaagtgctttaattgcctaattctcataataactatgtgagataagagcaacatttatttagtgcttatttctactggcagtattctaggagctttatatactacttcatttagtctttttaaccattagattggtactattacttttccggagtctcagagaggtaaaatgatttcttttgctcacaaatctagtatttgttttagccccggtcacctccctgtaaacatataaatactgttgttttcagattggtcatattttcagttattctgcacagactcgaggaaaaaaaaaacctgtaaaataaaaatacgtatctatttaagagtaaggctttccttctgatttcctttgcctcaatcactgtgttttgtttcttaacattgctggctcccagtccacaatatatggtttggtatgtatctgctttctctcttgtcttaaaacacagattttctctatgagagcttgtccattctgcattcttcatcatggcctctggtagagcctggatttccacgtcagagaagcctaatggtttaaaaaatatcacatagaattggaatgccagtatctgtctccacaattgccttagctgaaaataaaacaaaatgtgttgggtgggcactggcaatggagcctgaggaaatatttggcctgattttttctgactttcctagataattagataggatggaacagtctcagtaatactgagaaaattatcgagttatatattttttttcacttcaaaaccaaagcctttgtgagtataatacccagcagtggatgggcagggtgagcagatctttgtaggctgtatgtattcaggcagctgaaccatcttgggtttctcaaagagcagtgactatgttattagtcttagacctatgaagacaggaagagaagtcacaaggaagggatagcatgcttatggtatgcttgggtatcgaataataaaatatatctgcaaagaataatttgtcctaatggcaccttcagatgatataatcttgaattctctgtagccttagagatgtaaggaaaaagcaacttacatcacaaagatcagtattactgtgtactatgatgccactgcatggggtccaggatgagagtctaagtgattggttctctctagctgaagacaagaagacactcaggtatgtggcagcacttgggataggccacagggcggcgagacatcattccttccaggacatctatcctgaaagatccatcaggaatactgtgatggagatagtgatggagactccttgtctctggcagttagagtgaatgctgaagaagagcccttttccagcaggccagtctctaacatgatcttgggtcttcacccagaccatgacagctctggtgctcaacacacacaaccacctccagggtacaagttggccccaagagagctgagacaatattggctctaatttttatcatctcattgatctcaaaccacagactctgaccaggacaattgggggccctctccttacagctacactcctaggaaagaccaccatcaatgattaccaggtaattttctacccctacctatgaagggacttggttagtaacttctcttgctagaggaaaaatgaagcttattatataagattaatgtttgtgaaacacctgttatttggaattggtttggtacttcctcttgaaatttccctcattaagtttaggtcgtggctgtttcataaggaaagaaaagggcatgtattgactggaattaaaattaacaaatccattgagtaggaggatattaaaattgagtggctcccattggacaggaaaaggaagagaaatccttgtggcaggtaaaggaggagagatttagatccttgagaactccattcccgacccgctgctcctgagcccacaggaagaaggccatcacaagacaataaaaatcccagagaaattgaaaggcaaacagaaagacccagcttggagtgataaggaaaatgaattccagcagggaagtcattgtatctaaaaggaatggcaaaaatagggcttagttaaggcaaacaccgagaatgaggggatgggattcccacaggtagaacaccaccctttagcaatgatcctgaccctaaagcaaggtcatttaagtgattatgggccaatattctagaaataagcttcaaattatagtagcaacagatggggctaaacaggagccgataagaacatacaacttcagaaagggatgcccgtgatgggttcattaaggaaaaagtggtcaggacatcccgctaaagcaacaggattcagtggatattgaggttcttggggggcaatgagacaggcaccaagagaccactactttatggaagactatgatcatactgctaaataccataaaagaatagacagagccaaataagagactgtttctaatgaaacaagcagacaatgtcccaacctcaggaatttaactcagtctgagaaagaagagtagtttcaaaactgtcaagcctgctttagtgctataatttttttcactgacaagagagaatgaaagactgatagctgggggagtggcggggggtgaggggtgttgacaggtagtaagcatgctggaaacagtgtctcaaggtgtggggtgtatggtgggtatatttgggaagtagtcattttctctcaaactggaaagcagatgggtgtgttcagacaagatctggaagctgtggcatggcagacaaagggagatgggacccctttggcactggaggaagaggcttgaatcttcttggagcactaaaagttccaaaagacctaagcagaaaatgcttccgaagaggaggatctgtatggcaagatgactgtaatcaggctcataaaagtaagaccttacataaatacaagtgagggggaagacatttcatgtaggaggaatggctcaagcaaaaataccaaggcgggaaattgcgtggtgcgtttagaagtagaaaaggctagctggcagggaggggatacataggaaggcaatggaaatgaatttgcaagaatgaaaacaggctaagaactttgaacaattttctctagatagttaggggctatagaagaggatggcatcttgtacaaagaggaatttaaggaaaattaatctgtcaatagtgtgtaaatacataagctggatagtaaacttaactaagttagggactatatctgccttatgcaccattgtctccaaagtacctaccatagtgcctggcatgaagtcagactcaatagagatgattaataatagaataaatgaaagaagtatagttgtcccttgggacccatgggggctttgttccaagaccctcacagataccagttggtggatggtgcaagtcccttatatgaaatgctgtaatatttgcatataacctatgcatatcctcccatatatataaactcatctcttgattatttataattttaatacaatataaattctatataaatagttcttatgctgtattatttaggcaataatgacaaaaatctgtgtttgttcagtgcagatgcttttttaaaaaatatattttccatctgtggttatctgaatccacggatacagaaccaacagatacggtgggccaactatacgggcaaaagttcagttccaatagaagacaagactactgcacaaaacacctagtgttatgagctgagactagggtggtaaagaacagacacttcacatgggtaccaaggaacagacacttcacatgcgagaatctaatgtgtaaagtgactaattccatatctaggtaggggaaaaatgattcatcctggacgacttgagtgcctgggagataaaagtacaaaaaagtagctgttttgggtaaaatgacgattaatttgatgttattcataaatttaattattcagcattccttcatgttttttttattcaactgacagcatcttaggcagaaggaaagataaaagaactaatgtaggaaatagcttggtaatttcaagtcattgaaagaatggtgaagtggagcttggtgttcaaggtggaaagcacagtgacatgagattggatagcatgtttgtaggtgctgagctgcaggtagtgacagatggggtcatgctttattactagcagagcttctagacaatgactcaatggctttcagagattccttggctctgtggaaaccggtttaaatgctttttaaaaacagcttcaaaattgaatttaacattgttgcagggcaatgaagcagggattgctgtgtgaatagcagtttggtaattcctcacaacgatttggtcataggcaataacaatgtacttttgtgacgatgtgtcaaaatttttaaaccaacaaaactttatacataggggaggcaagatagtgtggtaattatggttatgagctctgaaaccagactgcctgggcttaaatcccttctctgtcacttcacagctgtgtgaccttgggcaatttacttaacttctctgtgcctcatattctttattttgaaaatgatgataataatagtgcttcctatatagattatatgaaatattggatgaaatgctttatctcaagtgctaagaacagtgcctagtacataataaatgttaaataaatgttagcattagtggtttagtaaccttatgcaaaatataagttggagtattattttaatgtatcatctggtgtgattttttattccagtactttatttttgtcctctggtctttcaaatcagacaattaagcttccattgccttagagaaggaatactaagcagaaaataaattttagtttcaaaagccaaaattttgcaataccaagagccttggcattccagtttcaaaagactaaatgctaaaagaccaaggatataatgttcatgctagaaagggttgattggatgaatgcagagtgatggaggaactagcttttctggctcagggagataaatattttgtaggctgggagagcaaaatgggactgattgatgtctatgctcagataccagtttgcatccccaaaataaggccctgtgtccaggccccagacatgggaagttcaggagagtagaaaatagaacctgcaagtagttgttgagatccaagaaaaaaataatacctgtgtctcacagtcccctaaagggactgtaggaagcagcatgcttgtccatcatggcaagtgaacagtaaaacagagatggattgatacacaggaatttaggcagaaagccttgtagaattttctttcatccagcacaaggggaacccagggacagcctggaagaacatgagcaagcaccagttcatcttgtgagcaataattgcacaaactaatgacagtgatgacttgaggggaacacagccattttagccaatgctagcataatagatggttgtaatcaatgattagcagtcctgtctcatagctgggcattttgtgaacccccaggaatgcagacgcaatgctagagattgggacaattatggggtattgtggtggtgagagaccgaattgacagagattatgcaacattgactggaagtaaattcttatctccagttggaatagtaacatgaaataaaaataaagttcagttaaaaacagataaaagttacattttttaatcatctctattatgaaaaatagaaatatagaaaaaataaaggaaaaagttacatttctttcctttgccccttagtatgaacatacatacagaccaaatgaatggatccatggcactattgcagagaaaagttcaactgcctctaccctgacttaccattttatagaaggatgatgcccagaataacattatcttaaatactcccaagatttataagaaggccgaggatttctgcccagacacatgaaaataaggcagcgaaggaaaaaattaaagccagtactgctttttaaaacatgcggtttatatgtaagacttgcagaattaaaatcaacctcaacaaaattcaaggcagtcaaataatgttcttgaaaagtggttaccctctgagttacatttttaacattaacagacatggattttttgggaagtggatgctgtataacatttgtattctcagcattttctgtatttatttaagccagtttgttttccccagtgatcctatgtctttgagacctcgctcttgacaatggtggcttcaggagcaaggccccaaattgccaggcattgctattgtgttgaagccacgctgctgcttttccctcctcatatcctgagcatgctttattatttcttccatttataacctctagttctgctcccacaaaaccaaaattctctggggggtaaaaacttagctacaacattccctgaaatagcgattagattgaagcttccttggatttgtccaaatccaaacccccatttctgtactttgctttctgtcttcaggtgatcaggatgcccttctgtcatctgtctacctacagcctggtttgggtcatggcagcagtggtgctgtgcacagcacaaggtaaagaaactcaattcccctgcttggagcccagcaaacacaatttctggggtgaagacatttagccagtattgtttgtgctgtgctggttccatttgtctgcatgaattatttaagcatgtaatctgcctggaggagtggacgtggttcttttctgctttccacacttttgttttcgatgtagaagcacactttgatatttagaagccgtttccatagtagatgaaccagacttatgaaaaaatcctctattttaaaatgtcatttttcacttactgaattgaattacaaataatgatttcctctgtttttaaatatatgggaatgcttgaattcaatctttttggaaagtaatctggcagtaagccttaaaattggatgtgttacctttttacagaaaaatgtagaaaacaacgaaatgtagaagttatataggctcatattatctattgtataacaaatttggttgtgccatctacttatctaccgaagttcctagctcttcctcctcagttccttgggaggccacactcactttaccactggccttttcatcacttgcagtgtgatgttgctcagcatcagcctccacagatgtgggtgatgctggtgaagatcagataggcttcatgcccatctccaactctggtccacaacattcatcacaggacttatcaactgaccagaggaatctaagtgagattccatagaggtagcaaagaattgtaaatttcccctaaatgatttcttccatagtcaaaaagaaaaaaaaaactcatctaagagttgaactctagttttgagttccctttcactctgccttctaaaagcagtaatttcttcaaatctctggtctgcaaaatctcaacaaactttcaagaagtctcaatcctagcaacagatagtttcccagaacttttcacatcttccaagagtatacatcatttagttttgaatcatgcaatactcaggggttccagcctgtatttgttatacccatctttcattccctgatattgtgggggagttgcttgtttactcagccttctttttttttctacttatacttgtcaacattttatccagattatcagtctttaaaaacaagtctggatgcttgtgatcaagtttacattttgtgtaccattttattagtgtctttatctttattaattcactgcgatatttgaaagggagggtaggaggtttggaagtaagtgtattcatttttaagtatctcttcttttctattaaatgttgctagaattaaatgtttctctgagaactgtccttccttccttccttccttccttccttccttccttccttcctttcttctttctttctttccttccttccttctttctttctttctttctttctttctttctttctttctttctttctttctttctttctttctttctttctttcttctctctctttctatgaaatttcactctgtcacccaggctggagtgcagtggcacagtctcagctcactgcaacctctgcctctcgggttcaagcaattctcctgcctcagcctcctgagtagctgggattacaggtgcccaccaccacgcctggctaatttttgtatttttagtagaaacagggtttcactttgttggccaggttggtctcaaactcctgacctcaagtcatccactcgcctcggcctcccaaagtgctgggattacaggcgtgagccactgcaccaggcatgtcttacccatgtctacagatttttaacatattatgttctcattgtcattcagttataataatttatgattttttctatgctttctggtgtacctaagacttaaaagaagtatgtaaatttctaattttattgtattatgaacggtgttcaaggccatacgtagggtagcaacttttttgtagcctattttgagttcaatttggaggaaggttttatgtgtgttttgaaaagattatacatcctttctttgagtaaagtgtgtgtgtctgtgtgtgtgtgcgtcttatgatatctttattcatttagtatgagttattgggtttgtaaagtttattatttctctttcattgtattgactttctcaaaagtttggctatttgttattatgttgagattttccatcaaataatctatcatagtatgagttgctaatattggagaagggaagaaatttatatttatacacacacagagagagagatctacatacatataaagtatatagtgtatataatataaacatataaaacattgataatattaaatacctaaagtatatatataaatatatattgtttttcaaattttcttttctttgtgacatggacttaccttgtcacccaggctggagtacagtgctgcaatcattgctcactgcatccttgaactcctggccttaagcaatcttcctgtcctatgctcctgagtagccaggactacaagtacatgacgctacggctggctaatttttacatttcttgtagagataagggtctcatctcttgcccacgctgatcttgaactcctggcctcaagtgagtcttctactttgacttgccaaagcaccaggattacaggcatcagccactgtacctggtagtttttcaaatttcctatttaactttttttcctttaacaattgtttaaatatattaaattgaaggttctcggtatgattgtgaatttaaattttttctttgtaattctattagttttgctttatatatttcggagttatgttgttaggtgcataaaggtttatgattactataatagatctgattttagattgttccttttatcattttgaaaaatactttttcccttcttacagtggttttgtcttccattcttttttttctctccttttagaagtcttactgaactgattttggaacatttaagtctacttattgaatttttccttaatcccttccatatcattatctctttgtgctacattctagaataatttcatggcctgttcttactttttactaatttactcttcagtagcatctattttgctaagtcagcctatctgttgcaattttatattcctcattttctatatctctaatttctactattcataatgcttttattatgcttttattacacttatatcatcacatgtattcatgtcaaagtatttattgtcttatgatatctttattcattttgtatgagttattgggtttgtaaagtttattatttctctttcattgtattggctttctcaaaagtttggctatttgtttttatgttgagattttccatcaaataatgtatgatggtatgagttgctaatattgaggaagggaagaaattaccaccatagcatttagtgaaagcagagggtgtgtaaattattatttgtgtgccaaagcttcaagatcctccagaacattgtcccataaatctctttacctatgttcctatatacactcttgcttcctggaggcagacacctctgttccacctgcttcctttgtagattgtgtatgtgtgtgtgtttgtgtggaatgcatagggctgctccatcttctttacttacagtggtcactctgcctgttatcctttggccttttcctgctctttgcttctacttctccaagcatggacaagattggtgaagctgctatcctacctggcagtttacatcttcaatctcattgtacctacttcgcagcttacacagatcattcacaatttcttgtaactgagagttgctctcttgtttttctactctgctactattctttaaagattgaacaaacatatttgaacctatatatttctctggcatcacgtaggaatgtaaataaatgttttcttttttgcattttctctttcttttctatatttgacattgaatatacattttatttataatcaggaaaaagtgtatgtgtgttacaatctttaaatataaatgttcttttatgattccatagtgcaagtggtgacccaggatgaaagagagcagctgtacacacctgcttccttaaaatgctctctgcaaaatgcccaggaagccctcattgtgacatggcagaaaaagaaagctgtaagcccagaaaacatggtcaccttcagcgagaaccatggggtggtgatccagcctgcctataaggacaagataaacattacccagctgggactccaaaactcaaccatcaccttctggaatatcaccctggaggatgaagggtgttacatgtgtctcttcaatacctttggttttgggaagatctcaggaacggcctgcctcaccgtctatggtgagaatctctgagaatcattgtctgtgtctggaaatactatttgcaagaatgtttggaatatagccgtagtgcccagtttttcaggattttaaccacagaaagggtcatgagaagatagcctttcttgtctactatagctgtgtttatgattatttggagagttcatggggctacactgagttctttggctggagctatcttttctctgagggatcctgctgtcaacaggagaaatttgaggacaatggtgagggcaacaagttctccataagcagtgatatgattcagacctggctttgccactagcaatcatgcgaccctagaagggtcctatccactctccactggagtttcattgtctggacaataagtgtgaaatagatcatttctagggtcacttacaacttcaactttttggatctaaggacagagccagattggactagccttgagccagttatgattgtccttctcattttgcttggggagatagctctttcaggattgataagggcacatagagcagagccttttcatacctgaaatgatgccaactaaagatataactgggcaagagcattcttttaaaatccaagaggcttagtttgatgagattcttcttaactgacacacagctgagatgaaaggttacgaagtgagaataagagattactttggagcatggcacacacggcagtagttttagggagtagaggacaaacttatggttattaggaatattaaggtgagagtttactcatgccaataatattgcccacagggaagaaaaggcttagtagatgagaatgcaagggagataggacaatgccaaaagaaatttcttttacttattttgcctataatgagtcctctctgtagaatcttatgtcgtttctttcttgatttccacttctttcattctctctttttctgtttctcctccctgtctccttccttcctttcctccctctctgccttcctttcttctttccttcctcctaatttttctttttttcttgtgtcatatttttctcatttctgttcctttttattctcttcctcttttcctcacctttctttctcttagtctccttccttcactcatagacttttgtatttttttttcctgaaaagcagatgtctctgagctttaaagctagtaattctgttctcttccacactgaccttttccatccacaccttataactttcatgtaagccattctttgatacatcataggacctcaacactcagtctggcccatcaaccatattcatgttttttcatttctatttcccttcttccagcgctcacagacattagtaatcttagccagagccacagttccatagccctacttccccaacccagtgtcttctacctgcctcttgaacctataagcacagcccaggctttcagttagcactgccatttaattatcttgtgggctccaaagccaaccacagaaagacaagtttacatctgcttagaaaagaaaaattctatattccaacaaaaatgaactcactagtcaggggattttcaaaagggtacaggctacagctggcctgaaaataattttctcatgagctagttattttttcttaacttaatcaggtgatttccagtgctcttccaacagtcatttcttaatttttatggttattagctacgaattccaaattttggttttgtgtagctcttccaaaaagggattttccagctctcctccatgcgtctaattcactggtccagctccaagaagaaaatggctatctctaagcagtagcctttctatgggctctctgttccaaaactgccctagtgcctctgataggtacaggacacatgtatggcagtgctggttggtggttattgtcaagccatttaagaggaatacccagaagaaggcagagatgctgcattgcccctggaagagaacagaataattaggactgtttgggagagaaaagtatcatctgacttctcaatactctatgtccatctgacaaattctgctacagacaaattacagaaagggtctttctcatcacaggtttgtactaggccagtctccatcctgcagatcagaaaattagacacaagaggaactggaaagtccccagtttgggtaggatatgccatgctatctttctagcctccattatctttctataagcatatttccttcatctctctgaggagactgcaggactcagatcaggtgcttaactgataacagatcatatttattttttgtcccagtacagcccatagtatcccttcactacaaattctctgaagaccacctaaatatcacttgctctgccactgcccgcccagcccccatggtcttctggaaggtccctcggtcagggattgaaaatagtacagtgactctgtctcacccaaatgggaccacgtctgttaccagcatcctccatatcaaagaccctaagaatcaggtggggaaggaggtgatctgccaggtgctgcacctggggactgtgaccgactttaagcaaaccgtcaacaaaggtaagagaaagtgagcaaggtggctgtggttgtgtctgtgtgcatggacctggaaggcagtgaatgtcctgcagaggttttcagcctcttagcataatctatttggagaaagaatggggcaaataaggaaaaaacaaaacaaaacaaaaaaattgaagaaacaaataaagcaagttttactttcataaatgtggttcattgcccacacaccaaatgctgttgctgagatccattcatttatttatttcttccactctactaacatgtaattaggtctgttctgggccagactttgagttgaacatgaaaagtgcagcaatgagcaagactcagtctctaagcttctagtctactagggaatagagataaatggatgggcaattgtaaaactgtgcattacattctccagtgagaaagctatgatgcaccatgggaggattggaggttgggaaggaggggctcaatctgggggagatttggaaggtttcctggaagaagtgacatctgtgatgaaatcactgtataatctacactgtgagaaagggacctatctgaagtgctttcgtcacccattttaaagcaaactgcctcctatccaagtccctttttgctttcttgtgtttttgtctaaactactgtctctgggcctcatagatacaagaggtgagaatgacagaggagagaataaagggagcagagattataaagacaagaagttttagcaataatagccacgtcttttctctgtgtagttttgaagcactgactcttgatgaagcagtcagtaataagcctcctaaaaagtattactcctcttttttggagaaattatgagcaataaactggaaaagctttctacacccacctctttactgcagtggcctttggagtccctgaaaacccagactaatcagaaattggatatttaggtaaatttggtcaaaaagacttgcagtgttggatttggatatttgtgaagagatcttcagattcagccatggcaaagggtatatttcctggaaaaacaagaacctaaaaaagaatgtcttttttttttccaagaatgccagatttgacttatatcacttacagtgtttatataccacttgtataatgtttgtttattctgtgaacatttaccaaaaaacttccatgctcaaagcattatgtcaagcactggacctatagaaatcaaacaaatccctgtctgaggtctgaggtgttcagttaaataatgatgtgacgaatcccattatggaggtatggagaagatgctagagactcccaaatcaaggagttcctcatttgcctttgcattgcaacatgacactgaggaactgatatttaggctgagttgcattaaagacatagataagaagaaatttaccaaaaactttgagatgagttatctctgggggttgaaatcatgagattcatgtttctttctttgtacttctctgcattttctacaatttcctcaataaacatgtattcttttaatagacagtatggcaaagaatatgtgctttagaggcaaacagactttcaaccaaaccttggctatcccatttgttattctgattttgatctcagttaaattctcaaagcctcagttttctttaaatctgttagaaaataataactcttgggatgttttgaggattaaatgcgataagtttaaagctcttattacacagtagccagataaaacatgttagctattactaatatctaaatattattaaaataatatgaagtcatacgtataaacctacatatgtatgtatttaaggataattttaactaaaagctcaactctttttgcctcaacaatttcctcatgtgatgtcattttcctttttctttcttcaatatctataggctattggttttcagttccgctattgctaagcattgtttccctggtaattcttctcgtcctaatctcaatcttactgtactggaaacgtcaccggaatcaggaccgaggtgagttgtcacagggagttcaaaaaatgacataaattaaatttgatttttaatgacaatttgtgagtcatttgaagatataaataagggaatggcaacaatgtgttttgtctgttgtttccaaaatacagaaatgttgatactgttttaaaatgcgtcggggcattttcttgcaattggacattaaattggacatttctgctttttgtttgctaagatgacttaaaagctaatgaaatcaaacaatggtgatagaaccaaagaaagaagtctggcacccacacaaggccctttccactcattaatgagaaggacaatctcttctctttttatagcacttcacaggccatctgcccatttaagaggctgcaattgtccaattatgatcactcattaagtagcactttctgtgttatactcccacgatgcctcaaacactgtcagcatcatgcaagtgatccatacttacagtaactgtgtcatgccattctgcccttctagactgtaaatttctctttatctttcacaactatcacagcatcttatatataccagggtctgaataaatgttaatttactgaatgaactgattgaggacagagagaatttttaaggtgattttttttcaattccttttctcctattgtaaattcagtacctcataaacgccagtctaagatattgtgagaaattataaaacaagagacactataaagcaagagcagcgagctagatagagaggaagaatgaaatgtctttggtttctgtagacaatgcataatttattgaagtgaatacaaagctgcagtttatgttttgtgtaatggaaatttgctagggtgaaggggtgatgtgggagtttcctccatttcagatgactgaaggataggggtcaggaaagaaaaatagttggctttgatagctattttagactttgattaaaaatctcactcattttttcctttcattttatgttttatagaaatgtttccttttagttttatgtttatatttttatgtttttgttcatgtttcaagtttttgaccactttattttgcataatcctccagattaattcttgtgaatttataaaacacacaaaaagccttcagtgtcagtggcccttttgcaaaagtaggtaaaggtatttgttctgtccaggagagaagattacaaatcagttaatcttactacacataattataagcctgtggaactcataagtgctccacagaataaaatcaaagacagaagtctgtttgattctactttatccagaaatttttcttttccaattaggaaactgaagcccagagaggaaagctgacttccttccataagtttatacagcttcttaagagcaggctaggactaaaccccggtgccctgacctctgccaaatgttcttcctgtcattactgatgccccaatgtcttgacttgaaaggagctattgttatcagttgcattcacttaatctgtgcttactgtctatgcccttttcttttggaaagtgcaagcagacaagtataaagtatttaaattcatttttaaaagtccctcttgggcaaaagttttataatgcaatctactcaagcctgaggtaaattctactggaaagagacatgcaaaataattacccttgttcattaagcaagtcttggattttctgggaaatatagtcattcaatgccaatatccgattcttgaagcaaaaagaatcagcttatttctctgcccaagtttttcctcacttatgaaataaatacctgaacctatgttgactcctgtacgcatcaggattgccatttactgatgatgtcttcatgacatatggccaattgcattaattcctctgaatgactggatttttgactcatttcttttccatttgtgttgcttacaaaagatacctgaggctgggtaattcataaagaaaagaggtttatttggtttctaaaggctatacaagcacagtgccaacatctgcttctggtgagggcctcaagctacctccactaatagcagaaggaaaaggggagccagtgtgtgcagagatcagatggcgagacaggaaaataaaagagggtgctgccaggctgttttaccagctctcatgggaactaacagagagagaactcactcacaccaagggcattaatcaattcccaagagatcttccccccttaaacacctcccattaagccccacctctaacaccggggatcagatttcaacataaggtttgcaggatcaagtatccaaactataacaactgatttagaaattttaacagccagataatttggcatcatacacttacagctagtaaaaccaagagtcaattatgctgaagcttaaatgctcagggctaagaaaatactggttcatttagaccaaacttcaggttatacaaaaagttgggacaaagaaaagagaaaggacagaaaatcatagaagatagtgacactttttatttctaccattaacaaattctgattgatattttgtgaggaagggaaatagtcctacacatttgccttccagttgttttcatccttacttttcttttctttctctttttctttccatcatctgaaaacataaaatttccagatgagcaggaaaacacgattggtccaaaataatacaaaggttttgaacttggtgacctcaaaaatggtgatgtgaacagaaacactaaagtgaagaggtggaactgagagagagagagagagtgtgtttagagtttgtagaaaagcaaaggagacagacgaataagtttttgaatttattgagtgtgagcagacaaagaattatcttcatgtagctgtccagtagccaataataaaaatctggtaagtaaggtaagtctgttaagtaaggtcaattctgtgttgggtaaatgaccaaaacagaagtttattttaggtcataatcactactcattgctcacaaatgcctaagcaaacttacagttactgatgttatttttacgtagaagttagacgaccattacttttcttgtgatcctgtataatgccagagagcatgacgttggttttttccaactgcgaagtagcacaggccatttttctgcaccactcctgatagaattatcacagttacttcccaggagactaccacagtaagactaataagatttcaaagagcattcttttctctttcatgaatccagcactttaaaacagtaagtgagaaagaattaaaatttcctagggaaattttccaccatctcaaaatacttctttctattaaaacccagtgatttcataaagttgtgcctttcacatatatgacaatgtagttaaatttggagttgagaataatcttttatttctcattagtcaacccaccaacaattacatgcttctcagaaacaataaaagaaacttcactttgaggcagatcatcatagacaatcacctaatttttaggcagctttattttgtttggtttggtttgctttgctttgctttgctttggttgagtcaaggctgctttggagaacaatagaaaatttagacaatcatgtaaaatttatatgccaaacttcacttttttttcaagtactagctaatccatgtagaaatatgtttttcatctgcatgagctgtatatttatcttgatttccttctcttcacaataacctaacctatgaatgggttcctgacaatgtataataacaatggcaaagttaataaaacgtcatttcctggataacagcatgtgactttctcctattttaagcttaatacttaatacaaacaacaaaggtgttcgataaacaatttgtgatgatgtaattggtcattagttgaaaggggaaaacaaaaaatatctgttcacccctcctgaaataacaacctttgctttgctcagaaagcacttttgcttcccgtacaaaggctgtatgaatgggtagccctctgtccgggctgtatgaaataagaacctttgctttgctcaaaaagtacttatgcttccctgacagaggctggatgaatggcactacccaaccccaccccagtatcctcatagttccaggaggaagaaatgtgatgctttacatgagaaaagcgataaagaatcttcattgaactgcttagattagtattgctgggagaaatcttgaattccaaggctatttttccgcaaaaattttacaaattttgggtgatttacctatcatgccaagatgacatacataatcttatcacaaaacccttagcaaacagattcttaatgcctggacattcttgcctggagctgttctttgctgttctctctaacatctaaacccacatgggcatagatgacccaaattttaagtttcagtctgtatgtggggaacatctctaggaatcaagacatctcagtctcctgcccaataattcaatgtgtgttcagtcctcaaatggcatactcattcccttggctttttggccatggggcttttcaaggagattagctgtcagtacctgttagactaattaatgaaaaacaaaaaggatgagaaaatctaccagctgaaaaatcctctaacatctctgctccttttgcattttagtttgcatatatcctttacctagtgtctattaccagtttgttttgtttggaaccgctgtgcatttcagtagtatagtcacacccatataataagcttgcctctaaacagatacaaggccctagcctctactaacaaaagaagccaactgtatgctatttaaactggaaaagattgggccacttaatgaaagtactcaacatggcttacagagggggaaacatcataaatgctgtattaattttctattgcttctttaccaaattatcacgcacttagcggcttaaaacaacacacatgtattatactacagttctagagatcagaagtccaacatgggtctcactgggtttaaaacaaggtgtctgcagggagacattcctttctggaggctctaggggagaatcctttttttccctctctctcttttcctgcttttagagactgcccactttccttgccttgcatcttcaaaaccaacaatgtccagttgagtctttttcacactgaatcactctaaatctgaaacatttgtctcctttttcctttataaggatccttgtgattacaatgggcccaactgcataatccatctcagggtccttaatttaatcacatcagcaaaattcctttagccatgtaaagtaacatattcacagattcctggaattagaatatggatgtctttggggtgccattattatgcctatcacaaatgctaagagggtgtgggagtgacttacttagagggacagctgtttactgacctcccagtggaagcattctctctctgtaattccatagcctttgctctgttcctggcaactctctatcaaagaacttcaaaacctggtcagaacctctgaccaggcaaggctttattttccaagattatattttgtctctcaaatctgtaatgcaaaagcatcacttctgctttcccttgttttctatcactaactataatattacaaaaactttttttttcttagaatctcagttcttgcatatttcctgcttctccctattcctaccttttcatagaggaagcaattctgacttcctttgtcttagtcataagcatcccgtaaacacaagttcacagggaactgtgaaagttcaaagtgggaaaattatatttatcactatgtgtggtgatagtaggcataaccaaattggtctttccccgcttatcataacgtattaaaccgggaatttctcagtccctaatacttgcagagtcatgttatgaggtcattatagattttaggagcctcaatgcttttcatatcattttcagaattcttcaaaattcccatttataggccctaattgatttcagcagcccatatgcagtagagcagttcttccactaacaatttttttaaagacagagctaaaaatatatatatttgatgaagtatattaagtagaaaaaaatgttagaaatatcagaaaaaaattaagaaataaaagtatgttcagtacaatttaatttgaataattttaatctctaaaaataaccatattgtaaaaaaatacaatttcttctagtaaataaagaacaaatgagattttgtactacaaacatgcaagacatgttttgcaaaaatcacaacttcaatgttaagttccgcatttcatgaggcacaattatggaaatttatcaatgagaggctattaaaagagagcctcttgcccctctcagtattcaaactagatagaatttcatgatgactgaccaatgctgctagttctgagtctccgtggacactagtttataaatactccaaccttctcccttttttatacaataaagagaaatgctagccttgtgttttgaaagtcctttaaagagttcatgttaaatacctttgggattatgtacacaattgtcttttaatcttttttaaaaatattacttattatgaatttttgtttttattttttaattgaaaacagaaattttatgtatttatggtgtacaacatgatatttctatatatgtatacattgtggaatggctaaatcaagctacttaacatgtgcattgcctcacatacttatttttttgtggtatgaacacataaaatctactctgtcagtaatattcaagtatacaatacattgttattaaccgtagtcaccatgttgtacaatagatctcttgaagttattactccttatctaactgaaattcttttcatcttaaccaaggccacatgaaataagaaactgaatgtgatatttatttctacaatatttctccttcaagagtttaaagttttcaactgttaattttagcaaggatgattgcaaatgacatataaaattggttgttttctagtcagtcatttttatttgcaacaatgaacaattacttagcgattttgatcaaatcctgtcctttacaaactagctgagtacctttggacaagccatttaacttccctgtgcctccttttcctcactgagtaagatggggataataattgtaactgtatctcataagaatgttgtgaagatttaaacgggaatacatacaagtgcttagaaaagtgcctaatggccgggcgcggtggctcacgcctgtaatcccagcactttgggaggccgaggcgggcggatcacgaggtcagaagatcgagaccatcctggttaacagcgtgaaaccccctctctactaaaaatataaaaaattagccgggcgtgatggtgggcgcctgtagtcccagctactcgggaggctgaggcaggagaatggcgtgaacccggaaagcggagcttgcagtgagcagagatcgcgccactgcactccagcctgggggacagagcgagactgtctcaaaaaaaaaaaaaaaagaaagaaagaaagaaagaaaaagaaaagtacctaaaacgaatgatacgctggaacactggcctgggaggagaggggcggagtgtttatttgtagctgtcgccaacttccgtgaagtaaatgttcccaccatagcaaatttcaagctactaacaatttaaccatcagcgtgagccaataaagctctagcacaccaatgcggcagactaagtttgtaacagatgttcatttttgtaattactaaatctaaggtcctttttcgttttatcgaagtatattttcatgaattgtacaaatgttcaaggaaggacgagtttagagaggtaaatggagggacaaacaggaattgtagcagttaggacagctttgaggaagatttcaggttatctgaatctgtttggacacatgggcatgcgtatttggaagggagaagaactcatacagtaagtcttaatgtcatcaataggttcttggaaactgcgagtttaagcaaaacggcatgtaacagaaccaattttaccacaggctaaatgatagaaacaagacttaagttcctatggcatatttctagtcacaaaaacatcaccaaacttttaataaagaccaaaacatttctaatattaaacattgaaataaatgtgagttatgcatacatttaagaaacagtaacaaaaacaagtgagataattattcactcaatgatttcagttcaagggtatgggtggccagagcctatgcagctcaggatgcaaggcagacactgacaccagacaggacgtcatcccagagcagggggcattcacacacgcgcacacacacgcacacgcacacacacacagttactcagactgggaccatttggacatgccaatttacataacatgcagtacgtgtttgggatgtggggggaaccagagtatccagagaaaacccatgcagacttggggaggatgtgctaactcccacacagtggtcctggatgggaatctgttgtttttctcatcaacattataacaacatgttgtggaatgaaaggatgttgttggaggacctgctatataagtcatttcagtagcagtcttgggagaaatttgtggggacccactggggagacagaatcagaaaatgtgcattctaaaaacgtgcgtatactgaaatgaacttgtgatgcctagatgaacttgtgatgccgaaaactgatttcctgctagtgcattgtatctctttttgaagaagagaaactgttttgcttagacattttttttgtaggagtatgggaagaaaaattactcactgaagctagtgcgagtcaagggtagagggagggcagttcctgtaaggatgtgtataaatggatttccaggaaactaaactgccgcatgtcaggaagagcggctacctgaaggtgcttgtagatgcaaagctgccctaggttccccatcagcactgattcatgagttttgcatctagtttctcctgtccttaccacaacttggctgttctgtcttctctgcactaaacaacttcttcactctctattccatatgttttaaacttatcttattatttttgttccctcatacattgcttcatagattacccaatccaaaaagaaacaaaagagagaaagaaagaaaagtgagaggaaagaaagagagagaaagaaagaaagaaagagagaaacaaaacgaaagtatattcctgaaccacacaaaaagacaaagaatcacattctctacaagtgtcctgtgtttttgcaaattaaccagtacttcaggtagtccttaggtattctagagtttagaagaatcctaaaaggttttgctctggcaagggctgtgagtaaagcatggcctttgaggcgatggagggagaaagatatatagaaactttattttagagaaaggaaaaacttcttcagaccagatgcagaatcaagtaatcaaccattcaaagtcaggcttaatttaagagctttttttaaaaaaaacagacaaacaacaacaacaaaaatgtatactggtgatctttaaaggaaaccatgttatcatttagattattattctggattccagtcagtagttaggtcatataagaattataaattctgttattggtaattgatagttcaaaatgcatagtctcaccactcagggaaaacagcacttagatttcagaagataaaactttgtaagtggggatgaaaggagggcatggagggtgttcttaaatatttaaatggatgttcatgagacaaaaggactagacctggaagttattacatttaattctcatagcaattgataaggaaagtatttccatctgataggtgataaaaccaaattcacagaagttcaattcattcattttataagtatttattaataatttttaatatgcttggcattgtaattagcactggttattatactataatgaacaaaacaggaccttgccattttaatggagatgacaggttagggggacagcagagaaatgacaagttaatcaacaaataaggatactattaaaacacgagattttagcacaaggaaataatatagaataatataaaatatagcttgcaatgaaagaggataacttgaaaaggataccagttacttggaatttgttagggaaggcttcgctgaggtggtgacttttaatcagagaccttcaaggagagaataatccagccatggaggagacaagggaagagtggtttttagcaaattaattgcaaatgtgaaagctctaaggcagaaaagtcattaatattttacagggtgcggtggcacatgtctgtaatcccagcactttgggaggctgaggcaggcagatcacctgaggtcaggagttcaagaccagcctggccaacatagtgaaaccccatctctactgaaaatatgaaaattagccgggcatagtggcatgtgcctgtactcccagctactcaggaggctgaggcaggagaatcacttgaacctaggaggtggaggttacagggagcagagattgcgccactgcactctagcttggttgacacagtgagacttcatctaaaaaaaaatatatatatatgtatatattttagagcagtgttactcccaatatggtctgaggatggttgcccatccaagaagtgttctttactagtctgaagcaaaataagtacataactgagaaaaaatgcttacactctatgataagaatttgacagtaattttatatctgttaaatctgataatttaaaaatgaattatgttattttatgtcatttttctagtaattcgtttttactattgaaattgaaaaatattgagccatgttgggagaagatggtctttccatggtccattgatatctgatgttagaaatattgggcaggatctaaaatgtgcagaactttgcaggttctggtaaacaattacctaagattacacagagagtgagaggtggtgccaagataaaattccaagtcttttacttcatcgtctatgtgccttgagaggtttccaggctcccagtatgtgccagaaagccactagcccttggaaaggcctctacttcgtgaccatttctaccctgacatccagtgagttattatccatcagaaacaaagcttacaaatagaatacattttgatcatttggaatttttgaacatgttcctttacttgtagtcttaggtgattttctaaaccattatttcctaggattacgtatgcccttttatttttgcttcagttttgtttttgtttttggttgttgttgttttgttgtttttagagatagtgtcttgctatattgcctggggctggagtgcggtggtgagatcatagctcactgcagccccaatctcctgagctcaagttatcctcacgcctcagcctcctgagtagctgggactacaggtgcatgccaccatgtccagctatttttttttttatttttattttttgtagagacaaggtcttactatgttgcccagactggtcttgaacttctgggctcaaatgatcctcccgcctcggcctcctaaaacactgggcttacagccacaagccactgcatccagcctacttttgttcttgtaaagagtcatagtcccctcacaccatactgggtcagaaatgtctacttctttatctttgccaatgaatatacactagaataactctgtgtttcttaatctctctattatttatcttcttaaaatgtattatgatagaatttacaagtgtccaaagttaataccttgtttttcttttatccagagccctaaataagtcacacagcaccctgaaagtgattccctggtctacttgaatttgacacaagagaaaagcaggaggaaaaggggccattctccaaaggacctgaaagagcaaaagaggtgggagcgaaagccttaaggatcccacgactttttactgccatctgagctactcagtgtttgaatcccaagaggaagtcagtttacctctcaggtctgttgtaggacttgattttgtaaagcaatgccatgttatgtggttgaaagggcactggacttagttagtatcaggagcactgagctcacagactgacttgggctcctactggtggggacctctgttagtcactttacctcatccaaagtataaaggaattggaccaaataatttaccacatagctctaaaacttaatttaaaatgtaattccagaaaaaaaaagggaataagcaaagggggaagaattgaaagagagagagaagaaagaatacagagagcttaccttttgcctttctgttgatgttacatctcttcttcctatgttcttaggtctatgagtctgtttccccatcatttggtatctagtccagttcctgcttactgctttgctaatagctggccttgctagaatccttggtttcactgctgttcttcatgtgcttctatgagatttactccaacacaaataggactgaatttattgtgaagtaacattggcaatcttaacttattcatttaacttatttttatagctagataaatattgttagtcttagacaatagctcacattttttgagaagcatgccctccctgtccatttgtcttataacatgacccagccctattttacgtcattctaaattcagcctcatataatgaaaatacattatgaaaacagatgtttaggagatttcctgtatagcagtcagccaattcatatgctttgtctctgctggcttctttttccatgcgttaacttttcccaatagcagaggaggcaaatatgagcatacaatccctttgttctaaagatattgttccagctagtggaatgatgttgaatctttaataaccataattagttgctttttcagtatcttctgctttgtctgtgtctatccagtggcctaggaattaaagtgtaagttgttttcgctgttaaattggatatttatatatatatatatagcaagattttcatgtgttatttaattctgtattgtttcttatatttgtagtaaaatattgaacaattaaaagtgttgactccaaa

SEQ ID NO: 126 - DNA, 14829 bp, Homo sapiens
tgtgataaatatgccaattcagtggaagaaaagatgggatgtgtggaaaaattagatcatacatggagaaaaatagactcctgtctcatctcacaaaggtggactccagatggattaaagtctaaatacaaaacctgtgcaactatggattatatctataatattaagtacaaggagatcctgcaaatcagcacaaaaaaaggtaagaaacccagtttaaaaatgggcatggcatgtgaacaggaaatatatacaaaaaggaaaacttgaatgccaacaagcatatgatcagatgtttaacctcacaagagatcagaaaaaggaagatcaatagacaatgatatataactttatagtcataatgcttggcaaaaattaaaagatggataataccaagtagagctgaggacatgggataaaatagttcttcataattggtggtggtgtcaaggcattcagccactcagatgagcatccagaaatcacttagtgaaattaaatatataggtgcatgtctgtgaataaacaagtttcagccaggagtgtactggggtctttgctcacctctggggaaaatcagatttggttttcttactaacttggcagatgcccttggaagtctcctctaggtttgtgaaggttcatagcagctactttccttatgcatcactctctgcgactgctgagtcacctctggagctctgagttacaatccactgagtccacttccctggtagaacactggatggaagaaagaaataggaactttgttctctttatgcccctgtccttttaacctccatgtgactaacatcctaggtgtagcagcatacttaaatcctttttctagatggttaagacaatttctgaaggaaattacaatgccggagggaccacaattgaagataacttgaattttcatctaatgagaagatactttaaaaaatcataacaactagtataaggtggtttctcctattttgtcctccatataaatttgctcccaagtgtcacccaggtccctttgaatctctatggctcgtaagcccccccctcctcctcaaagagattgtcagctctgaatatttctcctttgtctatcctcaaggaacctctggttccagaatcagagattctgcaggtgtaattggggagggtagagcaagacttagcaaagctcttccatttaaattcccagcctgtgggagccaccagcctttgatttatttattaagttcatctctcaaaggcaaatattattttctttagcattcacagaactaagatctacttgtaacaattatttctccttattaataaggtttggaaataccccaaaacataatgggtccctgttattgtacatagggttgttttatttttccatgactttgccaacctgtgatgtttggtggcattactgaagatcaatatgagtgtttaaagatgctctctttgactctatttcagttcagattctctctatgcctggaggatagcaggagagttgggggcaggtatggtcttctaatttcttctgttgcagaatgaatcaacagaagcagcagggaagaaggagatgtaaaaatacagcataattcttattggacagtgctggaagttcctgtgggagtttgagatagaaaaatcgagaggtactagtctgaaatggagaggctcatcagttcagggtaggatctcagagagatactggatgtcaaacgtggactttgcactttcccctgcaacctctctccctctctccactttgtttgtccttaatggaagaaggtcgattatattaagtctacagatgaattatttggggaaagattcactaaatttacaacatcttttgagtgcctaccttggtgctagggactattctaagtgtatcaaatgctttgactcatttaatcctcccaacccccgtaaagcaagtactattatgatcatgcttatttttataaatgagaaaactgtggcaccgaggagggaaggtgacctgtccagttcacacagttaatgagcagaagagctgggattggaagtcacatagtttaactccagaaccacccactcccaaccatcatgcaacaccacctccccatccaggatatggtgtatttttcccacttatttgaatctttgacattttcagtagaatttttataatttaaaagatatgggccatgcacggtggctcacgcctgtaatcccagcactttgggaggctgaggcgggcagatcacgaggtcaagagatcgagaccatcctggccaacatggtgaaaccccatctctactaaaaatacaaaaattagctgtgtgtggtggcatgtgcctgtagtcccagctactcgggaggctaaggcaggagaatcacttgaacccaggaggcagatgttgcagtgagctgagatcacgccattgcgtgccagcctggcgacagagtaagactcaatctcaaaaaaataaaaaatacatatatatggttctggacaatttttcttacagttattttgggattttcaaatagtttttttgttgtctatatggtagaattcttaaaattacaatttctgattatgaaatttaaagttagttgaattcataacgatgaataaagtcacatgtgagttcaaaagctcgttaactcgtgcactgtcttgctagtctcatcccttgcactgtttaagagtggagagtgggatgtgagcaggagtgcctggtaaggaggcagtaggagctgacgctaaagagccttgggtaacggtaagaagcttgccttgctcctgaatcaaaccttccaacagaaggcagggaagtgagatcattaggtttgttttttaaaaagcatttttcttatagcattttactgggtgatttgggcttgggaaaggtcggtggcagggcacctgttagggcattattgcagtatttcaagtgagagataaagtctgaccccaggcaacagcagtaagaaaaatgggagtagcgaggatggggcaggggggcagtacactgaggggagggtaaggaacaggtctcaggtaaaagcgacagtcctgggtgaccagttaaacgttgggggagaggaaaagaaaggagatgaagttgccaccagaaagagtggcctcaggagaaaggggttgtacagggttggggatggggtgggatgggcagaatgtatcagagaaatagtacccttggggttggtgtggtacaggtgagtgggcctgaatgatagtaatggaaatcccaaagcatgatataaaaactaacgaaatttgaaaagtaataaacatgaaaaaaatgaaaaattttgatttatgggggatttatttggcttctattggagaatgattaggactgaagctaactacttggaagttattggcatataaatggtatgtgaggccagaggaaatggtgagatgagcccaagtagagaggccagctttaaacaacaacaacaactaccaaaaaaaaaaaaaaaaaaaaaaaagcaaaacaaagaaataggacacagagttagatttgaatgtcagatatatgatgaataattgtttagtatattccatgtttggacatgtttaagacctacttgtcctaaaaataatgtattatttatctgaaattcaaatttaactgggcaccatgtatttcatctggcaaccataacaggggcagcacacagaaatgttggcagggaccaggcaggcctttcagatagtgaggtggtgtgtgagacacaggccagtggtggaggctgggtgacccagaactcctgtctctcattctcaggggcagctgctacatacttcccactgattgggtttgcagagatggcctcgagggtgtctgatattctgatatttcaaaacagaatagaaatatgtattttaatgtgtatttaatatatatattttaaaatatttttaatgttgatattaaaagtggtcagttgggctcagtgcagtggctcatgcctgtaattccagcactttgggaaactgaggcaggaggatgtcttgaagctaggagtttgagacaagcctgggcaacatagcaagtccccatctgtacaaaaaaaaaaaagaataaattagtcaggtgtagtgacttatgcctataatcccagctactcaggaggccaaggcaagaggattgcttgagcccaggagtttgaggctgcagttaactacgatagcaccactgcactccagcctgggtgacagagtgagactctgtctctatttaaataaataagtaaataaataaactgggcaaacaatgaaaatgaaaacattgcgaaatacaaagcagctctgtgggttccactggtttgcagcctctgatctaatttctaaagtgggtgtagcaggtttttaacctgtaaattatggtgatcggcaggtcagggtaaatggtagttgtgtgtggggaagagatgatggcaacagatgttttcctcctgcaaaaactaaaatgtgggagtgacttctctggccatgactccatggtctctcccctcagagccattttcagtaaaatttttatagtttaaaaaatacggttctgaacaatttttgttagagttattttgggatttttaaatagttttttggttgtgtatatggtagaattcttaaaattatacaattataatgtataaaaaagcatgcaattataattcataaaattatagccccactgaccattctcctgtagctgggagcagttcacactaacagggctatacccccatgctgacctgctctgcaggatcccaggaaggtgagcatagcctactaacctgtttgggtagcacagcgacagcaactgaggccttgaaggctgttatcagaaaattgtgggcggaaacttccaggggtttgctctgagcttcttgaggcttctcagcttcagctgcaaagtgagtgggtgtttctttgagaagcagaatcagagagagagagatagagaaagagaaagacagaggtgtttcccttagctatggaaactctataagagagatccagcttgcctcctcttgagcagtcagcaacagggtcccgtccttgacacctcagcctctacaggactgagaagaagtaaaaccgtttgctggggctggcctgactcaccagctgccatgcagcagcccttcaattacccatatccccagatctactgggtggacagcagtgccagctctccctgggcccctccaggcacagttcttccctgtccaacctctgtgcccagaaggcctggtcaaaggaggccaccaccaccaccgccaccgccaccactaccacctccgccgccgccgccaccactgcctccactaccgctgccacccctgaagaagagagggaaccacagcacaggcctgtgtctccttgtgatgtttttcatggttctggttgccttggtaggattgggcctggggatgtttcagctcttccacctacagaaggagctggcagaactccgagaggtaagcctgccggcagactgctgtgccctggaggcaccaggcataaggggatggagggcccactgcctggcttgcaaagtgcttttcaatcctttttttttttttcttagaaatgggttgttctagttattctattctatagacagagaaatggaggctcataggggtgaagggaatattttttactttgtaaagaatcagagcccaacagtttgtctttcctcagttgtcaactcagtctgttgaaccacttactgcctcatacctattgatttaaggagtaaaaaacaaacaaacaaaacttttaagttttgcttggaagtaaatttgttcagatgaccaaactaaatcttgaaaattggtacgattccaaatcacaaaactgagggacagtgaggcaaaattgcttggccaaagcccaagttagcagaacttctgaggtatttggattctctttccagggcttggtttatttgacgattctgcctcttttgcttaaagaattttatttttattatacatcttttctctttctgttttactagtctaccagccagatgcacacagcatcatctttggagaagcaaataggtgagtcttttttcgcatgtacattgagttcccaaagatgatcctcagcacagaactatgttaatggaatgccttaaattctgtcccacactttggtttctgtacactataagaggaattctggctaattcagaatctctggtctatgattccttgagctgctttaaaaatgtgaagtgaattgaattgctcacaatcaatatagctgagcactgcacacaattcaaaatcacttccacttgactttggaaagagacacacatatacaaccctggacctttgccccctgagaagtggctccaggcctgtccccttccacagacatcctggtcctggcacacacgccagtggctgtaactcctgggaagagaaggcgaaatgaaggcagagacagatgtttctgagaaacgtcctttcctcttttaaatgccctaaagagattacattgaagctttatttacaatatgttttataggatgtaagcttttaaaacatggataatcaattccccacattagaatggtttttcttgaagtcactgagaagcttaagggaagacttcagccatcattcaaaggatgttcagcttcacccacagaggcagtcatgaggtacccttgatcacaaaggaaaatcctttgtatgggagctagggcaagaagatgataggttgaacactgatctcatgaaatttcagggcatagttgtgcaagctctgtacagatagattttacagtgtgcttcactagtctaagattcattgaaattttagatttagatttacaatgagattcattgaatttttagaggtggaaggaagcttagaatatatctagtccagccatttcattttttggaagagaaacaaaataccagatggattaggtgaatatgcccaagaccactggtatgccattggggctagaaggaacacaggctcttgatgccttgtcacctacagggctttccagtactggactggtgcctcatgaaggagggaaactcctgtctctctgtagtgagcatggaataagaaatagatgaaaagcaggatacctctgcaaagactataggtagatctcatccttagaaactcagaataaagaagaaggcagcactggccatctattaagtgaagcaagttccagtttctccggatgtggctactgcacatgacccagtcgcctgtctactatcctagactgttaagttcttatatgagccactgttgggatgctctggcccctgcttctgtgtcctaatccttaaatgttagaaagtccatatgtacacaagcagagtggaaatatttgatagaaaagtaaagcctctggaggacgtctgaaataagatcattttgaaaatctgtggaatgtgcctggtctgataggtatctaattattcatctttcacccactctctggagtcaatttagtgattaaaagtcaaatgatcaggtgagtcagatccatctaccccagtaagcaattatgttctctagatcaacccaaataaaccagaaattggtaaatcatcacatggaaatcaaatcagtaactttactaataagaaaagagatgcaaattaaaaatagggaataccaattttcatcaaattaataaagattaaaaaaaaacccagactgacaaagatttgagagaaaagtgcctgtgcaaacattagtttcagccagtatacatgtaaattgctataactttttttgaagagcgctttagaaatacatagcaaaagcttgtaactagtagtttacgttgtaatcctatcgctaagaatttatcttaagaaaataatcagagatgtgcccgaagatttatatataaggatgttcattacttccttatgagcctatttataataaataattcagaagaaacttgaatgcctcatacgagattagttatatacattatggtgtattcagacaagcacataataagcagcaactaaaagtgatgtatcagaaggacatttaatgctttggggattgtgcactctgtattgttagataaaaagacaagttacaaaagggtatgtttagtttcatactgactatgtaaccatgtatgcacatttttatattcttaggaactggaaagttttacacactgacatactaacagtgattttctctgggagatactattatagattatttatactttctaatttgctcctttttacattttccatttttttccggttaactaattaaatcttttattcattcacatgtgtgaggctagatgttgggtgttggatgctgattgaggatgagccagcccagggggcctgtccttatggagcttttagcagggggaagaagctaaacaaaagcaagcaaacagaccatgcaattccaaagttgtactaagtacaacctgagaatcttctttaaggaggtgttgtttacgctaacaccttgggggtcagaagcccgatgagtgaacggctgtgtgcatgtgctgtggagtggggtggggctgggggcgggcatttcagcgctgggaacggcacatgcaaaaaccctggggcaggaagcactcataaccagtgtctaggatatccttgcaatttatttattccagtataggtacagcagatattcttagtgagcatgttctgtaagaataacgttgtactaggctctgtgagggatataaagacatgcaacatgtggttctctgttccgtttcctgccggtgcgtggctgagtgccagattagttgctcgggtggtttgcggtgtaggaatgcagaggagactcagttttgcctttcctgttcgctaacacctcctacatggtgggttgtcttggatgactagagaggtcatgattgctcagctaatgttctctgtggattgggccattctcataccatatgtgagttttacaggctaatcacgaacctgacatagacaagcatctcctgtgaatatttattgaagtctcagttagctgaattcattagctaagtggcagctgtctatcttgtctgaaacagttgaagtcagagaaatagtgtctgttttgagaactattgtctatccttccatttaatttgtccaacatttgctagtaacatgccctgtgttagaaactgggcttctctggtgaactcaacgtatcgtaaggaacttgtcgttatttgaggggaataagataatttatgtagttaccccagagacaagaggagtcagaaatgacaagagaggggaagacagagggatcctagatttcaaaagaggggcagatcatatttgatcaaaaaaggcttgagagggtttcccaaagaaagcggtaataggtagtccttatgggatgtttaggaatttgacacagaaatctgcttgaaagaacattacgggcagaagacatggcatgaacaaaggcacagaggaaggaaagtgggggtatgtgtaggccacagaactcccgttgtgtggcaatgctcagtgggagtaaggagataaggctggacagatgggctgggattgtgtgatggggatacttgtatacgacggcatcttcggggaggagcaattaaaggtgtttgaggactcagtgatatgaggtgattgtcagaggtcttaagataattaatttggggcagattgtaaaatgtcactgggggaagatctggtgacacgcagacagatcagtagtcttcttcagcctctgtcctgtaagtctttggaacaagagaatcagagtggacagagccctggaattggcgtcaggagagcagggtgtggtcccatctgtgatgctgactagctgggtcttcttggattagtcacccaacttctaagctcattttgcaatcgataaggtgggacaataatacctacctcaaagggttgtggtgagttctaaatgcaataactgacatgacagctctttatgaataaatgtttatattaattatgttttattgtaacattataatttatacatatgtcaatcttaatttggtttatttttttacctctttgtttctgaaatatagactactatgaatcctgcagttcagacctacatgattagtatatgttagactgttgccatttacggttttaaaatcttttttttaaaatgattggatttaaattcccaccaaaataatagttgctatttcattttaacatatatttttcctctctctatgatacaggccaccccagtccaccccctgaaaaaaaggagctgaggaaagtggcccatttaacaggtctgtatctggaaggtacaggtgagatttgggaaagcttttggcaaaaggcaaaatggctgcctggtttccattaccaggctctagagagttgttacttcctggagacagtatttgaacccaataatactaacacttttcttgtcgagtcagtcttatgaacacactttgatcccctattctctgggcaaggccctcttctaaattggagacctggagctgattatgacatgacccctgcccataagaagctcattcttttcagtcattccaaagtcccttgggggattgggggcacagatccaagaggtgagagatgagaaggtgaggaatgaggacttattttagtcagtgctttcttggttttctggaagagcttcactctgctttgtccaagagtataaataggagtgagaagggagactcaatcctggcacttgtagagtttcaatatgtgcctgtaaagtggagtgaaatgatatggtatgggcactcccattataaactacataaataatggaataaatatcacagaaagtggtaggctattgtccctggaattatttaggcagaaattatagaatgatccggtcacatcagcagacacttactgaatacctgctgtgtgccaatttggggatgtgaagatgagtagcacatggctcttgcccccagggagcttgacatctaatagggaaagacaggcacttatgataatttcagtacttcagagatttaggttatgttcatgatgttgtggggatacaaagaaaagggaactgtaccccaaattgaggggctgaataattcttactgcaggtgagaagatggaccagatggtccctaagatccttcccaactttagaactttagagttccttggatttggctttttccttcaggaaaggacttcaaagcctagcagatttggtgctagttctgaagatagtaaaatctttgttccagagagcaaatattttctcaataatttcttactgcaatggattacgggtatatactattgttccaattgtgtggatgacaaaataggacaacgttgttgaggaaattctgtgatggatcaagttctgacccctcagccagttctataccagctgtcattctgggtgaaacatttgttgaaggaagggcccacagttttgccttagaaacttagtttgttggatgcatgactattccttgttgaaagctccttttggatttatttcaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctctaagagaagcactttgggattctttccattatgattctttgttacaggcaccgagaatgttgtattcagtgagggtcttcttacatgcatttgaggtcaagtaagaagacatgaaccaagtggaccttgagaccacagggttcaaaatgtctgtagctcctcaactcacctaatgtttatgagccagacaaatggaggaatatgacggaagaacatagaactctgggctgccatgtgaagagggagaagcatgaaaaagcagctaccaggtgttctacactcatcttagtgcctgagagtatttaggcagattgaaaaggacaccttttaactcacctctcaaggtgggccttgctacctcaagggggactgtctttcagatacatggttgtgacctgaggatttaagggatggaaaaggaagactagaggcttgcataataagctaaagaggctgaaagaggccaatgccccactggcagcatcttcacttctaaatgcatatcctgagccatcggtgaaactaacagataagcaagagagatgttttggggactcatttcattcctaacacagcatgtgtatttccagtgcaattgtaggggtgtgtgtgtgtgtgtgtgtgtgtgtgtgtgtatgactaaagagagaatgtagatattgtgaagtacatattaggaaaatatgggttgcatttggtcaagattttgaatgcttcctgacaatcaactctaatagtgcttaaaaatcattgattgtcagctactaatgatgttttcctataatataataaatatttatgtagatgtgcatttttgtgaaatgaaaacatgtaataaaaagtatatgttaggatacaaataatttttagaatttactaatatttgaattttttgcttattatttctttcctccttgatttcttcattcattatgatatcagaccacttttaagaataatttactggagtgagaagaactatgaatcaaatctttctgcctttgctaggccttccttctctctatatattctctttcctcttcacctcccagagtttccattaccaatattatgaccaatgttaagcttagccaactttccatagatttgatgagaaattatattttcctgaaggcagaaccatggcacagtggaaagggcacatattttggactaaaaaacttagatttgaatctttgttcttccacgtactgcctatgtaagcactggttaggtcacttgaccttccttgaacaaaaaaagagctgaaagtatcagacacatagaacatcaaaaaatagtgtctgccactactaagccactctattagagccacttcattctccagaggccactgatctgtgggatccatctaagcacctggtttcttgtattctcatgcttgtcattcacaacggccttttcctattttcttcaaccactcactatcatgggctctccctagactttgttatcattaaaaattatagtaactcctttggaatttgattttaggtgtcttattttcacaacatcatcttctattattccagcggaattggtccaacattttcttttctaaaatatttttgaaaatattgagacttctaattaataaactttattattttcttatatacacatcagctccttccacgtcttcacttctctctatgtccagcataaatgtcatcatctgtcatgacaaccagcaccttggaaagactctcaactccaaaatactctcttcctccaatgtattccccttgcaaaaatcctattttctttaagctgtcatcccagaagctggatgttgctggacaaaaattacacaaacaggctgcctagttttagtttatttttatctaacatgaatttaaatcaatcaatgattgatctgtcactgacttagttttattttttgattgatttgaataggtagtacatgtacatggtacaaattttaaaagatacataaggatatatgacaaaaataagtcctcgttccaccctgtctagtggctcaatttttgcccgcagagaggatcactgtttcctaacattgtcttgcagagatgatatatatataaatatgaaatcactttactttggagtttctcaaccatggatctgtgacattctgagctgaaaaattgcttgctataaggtcgtcctgtacactgtaggatgtttagcagcaccctttctctctaccaactagatgccaggagcatccccccccccgcaaaacatgtgacaaccaaaaaatatctccagacattgcgaaatgtccccatggtgtggagggggccaaagttgcctctggttgagaacctctaccttacactgatgaactcagtggtcattcaacattgtctagcacttttcttcagttcaatcttaagcatttttctttacttatagagtcaataacattatattttttcctcttgcaaaccaaccacatgccttcctcccactatgcaactctcttctacacaaaaacacaattccacctgcatctgcatgatctcttccttccacctggtctcagtggagaaataggacaattttcacttgtctgctagactcacctttcctcttcccttccttgtccctttttaacattttcttctctcctctaaatcctcaaattatccctgtatatgaaatcatttccaacagtatgtacctttgttcaagtatgtatatatcttttccaacactatgtacatttgttcaagtatgtacatatctttatatgtatatatgcacatatctttaagaataatttattgctgggcacagtggctcatgcctgtaatcccagcactttgggaggccaaggccggtggatcac

SEQ ID NO: 127 - DNA, 1132 bp, Homo sapiens
acagacccccaacttgcagctgcccacctcaccctcagctctggcctcttactcaccctctaccacagacatggctcagtcactggctctgagcctccttatcctggttctggcctttggcatccccaggacccaaggtaccaaggcagggaggggccttgcatggggctaaggggatcaagaggcctggataggagcttgccagcagcccctggctccctgtgaatcccaccctgcaggcagtgatggaggggctcaggactgttgcctcaagtacagccaaaggaagattcccgccaaggttgtccgcagctaccggaagcaggaaccaagcttaggctgctccatcccagctatcctgtgagtggacacaaaggggtgggtactggctggtgacggggtggggagggcatggtgggcaagactaagaaggcttactagcccccacccgcaggttcttgccccgcaagcgctctcaggcagagctatgtgcagacccaaaggagctctgggtgcagcagctgatgcagcatctggacaagacaccatccccacagaaaccagcccagggctgcaggaaggacaggggggcctccaagactggcaagaaaggaaagggctccaaaggctgcaagaggtgaggaatctgagggatgtgggtaaaggggagcctcagtcagcccctcacacccctcttctgccctcacaggactgagcggtcacagacccctaaagggccatagcccagtgagcagcctggagccctggagaccccaccagcctcaccagcgcttgaagcctgaacccaagatgcaagaaggaggctatgctcaggggccctggagcagccaccccatgctggccttgccacactctttctcctgctttaaccaccccatctgcattcccagctctaccctgcatggctgagctgcccacagcaggccaggtccagagagaccgaggagggagagtctcccagggagcatgagaggaggcagcaggactgtccccttgaaggagaatcatcaggaccctggacctgatacggctccccagtacaccccacctcttccttgtaaatatgatttatacctaactgaataaaaagctgttctgtcttcccacccaa

SEQ ID NO: 128 - DNA, 14697 bp, Homo sapiens
agaaccccgcggggtctgagcagcccagcgtgcccattccagcgcccgcgtccccgcagcatgccgcgcccccgcctgctggccgcgctgtgcggcgcgctgctctgcgcccccagcctcctcgtcgccctgggtgagtggatcgcgccgccctctgcccctcgcccctcctccccgcgccgctcggaagtttgcccggcgcccgccctccacctccactgttgacaaacttagacaaagccccggggaccgggctgggcagaggggcggcttcttccgctgcgccctggcgggacagggggatgcggccctgctgtctctgcgctggggcttttgggctgggactcgggacatcgggtgacagccctgccgcccccagggatgcggcttacagataatgacaaaggaatccgctgtgtcgggcctctcttttccctggtgaaaaatgaggccagggaactgcgtttgactttcgaaccccttccacctgggagattctaggactctagtatggataagtcttgtctggataactttgtcctggccatctccctgccaactccagttggctggacagttcattggatttttgcgctcccaattgtccgtgcctggtcacataagggaagggccggggagtcggtgcaatggacgcaggccgtaagtggggcccgggaggggacccagaggcttcgaggagcttggaagagggctgcctgctgatgggagtctcctgactccctccctcccgcggccttggccggctgctgtatcttccccggtcctcctccgcctcccaggaggcctccggaggccagctgggccccttgcaggctggacttgcggatgccccgtgccattcaccgtggagcgctgggagggagtcagggccaggactctttaggtggcccctccatcattttctcatagaaatgggattgactgaagcaaggtaggtaacagctgagccccagccctatgtcctgtgatctgcctgaccctcggggctagagcttccagagtgctccaagctcccaggaacttgggagctcctggccctccccggccaccatgaaagacagctggatcttcttagcccctttctactcttccctgtaccccccaacctgaactctggccaaatgttactggaaagtccccgaaagagcaggactctgaagtcccaaagatgtttttatacagggtgatgtggccttttccagaaggaggaaacaccatacctatcttacacacaggtaacgtggctgggtagagtacacctttccctttccctttttcccactggaccgttttgccctggagcagtgtagggagagggccggtgcagttgggagggaagagtccattcccaacccaagcctctgtctgtgtcaatgctcgcccccgcctgccaggtcagaggaaggattacctgctatttaaagccaatgactaatagctccttgggagccactttaagctcctgaggcccctggagggggagctctgagggcagatcgctcattaatggtgttgttgccttccctggagtgggatgaaggggctttgagatttcaggaggactctccagccttagatgggtgactctgagggggaggcaaaaaggtttttttttttttcttttttctttttgagatggaatcttgctgtgtcgcccaggctggagtgcagtggcgcggtcttggctcactacaagcttcgcccgctgggttcatgccattctctttcctcagcctcccaagtagctgggactacaggcacctgccaccacgcccggcccggctaattttttgtatttttagtagagacggggtttcaccgtgttagccaggatgatctggatctcctgacctcatgatcctcccacctcggcctcccaaagtgctgggattacaggtgtgagccaccatgcccggccaaggcaaaaagatgtctcatgtcttgctccaaatgacagatttgggtgtagggtctggggcagcccttctgggatgctaatgtcggctagaggactcctgttgggcccggccgacccaccagagtcaagggattgaggacaggccctcctgttccttctcctgtgttctccaccaccggggctgatagtgtacatctgtcatccaggttcccatccagaagccctggtggcctctccagcccttgccccctggtggttctgaaatcacccttccttttcactcctctgccctgacttcctctctcaccatgctctcctgctctccaccttcaccctcttcagtgggatttctgcatttcagccagagggacctgtctgcagctaccagatgctccccaccctgggagagctcagcggcatgtctgcacatgggccttttcagccttcttcatcccgcaattggaactgactttcccactcccatctcacacttgagaatctctgtttcttgtttcttgttttttattttatttttattttttctcccccctggtggacaatggtgaggagggtgaaggaaggttaaaagccactgctctacgggatgaaaaactaaacttgtcattcttcagatccctcccagtctggccccagcctacccttccagctgcatctaactgtgctccaacataccacaccctctgtgcatctgcttttcttggaacattctttactcattgcccagtctggcaccccccttactttgtcttctagacccagctcaaatattccctcctctggggaacttctaattccccagcagaaatcatgcctccagtgacggtccatgcaggcttctggagcgttgggcaatcttcctcatctgtggacttgctggcctctttccccttggcttcttgaggcctgcgctgcatctgatttctctgtctcgggggcctagcatggtcattggccctcccagtgtttcctggatgatcatcgtgctgttcctgagtcagggctgccattggaggtgacatctgtgactgcaacctgtgcctgaattggtgggcggaacctgctcaatgggccagaacaatgcttccttccactgcccctgcctgaaagtcctcatgggcctcctttgcaatcaaactgctgccaagagacctgccctggaccttgcctgcctctccagaccccagctgccagctcttccaccagtgggcagtggtccccagcaccactgccattgtcagccagcacttttctggacactcccaggccacttggaaagctgggcgcttcccttatgtgggccgattcgcctacacatggacttccttggctctaggcggactcaggctccatccagcacacgggggaagagggagaggaagagataatgggcgagtttgagtggagggtccttttgggctggacccatctgctacttctcacctgtgtccccagctccttcctgatcatctgctcatcttacctgtctctgtccccagatatctgttccaaaaacccctgccacaacggtggtttatgcgaggagatttcccaagaagtgcgaggagatgtcttcccctcgtacacctgcacgtgccttaagggctacgcgggcaaccactgtgagacgagtgagtatgtgggggtgcttgttgcctgtttctagcagtagaccctgagccacagccctggtggcataggccatatgctggccttattctctgcctgtgatccccaccccagtggaggtgtcaaggcctcctctggggagactcagagagagagacagagcctcagttagaccgaggggggaaaagggtcctgttgtttcttccttctttctgcctggccctctttcctgagactgccggccaggcctgatgctgatgcagaggagtgaggggttgtggtcaccctggaggtcacgggagtgctgtttcttgagtcaccatgagccagacacacagcattagggttaagttgcaaggatttttctacctaagaggggaaaacaaacacattcttggtcttttgaggcccaggaactcagtaaatcagccttggtttcagagtctgacagttcctggcccaagtttgacctgcagcctcatcgcagagctcggggagaagggagaggcctgggccagctgaaccatccccacgcggtcaggtggtcagaggccttgcatgcccagaggggctgtccgcgtgtgacctgcctggttatccactcggccacagaatggccctaggtggggtggcaacagcagttcccctgctgtcctccctccaggcccagctggagtccctcccacttagtgctcccaccccactaggactcctcatggggcaggcgggttgcccacatgtgggcccagggatgggtggagcggggggtcctaggtttctgaatccagcaggtcaatcgctgagcatcttgggtaggggacaggggacatcttgccctcctcagggagatgtggggatgggttctgaggtttgctggcctccagaaggagggggcggggggcttgagccagctgggtggcagcaggggccctgggagagagcaggtcaagtgagtaggatgccttttacctcccaagtggccctggggcctgtgccttggaccctgccctgaggccagatgtgtttgagatcctggtggaagggtgtgtgtatggtttggcggacgggttgctaaggcaatcagagccctgggtagggccaggtggagccactaacaagtatcctgggagctgaggtaacagtgagtgtgcctgagtgggaattacccaccttgacttctagcttctgggcctgggatcttccccaggctggatatggctgcacccaggaagaaggcaggggtgaccacacagcggaggaagccctcagagggaccttcgcagctcttccttgccttttacgtggtatgtttccttggagccaacaaggaccctgcgggcccaaagaatgcacactggactccttcagggtgtgagcgtggtggggtatggggatagtcttggaggcagactggggtcaaatctcagctcagccactggccgagctctggtcttgaggctctggcctcactttgcgcgttcccctccctgccagagtgcgctaacactgagctcgtccgcgcctgtgatgttggaggaagactagatctgtctgtgggttctctgcttggcagctggaaggcccaggcacacgctggggggatccccaggtgatgagagaagcttggcctgtaggtgggcctggtagctgtcctgggttcctgggaccacactgctggagtgatggtgaggggggtggggccttggccacgggcatgttcacagatggctgggtgagggcccacaggtggaaggcgtaaagagggaaagcagaatggcatttgtgccccacccagagaactcctgccagtgggattctagaaaactccaggcagattagcttctttctttgtgtacagtgctggtactgttcactgctatccgaggtagcccttctcccaaagccaccctcctgaggaagccaggggctgggctgaggatggcgtcccttcctggtggggggaggggacactgaggaagagggggtcacaccggtattctgtgccaccagtctaacccctgcaccagactgattgctgccagatgccctatatgaacgaggccacctcccacagcctggcgggggggactctactttccatgagtgccaatcatggctgtccccacagtgctaccctggctgggatcctctggcagccctgggtcccagcctcccgtgactctgcccacccccctcatcctcaccacccctggccacataccttttcttggcacctcgtcctgggcactgccttcattgtccctttgtggtctttgttctgagagaggctctgaggttctccctggccctggggcttaggcagaggtcatacagtcttattttcttgtccgctgggtcccagctgggcccgggaacctgctggggtagccacctgactgccacctcccttccctcagaatgtgtcgagccactgggcctggagaatgggaacattgccaactcacagatcgccgcctcgtctgtgcgtgtgaccttcttgggtttgcagcattgggtcccggagctggcccgcctgaaccgcgcaggcatggtcaatgcctggacacccagcagcaatgacgataacccctggatccaggtatgcctggggatggccctggggcggggcaatggagagaaggctcgatgaatgtttctgcatttgggggttaccccaggagggttcatcactggggttcaggtggcaaggtaacaggttcccatggatgaagtatctgtaagtggatggcatccctggaggaacctggtatggggtcaaggtgaggggggaggaggccccaggctgtggaggagctgttggtgctggatgaggcctctggaggcctgggcagaagaagaaagccctttgtgcgggcagtgtgctcagctacccctcctctgttttgcttgagttgttcccccacccagtgggggagtgaagggatgggtctgcttggatcttgagtggaaagaacccaggagataagggtgccatctcccctgccctgtgtccaggtgaacctgctgcggaggatgtgggtaacaggtgtggtgacgcagggtgccagccgcttggccagtcatgagtacctgaaggccttcaaggtggcctacagccttaatggacacgaattcgatttcatccatgatgttaataaaaaacacaaggtaggtcttgttggggcccgaaagaagggctgtggtccataccccctaaggttctagcattgactgaggacccccagcgagtccagagaaccctaggtgattggctatgatgtgggtcttcgggtgcatcttggcttctagaaattgggaaacccagactgtgtccatgttctgtggtttataagcaatagtcccactttagagatgagactagtgagattcaaaatggcacagtgacctgttgagggtgatagtggtggtggagctgggtttcattctgagccttcatgggtacatgtacagttgtgtaggttgtacactgctcaaggatgtcattgctaagcaggcaccattcacagtacaggcattgtacatttgtggaactctgtcagaaaattgtcataataaactatttgagaaaggagtgccttttcctaatttgcaaagaggtaccctgtaggttacaggtggccctgtgtcagctataggggtttatgcccaggctccatgatgattaaggaaagtgagaggagaatctttgtggagggccgtggggtggccaaaggagaggctgctgtgtgagcctctgctggctcttccaagtcctcttcaccctcccagccccacctctgcctcccaggtctgaagaggaagatgaggtaaccccagggagggggactgctcagatggacccccttctccagtcttgtcccttccctgctaggagtttgtgggtaactggaacaaaaacgcggtgcatgtcaacctgtttgagacccctgtggaggctcagtacgtgagattgtaccccacgagctgccacacggcctgcactctgcgctttgagctactgggctgtgagctgaacggtgagtgctgggggtgcgggtaggggggcactgttggccaatcctaggaggtggccctccctgctgcctcttttctcagccaaactgagctaggtagggcagcaacaaggcacggggaccaggcttcactcgggcaacgcactctagggggtcacctggccatagcagccccacccaggtgtttgctgtctgccaacccatgacccagggcattctgattgatagagaaaaagacatcagttttcaaagtattttcccttcttctggttggctcctctcctgtagcttctagaacatgtagcgttaggtgtgtttggcagatgtcagctttacgagggcagagaatctgtctgttttgtttgcttcggtattcccagggcctagaatggtgtctggtttataggtactcaataaatatttgatgaatgagcgaatgaatgaatgaggggcgtggggccccgatgaatgatttattaagggtacacatctggtaggtattaagacttggtctagatctctgggctcccgaccacacccatccagagctttccctgccaaactcttaagagtctgcactccagagaaacacaggggcttcttggaccacagggcttggagctggtgcttcctgctgccttttccaaagagattcccagatgaagagctggggagaccataaagggacagatttctgcagagccaggggacagcctgggcgaggtcagagtgccgggtttgggctgacagacattaattgtctcagcccagctcaacccagctttcctgaggtctgggtatttctctctctttgcagccacaatgccacctgctggccatcagtggaactgtagggctcgctaagcagagctgcagagtggaggttcttatcttacccgctggccttttgatcttacctgccctcctgtgccccgcctttcccctctgtgacacctcttcctgttgtctcatttcagacaccacccaaatacccctcagagctgagttcagtgcctggctgggttcagtgtttttagtatgaacttgagtaggtatgcttcctccaccaggggcccttttgagccgtattgtttcagtggggagtataagactttgccttctgctagaaggaaatgtaggctactgggaaaggacagagccaggtctgtgtgcctctccctggagtccctgacacttgctgttttggcttgaagcctttggccattaaagttgcctgtatccaggcatgactggcccctgctcagggccaagagagggtgagtcttgcaaaccaaggaagcccaggtaccctgtctgcttccttcagggtctggaccactcctttccctataggccttccagtcggctggagagtgttggtgcatgtgtgcatgtgtgcacgtgtgcatgtgtgtgggtggggctgaggtctgggggtacctgggattccacaagggattcctctttgcagacatatggtgtctttggtaaattaattatttaattcgatggatgtttattaagatattgattaagcccctcctgggtgcttggctctagggaatgattccttgacctcatgtaatttgcattgtagcaaggaaggcagacttgaggcgaggagcagatgacagagagcagtaacagctctgaagaaaagctagaggctgatgagatgaccatgacttggggtgaggggagagagcaagacaaggactgctctgtcgggaaggcctttgactgggggcctgaatgatggagagccagccacatgaaaaacttaaaaacagcactgcaaaaagagcactgcaggcaaggaattggtaaatgcaaaggccctggggcatgttgaagggtggaaggaaggccagtggagctggagccaagagatggaggggcagcttggtgggtgaagagccccagggacccgcagtgtcatcacctaggagcttcttagaaatgcagactgaggccgggtgcggtggctcacgcctgtaatcccagcactttgggaggccgaggcgagcggatcacctgaggtcaggagatcaagaccagcttggccaacatggcgaagccccgtctctactaaaaaaatacaaaaattagccaggcgtggtggcaggtgcctgtaatcccagctactagggaggctgaggcaggataattgcctgaacccggtcagaaggtggaggttgcagtgagccgagatcatgccactgcactccagcctgggtgacagagcgagactgtgtctcaaacaaacaaacaaaaaaacaaaaaaaaagaaatacagactgaaagccccagcctagacttgctgaaacataagctttttttttttatatcaagatccccaggtgatctgtgagaattactcccctagtatctcattttatagatggggagactgagagccagagagaggaagggacttcctagagtgctggagctgacagtcctgtccctgccctggtgccccttccaactgcattttaccagaaaggctgtctgacttcctggggaggctgagagtgaggagggtatgaaccactagaagcagatgatctgtagaggggtctgcgttgagcacattgtgcagaattggtgtcctctgcaaagctgcccaggttttagcctgcctcaggcctctcctcttctggggccaagggacaaccagctctcagcccgggagcctctctttgagggggaaggtggtggttccctggtgcatcaccatcaggagatgatggtaccatcagcaggcattgtcaacacaccctggaggcaaatgtcttgagtggtgagagtacaggtgggcagtgatataacttagctggctcccagtactcttgagtggtgagagtattggagggcagtgatataacttagctggctcccagagggcagggggctctccagtcccaccgtccactcctgcctccctcaattccccagagggatagacacttgtggagtgaggattcttccaggtgggagacacccccatcattctcttgggagttgtatctcttgcccatccccaactgttctatccatatgcaggtgcagatacaagtgcaaggcagcccggcacattcctgagtgtttggggcagggctgagtgaaacaaaggccagcttcccgggccagccctcacaggcctgtgtgacagcatccccagaacatttgagtgtgcgaaggccctctgggagacactaagtcagggggcttctggggaatggaaatagagatgttcctgtgccccaggagttgtctgatttctctaactgccagctgctggagggcagggatgttgtcctgttcatcactgggtcgtcactaggcaggccttcatcatctagctgggatgtgtagctggtagaggtgacgtcatagtgtgacattaggagtttgtatgtgacttgagcatgatgcttaatgtctttcagttttctcatctatagaatgggcttactaatttctacttcatttagtgtttttttttctttttttttttggtggcaggttaaatgactgaagtaagcactgaataaacaatgtggttgttattaaagtagatagtgactgtgccatgggacggatgggaggccgaggcgatgggggcagtgctgtgagtccagaagaggaagaaatcctgcccaagtgaagtagtcagggcagacttcttggaggaggtgtctccaaagtcaggcattgaaggagggcaggaggtggtggagcaggggccttccctggtgcagaggagaagggtggtgagtgggaggtcttgggtgatctgtgaggagctgagcccggctggttgaggtgaggaggtggtggggtcggcagtgagaaggtcgcacagttggccctgcaaggcggatggagctgctttctgttgcccaagagcatgacaggggctggtcaggatccacagagttctttctgcttcatcgccttgtgctcctgcccctgggagctgtggatcccagatccagatgacagcccaccttgctggcaggatgcgccaatcccctgggcctgaagaataacagcatccctgacaagcagatcacggcctccagcagctacaagacctggggcttgcatctcttcagctggaacccctcctatgcacggctggacaagcagggcaacttcaacgcctgggttgcggggagctacggtaacgatcagtggctgcaggtgggtcagccttcttgggatatggggctggggttgggtgggatgaggtgggactgccagatctttgacctctctccagcccaggcttctcttctgctccccaaaagagttctggaaaagccttgtttttcttcggcttttctttcttcttcccacctgtactcccttctcttgatcctgcttccaccaaggtggagaaagggtgtgtgtgtgtatgtgtgtgtgtgtgtgaatgtgtgtgtgtgagtgtgtgaatgtgtgtgaatgtgagtgtgtgtgaatgcgtgcgtgtgaatgcgtgcgtgtgaatgtgtgtgtgaatgtgtatgtgtgaatgtgtgtgtgtgaatgtgtgaatgtgtgtgtgaatgtgtgagaatgtgtgtgaatgtgtgtgtgagaatgtgtgtgtgaatgtgtgtgtgtgaatgtgtatgtgtgaatgtgtatttgtgaatgtgtgtgaatgtgtatgtgaatgtgtatgtgaatgtgtatgtgtgaatgtgtgtgaatatgtgtgtgtgagaatgtgtgtgtgtgtgtgaatgtgtgtgtgtgaatgtgtgtgtgtgaatgtgtgtgtatgtgtgtacacaaggggagagccattccatgggctgccacaggattgacaaggctcagcctatggacagacagcagggcggaggcggaagagggagacatgacccaggcatcttgggacctctcccttgagcacagcatacatattctctgactcagtgtctaagaagccaccatgtgacagcccttgtctgtcattgaaactaggaaacaaaagagagccatggaaaaccggagcttgttttatagatgaggaagccgagagctggcaggacgatgatctgcctagtacccacctgctggctggcagtgagttgggtttacacctggatgttggggaactgtgtgtccaggtgcagctggcaggttctccagggaggtcagggaccggggacaggaggtctgtctcagggccccatggagcacactgcggagctgggttccctgaggagctgcctgcctgtggacaccctcaggaccagtgtcagactgtgggcggcgccgcagggagcctctagagcagttcatgcccctccaggctgtggcctctcctgtccttcttcccatcacagtgcagtgcaggtctcaggcaggggtggttggcgtggagttggccagggcccagaccctatgagctttatagcagtgaggtaccgaacaccatgtgtggcaaagtaggtggtattatttatgccccctcaccgctttttttttttttttttctgagacagagccttgctctgtcacccaggctggagtgcagtggcgccatctcggctcaccgcaacctccgcctcccaggttcaagcggttcttctgactcagcctccctagtagctgggattacaggcagccaccatcatgcccagctaattttttgtatttttagtagagacggggtttcaccatgttggtcaggctggtctcgaactcctgacctcaggtgatccacctgcctcaagtctctcccaaagtgttgggattataggcatgagacaccatgtccggccttatgcccattttatagccaaagaaactgaggctcacccaagtagtttttttcaaaactagaattcatgcctcagtgtgtctgcctcttttcaccttactggagatggtccaagcagagaaaatgtggttggtttcttctgtaggtggacctgggctcctcgaaggaggtgacaggcatcatcacccagggggcccgtaactttggctctgtccagtttgtggcatcctacaaggttgcctacagtaatgacagtgcgaactggactgagtaccaggaccccaggactggcagcagtaaggtgggtgtctgtccagctgcccaccctttgccattccttcattacttccctgggagtctggcctggggctctgaggggaggggggctggctcggggtcctcctgacacccgctctgcctctagatcttccctggcaactgggacaaccactcccacaagaagaacttgtttgagacgcccatcctggctcgctatgtgcgcatcctgcctgtagcctggcacaaccgcatcgccctgcgcctggagctgctgggctgttagtggccacctgccacccccaggtcttcctgctttccatgggcccgctgcctcttggcttctcagcccctttaaatcaccatagggctggggactggggaaggggagggtgttcagaggcagcaccaccacacagtcacccctccctccctctttcccaccctccacctctcacgggccctgccccagcccctaagccccgtcccctaacccccagtcctcactgtcctgttttcttaggcactgagggatctgagtaggtctgggatggacaggaaagggcaaagtagggcgtgtggtttccctgcccctgtccggaccgccgatcccaggtgcgtgtgtctctgtctctcctagcccctctctcacacatcacattcccatggtggcctcaagaaaggcccggaagcgccaggctggagataacagcctcttgcccgtcggccctgcgtcggccctggggtaccatgtggccacaactgctgtggccccctgtccccaagacacttccccttgtctccctggttgcctctcttgccccttgtcctgaagcccagcgacacagaagggggtggggcgggtctatggggagaaagggagcgaggtcagaggagggcatgggttggcagggtgggcgtttggggccctctatgctggcttttcaccccagaggacacaggcagcttccaaaatatatttatcttcttcacgggaa

SEQ ID NO: 129 - DNA, 4283 bp, Mus musculus
tcccattctaattggttggcgtgtacttagcgaatctaatcagggctaccacgggaaggatggggtcctcaagcaaccgcgctctcctgcacatggtggtggtctctttggctgttacccaaacaggttctggtgagttcaggggtcgggaagtaaataacctctaaagagaggatcacggcatcgggaccaggtgctcggggatcccttttctgccgggacctgagttccgctccctgccccacccccaacccaatgcttgttcactggcccggggttatcagggaagagttctgaagttaccctgcctcgcccccaggctcacattcactgcgctatttccacactgcggtgtcacggccgggccgtggggagccccagtatatctctgtgggctatgttgacgacgtgcagtttcagcgctgtgatagcattgaggaaattccgaggatggaacctcgtgcaccttggatggagaaggaaagaccagagtactggaaggagctgaaactcaaggtcaagaatattgcacaaagtgccagagcaaaccttcggaccctgctccgctactacaaccagagtgaaggcggtgagtgaccctaagcctgggtcagactccttcctgtctgtctactgccaaaccagagtagccctgggtgccaggttagatgtttggagttgggcttcagagctctcagaaacctttaaccggtttcactttcagtttaggtccaaatcctgttggcgggcggagcagggtgaggtggctaaggctgacaccctgtttgtggctaggctctcacatcctgcagtggatggtttcctgtgaggtagggccagacatgcgccttcttggagcccactatcaggctgcgtatgatggctcagattacatcaccctgaatgaagacctgagctcctggactgcagtggacatggtgtctcagatcacgaagagccgtttagagtcagctggcacagcagagtacttcagggcctatgtggagggagagtgcctggagctgctccacagattcctgcggaatggcaaggagattcttcagcgagcaggtatgaggaacaactaggaaccacttttagtaagcaaagataaccccctcaaacatacaagtgaggctcttgatgtctggggagggaggaaataccagatccgctggaatactgcacaaggaggaggaagagtggtactggccttcctgatccaacatcagagagtgactgtccccagggctgaccctactttcagaacatttgaatctgtcccagaatggaaggagaaaacaccctgagatacacaaccaaccacttccattcagtctgcagctcattgtgtaccatggcctctttcaggtctggttctctcaacacccctaatgtctttggaagtcacactgcactaatgctgtgagtctctgctcaacctaatcaggactagagatgggaaatgtgaagctccctactagattttctcttagggcttctattgctacaaagaaacaccatggccaaaagcaagttggggaagaaagggtttatttggcttaccctttcagcactgtttgtcattgaagaaagtcaggacagaaagtcaaataactgatctacatgacatggaggagtgctgcttcctgtcttgcaccccatggcttgctcagcctgctttcttatagaacccagacctcaagcccagcgatggaaccacccacaatgagttgggccccacagcatcaatcactaattaagaaaatgacctctagtcttgcctacaatttgatcttatgctgtcattttgttaacggagattccctcctctcatataactttagtttgtgtcaagttgacataaaactatccagtgcactaaccctgattttagaacattccaaggaatggatcatgataggtccctggatccagactcccgtatggtgcttacatccctcctgtgccctaattctgtccatccagaggatgctcctatgatctccaatgactctccaaaaataatgcaaaatacttgaattttccacctttcccccacccagatcccccaaaggcacatgtggcccatcacccgagacccaaaggtgatgttaccttgaggtgctgggctctgggcttctaccctgctgacatcaccctgacctggcagaaggatgaagaggacctgacccaggacatggagcttgtggaaacgaggccttcaggggatggaaccttccagaagtgggcagctgtggtggtgccttctggagaggagcagagatacacatgttatgtgcaccatgaggggctaaccgagccccttgccctgaaatggggtaaggaagagaagggtaacagagtcaaggaaagaaggagcctagattaagatcagaactgagaactgggcatcatgactctcaccctccactcctttcccaggaagatcgtctcaatcatctgtcgtcatcatggtcatcgttgctagtctggttctcctgggaggtgtgatcactattgttgtggtgtgcaagaggaggggtgcaggtaagcaaaagggttgactgagtcctttttctcacctctggactccaggtcttagttggaattgttcctgcctgttcactgggtagacatttggcccaccatctcaaaagacacaatagtttattttatgttattgtctatctatgagtatatgatgtagatgctgtgtttataatgtaggtatacgctagtcatatatgtatgtatgtatgtacatacatatgtgtgtatgtgtccatgtgtatgtttatgtgtatgtatgtgtaagccagtggtcagtactgtatgtctccctccatacttcttatcttttttttgagataggatcttactaatttggttagatcagctagccagtaaactccagggattctcctatttctgcttccctagtactgagattatagatgtgaatcaaccacatgcagacagactcaatatacacagtttagacaacttacatctaaatcacaaataactaaatgttaaaagatcaaatttccaaaattacaattctgggaaaatatttttttgattcatgtaaagacttttagctacattttgaagaggtgtttacttaagatgcataaagtatcagagaggtctccttattcatgtttgcatgggtatagtaatgaaagtcacactgatgtacatttatgaacaattaactaggaagaagtatcacaaacaaatgctagccttgcctgagtctctgtgctgacaatctgctgtgcataggatcgtgggaaagaaaagacataatcagagtggaaggttcctgctgagaacagatcctccaaaagactttattacacttcctttatatcttacatgttctacatccttccctgagcctgcagtcctagttctatcggcttccctggggctaggattaaggcttttcatagactttcatggccctgctgcctccctggcctcttacccactgttttctttccacaggggaaagatgaatcagttatactcagactgccagtaagtatggtgggatatggttcctgatatgagataaggtggacaggagcccatggggcaacacagccagctcatcttgtggtgtctgatcaagtctcatttgttctgttaaaggcaggaacagtgattaagttctgatgcttctttcaccatttagaaaggtaagaagttgccgggcctaacctgtagcatgtggtggaacagaggatagacccctgggttatggggactctaacaatgggacatttgagctgagtactttagagtatcacacctcccaggaaagtttgaatttcttctattttcttccccagtatgaaagaaagccttgccttgtgtgggactgagtttacagcatgtgttctgatctctccttctgtactataattccaaaaccctcaatgtctctttctgcagttggcatctcatggtgtgtctcctgtcagcacagtgtgacaaagtgagcagcccagggcatccctgaggactgagtctcctctccatacctgtgtctcctccttttctcacctgtaccacagggggatccaggacttcttcatcccttgtggaactttaggtttctatgggcagcttcttccctattgaaaaataaggatgtagatatagctttattattattatttttttttttaccttctcgataaaagctgtacttcatgctgttaaaagtgtaagatttgtacattggaaaggaaataaaacatgaaaactca

SEQ ID NO: 130 - DNA, 20895 bp, Mus musculus
catactcagacagcagagccaacccatgcaggtaaggagcttagtgtgataatttttaaccctggacagtccctatttatctgtgatgaaccctgagcaaagggccagaaagtctgtgagttctggctgaggccattgaaaaacatgctgagaatttacatagttacggtggtcaccaggacaggctgaagatttcttagctaaaattcttggctcaagaagggttgtggtttatttttttttcttggtattgaaacattttcagtatgtattgtgttatcttggcaataccttgaggtctgaatatgaaattaatttttgtttcacatccatcccatgcacatagctgatggtaatagtatgtggtgctttaaataattttgtaaagcaagaaatgttttggagttaggatgtgttgtctccaaggaggagtgcttagcctttgatgctagatgaataccatccagtgtcttttggtgagcatgggtatgcatggctattgagtaccctagtgttgaactgctggtgaggaccaatgttaatgcttccaagaaagaattacaaaaatatgcaagtatcattccacattctgaagggaagggcaagctggggtctccataattatgtgacattagcatgtatttgaatcactatcaccatgagctaaattatgtcccttgatattgatattttgaagtccaaatccttggtacctcaagaagggattgcatttggagtttgtctttgtgaaaatgattaagttaaaaaaaaaaaagataggatagtggtgtctcatccagcatacttagtgacttagatcctatagtgctcctgagtcagcatggctcctgatagtacccatcgccagcacatcctccttgatgatctgaatcctcctctttcccctttctccttcctctggttttcagaagtcccaccctattctctattctgcctgggaattgggtgattagcatttagtgacaaggcagagaattaatggtaagaattgtttacacaaacatgagacaggacattcttggtataagcattatgatgccatgtccagattgaaataagatatgagggcagagaaatcagcatttgaataacacaagggtaaaatttacacagtgtataaaaacattatgccttataggaactaacttctgagcaaaccagcagcatcttggtgagcattatggtgagttctcttctccatcaaaaggtaacacaattcatggacattctgatttctccttttcttgtctttgtttcaaccttatacaatcaacttgtagcagcttgaatggcctaatgactttgtttattgtgtgttttttaatagttgtatcaggcattagacccttgtgacataataaaggctcaaatggttgaagtttgagactaagaacatttacagcaccctagaagggctctgacctcatctgtataccctagctgtgagaacagttcttgggagctttctagaatgtaactctgtgtatccctatgtacagcaaaaatgtgtttatttgttctctcaatttggtcagtgtcacaactagagtggtctggctaggtctcctggtttgttgtgttcttattttttctgaacttaaataacttcttttgggaaggttcataaatttgaaaataataaataaaatttaaattaaaaaattcccattagtgcttactttcattaatcaatgtttttcattttgaattataatttgcagttttctggttcttttggtaaaatcagtgggtatccttaagacatggaaatggctgtgtgcttattctctaagccccagtccatttagggaaaggttttttagtaactagaaaagggtgtactgtctgttagaacagctctgggtgaaatgtcttaggaaaagaaaagcaaaatctttctacagattcaattccacaaggcttttttgtgctccatactctcccatatgaccattcttccctaagactggatgacatgttgagtatttcagcctgttacatggtttagaagtgttgctggattgtgtgatttgtgttctcatcatcatgtgtttatggtttatcagatttcagtagcagttataatagcaatctatgttttataatactattaggttaaaaactgaatgtagaggggcagtgagacagctcataaagtttgatcttggaccatggtagaaccttaagaacctgaagcaggagcgtcatatgttaaagccaacttaggcttcaaggtgacaccctatctcaaaccaagaaggtattggtcaggaaagctgttacccaagtggccaaagccaagaagttatagacaaatgtttctgattcctggtctcctgacagtaactggatgagttttattcttggtttccacttgtattccttttatggcatttgttcagacaaaggcctattataccctaaggctatagtgttagaagcaagtagatactgtttccagactgttggacaaagataccctaaagggacatttgtacttcatttgactaaaaactatttaaacagtgttccccagtgaatttccatttatctaggaacatcatgctgatggatgtaaaatcccagagccttccagagaaaaattgtgttccagagagagatttctggtttctctgaccaccatgcaggacagaatggagtcaaacagcaatgggacaatgataggtaggatcacatctcgactgcttagctctatcaactcaagtcttttgttttctgtaatctcatatcatgaattccacttatggacctcagagcacactgtataattagtacctctcccctatgtggcctcattgactaaacctcctttgtcttgtgttactatgaatttacctctttaaatttgcttttaaagaacaactggccaagccaggcttgttagggctgccagaacacaggctctaaccctaacagtttattgatatgattattgttttcttttgtgtgtttgtgatagagttccactttatagtccaggctgtctggtacttcatatgtagaccaggctacttttgaatacacagagatatgcttgtctctatcctttgattctagcatggcaaatgtgtatcactgcatgtggtaggattttctctgtccaattacgttaaaatattagtgcagcaggagacctgtgattggacagagaaagagagagactaaaagtagggcgaggagaagccaagatggaaacagacagacaggaagaagatcctgatcccgtgtgattttaatagccataggtaactatgaatatcataagggatagaatagttggggtaacttggttgatgtaggtgggcagcttgtatcattatcaattggctctgaaatcattgtgtgggtatcttgtgaattgaaaatttatatataaataaatatgacttgttaattataagattctagattgttgtttctagaagggttgctggatgtggtggctgaccttgcggtgggcagtcattgcatggggctggcatcacagtgaagagaacttgggagctctggccccaaccagagagctggtaggcagagagtagctggatggagcacgggaacttggctgttgtttttttaatatttcccacaacaaccagcacccaactgtgactgatttatttgttcaccatactttatgtctctgataatgactcttgctggggattcatagttcacagactgttgaactgttttcacagatcaaccacctcttgaagtttattaaactttcctgtattgtttcttacataccacttctttcaggtactctctttctacccaccaaaggtggagcacttcccagccggagctaccttcctgccactgtgtgatccacattctgattctgggacaggaggagatcagaagaggttggaggctgaaaaatgtaaggagtcatagacaaataagacatatttattcactgtggggaaaaaccaccaggctgataacttccagcagggacgcaggcccaggatgatctacacagtacacacacaggaacatgtgggcctagacagtccagtacacaaaccacaggaatgtagggatagagatctacaggcaatctggggaaggcagtctcagggtccagcctcccacctacaagaacaaaaagaaacctaggttaggacatgccactttttttaacagaattctaaaactttcacctctaaagtgagagcaacttcattggatccagtcaacccaaagaaagatttcacagcgaaccaatcaactctcagattattatgtctgattaaaggggcttaagtgagagctatggaggcatgaaaaggagttttctcaatagaggccacacaggttccaggaagaggcagaccagagctgtgactacggctaggaggaacttttcatgaatcacatctcacggaaatgacagtcacctgacgtatttttggaggttttagaatttaccacagagataaaaaggtaagcagagaacgcaattaagaaattggtgtcggggctggggagatggctcagtggttaagagcactgactgttctttcagaggtcatgagttcaattcctagcaaccacatggtggcttcttctcctatgccctcttctggtgtgtctttagacagctacagtgtacttattaaaataaatacataaatctaaaaaaaaaaaagaaattggtgtcatgcaattcatttgcaatatgatgatgacgaagatgaagatgatgatgcctcaagaaaaatgggaagtaatgtgagtaggcagtcttatagaaaggtttttaaaggttcagtgagcacatgagcttatgattgacatcattactcatcagggaaaactcaaaaccgtaaatgagtaaccttatcacaccgattggctaaaataagaaaagaaaaaacatgcacgggaagaaactgaaacttatagcctgttggtggggcatataatacacagaagacctcaaaagcattaggtaggtagttagttcttcagagtatgaaatgtagagttgtcataagacccagaaattccttgggctataccctaggtaacgccaagagagcatatatctatacacaaatttatacatagacattcatagtaaaattaataccagataaaagatagaaacaatctaacgctgactgattgaatacaaacttaatttagataatatctttaaccagacaaagaaacaaatgtctgacaaatattgtcttgagatactaaggaaagaacaaaatagttaagcagattcaggaaaggaatgaagtcctacattcccgggtctgattaagactcttgccagcccagttaacaggttctgtgacggttgctgacatgcacacaatactcattctctgcatggatatttcaatgaagaagagtaaaacagcaatcatgcttacagcctaagcatacagagtcattctccttttccagatgtgagcatgcaaattcctaggactcttagaagaaaacctatttatagtccaagagatgccttggcagccttctaacccatgctttatcgtgcctgactataattcctatgcaaggtacccataaagtcatatttatttaggaaacacaaggaagggaacttaggcatagggggttgctgttgtagctctctgggatagaagccccaagcagcaaaaatatggaaaattttgactccataaaattctccaagtgagagaagaatgagagtccatgccatcaagcaggaagtgcactgagaatgatcacaataacttacttaagggagacgaagtccatggagaagcgtattgctgtgggacctgtgctctgtagagtgggtgctgttttcaagggcaggtccatgtagtaatagactacatccagaaaaccaaattggggggtgagtgagtagataaaaggctagaacattctagccaagaataaaaaaataagttggttcattgttgaatgagatggagctggggtgaggaaggagaagcagaacagaaaggagatactgcgtaaatgaggaaaatacctaattaaaaaaaaaaaaagatacaaggacaaagaggaagatagcagtgggccagctgcttcagtgacgtgggatcagggggaaagtgaccccaagaacaaaagttaagacaggaggtggtcagactggctgttgatcaaaggagagaaaatacggtaacctaaagggacaggaggttggatctcctcctgctggtggaggacagagggtgaggaagcagatgttgcaggtaaggaagccaaaccttgcctcaagcaagatgatcaagaaagccaggagctaaaggtcctgcagctgctgtcacttcctgctgggtcaggggttagaggagagtggtgtgaggagattcttgtggaaaatggagctgagaggatgagcaccttggagaagccaaaaagcaacacagcaagcttcagggaagaagagattggagaaaagccatgagccacaaggagaacacgtgggaaagaaagctttctagattgagtataccacagggtttaaaattgaatggactctcaaggttagcaaaatgggtgactcatcctatagacagggtgagcctctcctgtgtctgagaagtcttcctaatgttctttcctgtttcccaccaaggcttcctatgttgaagttctacttatgcgtttggcctcgtatagtttcattctttgtcattttaaaagcttgtccttaaataactgaacaaagaaatactgaatctctattaactacaaaggagaatgaggttttctcagcaactttgtaagatgtagcatctgagagccaccaaagccacacatgaggctactgctgagtcactgcagccatgctcgtcttgagagccctgcatgcttaggtgtgtctcattctctcttttcctacttttcttccctttctgtattactttttaaaaatatatttgtgtttgcttctcaataaatccaactccatctcatacgatctctttcagaaatttctttctctggcaaagtcaagaatggtggtttcacttgagttgagttttttttttttttttttttttttaaacatattccaatgcccacagctcacatcgtgtggcaagcctgtcgcctgtgccccggtcactgccaccttgaacacttaaggctgagcctcagaaacatctgtttagcaaaagaccacacacatgattgcatactctgtttaaacaaagtgctcatgactggttaaaaaagccagaaaatgggttagtgtttgccaggagaggaggctgggtgtgtggaaatggatttagacacggggcacccttcacatctaccacttgccaccccgcaggcccagagttttattaaaatccctgacattctcccttggtgcacagcgaaaactcctgctactttcactttctctaaggacccagttttttcccaacccccactcatcctcttcctgctgtggtcgccagagccagaagcagcctgtctccccgcccctgagcaggagcacaaaaccagaagtccctgccttgtacaggcagctctgaaagcctactgggagcatcaaggctgcaaacgctggattgagaagccgcaactgtggtaggtgcgggcatctggctcggggtggcgctgtgggacccgcagaggtcatgtttacaaggcataggctgagtccaggagggaaaacaagactgcttagttagggaaagagacagctgggaggtaatgtctgatgagctttttaaatggtgactcctttaggatctccagctggccaatggcagagatgcagatcttagggctggtttctaagctgtgttggctcctcctgagccaggcctggggactgcaggatattggcggcctctccactggtatctttttatgctttctatcagtccttaccacctaggcaatttgaaagcctctgtcttctccctgtggatcttgcaaagtaatccctctcttacttccggaccctttaaacatctggggtctctggtacaaatttgagcactgctcattctcatttataaaataattcactccatgctgacagtgatcatatctttgataactttatctgagaaatcaaatacccctttatttgagtatgcttactattagagtgccagacatcccccttaccttcagagagctccagaatatggagtagaaaggacagcaatcagtaagagaaaggtgacacttaagagaaaggtgacacttaagagcagacctggggtggggaagctgcactagggaaggagagcagaggccctgaggctgtcctgttccaatgaagttgggaaaggggagtagtttgaactcagtgacagggatggggtagggaatgttctaggtgaaataaactgcacttcaaggactcagaagcaactttaaagtgacataagggcaggaatgcaggagaagggggcaagctgggttagatgagaggactagccagagagtaagtctttgaaataaagcagccccacctttcctccagagccctggaagtcataaggggaggagccagaggatccgagtacactctcaggttgctgtgtccctctgtctgaggaatgggaagaagtgtggtttgagtgttctgatgctccctaggctatgtgatctgcatgttaactcagtaggaagcactctgaacatatgtgagggttcaaaggtgaaccctggaacagtgagagcacagccatgggatgcacagggactcagaactgggttaaagtgtcaggagcagtcacttagtctgggccaagctgtccatccatctggaagatgagtcataatcagtagaaaagcagccagggtgcctgcagctgatgcaaggtcctctgagcagctactgcaggggaatgctccccaatacacaacacagtaataatgctagcttgtctttgggtctcatttagcatctcagtgtcttttctgtagcatctcgaaagtggggaggatactgtctaattcttcatgggtaacacaggagtcttcacaggcagcgtccaggatacagagttgctgagtcaggcaagaggacccaatgttcctggttgttttcttcttcgtttcctcttcttccttcttcaaactctattgtcttcagaagagaggcgttggtctgtatgggagaaggctgaataccacctctctctgctgtgttttgtgtttgtgctcctgatgatcttgcgttctccctgtgcagatgagaagaagctagtttggtttgctgcaatgaccacacttagctaataaccttaaaattggaaggactgtaatctagaatgaatttttgaaatctaatttaaggtacctatgacctttgaaatgacaaccagaaacagagtcccagatttccctgcctgagaacaatgaaccttcagctctacgttgatttttatatctcctttccctccagactctgcatcatgaatactctgtctgaaggaaatggcacctttgccatccatcttttgaagatgctatgtcaaagcaacccttccaaaaatgtatgttattctcctgcgagcatctcctctgctctagctatggttctcttgggtgcaaagggacagacggcagtccagatatctcaggtatgttctctgtcatagtcacacccgagtcacatgcataagaactgctgagaagggacagaaggaatgtgcacacactgtttcatggggcaggggctaaagaggaaggacagagtctcctccaataagacttctttaagggcaatgagatttcctgagtcataacttggggtgactctcagtacattgtctagaatctaattaacagtggtgcatgaataattttatactatacttggtgttacaggcctttaattacagctactaaggagactgagatatgaagataccaagtctgagactaccctgggcaaattagttctagaaaaattaagacgttaaaaaaaaaagatggagggaaggtcttagggttataacttaggtacagtctcaagtactgcaatttgagaaaataaaaagatgtttaaattatttgaaagagagtttgctgggatctattttattttttagagcaacagtctttgtaattaattgtttgcaaggaaagataattccttctctttattttgtctaatatgccttgcttcagctaaaatactgaagggggcaaatgtgtggagctagtccatgtcctaactggacattgggttcattactttttgacaagcatcctgagagcattccaaattaggaatgggttcctgacctcacagcgttcctgaacaggcaggagccatttgtctgagaaaaaaagcaagcaagcaaacaaacaaacaaaaacagaaacaaattgtcagcattcagcttcacagtgggaagtcatcactgttgctctgtggtctgccaggactttctttctcatgaagatcctttcgggtgtaagtgctcggtctcgtcatctccaaacatagtccacgaggccatgcatgtgtagagaggcttctccctaattggtcctcatggtcttttctaggttcaggttcactaatcaactcatatacagatcttatgtatgtatataacatgtatgctcatgtatagcgtgtgtgtgtgtgtgtgtgtgtgtgtgtgtgtgtgtgtgtacatgcccttgtatatacctcttgttgggggtgggaggagtcagttctggttgtcttactctctctcatcaggctcctcccagtgctgggatgacccccatgcaccactgggcccatcttctatgcttattgagactccaactcagatgctcatgtactttactttctgagtcatctcatcaccttgacaattcctttggaaacaaattgttttattactgaactcagaatgtttcatcttttccttcccaggcacttggtttgaataaagaggaaggcatccatcagggtttccagttgcttctcaggaagctgaacaagccagacagaaagtactctcttagagtggccaacaggctctttgcagacaaaacttgtgaagtcctccaagtaagttgtattagtagaataatagcagttgaatgcaaggcagttgttgttgtagagcctatgagaaggaccctagagaatacgagaaaattacaggtctagttctactcagtgtgaggctacatatctataatacctttatttagatggccaagagggtcacatgttctaggccagcttcggctacaaaatgagactcacctaaatgttctcccaaaatatcacaattctgtaatgatattttgaaatatttttaaattgactacttgtaatttttatttactcatatctcagttgcaaagtttcagtacttaaagatgttgtgggttttttttttattaagatttatttattttatttatataaatacactgtggatgtattcagacacaccagaagagggcatcagaacccattatagatggttgtgagctaccatgtggttgctgggaattgaactcaggacctctggaagagcagtcagtgctcttaaccgctgagtgtgatatctacactcagtttagtttagtttttggtttttaatgccatagtattcagtaggttggaaagcttgctgaaatgtcattttgtaatgttaccttactgaatattttctctccaaagacctttaaggagtcctctcttcacttctatgactcagagatggagcagctctcctttgctgaagaagcagaggtgtccaggcaacacataaacacatgggtctccaaacaaactgaaggtcagaatttcaagttacttcacacaccacattcttgtcctttccatccaccctctctctttttccttctctgcctttctcttcgttcccttctccccaccacagactgatctcacagccatttcagagtcaaagccattctgactatctagaccacctggtataatttagagctcagggaattcatggaggttctactcagacacttagatttgaaacacagagttttttggtatatctacctagcacaggtgaggactgacgcaaatgatttccagtctaacagcttactttagaagacatgggtggtttgcagcattcgcaggtcatgtgactctgccatctctgagaaggagattcatggatagaccttccccttaatcctgctctagggctgcttctttcacagtctgtgttgtcttagctgtcaccacctttacctctgaaggagactactgtttctgcttccacagttcctgctttagattggttacttacattcatcagaattagacttgatttccattgaagaccctgccagctgctcagttacattaacctgaacgagctttccatttggggttgaactttatttgtttttattccagagatgacaggttccagcattagtctttgagatcattgtctgatatttgaaccattcagatccctctctatcactaggtacattcttacttctgctaacacagagtaaagaataagaatcttttcttagactcatcaattatttgcacatttcttgttagagttttgcctcttgtatgatcttgatattgtaaattagcaaggattgccttgatcagggaaagactacccattaatagagaagaagggaatttgtccctttatccacattttctcttaggaatgaacagcactcctaagtgtggaatagtcatgcctcctacactagtttctttctgattttgagtttctgacgtgttgtggtatgtcagacctgggattgaccatggagcgctagatgagactgggtatttaaccttagtatattagtaagggttcactggaggaacagaatggcggggatttaggggggattcaatagatttgcatacacaatacaggttgaatggatagtgaccacctgcatatggagagactgagtatccggtagctgctggatctaggagaatggagaccttagcaggccaagtctggcatggaaggcctagaggattcttcagagccacaagtctgtgttctcatgatggccaaatcaattgtcagctaaaaatggcaggcacagtgcactcaccagcaaggactgaaggcaggcagacaaaagcaggctgcttctgtctcagacctttatatctggcagcacagcattgtgctgcccaccggagggagtgacctgtctatattctgggaaatgtccccacagaccagagacatgtcctctgtttgaatccagaaccaattaagatggtaaccaacagtagccaccccaggggtaaccatggttccctcccacattttctgcttggtttgttcatcgtcctgaaatctctttttttgctttgcccaaacctgctttccttgagggacctacaagacttccaccatcctcacctgatgatgttcctccagtgtgcatgacctcaagtcaaacgtgggcattagtgctttcgtttgaatcatttttaataggcctatccaaattccacaaatatgaaaacgtgatgcttagatgtcttatcagttaatgtctcatacacatatgaaaggaacctgaatgaagtctctgctttctcaataggtaaaattccagagttgttgtcaggtggctccgtcgattcagaaaccaggctggttctcatcaatgccttatattttaaaggaaagtggcatcaaccatttaacaaagagtacacaatggacatgccctttaaaataaacaaggtaagaaacagcacccagaattcaaaggacaattcccttatatttcaaaggaaagtggccgccacagtttctcaaagaatgtacaatggaaatatcttttaaagtagatgacgtaaggaacagcatctaaacatacatgtcagtgtagtgaagagactgtatggaaagctttgatcgtgattgtgtagctctgggctcagtggtaaagactgcatatcctatgtggggttctggccttgactttcagaccagaaagaatgaaatcaggaatgcacaaaaaccatttgagacttatatttcttaatggaagctttaaactccatagaagaatgatagatataaggaatatatatctatatgttgctgtgtggttttaccgtctatcggtggctcatgctacttttgatcatttcttataaaaacctctgtttctacctcattctagaaactacattttcctttgagttcaagaaatcaaaaacgtaggtgttaatgtagtataaaacctgtatggagcatgaggcagggtagaaaggaatatataaacatttatataataaataatttcttaattaaaataaacattttaaattactggtccacttatcattattaaatagtatgtatttatagctttctaatatttaaaacagtgactgagatctcagtctgaattagaatataacttgaattaaggtagatattcgaatagtttactcaatacctataagatataatttaatgcattcaggaacattatgaatattaaacttcccattctatttttattaacagtactctaaaaatgtttgtaaaagaatgagtttttttttaacttgggaatttaatctttattctgaatggtctgtagcaaattcttatgtttattgtaccactataatcaaaatcttcacacattattgaaacactttaaaagtttgaacaatactgattgactactagaactgaacctgctggcataaaggacaagaagatagatacatgagggtctttccagatggccccctagcttttaatgggttgagaagaattttctgctctgtgatgggttatcagactgagtcacatagaaacatgtccatgtggttccccatcttggagtaatttaacttgggatttcccttccctctctctgatgggatatcaagatgcagctctgccctaagcctgtgaatgtctgtgttctggatctttctgctttgtgatggtttaagaggttttcatgaacatttgagcctgtgtgctcctgttgtcttgttgtcaggcttttgtttgtttgtttgtttaatattgaatattttccttatttacatttcaaatgttatctcctttcctggtttcccctccggagaccccctatcctatccccccttcccttgcttctgtgagggtgttcccccacccacctgcctactcctgcctccctgccctggcatttccctacactgaggcactgagccttcacaggaccaagggcctctcctcccattgatgccggagaagaccatcctttgctacatatgtggctggagccatgggtccctctatgtgtactctttggttggtggcttagtcccaaggagctctggagggtctggttgattgatattgttgttcttcctatggggttgcaaaccccttcagctccttcagtcctttctctaattctttcactggagaccctgtgctcagtccaagagttggctgtgagcattcgcctctgtatttgtcaggctctggcagaacctcttaggagacagctacagctgttttctgagagaatgcatctgtccataggcctgtcacagcactatgaaaatgttccagtctactttatgatattttttctgtttttcatatttgactatactgttttcctagatatgactgttgactacattaaatgataaaaatcctgcatcagatatgttagttatcttttcattagtgtaacaaactctctgagataatcaacttaaaagtaggacatgtttattttggctcatggttataaaagtttcatttggcgtccactggcccaattattccaggttatgatggtacatcacagagtgacaggaatgcgtggtgaaggctagtttaccacacagtacccgggaagcaaaagaaagaaacacaaaggggctagagtgccttcaggagaactccctttcaaatatttagcttttttcctattaatccttacctcctaaaagtttgaatagttcttggtcgtggctatctggggaaaaaggctttgaaacatgagcatttgtagaatatttaaacccagttctaaggctttagaatattaatatgtgaatgctatgtttttttttctagataacacaattgacttttagcagttagctcctgtcctgattattttttgttcttggtgtgtgtgtgtgtgtgtgtgtgtgtgtgtttgtatgtgtgtgtgtatgtacttggcacaagctaaaaaaaggaagaactctaaattgaggaattgattccataagattcatctataggcaaatctgctgggcatgttctcaataatggctgctggaggagggcccagaatgctgtgggtggtgccaaaaccacgcaggttgtcctgagttaagtaagaaagcatgctgagcaaacactggggagatgcccataagtaacattccttcatggcttcagcctggttccagctctgaattaccccagcaatggatgtgacctgagagttgtttatgaaataaattctttcctcctcaagttgctattctggcatggtgttttaccacatagaacacaccctaacagaaacggtcccatatctacttaccttcaatattttcaatgaaatcaggaagaagagggcagacattgtctaaacagtcactggaacatgaatattgtttacattgtgagtgctgaaaattttattgtgagccctgaaattaactgcagctcatttggagaccctgaccactctcagtaatacgttttatgccatgtatttctgtgttcaggatgagaaaaggccagtgcagatgatgtgtcgtgaagacacatataacctcgcctatgtgaaggaggtgcaggcgcaagtgctggtgatgccatatgaaggaatggagctgagcttggtggttctgctcccagatgagggtgtggacctcagcaaggtgagccagaagagtccacaaaattcatcactctaaatgtcttccagaagaatccagtgtccctgttgtcctttgtcattagaacagaagtaaatgattttattttgtctagtaccagttgagtgtgttgtactcacagacccagcattgagttgtgtgtgttggcactgggctttgggcttaaatctgtgcccaagactcccattgtagcctttgtgcccttgccatataacataaattcatcactagaatagataatggctacctgatagaatgaaatctgggcatctcaaatgataaacatagcacagtgcttggaaaatagtcctcctccataaatataatccatgtcatgatctttgttagcattttattcaatatctgagaaaatgcacaaagaactgttaaactttgacctaataaataggaagaggaggggtgcagctggagagagcagagtcaagcaggtcattataacagttcttggtcttccttgcggcaggatttcccatggtgtgtgagccatcctctgaatggtgggcagtagattgtgcattacctaaggggtctttgtaagcagacctgcaccatagttctcagcatccagcaccctaactgtggcatagtttctctaggatattactgtgtttcatcactgacaaaatgcctgacatgataacctagggcaggaaggatttatataggctcacagtttcagagcattcatttcagttacctgttccatcactggttgatatggtggcagctgcacattgaagggaatacagatctttcagttaataggaagtttagaaaggagaaaagacacataggatcaaagatgagcttctaagaaacatctccagcaactataaccttcattcagctaaaaaataatttagctgcctcccaaaatgttgccacatgtttcaaaacaatggtctgctatgaacatttgggtggaatttcacattcaagtcacaacagctcaccaaacacattaattctaccctggattgacttcccttctggtgtggctcataataagagttaatttctttcagagctttgatttttattttagcagcagtgattcactagctttgaaacaaaatctagttcaagaaaacaaaagcaaatacaacctaaactattatcacctggacatggacacctgtcagaataaaagatgtcttagatgtaatttctgggagggcaaccaggtctttctatccctgggctgcttgtcaaccatgaccatcagggaagacacatacaagctagcaggacagtggactgtcacactgcctttttatgaataagcagtgtgccttgaatggtgttagaaggtactgtgaataggagtgctctgtggttctgaggaagacctcaaaggtgaatctgaaattctcaaattcttctttgttaggtggaaaacaatctcacttttgagaagttaacagcctggatggaagcagattttatgaagagcactgatgttgaggttttccttccaaaatttaaactccaagaggattatgacatggagtctctgtttcagcgcttgggagtggtggatgtcttccaagaggacaaggctgacttatcaggaatgtctccagagagaaacctgtgtgtgtccaagtttgttcaccagagtgtagtggagatcaatgaggaaggcacagaggctgcagcagcctctgccatcatagaattttgctgtgcctcttctgtcccaacattctgtgctgaccaccccttccttttcttcatcaggcacaacaaagcaaacagcatcctgttctgtggcaggttctcatctccataaagacacatatactacacagggagagttctctcttcagtatccctaccactcctacagctctgtcaagatgggcaagtagggggaagtcatgttctaagatgaagacactttcttcttgtcagcctgatcttataatgcctgcattcaactctccctgtcttgaatgcatctatgccctttaccaggttatgtctaatgatgccaaataccttctgctatgctattgattgatagcctagccagtaatttatagccagttagaactgacttgactgtgcaagaatgctataatggagctagagagaaggcacaaacactaggaaaggttgctgtttttgcagaggacacagggacatttcccaccactcacatggctgcttacaacctctggaaattccagtttctgtccatgacttgattcctttctttggcttctactggctccagcatcctgcacatacatgtatcgtcattcagttacacacaaacaagtaaaattttaaaaataaataaaaatttaaagagagagtctaaaattttagtaatggttagataatagctgctattgtgcctttttcaggttttaatgtcattattcttgtgtataaagtcaataatttataggaaaacatcagtgccatgctgtgatcttatcttcatcccctttcaaacacattgtttaaccatcaagcagtgactctccattcttcctccctttggacatataaccaccaatcttctctctgtctctgtagctttatctattttggacattttatatagactgcctctttgctatcttgataatgtcctttgatgcatacagattgctttttttccagtgatgttctccttatttaatactgttctgatcatgaggtatgcatgctgcagctaagaatctcactactaaagtcaagtatagaaataactgttcttatgttttcctccaaggccttcatggattataccttatatatctaggtcactgttccagtttgaattaaattccatatgtggtgtggtgttggagcctgatgtgattcctttatttgtgaacatccagttgtcgctgtacttgtaattgctttgaatcatgaggtaatgaccaatgaataggaaaaataatgttacaactagtggggtaggatagaagttcctcagtacatatattgaacattaacacaagtgtgaaataaaaatatcagaaatgagcagtaaacaaagaaggagagattaaacgttctgtaatcagggtcctcagaaatgactcaggggctgaagatgcttgctaccctgcctgacaacttgagttctatttctggaacagacatggtggaagaagaaaactgactctagcaagttgtcctctgacctcaccatgagcatgatgcacaaataaatatccctgttaatcagatataattatgcagtaagttaataatcagaaaaaatacacatataaaaaccaattccttatcaacaagttaatcttgctaggaagagccaggaaaaagagtaataggggaccatgtgatcctaaaagggataaagctaagccaggatatactcttatacatcaacatgtgtcctggtttccagaaggttcaatttgaggacatatcccaatgatgtcattactgaagtccacaaaaagacagtgactatgatctctgtagccatatttatagcagcaaataatggatccaggtgtcaatctcatacagtaaacaaagaagtcatgttgtacacttaatactgaatggaagttagaaagttgtaccagatagtttttatattaatgtgatacaaggtagagtcactggagaggaaagagcctcaattaagaaatgtctccatatgctctggagcattaatgtgagaaggctaggcccattgtatgtgagacaatccctatgcaggtggtcctctgattataaggaagcaggctgaacaaaccaggaggagcaagccagtaagcagcattcctctgtggcctctgcatcagctcctgcctccagtttcttgccccgttggcattcttctgacttccttcattgatgaacagtgatgtaaacgtataagccaaataaaccttttctacccc

(19) United States
(12) Patent Application Publication
(10) Pub. No.: US 2024/0335478 A1
(43) Pub. Date: Oct. 10, 2024

(54) MODIFIED ORGANS AND TISSUES

(71) Applicant: UNIVERSITY HEALTH NETWORK, TORONTO (CA)

(72) Inventors: SHAFIQUE KESHAVJEE, TORONTO (CA); MARCELO CYPEL, TORONTO (CA); MINGYAO LIU, NORTH YORK (CA); STEPHEN JUVET, TORONTO (CA); KUMI MESAKI, TORONTO (CA)

(21) Appl. No.: 18/288,241

(22) PCT Filed: Apr. 22, 2022
(86) PCT No.: PCT/CA2022/050625

§ 371 Date: Oct. 25, 2023

Related U.S. Application Data
(60) Provisional application No. 63/305,428, filed on Feb. 1, 2022.
(60) Provisional application No. 63/282,120, filed on Nov. 22, 2021.
(60) Provisional application No. 63/180,365, filed on Apr. 27, 2021.

(51) Int. Cl. A61K 35/42; A61K 31/52; A61K 31/573; A61K 38/13; A61P 37/06; C07K 14/47; C07K 14/54; C12N 9/22; C12N 15/11; C12N 15/90

(52) U.S. Cl. CPC A61K 35/42; A61K 31/52; A61K 31/573; A61K 38/13; A61P 37/06; C07K 14/4703; C07K 14/5428; C12N 9/22; C12N 15/11; C12N 15/907; C12N 2310/20

(57) ABSTRACT

The present disclosure relates generally to epigenetically and genetically modified organs and tissues and methods of producing same. In particular, the present disclosure is directed to organs and tissues that have been epigenetically and/or genetically modified at one or multiple loci to control inflammation-regulating or immune-regulating gene expression and thereby improve the condition of the organs and tissues.

### CROSS REFERENCE TO PRIOR APPLICATIONS

This application claims priority under the Paris Convention to U.S. Provisional Patent Application No. 63/180,365 filed Apr. 27, 2021; U.S. Provisional Patent Application No. 63/282,120 filed Nov. 22, 2021; and U.S. Provisional Patent Application No. 63/305,428 filed Feb. 1, 2022, the contents of which are incorporated herein by reference as if set forth in their entirety.

### FIELD OF THE DISCLOSURE

This disclosure relates generally to epigenetically and genetically modified organs and tissues, and methods of producing same. In particular, the present disclosure is directed to organs and tissues that have been epigenetically and/or genetically modified at one or multiple loci to control inflammation-regulating or immune-regulating gene expression. The organs and tissues may be used for transplantation.

### BACKGROUND OF THE DISCLOSURE

Organ and tissue transplantation is lifesaving for patients. For example, lung transplants can be lifesaving for individuals with end-stage lung diseases; however, the number of patients waiting for lung transplants greatly exceeds the number of available donors. On average, less than 20% of lungs from multi-organ donors are used for transplantation and the rest are typically considered unsuitable.

Currently, the use of static hypothermia is widely accepted for preserving organs after removal. However, there are drawbacks related to keeping an organ in a hypothermic state for a period of time. For example, the inhibition of cellular metabolism as a result of hypothermia can make it difficult to repair an organ or assess its suitability or condition during the preservation period.

In addition, many organs are considered injured or too “high risk” to be transplanted into a human. For example, more than 80% of donor lungs are considered too high risk for reasons including lung injury that typically occurs after brain death and/or complications associated with treatment in intensive care units.

Although non-optimal donor organs, such as lungs with suboptimal gas-exchange function or infiltrates visible on chest radiographs, have been used with success, increased primary graft dysfunction (an acute lung injury typically occurring within 72 hours after transplantation) has been reported in some studies. These injuries can affect early outcomes and can be associated with an increased risk of chronic graft dysfunction.

The techniques currently used to assess an organ or tissue for transplant suitability cannot adequately identify every suitable organ or tissue because of hypothermic preservation conditions and time constraints. As a result, clinicians tend to be highly conservative when selecting donor organs and tissues, and because of the relatively small number of organs and tissues that are deemed to be acceptable, mortality in patients awaiting transplantation is high.

Having an increased number of suitable organs, such as lungs, available to transplant is a promising means of augmenting the number of organ transplants and thereby saving more lives.

Lung modification holds great promise to address shortage of donor organs in lung transplantation (LTx). Immunologically enhanced lungs by epigenetic or genetic modification of endogenous genes could expand donor pools by repairing injured donor lungs currently being declined for transplant.

Further, local immunosuppression of modified lungs could reduce or eliminate life-long and systemic immunosuppression in recipients. The life-long and systemic immunosuppression to prevent rejection is a remaining challenge in lung transplantation. The daily intake of multiple immunosuppressants not only lowers patient's quality of life (QOL) but contributes to long-term mortality and morbidity. Unfortunately, there have been no alternatives for the current systemic immunosuppression.

### SUMMARY OF THE DISCLOSURE

The inventors have invented genetically and/or epigenetically modified organs and tissues; expression cassettes and delivery vectors for modulating expression of inflammation-regulating and/or immune-regulating genes; kits comprising said cassettes and/or said vectors; and methods for preparing, repairing, conditioning, maintaining, and/or optimizing an organ or tissue. The organ or tissue may be modified in vivo or ex vivo. The organ or tissue may be used for transplantation.

In a first aspect of the present disclosure, a modified organ or tissue is provided. The modified organ or tissue comprises at least one modified endogenous inflammation-regulating and/or immune-regulating gene.

In an embodiment of the first aspect, the modified organ or tissue expresses a phenotype that improves transplantation outcome compared to a corresponding organ or tissue that does not comprise the at least one modified endogenous inflammation-regulating and/or immune-regulating gene.

In an embodiment of the first aspect, the at least one modified endogenous inflammation-regulating and/or immune-regulating gene is selected from the group consisting of: interleukin-10 (IL-10), interleukin-1 receptor antagonist (IL1RN), Interleukin-4 (IL-4), Interleukin-6 (IL-6), Interleukin-8 (IL-8), tumor necrosis factor binding protein (TNFBP), cytotoxic T-lymphocyte associated protein 4 (CTLA4), NFKB inhibitor alpha (Ikba), Programmed cell death-1 (PD-1), Programmed cell death-ligand 1 (PD-L1), Interleukin-10 receptor, CD47, CD200, Fas Ligand (FasL), MHC molecule (MHC-Ib) H2-M3, Serine protease inhibitor 6 (Spi6), C-C Motif Chemokine Ligand 21 (Ccl21) and Milk Fat Globule-EGF Factor 8 Protein (Mfge8).

In an embodiment of the first aspect, the at least one modified endogenous inflammation-regulating and/or immune-regulating gene comprises at least one modification, and wherein the at least one modification is a transient modification, a persistent modification, a permanent modification, or a combination thereof.

In an embodiment of the first aspect, the at least one modification is a genetic modification, an epigenetic modification, or a combination thereof.

In an embodiment of the first aspect, the genetic modification comprises insertion, deletion, or substitution of one or more nucleotides in a regulatory region of the at least one modified endogenous inflammation-regulating and/or immune-regulating gene.

In an embodiment of the first aspect, the epigenetic modification comprises a modification of transcription, a histone modification, a modification of DNA methylation, or a combination thereof.

In an embodiment of the first aspect, the at least one modification comprises transcriptional activation or transcriptional derepression.

In an embodiment of the first aspect, the at least one modification comprises a CRISPR-associated (Cas) protein bound to a regulatory region of the at least one modified endogenous inflammation-regulating and/or immune-regulating gene.

In an embodiment of the first aspect, the Cas protein has nuclease, nickase, transposase, base editing, or prime editing activity.

In an embodiment of the first aspect, the Cas protein is nuclease-deficient or nuclease-dead.

In an embodiment of the first aspect, the Cas protein is Cas9.

In an embodiment of the first aspect, the at least one modified endogenous inflammation-regulating and/or immune-regulating gene is IL-10 and/or IL1RN, and the organ is a lung.

In an embodiment of the first aspect, the organ comprises a polynucleotide encoding at least one guide RNA (gRNA), and wherein the polynucleotide comprises one or more of the nucleotide sequences set forth in SEQ ID NOs: 1-10, 12-42, 44-59 and 61-79, or a complement thereof.

In an embodiment of the first aspect, the polynucleotide comprises two or more of the nucleotide sequences set forth in SEQ ID NOs: 1-10, 12-42, 44-59 and 61-79, or a complement thereof.

In an embodiment of the first aspect, the modified organ or tissue is a human organ or tissue.

In a second aspect of the present disclosure, a method of preparing an organ for transplantation into an animal is provided. The method comprises the steps of:
(i) subjecting the organ to a perfusate; and
(ii) modifying at least one endogenous inflammation-regulating and/or immune-regulating gene in the organ.

In an embodiment of the second aspect, the step of modifying the at least one endogenous inflammation-regulating and/or immune-regulating gene in the organ comprises CRISPR/Cas-mediated modification.

In an embodiment of the second aspect, the CRISPR/Cas-mediated modification comprises genetic modification, epigenetic modification, or a combination thereof.

In an embodiment of the second aspect, the CRISPR/Cas-mediated modification comprises insertion, deletion, or substitution of one or more nucleotides in a regulatory region of the at least one modified endogenous inflammation-regulating and/or immune-regulating gene.

In an embodiment of the second aspect, the CRISPR/Cas-mediated modification comprises a modification of transcription, a histone modification, a modification of DNA methylation, or a combination thereof.

In an embodiment of the second aspect, the modification of transcription comprises transcriptional activation or transcriptional derepression.

In an embodiment of the second aspect further comprising the step of immunosuppression before, during and/or after the transplantation.

In an embodiment of the second aspect, the step of immunosuppression comprises administering an agent to a recipient of the organ, wherein the agent is a steroid, corticosteroid, calcineurine inhibitor, cell cycle inhibitor, IL-2R antagonist, mTOR inhibitor, or a combination thereof.

In an embodiment of the second aspect, the agent is methylprednisolone, cyclosporin, cyclosporin A, azathioprine, anti-thymocyte globulin, rapamycin, tacrolimus, mycophenolate mofetil, mycophenolic acid, sirolimus, everolimus, prednisone, or a combination thereof.

In an embodiment of the second aspect, the agent is a combination of methylprednisolone, cyclosporin and azathioprine.

In an embodiment of the second aspect, the agent is a combination of methylprednisolone and cyclosporin.

In an embodiment of the second aspect, the perfusate comprises a polynucleotide encoding at least one gRNA, and wherein the polynucleotide comprises one or more of the nucleotide sequences set forth in SEQ ID NOs: 1-10, 12-42, 44-59 and 61-79, or a complement thereof.

In an embodiment of the second aspect, the polynucleotide comprises two or more of the nucleotide sequences set forth in SEQ ID NOs: 1-10, 12-42, 44-59 and 61-79, or a complement thereof.

In an embodiment of the second aspect, the organ is a lung and the step of subjecting the organ to a perfusate comprises ex vivo lung perfusion (EVLP) or in vivo lung perfusion (IVLP).

In a third aspect of the present disclosure, an expression cassette is provided. The expression cassette comprises:
(i) a polynucleotide encoding at least one guide RNA (gRNA) that binds to a regulatory region of at least one inflammation-regulating and/or immune-regulating gene selected from the group consisting of interleukin-10 (IL-10), interleukin-1 receptor antagonist (IL1RN), Interleukin-4 (IL-4), Interleukin-6 (IL-6), Interleukin-8 (IL-8), tumor necrosis factor binding protein (TNFBP), cytotoxic T-lymphocyte associated protein 4 (CTLA4), NFKB inhibitor alpha (Ikba), Programmed cell death-1 (PD-1), Programmed cell death-ligand 1 (PD-L1), Interleukin-10 receptor, CD47, CD200, Fas Ligand (FasL), MHC molecule (MHC-Ib) H2-M3, Serine protease inhibitor 6 (Spi6), C-C Motif Chemokine Ligand 21 (Ccl21) and Milk Fat Globule-EGF Factor 8 Protein (Mfge8); and
(ii) a polynucleotide encoding a CRISPR-associated (Cas) protein.

In an embodiment of the third aspect, the at least one inflammation-regulating and/or immune-regulating gene is IL-10 and/or IL1RN.

In an embodiment of the third aspect, the Cas protein has nuclease, nickase, transposase, base editing, or prime editing activity.

In an embodiment of the third aspect, the Cas protein is nuclease-deficient or nuclease-dead.

In an embodiment of the third aspect, the Cas protein is Cas9.

In an embodiment of the third aspect, the Cas9 is Staphylococcus aureus Cas9 (saCas9), Streptococcus pyogenes Cas9 (spCas9), or a variant thereof.

In an embodiment of the third aspect further comprising a polynucleotide encoding a transcriptional activator.

In an embodiment of the third aspect, the transcriptional activator comprises VP64 from herpes simplex virus, p65 from human NF-κB, Rta from Epstein-Barr virus, or a combination thereof.

In an embodiment of the third aspect, the transcriptional activator comprises p300 core domain of human histone acetyltransferase.

In an embodiment of the third aspect, the polynucleotide encoding at least one gRNA comprises one or more of the nucleotide sequences set forth in SEQ ID NOs: 1-10, 12-42, 44-59 and 61-79, or a complement thereof.

In an embodiment of the third aspect, the polynucleotide encoding at least one gRNA encodes at least two gRNAs and comprises two or more of the nucleotide sequences set forth in SEQ ID NOs: 1-10, 12-42, 44-59 and 61-79, or a complement thereof.

In an embodiment of the third aspect, an expression cassette is provided. The expression cassette comprises the nucleotide sequence set forth in SEQ ID NOs: 85 or 86, or a complement thereof.

In a fourth aspect of the present disclosure, a vector is provided. The vector comprises the expression cassette of the third aspect.

In an embodiment of the fourth aspect, the vector is an adenoviral vector or an adeno-associated virus (AAV) vector.

In a fifth aspect of the disclosure, a cell is provided. The cell comprises the expression cassette of the third aspect or the vector of the fourth aspect.

In a sixth aspect of the disclosure, an organ is provided. The organ comprises the expression cassette of the third aspect, the vector of the fourth aspect, or the cell of the fifth aspect.

In an embodiment of the sixth aspect, the organ is selected from the group consisting of lung, heart, kidney, liver, pancreas, stomach and intestine.

In an embodiment of the sixth aspect, the organ is a lung.

In an embodiment of the sixth aspect, the organ is a human lung.

In a seventh aspect of the present disclosure, a modified organ or tissue is provided. The modified organ or tissue comprises at least one expression cassette, wherein the at least one expression cassette comprises:
(i) a polynucleotide encoding at least one guide RNA (gRNA) that binds to a regulatory region of an endogenous inflammation-regulating and/or immune-regulating gene, and
(ii) a polynucleotide encoding an exogenous inflammation-regulating and/or immune-regulating gene.

In an embodiment of the seventh aspect, the at least one expression cassette is at least two expression cassettes.

In an embodiment of the seventh aspect, the inflammation-regulating and/or immune-regulating gene is selected from the group consisting of: interleukin-10 (IL-10), interleukin-1 receptor antagonist (IL1RN), Interleukin-4 (IL-4), Interleukin-6 (IL-6), Interleukin-8 (IL-8), tumor necrosis factor binding protein (TNFBP), cytotoxic T-lymphocyte associated protein 4 (CTLA4), NFKB inhibitor alpha (Ikba), Programmed cell death-1 (PD-1), Programmed cell death-ligand 1 (PD-L1), Interleukin-10 receptor, CD47, CD200, Fas Ligand (FasL), MHC molecule (MHC-Ib) H2-M3, Serine protease inhibitor 6 (Spi6), C-C Motif Chemokine Ligand 21 (Ccl21) and Milk Fat Globule-EGF Factor 8 Protein (Mfge8).

In an embodiment of the seventh aspect, the inflammation-regulating and/or immune-regulating gene is IL-10 and/or IL1RN.

In an embodiment of the seventh aspect, the polynucleotide encoding at least one gRNA comprises one or more of the nucleotide sequences set forth in SEQ ID NOs: 1-10, 12-42, 44-59 and 61-79, or a complement thereof.

In an embodiment of the seventh aspect, the polynucleotide encoding at least one gRNA encodes at least two gRNAs and comprises two or more of the nucleotide sequences set forth in SEQ ID NOs: 1-10, 12-42, 44-59 and 61-79, or a complement thereof.

In an embodiment of the seventh aspect, the modified organ or tissue is a human organ or tissue.

In an eighth aspect of the present disclosure, a method of transcriptome manipulation in an organ or tissue is provided. The method comprises introducing at least one expression cassette into the organ or tissue, wherein the at least one expression cassette (i) alters the expression of an endogenous inflammation-regulating and/or immune-regulating gene and (ii) delivers an exogenous inflammation-regulating and/or immune-regulating gene.

In an embodiment of the eighth aspect, the at least one expression cassette is at least two expression cassettes.

In an embodiment of the eighth aspect, the inflammation-regulating and/or immune-regulating gene is selected from the group consisting of: interleukin-10 (IL-10), interleukin-1 receptor antagonist (IL1RN), Interleukin-4 (IL-4), Interleukin-6 (IL-6), Interleukin-8 (IL-8), tumor necrosis factor binding protein (TNFBP), cytotoxic T-lymphocyte associated protein 4 (CTLA4), NFKB inhibitor alpha (Ikba), Programmed cell death-1 (PD-1), Programmed cell death-ligand 1 (PD-L1), Interleukin-10 receptor, CD47, CD200, Fas Ligand (FasL), MHC molecule (MHC-Ib) H2-M3, Serine protease inhibitor 6 (Spi6), C-C Motif Chemokine Ligand 21 (Ccl21) and Milk Fat Globule-EGF Factor 8 Protein (Mfge8).

In an embodiment of the eighth aspect, the inflammation-regulating and/or immune-regulating gene is IL-10 and/or IL1RN.

In an embodiment of the eighth aspect, the polynucleotide encoding at least one gRNA comprises one or more of the nucleotide sequences set forth in SEQ ID NOs: 1-10, 12-42, 44-59 and 61-79, or a complement thereof.

In an embodiment of the eighth aspect, the polynucleotide encoding at least one gRNA encodes at least two gRNAs and comprises two or more of the nucleotide sequences set forth in SEQ ID NOs: 1-10, 12-42, 44-59 and 61-79, or a complement thereof.

In a ninth aspect of the present disclosure, a kit is provided. The kit comprises the expression cassette of the third aspect and/or the vector of the fourth aspect and instructions for using same.

In an embodiment of the ninth aspect, the kit further comprises a perfusate.

In an embodiment of the ninth aspect, the vector and the perfusate are formulated separately.

In an embodiment of the ninth aspect, the vector and the perfusate are formulated together.

In a tenth aspect of the present disclosure, a modified organ or tissue having altered expression of at least one inflammation-regulating and/or immune-regulating gene is provided. The modified organ or tissue comprises a genetic or epigenetic modification in the at least one inflammation-regulating and/or immune-regulating gene, wherein the inflammation-regulating and/or immune-regulating gene of a corresponding unmodified organ or tissue is encoded by a polynucleotide comprising a sequence having at least 95% identity to any one of the sequences set forth in SEQ ID NOs: 111-130.

In an embodiment of the tenth aspect, the organ or tissue is a human lung.

In an eleventh aspect of the present disclosure, a modified organ or tissue having altered expression of at least one inflammation-regulating and/or immune-regulating gene encoded by a nucleotide sequence having at least 95% identity to any one of the sequences set forth in SEQ ID NOs: 111-130 is provided. The modified organ or tissue comprises a genetic or epigenetic modification in a regulatory region of the at least one inflammation-regulating and/or immune-regulating gene.

In an embodiment of the eleventh aspect, the organ or tissue is a human lung.

In a twelfth aspect of the present disclosure, a modified organ or tissue is provided. The modified organ or tissue is produced by a method comprising the steps of:
(i) subjecting an unmodified organ or unmodified tissue to a perfusate comprising a polynucleotide encoding a CRISPR-associated (Cas) protein and at least one a guide RNA (gRNA) comprising the nucleotide sequence set forth in any one of SEQ ID NOs: 1-10, 12-42, 44-59 and 61-79, or a complement thereof; and
(ii) genetically or epigenetically modifying at least one inflammation-regulating and/or immune-regulating gene in the organ or tissue to alter expression of the at least one inflammation-regulating and/or immune-regulating gene.

In an embodiment of the twelfth aspect, the organ or tissue is a human lung.

In a thirteenth aspect of the present disclosure, a composition for modification of an organ or tissue is provided. The composition comprises a polynucleotide encoding at least one guide RNA (gRNA) comprising the nucleotide sequences set forth in any one of SEQ ID NOs: 1-10, 12-42, 44-59 and 61-79, or a complement thereof; and at least one CRISPR-associated (Cas) protein.

In an embodiment of the thirteenth aspect, the composition further comprises a polynucleotide encoding an exogenous inflammation-regulating and/or immune-regulating gene.

In an embodiment of the thirteenth aspect, both the polynucleotide encoding the at least one gRNA and the polynucleotide encoding the at least one Cas protein are expressed from a single vector.

Other and further aspects, features and embodiments of the present disclosure will become apparent during the course of the following discussion and by reference to the accompanying drawings.

### BRIEF DESCRIPTION OF THE DRAWINGS

In order that the subject matter of the present disclosure may be readily understood, embodiments are illustrated by way of the accompanying drawings.

FIG. 1: Transcriptional activation of the endogenous IL-10 using dSaCas9-VPR in rat lung cell lines. (A) Genomic locus of designed gRNAs. (B) Each gRNA was expressed along with dSaCas9 by co-transfection of two plasmids in either L2 cells (n=2, independent transfections) or NR8383 cells (n=3, independent transfections). IL-10 expression was measured after 48 hours by qPCR (mean±SD). Values detected after 40 cycles were shown. Mock expresses non-targeting gRNA. Negative control (Neg) received transfection reagent without any plasmids. Student's t-test was applied to compare each group with negative control (L2 cells) or Mock group (NR8383 cells). (C, D) L2 cells were transfected with single plasmid or co-transfected with two plasmids using the same molar amount and assessed for IL-10 gene expression after 48 hours (C,n=2; D, n=3, independent transfections). gRNA8 was designed to bind the same region as other gRNAs, but contains 20-bp spacer sequence adjacent to NGG protospacer adjacent motif (PAM) and scaffold for Streptococcus pyogenes Cas9 (SpCas9). Each group was compared with P300 alone group (C) or rat IL-10 cDNA group (D) using Student's t-test. (E) Experimental groups used for in vitro assessment. (F,G) L2 cells were transduced with adenoviral vector at multiplicity of infection (MOI) of 250 (n=2, independent transductions). (F) IL-10 protein concentration of the cell culture supernatants was measured by ELISA at different time points (n=2, two different transductions). The undetected values were set as 0, which was a back-calculated concentration of the sample with the lowest OD. (G) Calculated average rates of IL-10 protein increase. Values above detection limits were used for calculation. (H) IL-10 transcript levels 48 hours after adenoviral transduction measured by qPCR (n=3, independent transductions). Student's t-test was used to compare with no gRNA group. ND; not detectable, NS; not significant, *; P≤0.05, **; P≤0.01, ***; P≤0.001, ****; P≤0.0001.

FIG. 2: Assessment of inflammation evoked by trans-airway delivery of higher-dose Ad-dSaCas9-VPR in the lung. (A) Ad-dSaCas9-VPR was administered the left lung via airway at a dose of 2.5×10⁸ infectious unit (IU)/rat or diluent buffer alone (Ad-dCas9-VPR at 24 h and 48 h, n=5; others, n=6). PaO₂ was analyzed using arterial blood drawn from abdominal aorta after 5 min ventilation at FiO₂ of 1.0 (left panel). Wet-to-Dry ratio of the lung was calculated by weighing the lung tissue before and after incubation in the oven at 85° C. Mean±SD were presented (right panel). Symbols indicate *; P≤0.05, and **; P≤0.01 (Kruskal-Wallis followed by Dunn's correction). (B) TUNEL staining of lung tissues fixed at 1 day to 3 days after the adenoviral vector delivery. Scale bars, 100 μm. (C) Immunofluorescence of lung tissue fixed 3 days after Ad-dSaCas9-VPR delivery. MPO; myeloperoxidase. Scale bars, 50 μm.

FIG. 3: Impact of triple immunosuppression on ameliorating inflammation after dSaCas9-VPR delivery to the lung. (A) Experimental design and immunosuppression protocol. Immunosuppressants were intraperitoneally injected. (B, C) Systemic PaO₂ were measured using arterial blood taken from abdominal aorta. Wet-to-dry ratio and dSaCas9 expression were measured using lung tissues collected at day 3 (Ad-dCas9-VPR without IS, n=6; others, n=5, mean±SD). Kruskal-Wallis followed by Dunn's correction was used for comparison. *; P ≤0.05, ***; P≤0.001. (D) Hematoxylin and eosin staining of the lung tissues treated with different dose of Ad-dCas9-VPR with or without immunosuppression. Scale bars, 100 μm. IS: immunosuppression.

FIG. 4: Dose-effect relationship of adenoviral vector-mediated expression of dSaCas9-VPR and two gRNAs in the lung. (A) IL-10 expression in the lungs treated with two gRNAs virus after 3 days. Lower-, medium- and higher-dose represent 5×10⁷, 1×10⁸ and 2.5×10⁸ IU/rat of adenoviral vector (lower- and higher-, n=6; medium-, n=10; diluent, n=5). Mean±SD are shown. Kruskal-Wallis followed by Dunn's correction was used for comparison. Symbols indicate *; P<0.05, and ****; P<0.0001. (B, C) Scatter plots present the relationships between gRNA4, dSaCas9-VPR and IL-10 expression at day 3.

FIG. 5: Analysis of protein levels and histology of the lungs treated with adenoviral-vector mediated IL-10 activation. (A) Concentrations of each target protein in the lung tissue were measured by enzyme linked immunosorbent assay (ELISA) using lung tissues collected 3 days after the treatment (1×10⁸ IU/rat single gRNA, n=7; 1×10⁸ IU/rat two gRNAs, n=10; 2.5×10⁸ |U/rat single gRNA and two gRNAs, n=6; and Diluent, n=5). IL-10/TNF-α and IL-10/IL-6 ratio were calculated using the concentrations of each protein obtained by ELISA. (B) Wet-to-dry weight ratios of the lung tissues after 3 days of the vector administration. Mean±SD are shown. Kruskal-Wallis followed by Dunn's correction was used for comparison. Symbols indicate: *P≤0.05, **P≤0.01, ***; P≤0.001. (C) Hematoxylin and eosin staining of the lung tissues fixed at day3. Scale bars, 100 μm.

FIG. 6: Biological distribution of cells expressing dSaCas9-VPR and IL-10 in the lung. (A) Lung tissues fixed 7 days after the vector administration were probed for SaCas9 (green) and rat IL-10 (red) mRNA using in situ hybridization. Scale bars, 50 μm. (B) Immunofluorescence staining for podoplanin (green) and SaCas9 (red) in lung tissues fixed at day 3. Blue staining indicates DAPI staining. Scale bars, 100 μm.

FIG. 7: Time course of parameters related to the targeted IL-10 activation and general conditions of rats under transient immunosuppression. (A, B) Gene expressions and transgene DNA measured by qPCR. (C) Wet-to-dry ratios of the lungs collected at each timepoint. (D) Body weight change was calculated as % of original weight. Two gRNAs on day 3, n=10; others, n=5 at each time point. Mean±SD are shown. Two-way ANOVA, Bonferroni's correction was used. *; P≤0.05, ***; P≤0.001, ****; P<0.0001.

FIG. 8: Impact of continuous immunosuppression on targeted IL-10 activation utilizing Ad-dSaCas9-VPR in the lung. (A, B) Rats were treated with two gRNAs adenoviruses under transient or continuous triple immunosuppression. Treated lungs were analyzed at day 14 for gene expressions and transgene DNA in the lung tissue by qPCR (n=5). Mean±SD are shown. (** P≤0.01; Mann-Whitney test). IS; immunosuppression. (C) Hematoxylin and eosin staining of the lungs 14 days after the adenoviral-vector administration. Scale bars, 100 μm.

FIG. 9: Impact of the number of gRNA cassettes on potency and rapidity of IL-10 protein increase. L2 cells were transfected with single plasmid vectors. Cell culture supernatant was collected at each time point and measured for rat IL-10 protein concentrations using ELISA (n=3, independent transfections). Two-way ANOVA followed by Bonferroni's correction was performed. Statistical significance compared to single gRNA group was shown. Symbols indicate ****; P≤0.0001.

FIG. 10: ChIP-qPCR and RNA-sequencing analysis of primary rat lung fibroblasts treated with adenoviral vectors. (A) Primary cultures of rat lung fibroblasts were transduced with adenoviral vectors at MOI of 10 and subjected to chromatin immunoprecipitation (ChIP) against SaCas9 at 48 hours. Bound DNA fragments were analyzed by qPCR designed for the on- and off-target genomic loci (n=3, biological replicates using primary cells established from different donors). One-way ANOVA followed by Bonferroni's correction was used at each target locus. Symbols present the significance compared to the diluent group unless indicated. NS; not significant, *; P≤0.05, **; P≤0.01, ****; P≤0.0001. (B) RNA-seq differential expression analysis using DE-seq2 (See methods). Differentially expressed genes (P-value <0.01 and log 2-fold-change ≥1) between each of the indicated conditions are plotted in red. IL10 is specifically indicated with the arrow for each group comparison. (C) Venn diagram between differentially expressed genes identified by RNA sequencing and genes associated to potential high-risk off-target loci.

FIG. 11: Feasibility of multiplexed cytokine modulation via CRISPR-Cas-mediated transcriptional activation using all-in-one adenoviral vectors. (A) L2 cells were transfected with single plasmid vectors and assessed for rat IL-10 and IL1RN expression after 48 hours (n=3, independent transductions). Values detected after 40 cycles in qPCR were shown. One-way ANOVA followed by Bonferroni's correction was used for each target. Statistical significance compared to negative control was shown. Symbols indicate ***; P≤0.001, ****; P≤0.0001. (B) Single adenoviral vectors were delivered to L2 cells at a dose of MOI 250 to express dSaCas9-VPR and two IL-10-activating gRNAs (IL-10-activating-Ad) or with additional two gRNAs targeting IL1RN (IL-10+IL1RN-activating Ad). Rat IL-10 and IL1RN protein concentrations in the cell culture supernatant at 48 hours of viral transduction were measured using ELISA (n=2, independent transductions).

FIG. 12: (A) An illustration showing suppression of IL-10 transcription through repressor binding to the regulatory region in the absence of stimuli. (B) In targeted transcriptional activation, Cas9 activator is recruited to the genetic locus upstream of the IL-10 gene by designed gRNAs and enhances IL-10 gene expression. (C) In the derepression genome editing approach using wild-type Cas nuclease, mutations are installed into the regulatory region of the IL-10 gene that derepresses IL-10 gene and increases IL-10 transcription.

FIG. 13: (A) gRNA 75 (comprising the nucleotide sequence set forth in SEQ ID NO: 30) was designed to bind the genomic region upstream of the IL-10 gene in the rat genome. (B) Delivery of spCas9 and gRNA 75 to L2 cells in vitro. (C) Targeted Sanger sequence of genomic DNA demonstrated mutagenesis around the cleavage locus in the treatment group. The Sanger sequences of the untreated (control) group and the treatment group are set forth in SEQ ID NOs: 80 and 81, respectively.

FIG. 14: Cas9 endonuclease and gRNA were delivered to rat lung epithelial cell line by plasmid transfection. Transfected cells were enriched by puromycin selection and analyzed for gene expressions and DNA mutations on day 14 and 28.

FIG. 15: Derepression genome editing in cell lines from different species. (A) SaCas9 and gRNAs targeting the region between 700 bp upstream of the human IL-10 gene were delivered to human kidney cell line (HEK293) by plasmid transfection. Human IL-10 expression after 48 hour of transfection were measured by qPCR. Relative expression levels to the non-targeting group were shown. One-way ANOVA followed by Bonferroni correction is used in comparison to the negative control group (Neg). (B) SaCas9 and gRNAs targeting the region between 1-500 bp upstream of the porcine IL-10 gene were delivered to porcine kidney cell line (PK15) by plasmid transfection. Porcine IL-10 expression after 48 hour of transfection were measured by qPCR. Ct values for samples which remained undetectable after 40 cycles were set as 40. Values in the untreated cells were used as 1. One-way ANOVA followed by Bonferroni correction is used in comparison to the negative control group (Neg). (C) Illustration of genomic loci targeted by each of gRNA 84 (human), gRNA 37 (porcine), gRNA 4 (rat). (D) SaCas9 and gRNAs targeting the region between 1-300 bp upstream of the IL-10 gene were delivered to human, porcine, and rat cell lines by plasmid transfection. Endogenous IL-10 gene expression at 48 hours were measured by qPCR. Ct values for samples which remained undetectable after 40 cycles were set as 40. Values in the negative control were used as 1. One-way ANOVA followed by Bonferroni correction is used in comparison to non-targeting group. N=2, biological replicates. Symbols indicate *; P≤0.05, **; P≤0.01, and ***; P≤0.001.

FIG. 16: Upregulation of the IL-10 gene by base editing in a human cell line. (A) HEK293 cells were transfected with plasmid expressing gRNA 84 (comprising the sequence set forth in SEQ ID NO: 36), SaCas9 and GFP. After sorting of transfected cells by cell sorter using GFP signal, clones were isolated. Cell lysates were collected for RNA and DNA purification. The expression of human IL-10 gene was measured by qPCR. (B) Cell culture supernatant of each clone was assessed for human IL-10 protein levels by ELISA. (C) Targeted Sanger sequencing of genomic DNA purified from untreated and clone 8. The Sanger sequences of the untreated (control) sample and clone 8 are set forth in SEQ ID NOS: 82 and 83, respectively. (D, E, F) SpCas9-based base editors and gRNA 110 and/or gRNA 111 were delivered to human kidney cell line (HEK293) by plasmid transfection. Human IL-10 expression after 48 hours of transfection were measured by qPCR. Relative expression levels to the negative control group (Neg) were shown. Ct values for samples which remained undetectable after 40 cycles were set as 40. One-way ANOVA followed by Bonferroni correction is used in comparison to the negative control. N=2, biological replicates. Symbols indicate ****; P≤0.0001, **; P≤0.01 *; P≤0.05. ABE; adenine base editor, CBE; cytosine base editor, SPACE; synchronous programmable adenine and cytosine editor. (G) Analysis of targeted Sanger sequencing of the genomic DNA in human cell line. Base editor and gRNA were expressed in HEK293 cells by plasmid transfection. Genomic DNA was purified after 48 h. The target region of the gRNA (comprising the sequence set forth in SEQ ID NO: 84) was PCR-amplified and subject to Sanger sequencing. Data was analyzed using EditR. A·T-to-G·C or C·G-to-T·A conversions were observed at the predicted loci in the editing groups. ABE; adenine base editor, CBE; cytosine base editor.

FIG. 17: (A) HEK293 cells were transfected with each plasmid construct and measured for endogenous IL-10, exogenous IL-10, and editing efficiency at 48 hours. gRNA 84 was used. (B) Time course of endogenous and exogenous IL-10 expressions in HEK293 cells treated with IL-10 gene editing-transfer plasmid. Symbols indicate significance compared to Neg (A) or the initial point (B). (C) Map of the IL-10 gene editing-transfer construct. (D) L2 cells were transfected with each plasmid construct (gRNA 4 was used) and measured for endogenous rat IL-10, exogenous human IL-10, and editing efficiency at 48 hours.

FIG. 18: Assessment of IL-10-activated donor lungs in syngeneic single lung transplant non-injury survival model. Two-gRNA adenoviral vectors or buffer without any virus (diluent group) was delivered to left lung of donor Lewis rats in vivo. After 24 hours of viral deliver, the left lung grafts were transplanted into recipient Lewis rats. Grafts were assessed on postoperative day 3 (POD3). Recipients received triple immunosuppression until analysis (n=5 and n=6 for diluent and two gRNA group, respectively.) (A) Donor PaO₂/FiO₂ ratio calculated from PaO2 of systemic arterial blood taken at FiO₂ of 0.5 (left). PaO₂ of blood taken from left pulmonary vein after reperfusion (right). (B) Wet-to-dry weight ratio of left lung graft collected on POD3. (C) Recipient body weight on POD3 were shown as percent of those on POD0. (D and E) Gene expressions in the left lung graft on POD3 measured by qPCR. Values of left lung from untreated Lewis rats (n=2) were set as 1. Mean±SD are shown. Mann-Whitney test was used for statistical comparison. NS; not significant, **; P≤0.01.

FIG. 19: Assessment of IL-10-activated donor lungs in allogeneic single lung transplant survival model. Two-gRNA adenoviral vectors or diluent was administered to left lungs of donor Brown Norway rats through airway 24 hours prior to retrieval. Left lung grafts were transplanted into recipient Lewis rats. Triple immunosuppression was applied to recipient until analysis on POD3. Mean±SD are shown. Mann-Whitney test was used for statistical comparison. (n=3 for each group) (A) Donor PaO₂/FiO₂ ratio (right) and PaO₂ of blood taken from graft left pulmonary vein after reperfusion (right). (B) Wet-to-dry ratio of left lung graft collected on POD3. (C) Recipients' body weight on POD3 relative to those of POD1. (D) Gene expressions in the tissue of left lung graft collected on POD3 were measured by qPCR. (E) Fluorescence in situ hybridization of lung tissues of left lung graft for SaCas9 (green) and rat IL-10 (red). Nuclei were counter stained with DAPI (blue).

Other features and advantages of the present disclosure will become apparent from the following description and from the exemplary embodiments.

### DETAILED DESCRIPTION OF THE DISCLOSURE

The description which follows, and the embodiments described therein, are provided by way of illustration of an example, or examples, of particular embodiments of the principles of the present disclosure. These examples are provided for the purposes of explanation, and not limitation, of those principles and of the disclosure.

### Definitions

The terminology used herein is for the purpose of describing particular embodiments only and is not intended to be limiting of example embodiments of the invention. Unless defined otherwise, all technical and scientific terms used herein generally have the same meaning as commonly understood by those of ordinary skill in the art. Generally, nomenclatures used in connection with synthetic chemistry, organic chemistry, biochemistry, molecular biology, cell and tissue culture, immunology, genetics, etc. described herein are those well-known and commonly used in the art. Nevertheless, definitions of selected terms are provided for clarity and consistency.

As used herein, the singular forms “a,” “an,” and “the,” are intended to include the plural forms as well, unless the context clearly indicates otherwise.

The phrase “and/or” should be understood to mean “either or both” of the elements so conjoined, i.e., elements that are conjunctively present in some cases and disjunctively present in other cases. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein, the phrase “one or more,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “one or more” refers, whether related or unrelated to those elements specifically identified. Thus, as a non-limiting example, “one or more of A and B” (or, equivalently, “one or more of A or B,” or, equivalently “one or more of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the term “about” is used in conjunction with a numerical range, it modifies that range by extending the boundaries above and below those numerical values. In general, the term “about” is used herein to modify a numerical value above and below the stated value by a variance of 20%, 10%, 5%, or 1%. In certain embodiments, the term “about” is used to modify a numerical value above and below the stated value by a variance of 10%. In certain embodiments, the term “about” is used to modify a numerical value above and below the stated value by a variance of 5%. In certain embodiments, the term “about” is used to modify a numerical value above and below the stated value by a variance of 1%.

When a range of values is listed herein, it is intended to encompass each value and sub-range within that range. For example, “1-5 ng” is intended to encompass 1 ng, 2 ng, 3 ng, 4 ng, 5 ng, 1-2 ng, 1-3 ng, 1-4 ng, 1-5 ng, 2-3 ng, 2-4 ng, 2-5 ng, 3-4 ng, 3-5 ng, and 4- 5 ng.

It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

The term “consisting of” and its derivatives, as used herein, are intended to be closed terms that specify the presence of stated features, integers, steps, operations, elements, and/or components, and exclude the presence or addition of one or more other features, integers, steps, operations, elements and/or components.

The term “inflammation-regulating and/or immune-regulating gene”, as used herein, refers to any gene involved in the immune and/or inflammatory response. As is known to those skilled in the art, there are many genes involved in immunity and inflammation, including anti-inflammatory and pro-inflammatory genes. For example, see Ischemia-reperfusion-induced lung injury-PubMed (nih.gov) and Gene therapy in lung transplantation-PubMed (nih.gov). These genes include, but are not limited to: interleukin-10 (IL-10), interleukin 1 receptor antagonist (IL-1RN), Interleukin-4 (IL-4), Interleukin-6 (IL-6), Interleukin-8 (IL-8), tumor necrosis factor binding protein (TNFBP), cytotoxic T-lymphocyte associated protein 4 (CTLA4), NFKB inhibitor alpha (Ikba), programmed death-ligand 1 (Pd-L1), programmed cell death-1 (PD-1), Interleukin-10 receptor, CD47, CD200, Fas Ligand (FasL), MHC molecule (MHC-Ib), H2-M3, Serine protease inhibitor 6 (Spi6), C-C Motif Chemokine Ligand 21 (Ccl21) and Milk Fat Globule-EGF Factor 8 Protein (Mfge8).

The term “regulatory region”, as used herein, refers to any sequence or segment of DNA that modulates the level of expression of a gene, such as an inflammation-regulating and/or immune-regulating gene. Regulatory regions include, but are not limited to, promoters (including core promoter, proximal promoter, and distal promoter), enhancers (including proximal and distal enhancers), silencers, or terminators. Regulatory regions may be upstream or downstream of the coding sequence of the gene. Regulatory regions contain various sequence motifs that interact with transcriptional machinery or distal enhancers or silencers in trans. Such DNA sequence motifs include, but are not limited to, E-box (CANNTG) and TATA box. In some embodiments provided herein, mutations to the E-box motif modulate the expression of an inflammation-regulating and/or immune-regulating gene. In some embodiments provided herein, mutations to the E-box motif modulate the expression of the IL-10 gene.

The term “expression cassette”, as used herein, refers to a DNA molecule comprising a nucleotide sequence that is expressed (transcribed) upon transfer into a cell, tissue or organ. Typically, an expression cassette comprises at least one gene (including a promoter, an open reading frame, and a termination signal). In some embodiments provided herein, the expression cassettes comprise, for example, polynucleotides that encode gRNA(s) and/or Cas enzymes or derivatives thereof, and optionally, cDNA(s) encoding gene(s)-of-interest as well as upstream promoter and downstream termination signal. Various promoters are suitable for the expression cassettes provided herein. For example, Cas genes may be expressed from a CMV promoter, and gRNAs may be expressed from a U6 promoter or an H1 promoter.

“epigenetic modification” is a change made to the DNA, which regulates how a gene is expressed, without alterations in the nucleotide sequence of the gene. Examples of epigenetic modifications include, but are not limited to, transcriptional regulation, DNA methylation, histone modification, and micro RNA regulation.

“genetic modification” is a change made to the sequence of the genomic DNA. This may include insertions, deletions, substitutions, etc. as is known to those skilled in the art. For example, it may include insertion of an exogenous gene, promoter, or reading frame. It may also include modification of an endogenous genomic sequence.

“immunosuppressive agent” is an agent that suppresses the immune and/or inflammatory response. Examples of immunosuppressive agents include, but are not limited to, corticosteroids, calcineurin inhibitors, and antimetabolites. Specific agents include, but are not limited to methylprednisolone, cyclosporin, cyclosporin A, azathioprine, anti-thymocyte globulin, rapamycin, tacrolimus, mycophenolate mofetil, mycophenolic acid, sirolimus, everolimus, and prednisone. In an embodiment, the immunosuppressive agent is a combination of methylprednisolone, cyclosporine and azathioprine. In an embodiment, the immunosuppressive agent is a combination of methylprednisolone and cyclosporine.

This disclosure relates to epigenetically and genetically modified organs and tissues or a cell of an organ or tissue thereof, and methods of producing same. The organs and tissues may be used for transplantation. Immunologically enhanced organs and tissues by epigenetic or genetic modification may expand the donor pool by repairing injured donor organs currently being declined for transplant. In addition to repairing organs and tissues for transplant, local immunosuppression of optimized lungs and tissues may reduce or eliminate life-long and systemic immunosuppression that has been a challenge in post-transplant life.

Certain processes for preserving, maintaining, repairing, and/or improving an organ isolated from a donor are known to those skilled in the art. For example, certain processes are described in U.S. Ser. No. 14/658,456, which discloses an ex vivo perfusion system and is incorporated herein by reference as if set forth in its entirety. See also XVIVO PERFUSION, http://www.xvivoperfusion.com. Additional methods may include, but are not limited to, IL-10 gene therapy, alpha-1 antitrypsin (A1AT), 10 degree Celsius preservation, and others, as is known to those skilled in the art (See additional references cited on page 16-17).

The ex vivo perfusion system also provides a method for assessing the organ to determine whether it is suitable for transplantation into a recipient. Organs subjected to this process may be improved candidates for transplantation compared to organs not subject to ex vivo perfusion.

### Organs and Tissues

Any organ and tissue may be used for transplantation. For example, organs may include but are not limited to lung, heart, kidney, liver, pancreas, stomach and intestine. Tissues may include, but are not limited to, cornea, bone, tendon, skin, pancreas islets, heart valves, nerves and veins.

### Donors and Recipients

The donor organ and tissue may come from human, or any animal source, including but not limited to pig. In addition, the donor organ or tissue may be engineered tissue (for example, re-cellularized scaffold). The recipient of the modified organ may be human or any non-human animal. Further, an organ or tissue from an animal may be removed from the animal, modified ex vivo, and reintroduced into the same animal after modification.

### Ex Vivo Organ Perfusion (EVOP) System

Any perfusion system may be used. For example, certain systems are described in U.S. patent application Ser. No. 14/658,456, which discloses an ex vivo perfusion system and is incorporated herein by reference in its entirety. See also XVIVO PERFUSION, http://www.xvivoperfusion.com.

An ex vivo organ perfusion system subjects a donor organ to a blood-less acellular normothermic perfusate. With this system, a donor organ can be perfused in an ex vivo circuit, providing an opportunity to assess and re-assess the function of donor organ before transplantation. The EVOP can be used at all stages of process to assist in the preservation, maintenance, repair, improvement, and/or assessment of an organ. For example, EVOP can be used soon after donor organ has been excised from the donor and can replace or supplement standard evaluation of process. EVOP can also be used after standard evaluation on both transplantable organ and injured organ in order to preserve, maintain, improve, assess and, in the case of injured organ, repair donor organs. With ex vivo organ perfused organs, organs are perfused, and in the case of lungs, are additionally ventilated, at body temperature to mimic physiologic conditions, which can preserve and maintain the organ, as well as allow the organ to be treated or to repair itself. Successful EVOP can restore the cell structure integrity and allow, for example, ZO-1 tight junction repair of donor organ. Organs subjected to EVOP can be preserved and maintained for 12, 24, 72 hours, or longer.

EVOP system requires perfusate, which may comprise or consist of a commercially available solution, such as Steen Solution™, Perfadex™ solution or other solution suitable for perfusion according to the present invention. Steen Solution™ comprises calcium chloride, Dextran 40, Glucose, Human serum albumin, magnesium chloride, potassium chloride, sodium bicarbonate, sodium chloride, sodium dihydrogen phosphate, and water. It is preferable that the solution has a pH of about 7.4 (±0.3) and an osmolality of around 295±20 mOsm/kg. It will be recognized by the skilled person that solutions that do not comprise all of these ingredients, substitute ingredients, or have additional ingredients, may be suitable to use as perfusate. Other components can be used in perfusate to either assist in the preservation, maintenance, improvement, or repair of donor organ. For example, heparin or the like can be used, such as, for example, sodium heparin, in an amount of, for example, 3,000 to 10,000 international units. Antibiotics can be added, such as, for example, cefazolin or Primaxin™, in an amount of, for example, 500 mg. Another example of a component that can be added to perfusate are steroids, such as methylprednisolone (also known as Solumedrol™), in an amount of, for example, and 500 mg. It is preferred that perfusate is acellular; that is, it does not contain or contains a minimal amount of red blood cells, particularly when donor organ is one or two lungs.

The ex vivo organ perfusion system is designed to provide perfusate to donor organ in a beneficial and temperature-controlled manner such as at a temperature between about 20 degree Celsius and 38 degree Celsius. Most preferably, the temperature is about 37 degree Celsius. The operation of ex vivo organ perfusion is known to those skilled in the art and shown, for example, in U.S. patent application Ser. No. 14/658,456, which is incorporated by reference.

During ex vivo organ perfusion, injured organs can be repaired during a treatment period using any one or a combination of treatments. In this way, an aspect of the present invention is the treatment of an ex vivo organ rather than the whole body. The skilled person would understand that repairing organs according to the present invention can be suitable to treat an organ of a living donor with the intention of returning the repaired organ to the original donor.

An example of a treatment that can be used in combination with EVOP is gene therapy. Gene therapy with adenoviral vector performed in the ex vivo system provides superior uptake and expression of the gene. For example, the injury response of the ex vivo lung is superior to gene therapy done in vivo in the donor. Gene therapy ex vivo avoids exposure of the donor and other organs to the vector and gene therapy. Further, gene therapy can decrease the risk of acute lung injury after transplantation and its attendant complications. In embodiments where the organ is a lung, IL-10 gene therapy, or other suitable cytokines, can decrease inflammation of the lung and can restore tight junctions of the lung. For example, inflammation and disruption of the blood-alveolar barrier in the lung are common in injured human donor lungs that have been rejected for transplantation. Such injuries can occur before removal of the lung from the donor as well as during the preservation period. Using IL-10 gene therapy in a perfused lung can result in a decrease of IL-1B, IL-8, and/or IL-6 concentrations in the lung tissue.

In some embodiments, a second-generation (E1, E3 deleted), replication-deficient adenoviral vector (serotype 5) under the control of a cytomegalovirus promoter, or other suitable promoter, and containing the human IL-10 gene can be used. The adenoviral vector can then be diluted in about 20 ml of normal saline.

In order to deliver the vectors provided herein (for example, adenoviral vectors comprising expression cassettes that encode one or more guide RNA(s) and a Cas protein), in some embodiments a bronchoscope, such as a flexible fibre-optic bronchoscope, can be inserted through an endotracheal tube inserted in lungs during the perfusion process. A fine catheter can then be inserted through the bronchoscope channel and can be used to deliver about 1 mL of the vector into each segmental bronchus. After the vector has been delivered, a recruitment maneuver (or an inspiratory hold) can be performed to an airway pressure of about 25 cm H₂O and lungs can be ventilated with a tidal volume of about 10 ml/kg at about 14 bpm for about 15 minutes which can facilitate distribution of the vector through lungs.

Treatment of donor organ can occur for a treatment period of time, the length of which will depend on the condition of the organ and the injury being treated. For example, when donor organ is donor lung and being treated by gene therapy, the treatment period of time can be from about 1 hour to about 24 hours, more preferably between 4 hours and about 12 hours, and even more preferably between about 2 hours and about 4 hours.

Process together with EVOP system as described above can also be useful to improve the condition of donor organ, whether donor organ is injured or high risk or generally transplantable. For example, in the case of donor lung, lung capacity values (PO₂: FIO₂) can be improved, pulmonary edema can be reduced, consolidation can be reduced, and/or atelectasis can be achieved. Such improvement can occur during the maintenance period without treatment or can occur during part or all of both the maintenance period and the treatment period, as is known to those skilled in the art and described in U.S. patent application Ser. No. 14/658,456, which is incorporated herein by reference. Improvement can also occur during or substantially during the treatment period. For example, such improvements can be achieved between about 1 hour and about 3 hours of the maintenance period of EVOP.

When a donor organ being held in the maintenance period is treated, the maintenance period may be shortened accordingly. After the treatment period, a maintenance period may be initiated, resumed, or repeated. Assessment of the donor organ can occur throughout, or through part of, the maintenance and/or treatment periods.

Organs that have been preserved, assessed, maintained, repaired, and/or improved by the processes and systems described herein also fall within the scope of this invention. For example, a lung maintained by the EVOP system for a period of time, such as 1 hour or 4 hours, or longer, can have improved lung capacity (PO₂: FIO₂) over time compared to a lung suitable to transplant without being subjected to EVOP, a lung maintained by standard hypothermic conditions, or an injured lung not originally suitable to transplant. For example, a lung maintained by the EVOP system can have improved lung function parameters, a reduction of inflammatory cells content, improved lung microcirculation, restored cell tight junctions, and other parameters targeted for preconditioning for transplantation.

The EVOP system may also be used for assessment of CRISPR-based intervention in human lungs, for example, to evaluate donor lungs declined for clinical transplant or explanted lungs incubated in the ex vivo lung perfusion platform. This approach overcomes the existing challenges associated with assessing interventions targeting the human genome in human organs.

### In Vivo Lung Perfusion (IVLP)

In vivo lung perfusion (IVLP) is a method whereby a surgeon can isolate the lungs (via cannulation to pulmonary artery and veins and connecting to organ perfusion circuit) from the rest of the body, and providing essential oxygen and nutrients to enable the lung to continue to function normally. IVLP can then be used to deliver targeted treatments that are designed to only impact the isolated lung, with minimal or no systemic exposure.

Isolated lung perfusion separates lung circulation from systemic circulation; however, unlike EVOP systems, the lungs remain in the body. This system shares the major advantages of ex vivo lung perfusion-including lung selectivity, organ pre-conditioning, and intensive treatment-without requiring transplantation. The feasibility and safety of organ-selective high-dose medication using this platform have been demonstrated with chemotherapy. In support of its initial aim of development, isolated lung perfusion has been applied to treat pulmonary metastasis of cancers, resulting in clinical benefits. Moreover, lung recovery in acute respiratory distress syndrome (ARDS) via in vivo lung perfusion in a large animal model has been shown to reduce the inflammatory response and rehabilitate the lung. These features are attractive for in vivo genome and/or epigenome editing using CRISPR-Cas technology.

In Vivo Treatment without Perfusion

The expression vectors of the present disclosure may also be administered directly to a subject in vivo without isolating or perfusing the lung. In some embodiments, the cassettes or vectors provided herein (comprising a polynucleotide that encodes gRNA(s), Cas enzymes or derivatives thereof, and optionally, cDNA(s) encoding gene(s)-of-interest as well as the associated promoters and termination signals may be delivered to a subject, for example, by oral inhalation, by nasal inhalation, using a bronchoscope, or by orotracheal intubation.

In some embodiments of the in vivo treatment and the in vivo perfusion methods and systems provided herein, an inflammation-regulating or immune-regulating gene is modified to control inflammation resulting from any type of lung injury or disease, such as, but not limited to, pulmonary fibrosis, cystic fibrosis, or adult respiratory distress syndrome (ARDS).

In some embodiments, inflammatory lung diseases are treated in vivo such as applying CRISPR therapeutics by way of an in vivo perfusion (for example, delivery of expression vectors through airway or circuit during IVLP) or an in vivo treatment (for example, delivery of expression vectors through airway using a bronchoscope), in order to ameliorate inflammation and recover the injured lungs. In some embodiments, the treatment involves simultaneous (i) suppression of lung inflammation and (ii) correction of a disease-causing mutation, or replacement of the disease-causing mutation with a beneficial mutation, in the lung of a subject having a genetic inflammatory disease.

### Target Genes

The target gene for modification may be any gene in the organ or tissue. For example, the target gene for modification may be a gene involved in the immune and/or inflammatory response. The target gene for modification may be a gene encoding a cytokine. The target gene for modification may include any inflammation-regulating and/or immune regulating genes. As is known to those skilled in the art, there are many genes involved in immunity and inflammation, and include anti-inflammatory, and pro-inflammatory, genes. For example, the inflammation-regulating and/or immune regulating genes may include, but are not limited to: interleukin-10 (IL-10), interleukin 1 receptor antagonist (IL-1RN), Interleukin-4 (IL-4), Interleukin-6 (IL-6), Interleukin-8 (IL-8), tumor necrosis factor binding protein (TNFBP), cytotoxic T-lymphocyte associated protein 4 (CTLA4), NFKB inhibitor alpha (Ikba), Programmed cell death-1 (PD-1), Programmed cell death-ligand 1 (PD-L1), Interleukin-10 receptor, CD47, CD200, Fas Ligand (FasL), MHC molecule (MHC-Ib) H2-M3, Serine protease inhibitor 6 (Spi6), C-C Motif Chemokine Ligand 21 (Ccl21), Milk Fat Globule-EGF Factor 8 Protein (Mfge8).

The genomic sequences of certain suitable inflammation-regulating and/or immune regulating genes, including the upstream and downstream regulatory regions, are set forth in SEQ ID NOS: 111-130 and listed in Table 1 below. Generally, variants of a particular nucleotide sequence of the embodiments disclosed herein will have at least about 70%, 75%, 80%, 85%, 86%, 87%, 88%, 89%, 90%, 91%, 92%, 93%, 94%, 95%, 96%, 97%, 98%, 99%, or more sequence identity to that particular nucleotide sequence as determined by sequence alignment programs using default parameters as is known to those skilled in the art. A variant of a nucleotide sequence of the embodiments may differ from that sequence by as few as 1-15 nucleotides, as few as 1-10, such as 6-10, as few as 5, as few as 4, 3, 2, or even 1 nucleotide.

TABLE 1
Target genes and accession numbers

| SEQ ID | | | |
|---|---|---|---|
| NO. | Gene | Species | Accession No. |
| 111 | IL-10 | Human | NC_000001.11 Reference |
| | (larger genomic | | GRCh38.p14 Primary Assembly |
| | region) | | |
| 112 | IL-10 | Human | NG_012088.1 |
| | (larger genomic | | |
| | region) | | |
| 113 | IL1RN | Human | NC_000002.12 Reference |
| | (larger genomic | | GRCh38.p14 Primary Assembly |
| | region) | | |
| 114 | IL1RN | Human | NG_021240.1 |
| | (larger genomic | | |
| | region) | | |
| 115 | IL-4 | Human | NG_023252.1 |
| 116 | IL-6 | Human | NG_011640.1 |
| 117 | IL-8 | Human | NG_029889.1 |
| 118 | CTLA4 | Human | NG_011502.1 |
| 119 | Ikba | Human | NG_007571.1 |
| 120 | PD-1 | Human | NG_012110.1 |
| 121 | PD-L1 | Human | NC_000009.12 Reference |
| | | | GRCh38.p14 Primary Assembly |
| 122 | IL10RA | Human | NG_016275.1 |
| 123 | IL10RB | Human | NG_012089.1 |
| 124 | CD47 | Human | NC_000003.12 Reference |
| | | | GRCh38.p14 Primary Assembly |
| 125 | CD200 | Human | NC_000003.12 Reference |
| | | | GRCh38.p14 Primary Assembly |
| 126 | FASLG | Human | NG_007269.1 |
| 127 | Ccl21 | Human | NC_000009.12 Reference |
| | | | GRCh38.p14 Primary Assembly |
| 128 | Mfge8 | Human | NC_000015.10 Reference |
| | | | GRCh38.p14 Primary Assembly |
| 129 | H2-M3 | Mouse | NC_000083.7 Reference |
| | | | GRCm39 C57BL/6J |
| 130 | Spi6 | Mouse | NC_000079.7 Reference |
| | | | GRCm39 C57BL/6J |

The target gene may be modified in either coding or non-coding or in the regulatory sequences of the gene, for example, the promoter, enhancer, silencer or terminator regions.

More than one endogenous gene may be targeted for modification in the organ or tissue, for example, the IL-10 and IL-1RN gene may be modified to control the inflammatory response.

### Modification System

Any modification system may be used, including but not limited to, genetic transformation, epigenetic transformation, RNA modification (i.e. RNA editing by Cas 13) and endogenous gene modification via clustered regularly interspaced short palindromic repeats (CRISPR)-Cas system. In an embodiment, only one genomic locus is modified. In an embodiment, two or more genomic loci are modified. In various embodiments, the modification is a transient modification, a persistent modification, or a permanent modification.

See, for example:
The promise and challenge of therapeutic genome editing-PubMed (nih.gov), Doudna, JA Nature Vol. 578 13 Feb. 2020;
Genome editing with CRISPR-Cas nucleases, base editors, transposases and prime editors-PubMed (nih.gov), Anzalone et al., Nat. Biotechnology July 38 (7) 824-844 2020;
The next generation of CRISPR-Cas technologies and applications-PubMed (nih.gov), Pickar-Oliver A. and C.A. Gersbach Nat. Rev. Mol. Cell Biol. 2019 Aug. 20 (8) 490-507 2019.

CRISPR-Cas technology unleashed the possibility of making gene therapy more precise and persistent by radically altering gene expression through DNA targeting. For example, CRISPR-Cas9 may be used. In addition, transcriptional modulation systems include dCas9-VP64, SAM, SunTag, dCas9-VPRmini, and dCas9-KRAB. Other epigenome modification systems include, but are not limited to: dCas9-P300, dCas9-TET1, dCas9-Dnmt3a etc. Genome editing prime editor, base editor, Cas-transposase, Cas9 ortholog Cpf1, Cas13, split Cas9, Cas9 nickase etc. Reviews can be found at: Casas-Mollano et al., CRISPR-Cas Activators for Engineering Gene Expression in Higher Eukaryotes, The Crispr Journal, Vol. 3, Number 5, 2020, pages 350-364, https://pubmed.ncbi.nlm.nih.gov/33095045/and Sgro et al., Epigenome engineering: new technologies for precision medicine, Nucleic Acids Research, 2020, Vol. 48, No. 22, pages 12453-12482, https://pubmed.ncbi.nlm.nih.gov/33196851/.

Suitable Cas proteins include, but are not limited to, SpCas9, SaCas9, Cpf1 (Cas12a), NmeCas9 and their variants, such as SpG, SpRY. In some embodiments, Cas protein-derived base editors are used to introduce specific mutations at specific locations in the genome. Suitable base editors include, but are not limited to, adenine base editor (ABE), cytosine base editor (CBE), and synchronous programmable adenine and cytosine editor (SPACE).

Individual applications include, but are not limited to:
Bohm et al, A gene therapy for inherited blindness using dCas9-VPR-mediated transcriptional activation, Science Advances, Research Article, Aug. 19, 2020, pages 1-10, https://pubmed.ncbi.nlm.nih.gov/32875106/
Matharu et al, CRISPR-mediated activation of a promoter or enhancer rescues obesity caused by haploinsufficiency, Science 363, 246 (2019) 18 Jan. 2019-PubMed (nih.gov)
Kemaladewi et al., A mutation-independent approach for muscular dystrophy via upregulation of a modifier gene, Nature 2019 August; 572 (7767): 125-130, https://pubmed.ncbi.nlm.nih.gov/31341277/
Liao et al, In Vivo Target Gene Activation via CRISPR/Cas9-Mediated Trans-epigenetic Modulation, Cell. 2017 Dec. 14; 171 (7): 1495-1507.-PubMed (nih.gov)
Schoger et al, CRISPR-Mediated Activation of Endogenous Gene Expression in the Postnatal Heart, Circulation Research. 2020; 126:6-24-PubMed (nih.gov).
Liu et al, Rescue of Fragile X Syndrome Neurons by DNA Methylation Editing of the FMR1 Gene, 2018; Cell 172, 979-992-PubMed (nih.gov)
   All of which are incorporated herein by reference in their entirety.

A guide RNA of the present disclosure comprises a spacer sequence having at least 90%, or at least 95%, sequence identity to any one of the sequences set forth in SEQ ID NOs: 1-10, 12-42, 44-59 and 61-79, or a complement thereof. In some embodiments, the guide RNA comprises a spacer sequence that is 100% identical to any one of the sequences set forth in SEQ ID NOs: 1-10, 12-42, 44-59 and 61-79, or a complement thereof.

### Epigenetic or Genetic Modifications

The target gene expression may be modified by epigenetic modification(s) and/or genetic modification(s), where epigenetic modification(s) may be broadly considered as not only including modification of histone status but also including the use of components of epigenomic manipulation to affect transcriptional manipulation of gene expression. For example, a catalytically inactive CRISPR-associated protein 9 (Cas9) that has been fused to one or more transcription activation domain(s), referred to herein as a “Cas9 activator”, may be used to upregulate the expression of one or more anti-inflammatory cytokines. Suitable transcription activation domains may include, but are not limited to, VP64, p65 and RTA (VPR). Other examples include, but are not limited to, manipulation of DNA methylation using a domain of DNA demethylase or methylase and modulation of histone status using histone modifiers, such as a p300 domain of histone acetyltransferase.

### Delivery Vectors

Expression cassettes comprising polynucleotides encoding Cas protein(s), CRISPR guide RNAs (gRNAs) and/or cDNA of a gene or genes of interest are delivered to the organ or tissue that is to be modified using any suitable vector, as known to those of ordinary skill in the art. In certain embodiments, the delivery vector is a viral vector or a non-viral vector. Viral vectors include, but are not limited to, an adenoviral vector, an adeno-associated virus (AAV) vector, a lentiviral vector, or a retroviral vector. In an embodiment, the vector is an adenoviral vector or an AAV vector. In an embodiment, the vector is an adenoviral vector. In an embodiment, the non-viral delivery vector is a lipid nanoparticle.

### Kits

Provided herein are kits comprising the expression cassettes and/or delivery vectors comprising polynucleotides encoding Cas protein(s), CRISPR guide RNAs (gRNAs) and/or cDNA transgenes. The kits further comprise instructions for using the expression cassettes and/or delivery vectors. Optionally, the kits also include additional reagents. In an embodiment, the kits further comprise a perfusate, for example, Steen Solution™, Perfadex™. In some embodiments, the vector and the perfusate are formulated separately. In some embodiments, the vector and the perfusate are formulated together. The kits may also be packaged together with an ex vivo or in vivo perfusion system as described herein. The kits provided herein are intended for therapeutic purposes, such as modifying organs or tissues for transplantation, as well as for diagnostic purposes and/or research purposes.

### Examples

The disclosure is further described by reference to the following examples. These examples are provided for purposes of illustration only and are not intended to be limiting unless otherwise specified. Thus, the disclosure should in no way be construed as being limited to the following examples, but rather, should be construed to encompass any and all variations which become evident as a result of the teaching provided herein.

### Example 1: Materials and Methods

### Vector Design and Construction

Plasmids expressing dSaCas9-VPR (Addgene #68495) and dSpCas9-P300 (Addgene #61357) were obtained from Addgene (Watertown, MA). gRNAs 1-8 were designed to bind the region between −500 to +1 (transcriptional start site; TSS) of the IL-10 in the rat genome using a genome browser. 21-bp spacer sequences adjacent to SaCas9 PAM were used for gRNAs 1-7 while 20-bp spacers and SpCas9 PAM were used for gRNA 8 (see Table 2, below). gRNAs 1-8 comprise the spacer sequences set forth in SEQ ID NOs: 1-8, respectively. The DNA fragments containing the U6 promoter and gRNA cassette were synthesized as gBlock (Integrated DNA Technologies, Coralville, lowa). Single activation vectors were generated using standard restriction enzyme digestion followed by ligation. Golden Gate Assembly was also used when appropriate. Plasmids were purified using the QIAprep Spin Miniprep Kit (Qiagen, Hilden, Germany) and correct transformants were confirmed by Sanger sequencing (The Centre for Applied Genomics, Toronto, Canada). Plasmids were then amplified and purified using the EndoFree Plasmid Maxi Kit (Qiagen, Hilden, Germany) to transfect cells.

In some embodiments of the present disclosure, the gRNA comprises a spacer sequence having at least 90%, or at least 95%, sequence identity to any one of the sequences set forth in SEQ ID NOs: 1-10, 12-42, 44-59 and 61-79, or a complement thereof. In some embodiments, the gRNA comprises a spacer sequence that is 100% identical to any one of the sequences set forth in SEQ ID NOs: 1-10, 12-42, 44-59 and 61-79, or a complement thereof.

TABLE 2
List of gRNA spacer sequences and corresponding protospacer adjacent motif (PAM)
sequences

| SEQ ID | | | | | Target | Cas |
|---|---|---|---|---|---|---|
| NO. | gRNA no. | gRNA spacer sequence | PAM | Species | gene | enzyme |
| 1 | gRNA 1 | ATAGCGGACATTCAACCCAGG | TTGAGT | Rat | IL-10 | SaCas9 |
| 2 | gRNA 2 | ATTTCCTCCACTCAACCTGGG | TTGAAT | Rat | IL-10 | SaCas9 |
| 3 | gRNA 3 | CAGACAGCTGTTCTATGTACA | GAGAAT | Rat | IL-10 | SaCas9 |
| 4 | gRNA 4 | TGTAAGCACAAGAGAACTGGT | AAGAAT | Rat | IL-10 | SaCas9 |
| 5 | gRNA 5 | TGCTGCACCTTCGGTATAAAA | GGGGG | Rat | IL-10 | SaCas9 |
| 6 | gRNA 6 | GTATAAAAGGGGGACACCGGG | CAGGA | Rat | IL-10 | SaCas9 |
| 7 | gRNA 7 | CATAGCCACAACGCAGCCTTG | CAGAA | Rat | IL-10 | SaCas9 |
| 8 | gRNA 8 | AAATAGCGGACATTCAACCC | AGG | Rat | IL-10 | SpCas9 |
| 9 | gRNA 9 | GAACGACAGCAGCGCAGGCTG | GTGAAT | Rat | IL1RN | SaCas9 |
| 10 | gRNA 10 | GTGGGGATCAGCAAATAGACT | CAGAGT | Rat | IL1RN | SaCas9 |
| 11 | Mock | AGCGAGGTATTCGGCTCCGCG | — | — | — | SaCas9 |
| 12 | gRNA 21 | CTCGGATCTGGTTTGCGAGGA | GCGGGT | Rat | IL1RN | SaCas9 |
| 13 | gRNA 22 | TCTGGTTTGCGAGGAGCGGGT | GGGGAT | Rat | IL1RN | SaCas9 |
| 14 | gRNA 23 | CCCGCTCCTCGCAAACCAGAT | CCGAGT | Rat | IL1RN | SaCas9 |
| 15 | gRNA 24 | AGAAGAAACAGTTGCTAGACT | CTGGAT | Rat | IL1RN | SaCas9 |
| 16 | gRNA 26 | AACACACCATTGCGACACCCT | CAGGGT | Rat | IL1RN | SaCas9 |
| 17 | gRNA 28 | CTCATTTGCATGACAGGTACT | CTGAGT | Rat | IL1RN | SaCas9 |
| 18 | gRNA 29 | GCTGTTGTCACTGTCAACCCT | GAGGGT | Rat | IL1RN | SaCas9 |
| 19 | gRNA 31 | GTCATTGTGACCTAGGAACAC | GTGAAT | Pig | IL-10 | SaCas9 |
| 20 | gRNA 32 | CCCAACCTGGGTTGAGTACTC | AAGGGT | Pig | IL-10 | SaCas9 |
| 21 | gRNA 33 | AGTACTCAAGGGTTCTCCTTG | CTGGAT | Pig | IL-10 | SaCas9 |
| 22 | gRNA 34 | ACGTGGACAAATTGGCCATTC | CAGAAT | Pig | IL-10 | SaCas9 |
| 23 | gRNA 35 | TGTGCGCAGAGCCCCACAGTT | GTGGGT | Pig | IL-10 | SaCas9 |
| 24 | gRNA 36 | ATTGGCCATTCCAGAATACAC | TGGGAT | Pig | IL-10 | SaCas9 |
| 25 | gRNA 37 | TTGGAGAGGTCTAGGGAAGGG | AGG | Pig | IL-10 | SaCas9 |
| 26 | gRNA 71 | TTTTATACCGAAGGTGCAGC | AGG | Rat | IL-10 | SpCas9 |
| 27 | gRNA 72 | TATTGTCACCCCGGATGGAA | TGG | Rat | IL-10 | SpCas9 |
| 28 | gRNA 73 | GCTGCACCTTCGGTATAAAA | GGG | Rat | IL-10 | SpCas9 |
| 29 | gRNA 74 | AAATAGCGGACATTCAACCC | AGG | Rat | IL-10 | SpCas9 |
| 30 | gRNA 75 | GTGTCCCCCTTTTATACCGA | AGG | Rat | IL-10 | SpCas9 |
| 31 | gRNA 76 | GTGACTTACGAGTGCGTGAA | TGG | Rat | IL-10 | SpCas9 |
| 32 | gRNA 77 | CTGCACCTTCGGTATAAAAG | GGG | Rat | IL-10 | SpCas9 |
| 33 | gRNA 81 | AATAATTGGGTCCCCCCAACC | TGGGAT | Human | IL-10 | SaCas9 |
| 34 | gRNA 82 | ACGTGGACAAATTGCCCATTC | CAGAAT | Human | IL-10 | SaCas9 |
| 35 | gRNA 83 | TTTAGGATGGGCTACCTCTCT | TAGAAT | Human | IL-10 | SaCas9 |
| 36 | gRNA 84 | CACAATCAAGGTTTCCCGGCA | CAGGAT | Human | IL-10 | SaCas9 |
| 37 | gRNA 85 | GTCACTGTGACCTAGGAACAC | GCGAAT | Human | IL-10 | SaCas9 |
| 38 | gRNA 86 | TGTGCGCAGAGGCCCTCAGCT | GTGGGT | Human | IL-10 | SaCas9 |
| 39 | gRNA 87 | TTTCTCAATCCCATTGTATTC | TGGAAT | Human | IL-10 | SaCas9 |
| 40 | gRNA 88 | CCTACAGTACAGGCGGGGTCA | CAGGAT | Human | IL-10 | SaCas9 |
| 41 | gRNA 89 | ACTGGCTTCCTACAGTACAGG | CGGGGT | Human | IL-10 | SaCas9 |
| 42 | gRNA 90 | AAGAGGTGGAAACATGTGCCT | GAGAAT | Human | IL-10 | SaCas9 |
| 43 | Mock 68 | TCGATAAGCCAGTAAGCAGTT | — | — | — | SaCas9 |
| 44 | gRNA 91 | CAATCAAGGTTTCCCGGCAC | AGG | Human | IL-10 | SpCas9 |
| 45 | gRNA 92 | GTGCCGGGAAACCTTGATTG | TGG | Human | IL-10 | SpCas9 |
| 46 | gRNA 93 | GGTGACTGCCTAAGTTAGCA | AGG | Human | IL-10 | SpCas9 |
| 47 | gRNA 94 | GTTCCTAGGTCACAGTGACG | TGG | Human | IL-10 | SpCas9 |
| 48 | gRNA 95 | GGTGAAGGTCTACACATCAG | GGG | Human | IL-10 | SpCas9 |
| 49 | gRNA 96 | AACAGCTGTTCTGTGCGCAG | AGG | Human | IL-10 | SpCas9 |
| 50 | gRNA 97 | GTTCTCATTCGCGTGTTCCT | AGG | Human | IL-10 | SpCas9 |
| 51 | gRNA 98 | TGCAAGTCTGTCTTGTGGTT | TGG | Human | IL-10 | SpCas9 |
| 52 | gRNA 99 | TCTTTTGCAAGTCTGTCTTG | TGG | Human | IL-10 | SpCas9 |
| 53 | gRNA 100 | TCATTAAAAAGCCACAATCA | AGG | Human | IL-10 | SpCas9 |
| 54 | gRNA 110 | CAATCAAGGTTTCCCGGCAC | AGG | Human | IL-10 | SpCas9 |
| 55 | gRNA 111 | GTGCCGGGAAACCTTGATTG | TGG | Human | IL-10 | SpCas9 |
| 56 | gRNA 112 | ATTAGAGAGGTTAGAGAAGG | AGG | Human | IL-10 | SpCas9 |
| 57 | gRNA 113 | TTTATTAGAGAGGTTAGAGA | AGG | Human | IL-10 | SpCas9 |
| 58 | gRNA 114 | GAAAACTAAGTTTATTAGAG | AGG | Human | IL-10 | SpCas9 |
| 59 | gRNA 115 | GCATCGTAAGCAAAAATGAT | TGG | Human | IL-10 | SpCas9 |
| 60 | Mock 116 | TCGATAAGCCAGTAAGCAGT | — | — | — | SpCas9 |
| 61 | gRNA 117 | GGCCCTCAGCTGTGGGTTCT | CAT | Human | IL-10 | SpRY |
| 62 | gRNA 118 | CTCAGCTGTGGGTTCTCATT | CGC | Human | IL-10 | SpG |
| 63 | gRNA 119 | AACCCACAGCTGAGGGCCTC | TGC | Human | IL-10 | SpG |
| 64 | gRNA 120 | CCCACAGCTGAGGGCCTCTG | CGC | Human | IL-10 | SpG |
| 65 | gRNA 123 | GGCACATGTTTCCACCTCTT | CAG | Human | IL-10 | SpRY |
| 66 | gRNA 124 | GCACATGTTTCCACCTCTTC | AGC | Human | IL-10 | SpG |
| 67 | gRNA 125 | GAGGTGGAAACATGTGCCTG | AGA | Human | IL-10 | SpG |
| 68 | gRNA 126 | AGGTGGAAACATGTGCCTGA | GAA | Human | IL-10 | SpRY |
| 69 | gRNA 127 | AAACATGTGCCTGAGAATCC | TAA | Human | IL-10 | SpRY |
| 70 | gRNA 128 | CATGTGCCTGAGAATCCTAA | TGA | Human | IL-10 | SpG |
| 71 | gRNA 129 | CAGCTGTCCCCCACCCCAAC | TGT | Human | IL-10 | SpG |
| 72 | gRNA 130 | GTGGGGGACAGCTGAAGAGG | TGG | Human | IL-10 | SpCas9 |
| 73 | gRNA 131 | TGGGGGACAGCTGAAGAGGT | GGA | Human | IL-10 | SpG |
| 74 | gRNA 132 | GGGGGACAGCTGAAGAGGTG | GAA | Human | IL-10 | SpRY |
| 75 | gRNA 133 | GGGGACAGCTGAAGAGGTGG | AAA | Human | IL-10 | SpRY |
| 76 | gRNA 134 | CACGTGTCTTCTTTTATAAA | TGG | Human | IL-10 | SpCas9 |
| 77 | gRNA 135 | GAAGACACGTGAATTTGATA | AGA | Human | IL-10 | SpG |
| 78 | gRNA 136 | AAGCCACAATCAAGGTTTCC | CGG | Human | IL-10 | SpCas9 |
| 79 | gRNA 137 | TCATTAAAAAGCCACAATCA | AGG | Human | IL-10 | SpCas9 |

### Adenoviral Vector

The recombinant adenoviral vectors used herein were E1/E3 deleted replication-incompetent human adenovirus serotype 5. Ad-dSaCas9-VPR and single-gRNA vectors were generated by Signagen Laboratories (Rockville, MD). Double-gRNA vectors were generated by SIRION Biotech (Martinsried, Germany). In the process of adenoviral generation, bovine growth hormone polyadenylation signal (bGH polyA) was replaced with Simian virus 40 polyA signal (SV40 polyA). DNA sequences of the cargo in the adenoviral vectors are set forth in SEQ ID NOs: 85 and 86 (for the single-gRNA and double-gRNA containing constructs, respectively). The amplification and purification of all the high-titer adenoviruses used for in vivo studies was performed by SIRION Biotech (Martinsried, Germany). The functional titer of the viruses was measured as infectious units (IU)/ml using a QuickTiter™ Adenovirus Titer Immunoassay Kit (CellBiolab, San Diego, CA).

### Cell Culture

Cells from the L2 cell line (CCL-149, ATCC, Manassas, VA) were cultured in Ham's F-12K (Kaighn's) medium (21127022, Thermo Fisher Scientific, Waltham, MA) supplemented with 10% heat-inactivated fetal bovine serum (FBS; Thermo Fisher Scientific, Waltham, MA), and 100 U/ml penicillin-streptomycin (15140122, Thermo Fisher Scientific). Cells from NR8383 cell line (CRL-2192, ATCC, Manassas, VA) were cultured in Ham's F-12K medium supplemented with 20% heat-inactivated FBS, 2 mM L-glutamine (25030081, Thermo Fisher Scientific), and 100 U/ml penicillin-streptomycin. All the cells were cultured at 37° C. with 5% CO₂.

HEK293 cells (CRL-1573, ATCC, Manassas, VA) used for adenovirus titrations were cultured in DMEM (11995-065, Thermo Fisher Scientific) supplemented with 10% FBS, and penicillin-streptomycin.

For primary cell culture, rat lungs were briefly perfused with PBS supplemented with penicillin, streptomycin, and amphotericin B (15240062, Thermo Fisher Scientific) and retrieved as a heart-lung block. Separated lung tissues were rinsed, cut into small pieces, and digested with collagenase A (10103586001, Sigma-Aldrich, St. Louis, MO) and DNase (D5025, Sigma-Aldrich) for 30 min at 37° C. Following centrifugation and passage through a cell strainer (22-363-548, Fisher Scientific, Hampton, New Hampshire), cells were cultured in RPMI 1640 medium (11875093, Thermo Fisher Scientific) supplemented with 20% heat-inactivated FBS, L-glutamine, MEM non-essential amino acids (11140050, Thermo Fisher Scientific), and penicillin-streptomycin.

### Animals

Male inbred Lewis rats (LEW/SsNHsd, 250-350 g) were purchased from Envigo (Huntingdon, United Kingdom). The animals were kept in a designated animal facility within a pathogen-free environment. Experiments were performed following the Animal Usage Protocol #6057 approved by the Committee of Animal Resources Centre at University Health Network.

### Plasmid Transfection

L2 cells were seeded the day before transfection at a density of 1×10⁵ viable cells/ml in 12 well plates and transfected using Lipofectamine 3000 (Thermo Fisher Scientific) according to the manufacturer's instructions. NR8383 cells were seeded at a density of 2x105 viable cells/ml and transfected using FuGENE™ HD Transfection Reagent (Promega, Madison, WI). A total of 1,000 ng plasmid was used in a 1:1 molar mass ratio for co-transfection, or 1,000 ng of plasmid was used as a single vector. Cells were left untouched after transfection until analysis.

### Viral Transduction In Vitro

Cells were seeded 24 h before viral transduction at a density of 1×10⁵ viable cells/well in 12-well plates or 2×10⁵ viable cells/well in 6-well plates for L2 cells, and the number of NR8383 cells used was doubled. Viruses were diluted in diluent (HEPES 100 mM, MgCl2 20 mM), mixed in the culture medium, and added to the cells. A multiplicity of infection (MOI) dose of 250 was used to transduce L2 cells. At this dose, over 95% of the cells were positive for SaCas9 at 24 h as determined by flow cytometry, with retained viability (data not shown). For transducing NR8383 cells, an MOI of 100 was used. The virus-containing medium was left untouched until the assessment.

### Viral Administration In Vivo

Rats were anesthetized using isoflurane, intubated orotracheally with a 16G catheter (BD382258, BD, Franklin Lakes, NJ), and ventilated with a ventilator (Harvard Apparatus, Holliston, MA) at a tidal volume of 10 ml/g, positive end-expiratory pressure (PEEP) 2 cmH₂O, Fraction of inspired oxygen (FiO₂) 1.0, at a respiratory rate (RR) of 80 breaths per minute in the supine position. The intubation tube was then advanced into the left bronchus, confirmation was achieved by observing the movement of the thoracic cavity. The rat was changed to the left-side down position and 500 μl of diluent (HEPES 100 mM, MgCl₂ 20 mM) with or without viral vectors was administered to the left lung through the intubation tube. After administration, the intubation tube was pulled back into the trachea and the rats were ventilated in the same left-side down position until awakening, which normally took 5-10 min. The rats were maintained with free access to food and water until analysis.

### Immunosuppression Regimen

For transient immunosuppression, cyclosporine (15 mg/kg/day), and azathioprine (6 mg/kg/day), and high-dose steroids (30 mg/kg of methylprednisolone), were intraperitoneally injected 2h before viral vector administration on days 0, 1, and 2. At 2h post-delivery, 30 mg/kg of methylprednisolone alone was intraperitoneally injected to maintain its effects in the acute phase.

In the continued immunosuppression group, triple immunosuppressants (cyclosporine at 15 mg/kg/day, azathioprine at 6 mg/kg/day, and methylprednisolone at 2.5 mg/kg/day) were subcutaneously injected daily following the transient immunosuppression protocol.

Sample Collection from Rats

After anesthetization using isoflurane, rats were intubated and ventilated at a tidal volume of 10 ml/g, PEEP 2 cmH₂O, FiO₂ 1.0, at an RR of 80 breaths per minute for 5 min. Arterial blood was collected from the abdominal aorta for blood gas analysis. Venous blood was collected from the inferior vena cava.

To collect lung tissue samples, the left lung was divided into four portions for formalin fixation, storage in RNAlater (Thermo Fisher Scientific), snap-freezing in liquid nitrogen, and wet-to-dry weight ratio measurement in the sequence from apical to basal part to be used for histological analysis, RNA assay and protein assay.

### Histology

Lung tissue was fixed in 10% formalin for 24-72h, transferred to 70% ethanol, processed in a tissue processor, and embedded in paraffin. Thin sections were cut and stained for hematoxylin and eosin (HE) and Terminal deoxynucleotidyl transferase dUTP nick end labeling (TUNEL) at the Pathology Core of Spatio-temporal Targeting and Amplification of Radiation Response (STTARR, Toronto, ON, Canada) at University Health Network. Bright-field images were obtained using a slide scanner (APERIO CS2, Leica Biosystems, Wetzlar, Germany).

### Immunofluorescence Staining

5 μm-thick paraffin-embedded sections were incubated in a 60° C. oven for 1 h. After deparaffinization, heat-induced epitope retrieval was performed in either 0.01 M citrate buffer pH 6 or EDTA buffer (1 mmol/L EDTA, 0.05% Tween 20) pH 9 using an autoclave. The sections were blocked with serum-free blocking medium (Cat. X0909, DAKO, Hovedstaden, Denmark) for 30 min, then incubated overnight at 4° C. with primary antibodies. After washing with PBS-0.1% Tween-20, sections were incubated with secondary antibodies, followed by washing and counterstained with DAPI. Images were acquired using Yokogawa CSU-10 Spinning-disk confocal microscope or fluorescent scanner (Zeiss AxioScan, Leica Biosystems, Wetzlar, Germany). Antibodies used in this study are listed in Table 3.

TABLE 3
List of antibodies

| | Cat # | Vendor | Host | Dilution |
|---|---|---|---|---|
| Primary antibodies | | | | |
| SaCas9 | ab20393 | abcam | rabbit | 1:500 |
| podoplanin | NB110- | Novus | mouse | 1:300 |
| | 96423 | Biologicals | | |
| CD68 | ab31630 | abcam | mouse | 1:100 |
| CD3 | A0452 | DAKO | rabbit | 1:200 |
| MPO | Ab208670 | abcam | mouse | 1:400 |
| Secondary antibodies | | | | |
| anti-mouse IgG Alexa | A32766 | Invitrogen | donkey | 1:1000 |
| Fluor ™ 488 | | | | |
| anti-rabbit IgG Alexa | A32733 | Invitrogen | goat | 1:1000 |
| Fluor ™ 647 | | | | |
| anti-rabbit IgG Alexa | A32732 | Invitrogen | goat | 1:1000 |
| Fluor ™ 555 | | | | |
| anti-mouse IgG Alexa | A28180 | Invitrogen | goat | 1:1000 |
| Fluor ™ 555 | | | | |
| anti-Rabbit IgG Alexa | A32790 | Invitrogen | donkey | 1:1000 |
| Fluor Plus 488 | | | | |
| anti-Rabbit IgG Alexa | A48289 | Invitrogen | donkey | 1:1000 |
| Fluor Plus 647 | | | | |

### In Situ Hybridization (ISH)

ISH was performed at the Pathology Core of STTARR, UHN (Toronto, ON, Canada).). Rat IL-10 and SaCas9 were stained using RNAscope™ 2.5 HD Duplex Reagent Kit (322430, Advanced Cell Diagnostics, Newark, CA) and RNAscope™ Probes (450481-C2 and 501621, Advanced Cell Diagnostics, Newark, CA) following the manufacturer's instructions. VectaMount Permanent Mounting Media (Vector Laboratories, Burlingame, CA) was used for mounting.

### Wet-to-Dry Weight Ratio

Lung tissues were dried for 72 h at 85° C. in an oven. The wet-to-dry ratio was calculated as the ratio of the weight before and after incubation.

### ELISA

The cell culture supernatant was collected, centrifuged for 1 min at maximum speed in a tabletop centrifuge, and stored at −80° C. until assay analysis. The snap-frozen lung tissue was homogenized in lysis buffer (10 mM HEPES pH7.9, 10 mM KCl, 0.1 mM EDTA, 0.1 mM EGTA, 0.6% Nonidet P-40 with protease inhibitor cocktail) using a tissue homogenizer. The protein concentration in the supernatant was measured using a BCA Protein Assay Kit (23227, Thermo Fisher Scientific) according to the manufacturer's instructions. The ELISA kits used for cytokine detection were as follows: rat IL-10 (BMS629, Thermo Fisher Scientific), rat TNFa (RTA00, R&D Systems, Minneapolis, MN), and rat IL-6 (R6000B, R&D Systems).

### Quantitative PCR

RNA was purified from cell lysate or RNAlater-stabilized lung tissue using the TRIzol (Fisher Scientific, Hampton, New Hampshire) method using the RNeasy Micro Kit (Qiagen, Hilden, Germany) or RNeasy Mini Plus kit (Qiagen), respectively. cDNA was synthesized from 500 ng (cell lysate) or 1000 ng (lung tissue) of RNA using the iScript™ Advanced cDNA Synthesis Kit for RT-qPCR (Bio-Rad, Hercules, CA). cDNA was diluted to 1:6-(cell lysate) or 1:10-(lung tissue), and 7 μl was used for 20 μl of qPCR reaction with SsoAdvanced™ Universal SYBR™ Green Supermix (Bio-Rad) and run on a CFX384 Real-Time System (Bio-Rad). The primers used for RT-qPCR are set forth in SEQ ID NOs: 87-107 and listed in Table 4.

TABLE 4
List of primers used in qPCR

SEQ

ID NO.

Primers used in Gene expression analysis

| | | |
|---|---|---|
| 87 | rat IL10 | CGACGCTGTCATCGATTTCTC |
| | forward | |
| 88 | rat IL10 | TGCCGGGTGGTTCAATTTTTC |
| | reverse | |
| 89 | rat ppia | GACGCCGCTGTCTCTTTTCG |
| | forward | |
| 90 | rat ppia | AAAGTTTTCTGCTGTCTTTGGAACT |
| | reverse | |
| 91 | gRNA1 | ATAGCGGACATTCAACCCAGG |
| | forward | |
| 92 | gRNA4 | ACCGTGTAAGCACAAGAGAAC |
| | forward | |
| 93 | gRNA cassette | CGCCAACAAGTTGACGAGATA |
| | reverse | |
| 94 | SaCas9 | AACTGACCAATCTGAACTCCG |
| | forward | |
| 95 | SaCas9 | TCTGGTTGTCGTTGGTGTG |
| | reverse | |
| 96 | IL1RN forward | GGGGACCTTACAGTCACCTAA |
| 97 | IL1RN reverse | GTTAGTATCCCAGATTCTGAAGGC |

Primers used in transgene DNA quantification

| | | |
|---|---|---|
| 98 | RPLP1 forward | CTAATGTACGATTTCCACCGC |
| 99 | RPLP1 reverse | GCAACCAAACTTATCTTCACACC |
| 94 | SaCas9 | AACTGACCAATCTGAACTCCG |
| | forward | |
| 95 | SaCas9 | TCTGGTTGTCGTTGGTGTG |
| | reverse | |

Primers used in ChIP-qPCR

| | | | |
|---|---|---|---|
| 100 | gRNA1 on- | on-target 1 | CCTGGTCAGCAAGAAATAGCG |
| | target locus | forward | |
| 101 | | on-target 1 | GGTTCCATTCACGCACTCGTA |
| | | reverse | |
| 102 | gRNA4 on- | on-target 2 | TCGTCGGCAGCGTCAAATAGCCGTAATGC |
| | target locus | forward | AGAAGTTC |
| 103 | | on-target 2 | GTCTCGTGGGCTCGGGCAAAGGCTCCTC |
| | | reverse | CCTCTTC |
| 104 | gRNA1 off- | off-target 1 | TCAATGGGAAACACAAAAAGTTTGA |
| | target locus | forward | |
| 105 | | off-target 1 | CCTCTGTTTTTCAAAGCCCTCA |
| | | reverse | |
| 106 | gRNA4 off- | off-target 2 | GCGTAGGACTCACCCTAACG |
| | target locus | forward | |
| 107 | | off-target 2 | CGCAGACAATGGTTGGCATC |
| | | reverse | |

ppia, peptidylprolyl isomerase A; RPLP1, ribosomal protein lateral stalk subunit P1.

For quantification of transgene DNA, lung tissues were homogenized and subjected to DNA purification using QIAamp DNA Mini Kit (Qiagen). 150 ng of purified DNA was applied in a 10 μl reaction. Real-time quantitative PCR was run using the same protocol as our gene expression analysis.

The sequences of cDNAs encoding the human, rat and pig IL-10 gene are set forth in SEQ ID NOs: 108, 109 and 110, respectively.

### RNA-Seq Library Preparation and Sequencing

Library preparation and RNA sequencing were performed at the Center for Applied Genomics at the Hospital for Sick Children (Toronto, ON, Canada). The library was generated using the NEB Ultra II Directional polyA mRNA library prep (New England Biolabs, Ipswich, MA). Deep sequencing was performed on a NovaSeq 6000 System using SP flow cell (Illumina, San Diego, CA), producing 150-bp paired-end reads.

### RNA-Seq Alignment and Quantification

Rat genome build 6 sequences and gene annotations were downloaded from Ensembl Genome Browser Build 104.⁵⁸ The reference files were indexed using Salmon v1.4.0 using the decoy index option.⁵⁹ The paired-end fastq from each sample was quantified using Salmon with default parameters. The quantification data was merged using tximport v1.2.0⁶⁰ and differential expression was performed using DESeq2 using the recommended workflow v1.3.2.⁶¹

Off-target sites with up to five mismatches were computationally predicted using Cas-OFFinder. High-risk off-target genes were selected as loci that contained an exact 6-bp match in the region neighboring PAM. Genes overlapping the region 500 bp upstream and downstream of high-risk off-target sites were identified using bedtools Intersect intervals in Galaxy.

### ChIP-qPCR

Chromatin immunoprecipitation was performed using the EZ-Magna ChIP™ A/G Chromatin Immunoprecipitation Kit (Sigma-Aldrich, St. Louis, MO) according to the manufacturer's instructions with minor modifications. Briefly, a total of 8 million primary rat lung fibroblasts were seeded and transduced with adenoviral vectors after 1 h at MOI of 10. After 48 h of viral infections, cells were cross-linked with 1% formalin for 10 min. Formalin was quenched with 0.125 M glycine. Fixed cells were rinsed and collected into PBS containing EDTA-free protease inhibitor (Roche, Basel, Switzerland). Cells were lysed by vortex, centrifuged, and resuspended in SDS lysis buffer containing EDTA-free protease inhibitor. Chromatin was sheared using a probe sonicator (VC130PB-1 with S&M 0704 probe, Qsonica, Newtown, CT) in 5 cycles of the 30s ON and 60s OFF at an amplitude of 40. Samples were diluted to 1:10 in dilution buffer to make the SDS level down to 0.1%. 50 μl of the diluted samples were stored as “input”. Magnetic beads were washed and bound with 5 μl of anti-SaCas9 antibody (C15310260, Diagenode, Denville, NJ) were bound to 50 μl A/G beads in 0.5% BSA in PBS overnight at 4° C. Cross-linked and sheered protein/DNA was bound to antibody-beads complex overnight at 4° C. After elution of antibody/protein/DNA complex from beads by heating with intermittent vortex in SDS containing buffer, reverse cross-linking was performed by incubation with proteinase K at 65° C. overnight followed by 95° C. for 10 min. Free DNA was purified using a MinElute PCR purification kit (Qiagen) and eluted into 25 μl water. ChIP-qPCR was performed using SsoAdvanced™ Universal SYBR™ Green Supermix (Bio-Rad) and CFX384 Real-Time System (Bio-Rad). 0.4 μl of purified DNA was used in a 10 μl reaction. Ct values were normalized to 1% input and enrichments were calculated as % input.

### Statistics

Data were analyzed using Prism (GraphPad Software, San Diego, CA). A two-tailed unpaired Student's t-test was used in in vitro experiments. For in vivo experiments, the Mann-Whitney test or Kruskal-Wallis followed by Dunn's correction was used to compare two groups and multiple groups, respectively. Two-way ANOVA followed by Bonferroni's correction was applied to analyze experimental groups at different time points. Statistical significance was set at P<0.05.

### Example 2: Transcriptional Activation Using dSaCas9-VPR Robustly Enhanced Endogenous IL-10 Expression In Vitro

To identify a CRISPR-Cas based approach suitable for in vivo gene therapy, two systems were tested with distinct mechanisms of action, dSaCas9-VPR and dSpCas9-P300. The former (VPR) induces transcriptional activation of the target gene by recruiting nuclease-deficient Staphylococcus aureus Cas9 fused with a tripartite transcriptional activation effector, comprising VP64 derived from herpes simplex virus, p65 from human NF-κB, and Rta from Epstein-Barr virus. The latter (P300) enhances gene expression by histone modification using nuclease-deficient Streptococcus pyogenes Cas9 fused with the p300 core domain of human histone acetyltransferase.

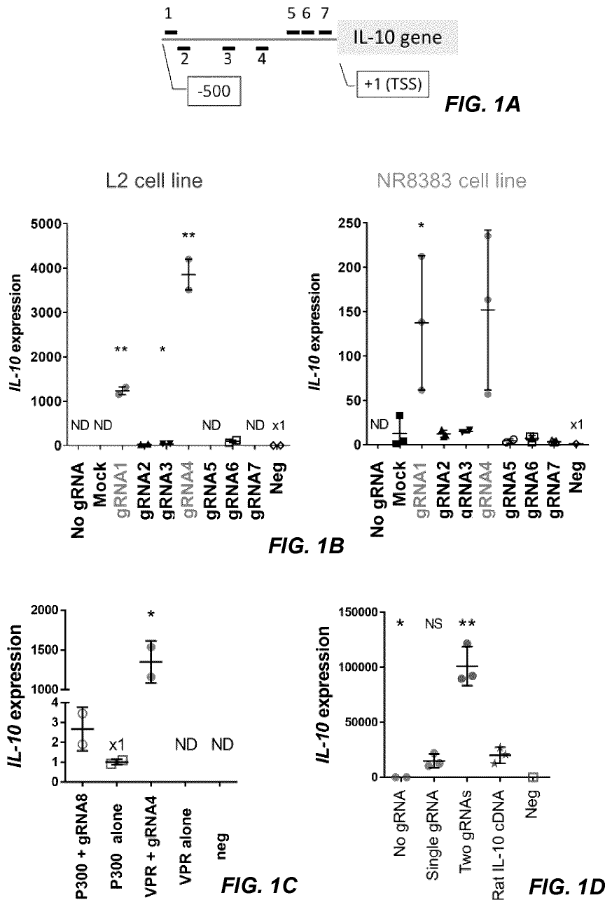

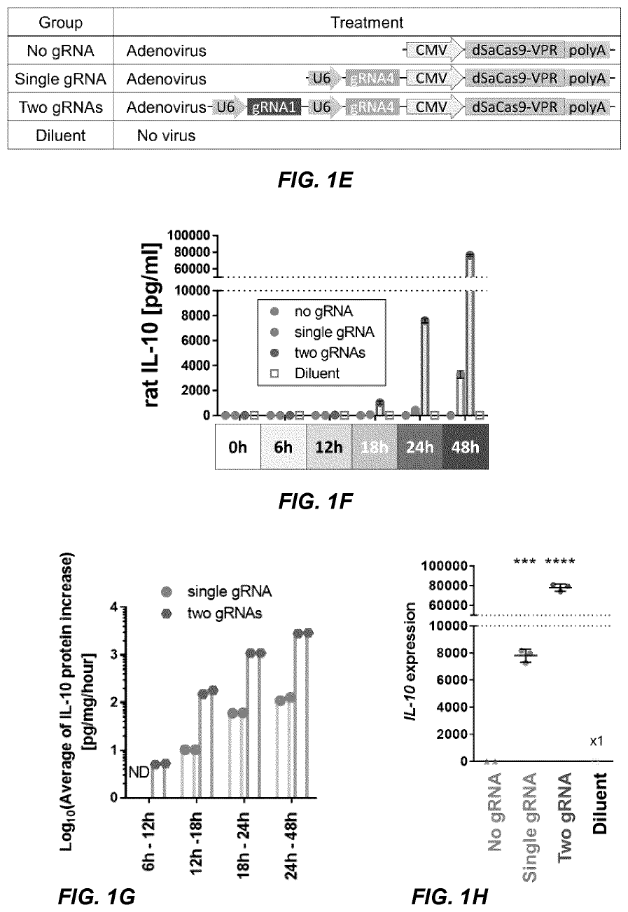

In vitro screening of gRNAs, that were designed to bind to the region upstream of IL-10 in the rat genome (FIG. 1A), revealed that dSaCas9-VPR strongly induced IL-10 expression. In a rat lung epithelial cell line (L2 cells), gRNA1 and gRNA4 (comprising the nucleotide sequences set forth in SEQ ID NOs: 1 and 4, respectively) upregulated IL-10 by 1,233-fold and 3,854-fold, respectively, relative to the negative control (FIG. 1B, left panel). Direct comparison of both systems demonstrated that when the most effective gRNA was used, dSaCas9-VPR induced a 500-fold higher IL-10 expression than dSpCas9-P300 (FIG. 1C, p=0.0189). To examine whether target IL-10 activation is effective in immune cells, a rat lung macrophage cell line (NR8383 cells) was used. The dSaCas9-VPR machinery also enhanced IL-10 expression in NR8383 cells (FIG. 1B, right panel, 151.9+89.9-fold increase compared to negative control).

These findings show that transcriptional activation using dCas9-VPR can enhance IL-10 expression in both immune and non-immune cells. Therefore, dSaCas9-VPR was selected for subsequent studies because of its potent transcriptional activation in lung-relevant cells in vitro.

### Example 3: Combination of Two gRNAs More Strongly Activated IL-10 than cDNA Delivery In Vitro

A comparison of dSaCas9-VPR-mediated target gene activation with the traditional method of IL-10 cDNA delivery into cells using in vitro plasmid transfection was done. This option is possible with IL-10, as rat IL-10 consists of 178 amino acids and has been effectively used interchangeably as human IL-10 cDNA delivery in multiple studies.^{9,10,12,13,17,35-40} The sequences of human IL-10 cDNA and rat IL-10 cDNA are set forth in SEQ ID NOs: 108 and 109, respectively.

For transcriptional activation by dSaCas9-VPR, two vectors with different putative activation potential were prepared encoding single-or dual-gRNA expression cassettes (FIG. 1E). The nucleotide sequences of the single-gRNA (gRNA 4) and the dual-gRNA (gRNAs 1 and 4) containing inserts for adenoviral vectors are set forth in SEQ ID NOs: 85 and 86, respectively.

A combination of multiple gRNAs targeting the same gene synergistically enhance the targeted gene activation.^{23.41} Employing this unique attribute, the two-gRNA group was designed to elicit more potent IL-10 activation than the single-gRNA group. A comparison of equimolar quantities of the plasmids revealed that IL-10 expression in the two-gRNA group significantly surpassed that of cDNA delivery driven by the RNA polymerase II cytomegalovirus (CMV) promoter (FIG. 1D, p=0.0019), while the single-gRNA group induced comparable IL-10 expression to that induced by cDNA delivery. Integration of the third gRNA prompted an early increase of IL-10 protein compared to the two gRNA group (FIG. 9).

These findings show that transcriptional activation can induce IL-10 expression significantly more potently than that attained with cDNA delivery, and its activation can be adjusted at the cellular level by selecting and combining gRNAs.

### Example 4: Adenovirus-Mediated dSaCas9-VPR and gRNA Delivery Increased Secreted IL-10 Protein In Vitro

To deliver Cas9 activator and gRNA to rat lungs in vivo, recombinant adenoviruses expressing dSaCas9-VPR and either one or two functional gRNAs was generated (FIG. 1E). These treatments were compared with the no-gRNA group, which received adenovirus expressing dSaCas9-VPR (Ad-dSaCas9-VPR) alone, and the diluent group, which received buffer without any virus. The adenoviral vector was selected because of its rapid and efficient transgene expression in the lungs. Furthermore, its large packaging capacity (7.5 kb) can sufficiently contain multiple gRNA cassettes and dSaCas9-VPR in an all-in-one vector.

The IL-10 protein concentration in the cell culture supernatant of transduced L2 cells increased in a time-dependent manner in both the single- and two-gRNA groups (FIG. 1F). For both gRNA groups, the IL-10 increase rates were accelerated, and the kinetics were similar (FIG. 1G). After 48 h, IL-10 transcript levels were 7,793-fold and 77,963-fold higher in the single- and two-gRNA groups, respectively, than in the diluent group (FIG. 1H; p=0.0002 and <0.0001 compared to no gRNA group, respectively). These results demonstrate a time-related and accelerated increase in IL-10 protein production of transduced cells after treatment.

### Example 5: Enriched dSaCas9-VPR Binding to On-Target Loci Induced Specific IL-10 Activation

To further confirm that IL-10 activation was induced by the Cas9 activator, the binding of dSaCas9-VPR to on-target loci in vitro was evaluated using rat primary lung fibroblasts transduced with adenoviral vectors. These cells were selected as they have the same genetic and epigenetic information as those tested in the in vivo study.

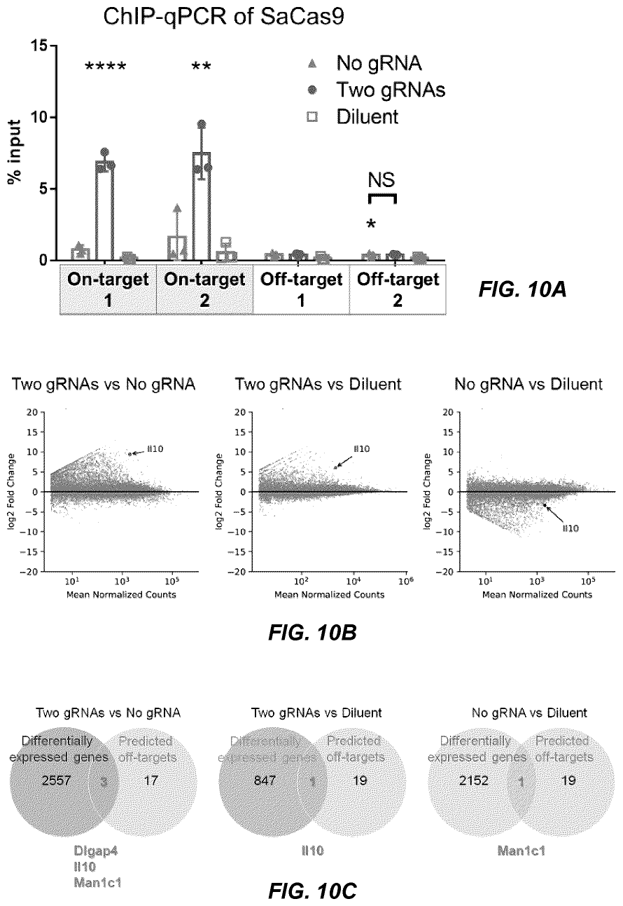

Chromatin immunoprecipitation (ChIP)-qPCR detected enriched dSaCas9-VPR binding to both on-target genomic loci in the two-gRNAs group compared with the no gRNA and diluent buffer groups (FIG. 10A). The treatment group showed no significant enrichment of Cas9 binding to the two potential off-target loci, which were identified using CRISPOR tool,⁴² relative to the no gRNA group. These results indicate that IL-10 upregulation is attributed to dSaCas9-VPR recruitment to the target genomic regions.

Off-target gene activation was investigated by analyzing the whole transcriptome using RNA sequencing. A large number of genes showed significantly different gene expression in the no gRNA and two gRNAs groups compared to the diluent control (no gRNA vs diluent: 634 up and 1,510 down; two gRNAs vs diluent: 773 up and 66 down; and two gRNA vs no gRNA: 1,972 up and 575 down). IL-10 was significantly upregulated in the two gRNAs groups compared to the no-gRNA and diluent groups (log 2 fold change of 6.00 and 9.39 respectively, Padj<0.01 DE-Seq2, FIG. 10B). Computational predictions using≤5 mismatches identified 72 and 181 potential off-target binding sites for gRNA1 and gRNA4, respectively. 19 high-risk off-target sites were analyzed that contained an exact match to 6-bp seeds and localized within the region between-500 bp and +500 bp of the genes in the rat genome. IL-10 was the only gene that was differentially expressed between the two gRNAs and diluent groups and overlapped with the selected off-target genes (FIG. 10C). Comparison of the two gRNAs and no gRNA groups identified IL-10, Dlgap4, and Man1c1 (FIG. 10C). Man1c1 expression was altered in the no gRNA group compared to the diluent group, and the potential off-target region localized 107,460 bp downstream of the transcription start site (TSS) of this gene. The putative off-target locus associated with Dlgap4 was within the first intron and 784 bp downstream of the TSS. As the off-target loci did not differ between the two gRNA groups and the diluent group, off-target activation or its adverse effects would be improbable.

Overall, the results support that dSaCas9-VPR binding to the target genomic loci induced highly specific IL-10 activation.

### Example 6: Trans-Airway Delivery of Ad-dSaCas9-VPR Evoked Inflammation in the Lung

For in vivo profiling, the immunogenicity of Ad-dSaCas9-VPR, which is identified as a potential hurdle in treating the lung was explored. The post-delivery inflammation induced by Ad-dSaCas9-VPR in the lung has not been previously described; thus, an appropriate management strategy is undefined.

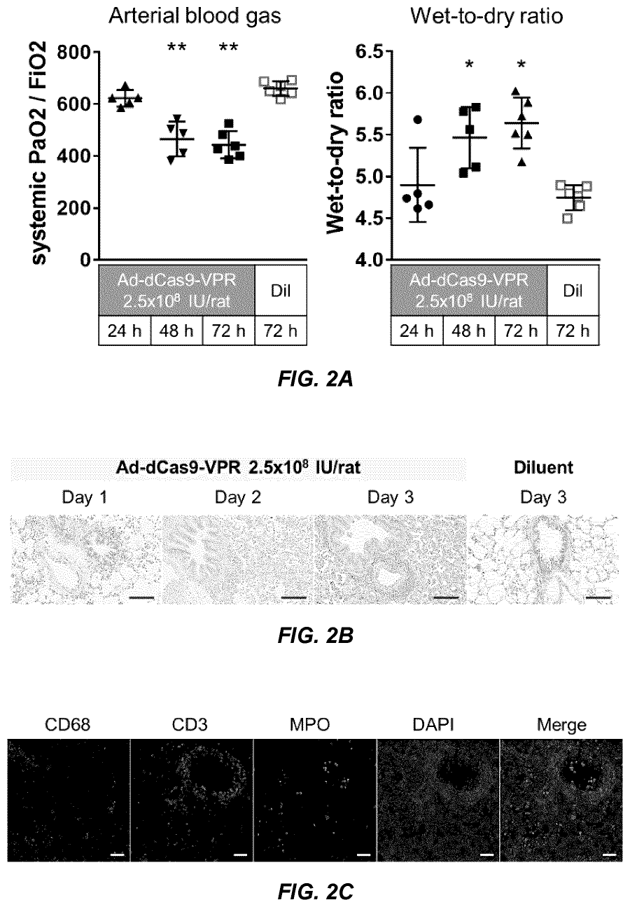

To evaluate pulmonary inflammation, Ad-dSaCas9-VPR was delivered to the left rat lung through the airway. Delivery of Ad-dCas9-VPR at high doses (2.5×10⁸ IU/rat) evoked a strong immune response in the lungs. Viral vector-treated lungs presented with progressive worsening of oxygenation and lung edema—as measured by the increased wet-to-dry weight ratio (W/D weight ratio)-which became evident after 48 h (FIGS. 2A and B). The Ad-dSaCas9-VPR group showed a significant decrease in systemic PaO₂ (443±53 vs. 660±28 mmHg; p<0.0001; FIG. 2A) and an increase in the W/D weight ratio (5.64±0.31 vs. 4.75±0.15; p=0.01; FIG. 2A) compared with the diluent group at 72 h. Viral vector delivery also exacerbated inflammatory cell infiltration in the lung tissue in a time- and dose-dependent manner (FIG. 2B). After 72 h, the high-dose cohort showed inflammatory cell infiltration spread to the alveolar space accompanying TUNEL-positive apoptotic cells (FIG. 2B). Immunofluorescence (IF) demonstrated the accumulation of infiltrating CD3-positive lymphocytes in the perivascular region, as well as CD68-positive macrophages and myeloperoxidase (MPO)-positive neutrophils in the alveolar region (FIG. 2C).

These findings demonstrated that the delivery of Ad-dSaCas9-VPR caused considerable inflammation in the lungs. The infiltration of various immune cells confirms that mitigation of the immune response is required to manage this post-delivery inflammatory response.

### Example 7: Immunosuppression Circumvented Post-Delivery Inflammation and Enhanced dSaCas9-VPR Expression in the Lung

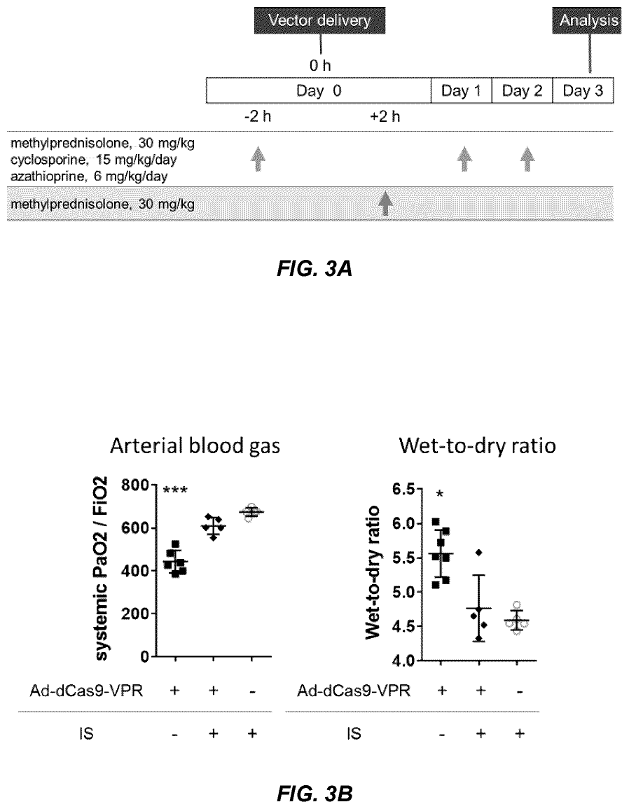

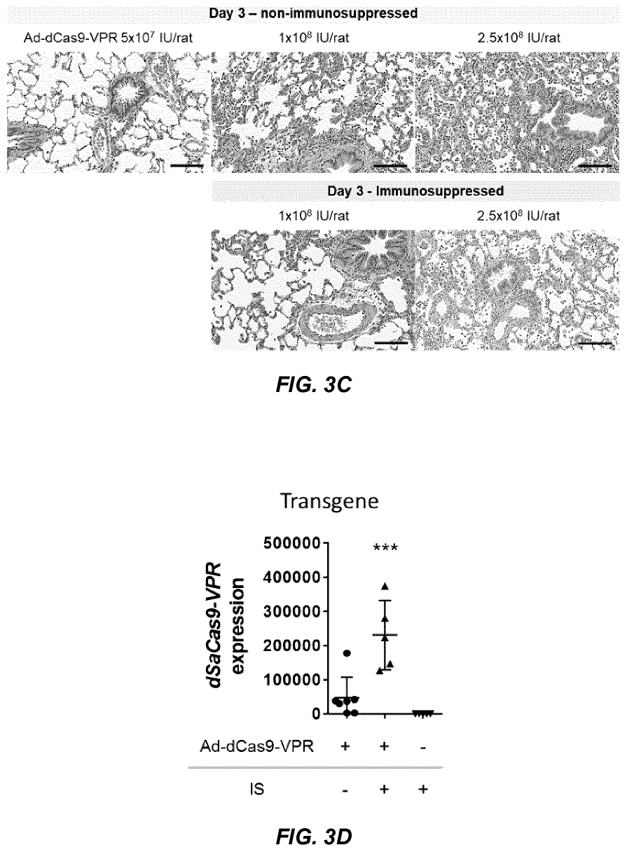

It was shown in a rat model that the anti-rejection immunosuppression combination used in clinical solid organ transplantation effectively inhibited the adenoviral vector-induced inflammation in the lung.^{8,43,18} Building on these observations, this example tested whether transplant immunosuppression could mitigate the inflammation associated with Ad-dSaCas9-VPR, thus facilitating the safe delivery of CRISPR-Cas machineries for clinical applicability. Triple immunosuppression, consisting of methylprednisolone, cyclosporine, and azathioprine, was administered before and after the delivery of high-dose Ad-dSaCas9-VPR (FIG. 3A). It indeed effectively circumvented inflammation 72h after high-dose Ad-dSaCas9-VPR delivery to the lung. The immunosuppressed group showed improved oxygenation and decreased lung edema compared to the non-immunosuppressed cohort (FIG. 3B). This was supported by reduced inflammatory cell infiltration in the alveolar space (FIG. 3C). Immunosuppression also increased the dSaCas9-VPR expression level 4.9-fold compared to that in the non-immunosuppressed group (FIG. 3D), suggesting that the immune response affects transgene expression in the lung.

These results illustrate that immunosuppression medications used in clinical solid organ transplantation can be used to advantage for the efficient and safe application of Ad-dSaCas9-VPR in the lung. Therefore, this triple immunosuppression was used in the subsequent experiments.

### Example 8: Adenoviral Delivery of dSaCas9-VPR and Two gRNAs Dose-Dependently Upregulated IL-10 Expression in the Lung

To evaluate targeted IL-10 activation in vivo in the lung, the two-gRNA adenoviral vector was delivered to the rat left lung at a lower dose (5×10⁷ IU/rat), a medium dose (1×10⁸ IU/rat), and a higher dose (2.5×10⁸ IU/rat). The diluent group, which received buffer without a viral vector, served as a control for assessing lung function and inflammatory status after intervention.

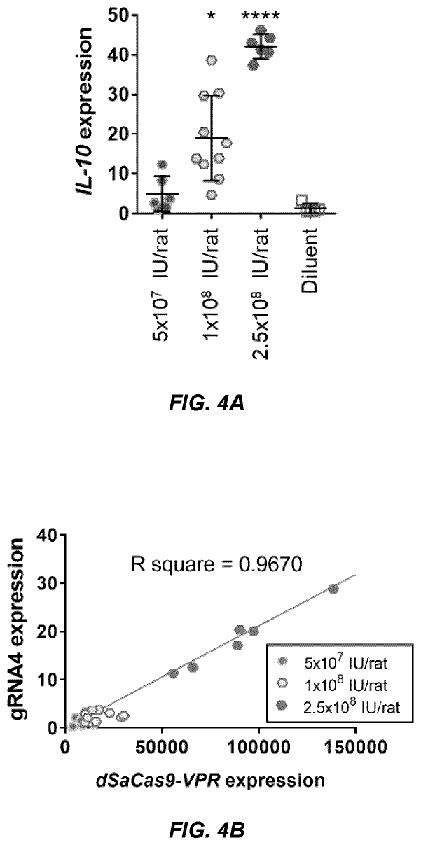

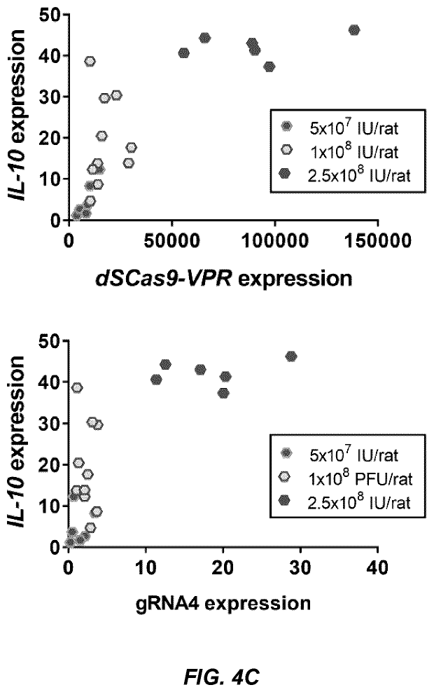

The delivery of two-gRNAs adenoviral vectors upregulated IL-10 expression in the lung in a dose-dependent manner at 3 days compared with the diluent group (Spearman's correlation; r=0.8925; p<0.0001; FIG. 4A). The medium- and higher-dose groups showed 19-fold and 42-fold higher IL-10 expression, respectively (p=0.0105 and <0.0001 compared to the diluent group). The expression levels of dSaCas9-VPR and gRNA showed a linear relationship (R²=0.9670 between dSaCas9-VPR and gRNA4; FIG. 4B). On the other hand, IL-10 expression leveled off in the high-dose cohort (FIG. 4C), suggesting an upper limit of IL-10 expression.

### Example 9: Targeted Transcriptional Activation Elevated IL-10 Protein Levels to the Therapeutic Window with Minimal Inflammation

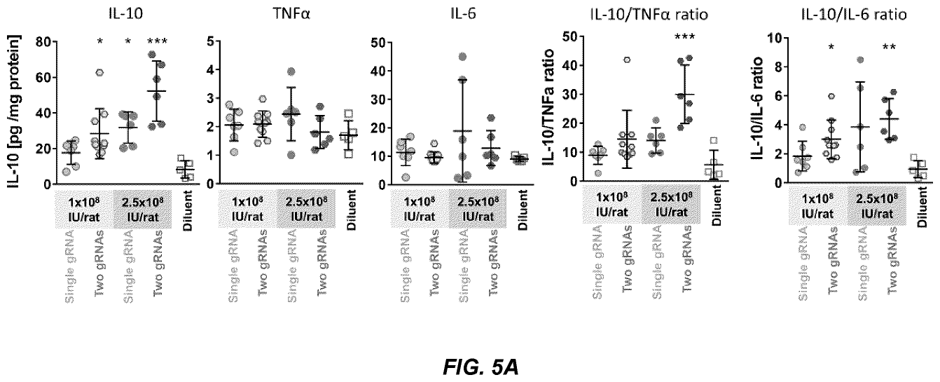

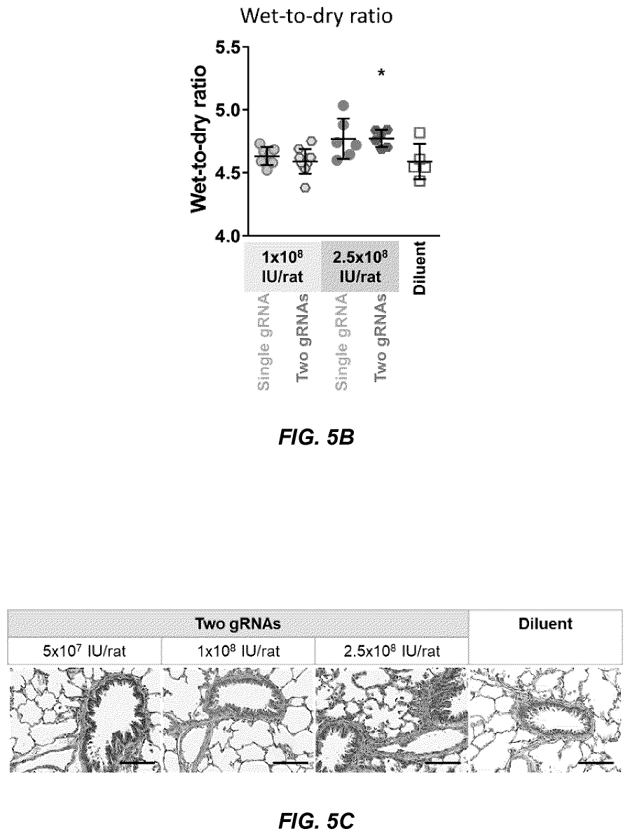

IL-10 protein concentration in the lung tissue was measured to determine whether this novel approach could induce sufficient IL-10 levels to achieve anti-inflammatory effects. Treatment with the two-gRNAs vector at medium and higher doses significantly elevated IL-10 protein levels in the lung tissue relative to the diluent group (FIG. 5A; p=0.0244 and 0.0001, respectively). In the single gRNA group, the IL-10 increase was significant only at the higher dose (FIG. 5A; p=0.0129). At medium and higher doses, the two-gRNAs treatment resulted in 28.4±14.0 pg/mg and 52.3±16.9 pg/mg IL-10 protein in the lung tissue, respectively. Both protein levels fell within the target therapeutic window, which may exert potent anti-inflammatory effects to inhibit post-transplant lung injury induced by ischemia-reperfusion.^{7,8,38}

Compared with the diluent group, there were no significant increases in the levels of IL-6 and TNF-α protein, which represent pro-inflammatory cytokines, in viral vector-treated lungs at either dose (FIG. 5A). Hence, the IL-10/IL-6 or IL-10/TNFα ratios, which reflect IL-10 increases induced by the intervention, were higher in the two gRNAs groups than in the diluent group (FIG. 5A).

The cohort treated with a higher dose of the two gRNA vectors presented with localized inflammation and a slight increase in the W/D weight ratio (FIGS. 5B and 5C). These indicate that a certain extent of vector-related inflammation remains at higher-dose even with the use of immunosuppression.

These findings suggest that adenoviral delivery of dSaCas9-VPR and two gRNAs effectively elevated IL-10 protein levels in the lung with the use of immunosuppression. Among the three doses tested, the medium dose (1×10⁸ IU/rat) appeared optimal to induce sufficient IL-10 production with minimal inflammation.

### Example 10: Transduced Cells were Localized Mainly in the Alveolar Space

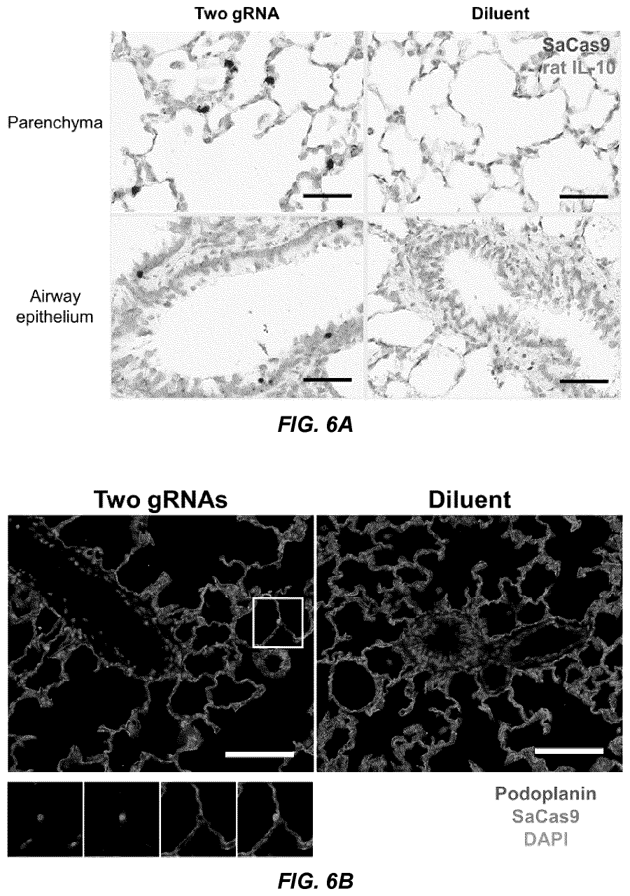

In situ hybridization (ISH) revealed that most dSaCas9-VPR-expressing cells were localized in the alveolar region, and few of them were also detected in the airway epithelium (FIG. 6A). Image analysis confirmed that approximately 1% of the total cells showed strong SaCas9 staining. Cas9-expressing cells also showed IL-10 mRNA staining, suggesting their contribution to the total IL-10 protein increase in the lungs. Double immunofluorescence staining with cell-specific markers revealed that a certain population of transduced cells in the alveolar space was positive for podoplanin, a marker for alveolar type 1 cells (FIG. 6B).

### Example 11: Targeted IL-10 Activation Declined by Day 14 Under Transient Immunosuppression

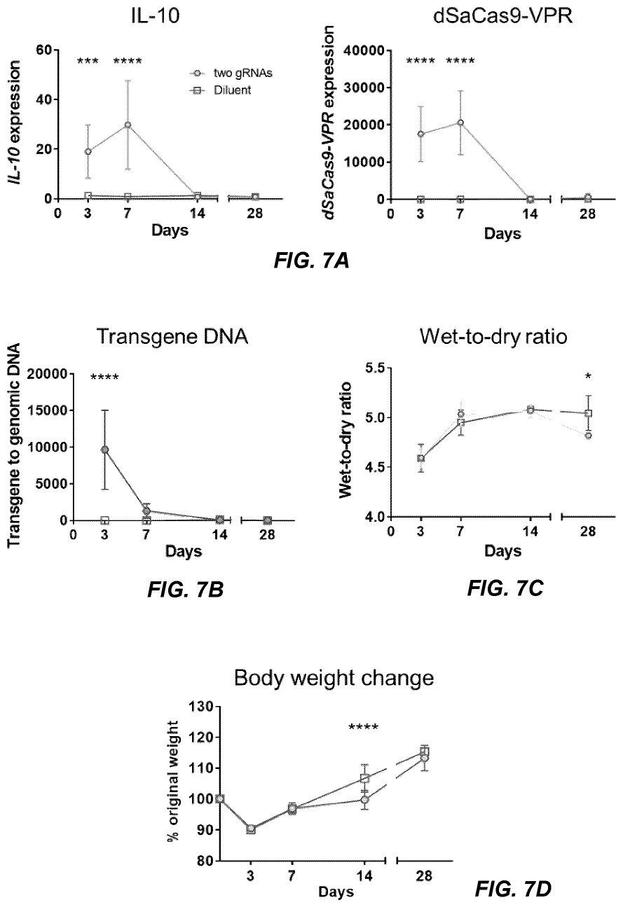

Time course analysis using transient immunosuppression showed that IL-10 upregulation was sustained for 7 days but declined by day 14 (FIG. 7A). Similar temporal changes were observed in the dSaCas9-VPR expression (FIG. 7A). The transgene DNA levels rapidly dropped by day 7 (FIG. 7B), possibly due to the degradation of viral vector DNA in the extracellular space or in the cytosol. Compared with the diluent group, the virus-treated lungs presented comparable or lower W/D weight ratios over 4 weeks (FIG. 7C). Relative to the control, body weight gain was slightly slower in the vector-treated cohort at day 14, but fully recovered by day 28 (FIG. 7D). Notably, localized inflammatory cell infiltration was observed around the airways and vessels of the two gRNA group on days 14 and 28, although it was not severe. These results suggest that immunosuppression withdrawal reactivated the immune response towards viral vectors and caused the loss of transduced cells in the lungs.

### Example 12: Continuous Immunosuppression Sustained IL-10 Activation in the Lung

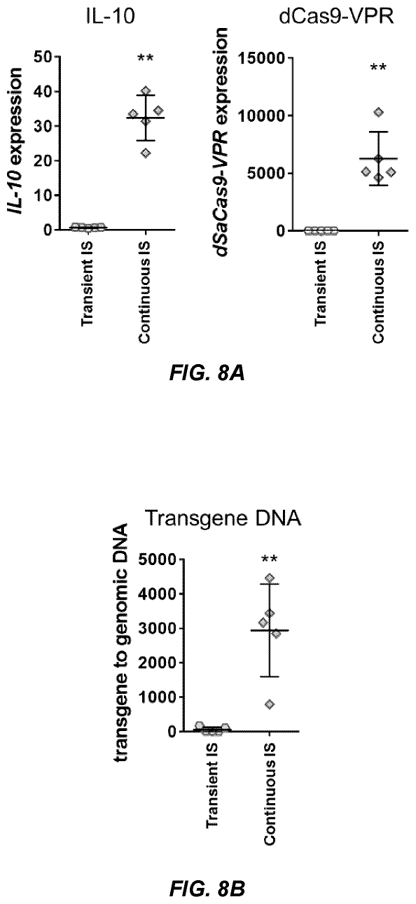

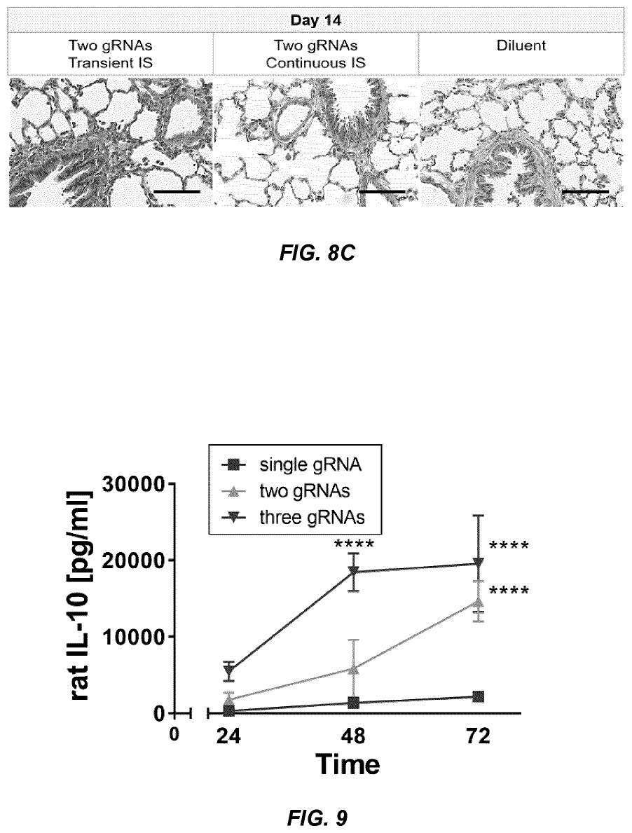

To examine the impact of continued immunosuppression on IL-10 activation, triple immunosuppression was maintained until analysis on day 14. Following the transient immunosuppression protocol, the corticosteroid doses were lowered, while the other two drugs were maintained at the same dose. This regimen kept the cyclosporine concentration in the plasma at 2,964±485 ng/ml on day 14. Under continued immunosuppression, IL-10 expression was sustained for 14 days (FIG. 8A), as were the dSaCas9-VPR expression and transgene DNA (FIGS. 8A and 8B). Histologically, the inflammatory cell infiltration that remained with transient immunosuppression mostly disappeared in vector-treated lungs under daily immunosuppression on day 14 (FIG. 8C). Persistent IL-10 activation indicates that immunosuppression prevents transduced cells from being cleared by host immunity.

### Example 13: Single Adenoviral Vector-Mediated CRISPR-Cas Transcriptional Activation Facilitated Multiplexed Cytokine Modulation In Vitro

The potential of multiplexed cytokine modulation via this approach using single adenoviral vectors was explored. Given that the adenoviral vectors could hold four separate gRNA cassettes driven by an individual promoter along with dSaCas9-VPR, it was hypothesized that potent multiplex cytokine modulation could be achieved using all-in-one adenoviral vectors. To explore this feasibility, two distinct gRNAs targeting interleukin 1 receptor antagonist (IL1RN) were additionally integrated to the two gRNA vectors to express a total of four different gRNAs from single adenoviral vectors. IL1RN is a naturally occurring protein that binds to the interleukin 1 receptor and blocks inflammatory signaling of both IL-1a and IL-1B. Therefore, simultaneous activation of IL1RN could reinforce the anti-inflammatory effect of IL-10.

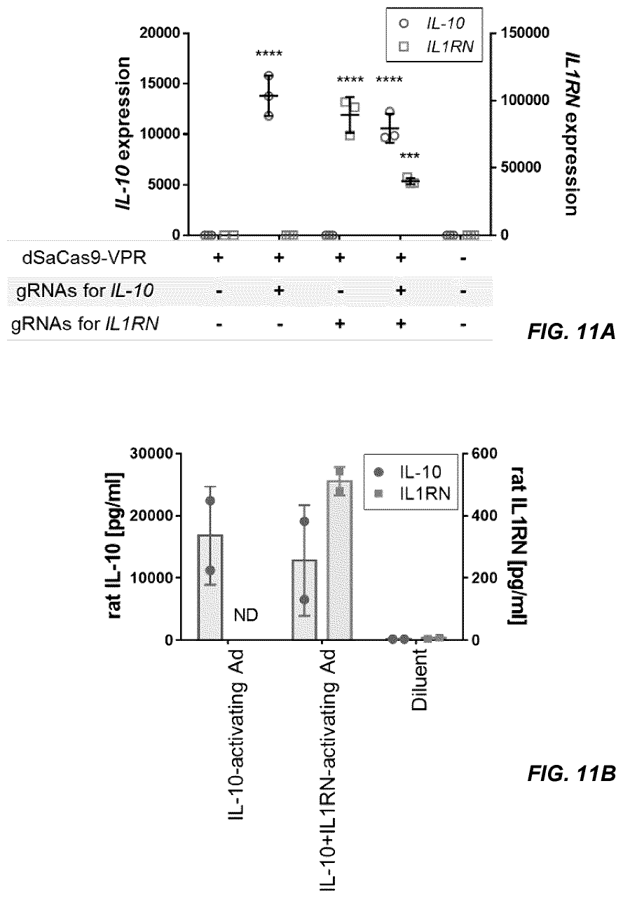

In vitro assessment using plasmid vectors demonstrated simultaneous activation of IL-10 and IL1RN expression as designed. Expression of four gRNAs upregulated both genes, while the delivery of two gRNAs targeting the same gene led to single gene activation (FIG. 11A). Compared to the single gene activation, the multiplexed activation showed retained IL-10 expression level, while IL1RN expression was approximately half of that in IL1RN single activation.

To assess the practicality of multiplexed cytokine modulation using an all-in-one adenoviral vector, adenoviral vectors for multiplexed cytokine modulation (IL-10+IL1RN-activating Ad) were generated and their functionality was evaluated in vitro. The results revealed an increase in both IL-10 and IL1RN cytokines in the supernatant when cells were treated with the multiplexed all-in-one adenoviral vectors (FIG. 11B).

These findings demonstrate the feasibility of expressing four distinct gRNAs from a single adenoviral vector and modulating multiple cytokines via targeted transcriptional activation.

### Example 14: Derepression of the IL-10 Gene Via CRISPR-Cas Genome Editing for Persistent Immunomodulation of Donor Lungs for Transplantation

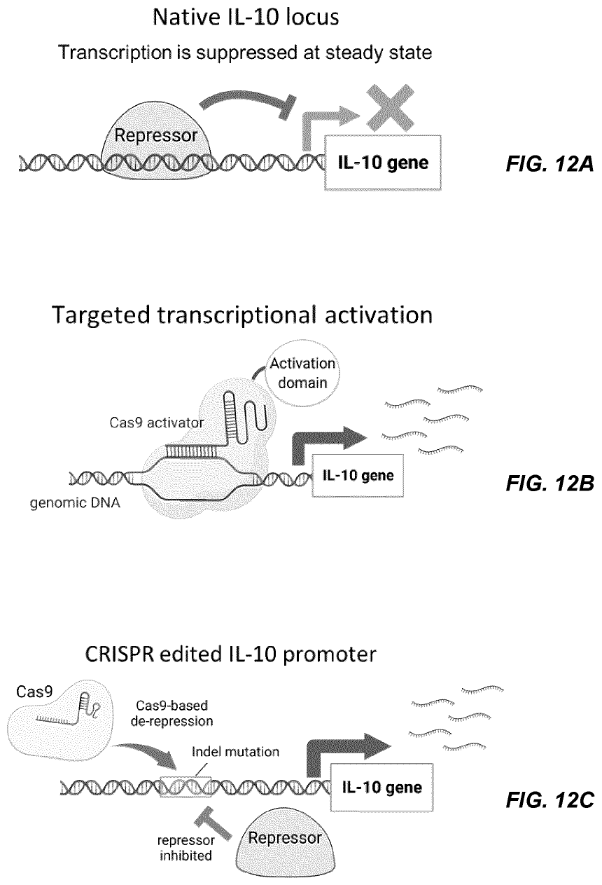

As illustrated in FIG. 12A, the binding of a repressor to the regulatory region of the IL-10 gene leads to suppression of IL-10 transcription in the absence of external stimuli. For targeted transcriptional activation, a nuclease-deficient Cas activator (e.g., dSaCas9-VPR) is recruited to the genetic locus upstream of the IL-10 gene by designed gRNAs and enhances IL-10 gene expression (FIG. 12B). In other embodiments, a wild-type Cas nuclease (e.g., Cas9) is used to introduce mutations (such as insertions and/or deletions, i.e., indels) into the regulatory region of the IL-10 gene that derepress IL-10 gene and increase IL-10 transcription (FIG. 12C).

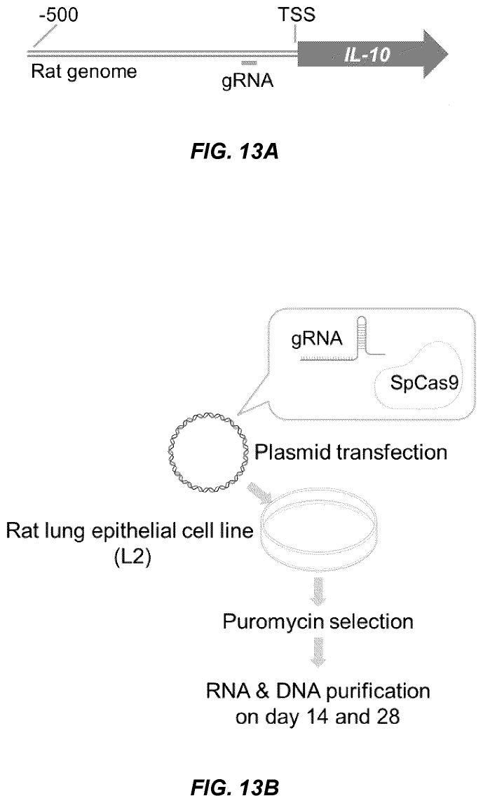

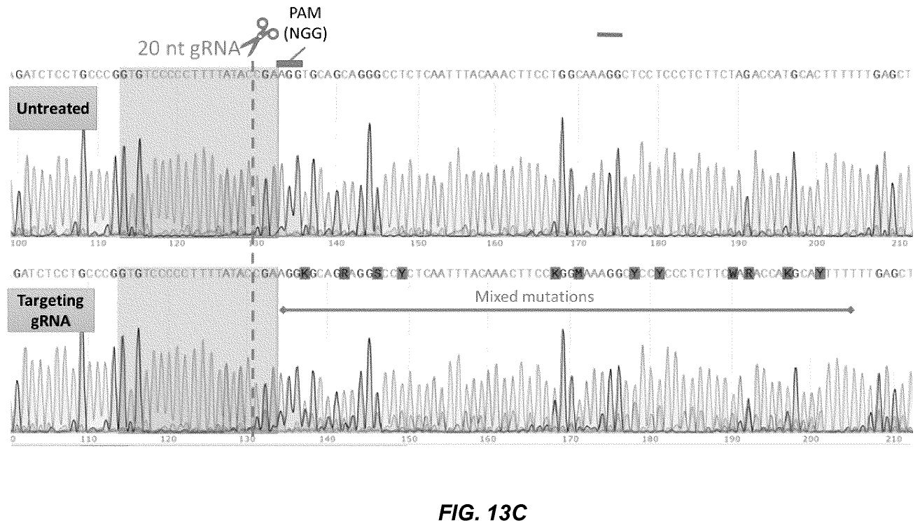

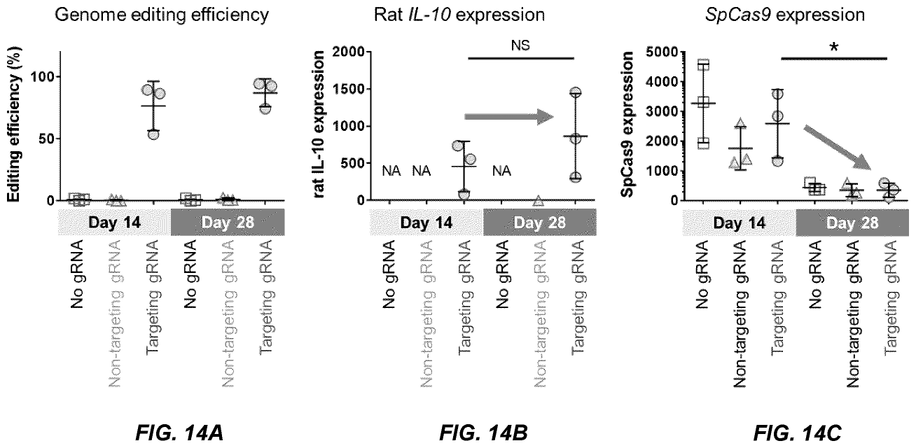

To assess derepression of the IL-10 gene, gRNA 75 (comprising the nucleotide sequence set forth in SEQ ID NO: 30) was designed to bind the genomic region upstream of the IL-10 gene in the rat genome (FIG. 13A). SpCas9 and gRNA 75 (encoded on a single adenoviral vector) were transfected into L2 cells, followed by incubation under puromycin selection for a period of 14 or 28 days (FIG. 13B). After the incubation, genomic DNA was extracted from the L2 cells. Mutagenesis around the cleavage locus in the treatment group was demonstrated by targeted Sanger sequencing of the genomic DNA. The sequences encompassing the target region of gRNA 75 in the untreated (control) group and the gRNA-treated group are set forth in SEQ ID NOs: 80 and 81, respectively, and shown in FIG. 13C.

Computational analysis of Sanger sequencing showed genome editing efficiency of around 80% in the treatment group on both day 14 and 28 (FIG. 14A). Analysis of gene expressions showed an over 400-fold increase in the endogenous rat IL-10 expression in the editing group, which is shown as targeting gRNA group, on day 14 (FIG. 14B). The IL-10 upregulation in the editing group lasted until day 28, despite a decline in Cas9 expression (FIG. 14C).

Insertions/deletions at the target site were detected in 77±20% of alleles at day 14 and 87±11% at day 28 in the treatment group, which showed a 457±338 and an 865±573-fold increase in rat IL-10 expression at day 14 and 28, respectively, while other groups remained mostly undetectable over time. Importantly, IL-10 upregulation lasted for 28 days despite a decline in SpCas9 expression, demonstrating persistent expression after one round of genome editing.

### Example 15: Derepression of the IL-10 Gene Via Genome Editing Across Different Species

### Introduction

Optimizing donor organs by genome editing is a promising approach to expand the donor pool and improve outcomes in lung transplantation. In particular, persistent immunomodulation in donor lung leveraging genome editing holds promise in obviating or reducing the need for systemic immunosuppression after lung transplantation. Towards realizing this ultimate therapeutic vision, an efficient and clinically applicable genome editing strategy to upregulate IL-10, an immunomodulatory cytokine, was explored. Given that IL-10 expression is physiologically suppressed at a steady state, it was hypothesized that genetic disruption of the regulatory region of IL-10 could induce persistent upregulation. To prove this concept, the efficacy of this genome editing approach was evaluated in vitro using cell lines that originated from representative animal species which are used in the translational path.

### Methods

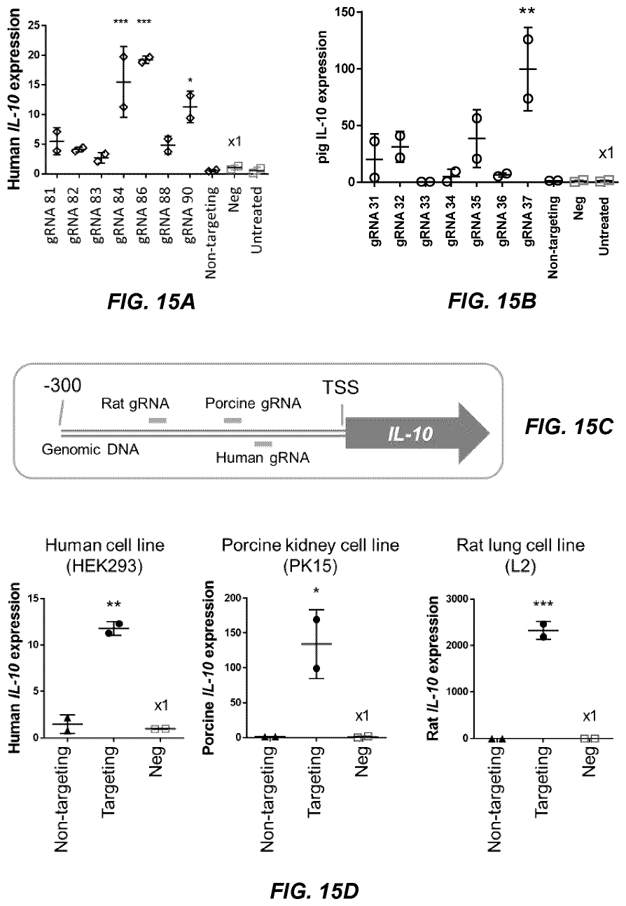

A series of gRNAs were designed to bind to a locus between 1-500 bp upstream of the IL-10 gene in the human, porcine and rat genomes, and screened for their activities of IL-10 upregulation (FIGS. 15A and 15B). Staphylococcus aureus Cas9 nuclease (SaCas9) and a gRNA were delivered to the human kidney cell line (HEK293), porcine kidney cell line (PK15), and rat lung epithelial cell line (L2) by plasmid transfection (FIG. 15C). The expression of the endogenous IL-10 gene after 48 hours of transfection was measured by qPCR.

### Results

Screening of five to seven gRNAs designed for each species identified one specific gRNA that effectively upregulates the IL-10 gene. Mutating the cis-regulatory region of the IL-10 gene using SaCas9 nuclease and the selected gRNA significantly enhanced the expression of endogenous IL-10 gene in cell lines derived from different species; genome editing increased an 11.8±0.73-fold increase (p=0.0013) in HEK293 cells, a 134.3±49.7-fold increase (p=0.038) in PK15 cells, and a 2329±194.8-fold increase (p=0.0005) in L2 cells, compared to the negative control (FIG. 15D).

### Conclusion

Upregulation of IL-10 gene through simple mutagenesis at the regulatory region was achieved. The potential mechanism underlying this finding is derepression of the native IL-10 gene that could be conserved among species tested. These findings make donor organ immunoregulatory optimization possible, with clinical practicality, thus bringing this innovation to the bedside in organ transplantation.

### Example 16: Precise Mutagenesis by Base Editors Upregulated IL-10 Expression

Since the mutations generated by wild type Cas nuclease vary between cells, upregulation of IL-10 gene expression by generating specific mutations by base editors was evaluated as a step forward.

Base editing is one of the innovative genome editing tools developed from original form of CRISPR-Cas system. By fusing deaminase domain to mutated Cas9 protein, one can make a single base change in the DNA without generating double stranded breaks. Thus, base editing allows more accurate and efficient genome editing.

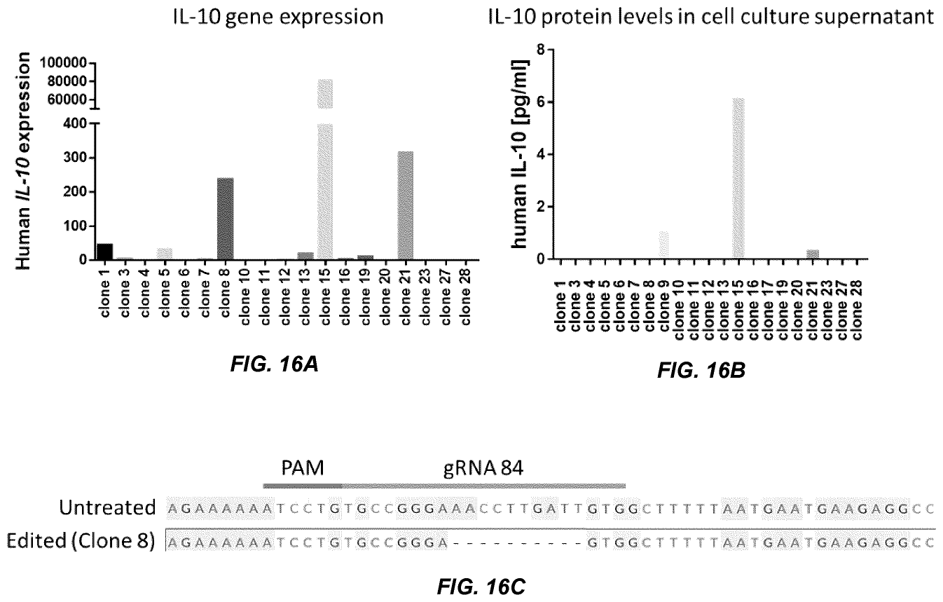

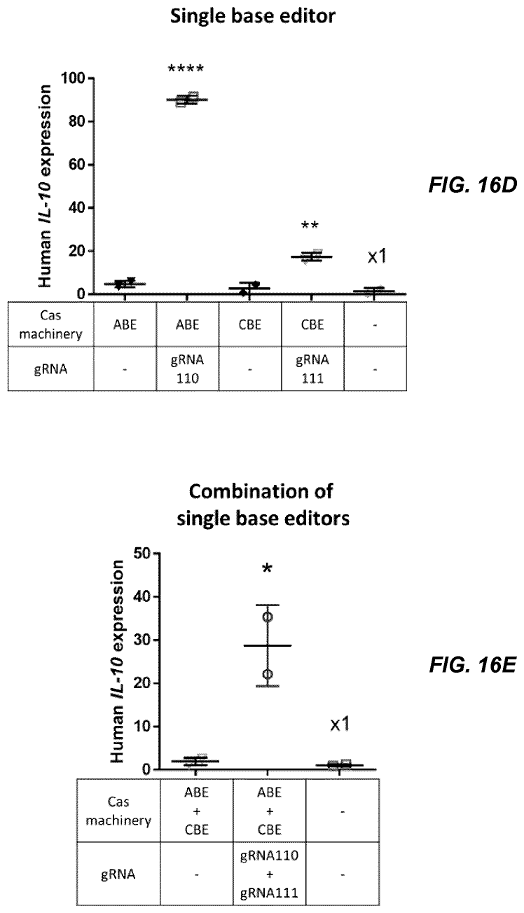

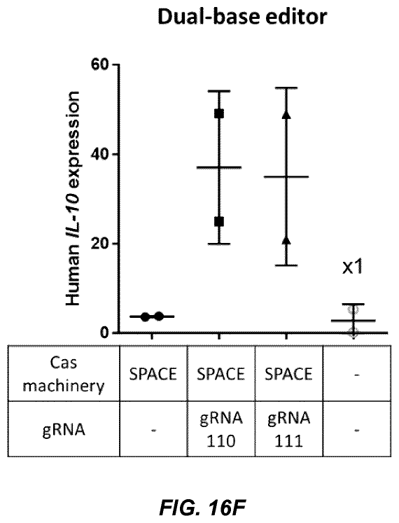

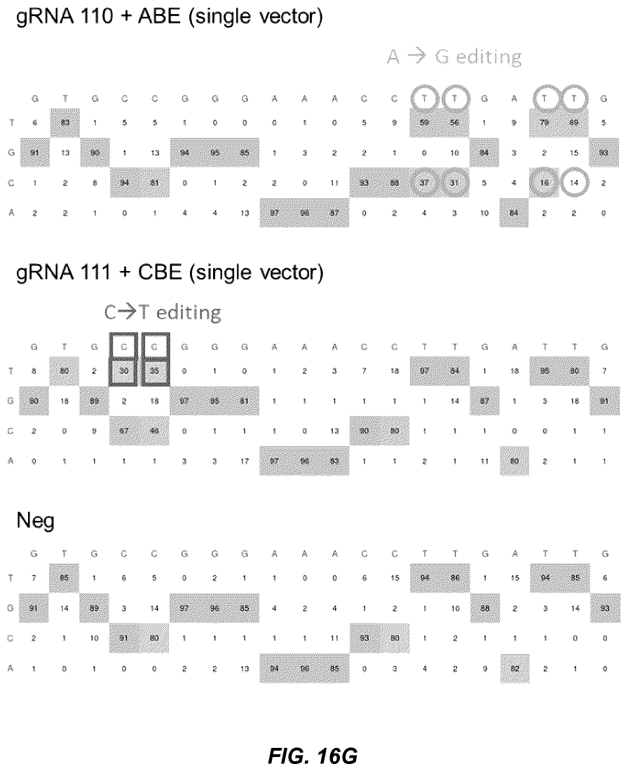

Three types of base editors were used: adenine base editor (ABE), cytosine base editor (CBE), and synchronous programmable adenine and cytosine editor (SPACE). Using single cell cloning, the genetic region which potentially contributes to the IL-10 upregulation was narrowed down. Then, gRNAs for base editors to disrupt the region were rationally designed (FIG. 16A-C).

Delivery of base editors and gRNAs significantly enhanced IL-10 expression (FIG. 16D-F). This strategy is an efficient approach for genome editing in organs and tissues.

### Example 17: Temporal Tuning of Inflammatory and/or Immunomodulatory Gene Expression Using a Combination of Derepression Gene Editing and Exogenous cDNA Delivery

### Introduction

Optimizing donor organs by transcriptome manipulation is a promising approach to expand the donor pool and improve outcomes in lung transplantation. In particular, tuning anti-inflammatory and immunomodulatory gene expression using a CRISPR-Cas system to prevent both early inflammatory lung injuries and late allo-rejection was envisioned. The goal was to engineer optimal expression kinetics of the IL-10 gene, which encodes this anti-inflammatory and immunomodulatory cytokine. The overall aim was to achieve rapid and high early expression followed by moderate persistent IL-10 expression in donor lungs. Genome editing is a powerful approach to induce persistent change in gene expression, however, the time required to exert this effect is a challenge to implement in organs prior to implantation. The practically available period of time for preparing donor organs using our ex vivo lung perfusion system is currently limited to around 12 hours.

It was hypothesized that genome editing combined with cDNA delivery (gene editing-transfer) could realize the objective of temporal tuning of IL-10. To prove this concept, simultaneous delivery of genome editing machineries, which can derepress the endogenous IL-10 gene, and exogenous human IL-10 cDNA was tested in vitro.

### Methods

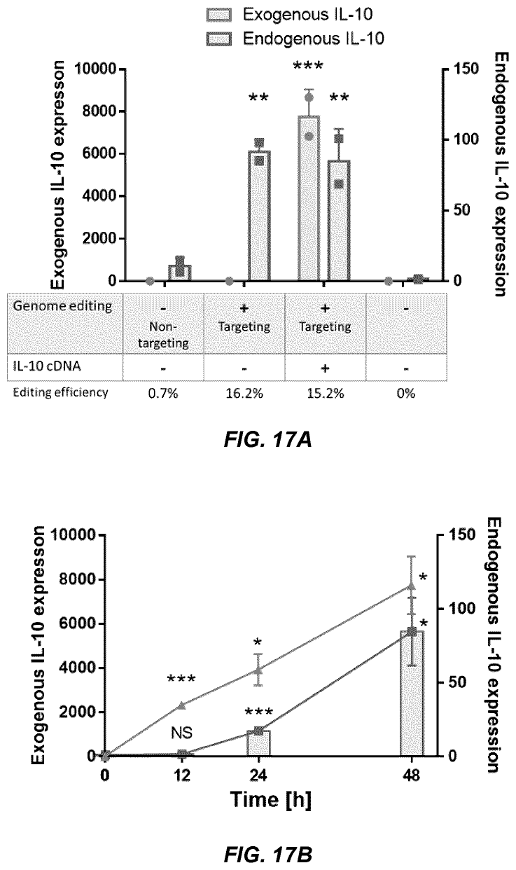

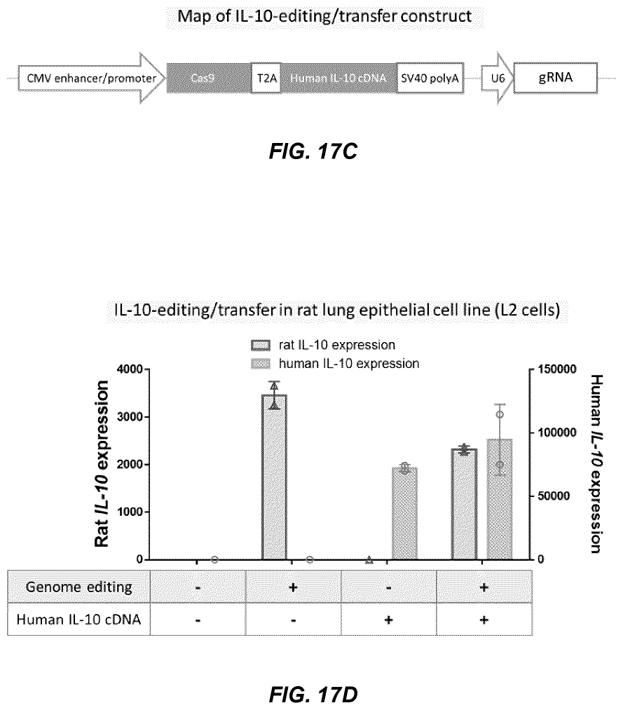

SaCas9 nuclease and a gRNA that binds to a locus around 80-bp upstream of the human IL-10 gene (gRNA 84, comprising the spacer sequence set forth in SEQ ID NO: 36) were delivered to HEK293 cells with or without the sequence of human IL-10 cDNA, which is set forth in SEQ ID NO: 108. To make IL-10 gene editing-transfer machineries loaded to a single adenoviral vector, human IL-10 cDNA was driven by the same promoter as SaCas9 using a 2A peptide sequence (FIG. 17C). The time course of endogenous and exogenous IL-10 expression was assessed until 48 hours after plasmid transfection.

This IL-10 gene editing-transfer approach was also tested in a rat lung epithelial cell line (L2). SpCas9 and a gRNA that targets the rat genome (gRNA4) were delivered by plasmid transfection with or without human IL-10 cDNA. Treated cells were assessed for gene expression and genome editing after 48 hours.

### Results

Mutating the promoter region using SaCas9 nuclease and the gRNA significantly enhanced the expression of endogenous IL-10 gene. The genome editing alone group showed 16.2±0.8% editing efficiency and a 91.4±9.1-fold increase (p=0.0063) compared to the negative control at 48 hours (FIG. 17A). High expressions of exogenous IL-10 gene were detected only in groups with IL-10 cDNA delivery. The IL-10 gene editing-transfer group demonstrated a significant increase in both endogenous and exogenous IL-10 expression (84.6±23-fold increase, p=0.0085, and a 7739±1304-fold increase p=0.0009, respectively) (FIG. 17A). Kinetics of the IL-10 gene editing-transfer group showed enhanced exogenous IL-10 expression as early as 12h accompanied by delayed upregulation of endogenous IL-10 (FIG. 17B).

The evaluation in L2 rat cells demonstrated enhanced expression of both endogenous rat IL-10 and exogenous human IL-10 in the IL-10 gene editing-transfer group (FIG. 17D) that is consistent with the observations in HEK293 cells.

### Conclusion

Optimized kinetics of IL-10 expression were achieved in vitro by combining derepression genome editing and cDNA delivery. These findings make donor organ immunoregulatory optimization possible, with clinical practicality, thus bringing this innovation to the bedside in organ transplantation.

### Example 18: Transplantation of Transcriptionally Modulated Donor Lungs

The feasibility of implanting gene-activated donor lungs in transplant settings was evaluated. Rat single lung transplant non-injury models were used to assess if the gene-activated donor lungs were (1) safely transplanted to iso- and allo-recipients for proper assessment, (2) maintaining enhanced IL-10 expression in recipients, and (3) devoid of worsening of vector-related inflammation after transplantation.

Two-gRNA adenoviral vectors or diluent were delivered to the left lung of donor rats through the airway 24 hours before transplant surgery to simulate pre-implantation donor lung modulation. The left lung graft was retrieved from a donor rat, transplanted into a recipient rat, and analyzed on postoperative day 3 (POD3), which is a timing of diagnosing primary graft dysfunction in clinical lung transplantations. Triple immunosuppression using with (cyclosporine at 15 mg/kg/day, azathioprine at 6 mg/kg/day, and methylprednisolone at 2.5 mg/kg/day) was applied until the analysis.

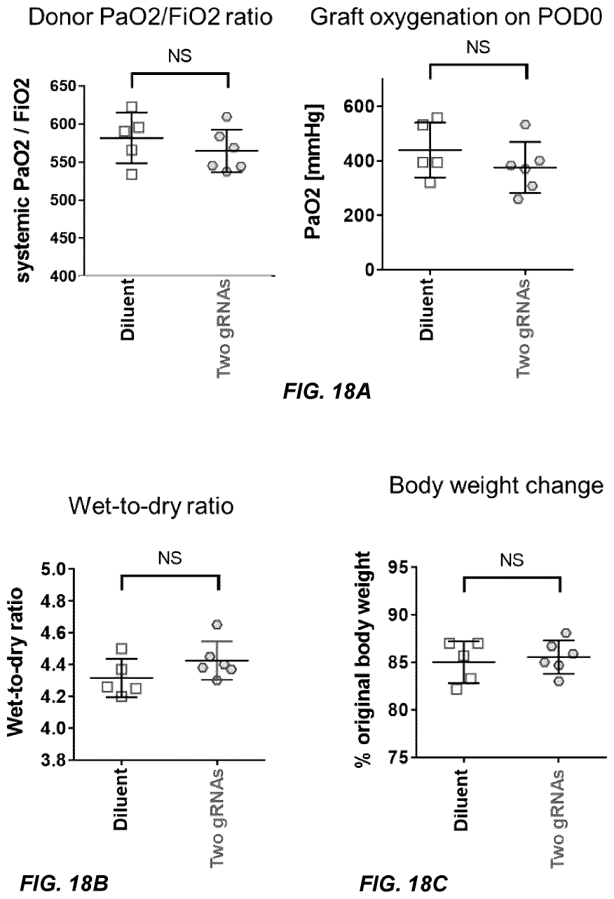

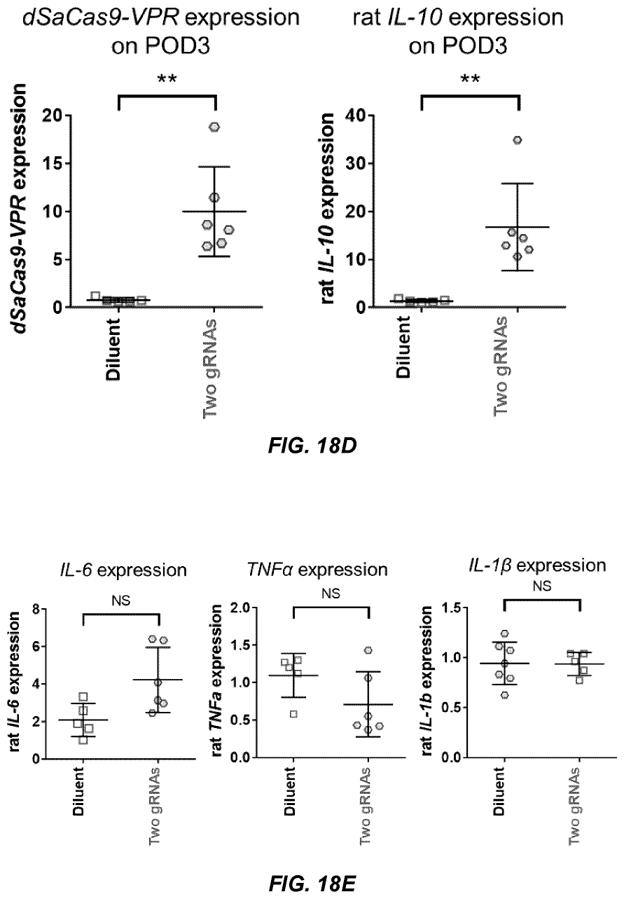

Evaluation in syngeneic transplantation, in which donor lungs from Lewis rats were transplanted to Lewis rats, demonstrated comparable donor PaO₂/FiO₂ ratio (564.8±28 vs. 581.7±33 mmHg; p=0.54; FIG. 18A) and graft oxygenation at the time of reperfusion in the IL-10 activated lungs compared to the diluent group (375.5±94 vs. 439.7±101 mmHg; p=0.33; FIG. 18A). The IL-10-activated lung grafts showed comparable W/D weight ratio to those of diluent control (4.43±0.12 vs. 4.32±0.12; p=0.14; FIG. 18B). Recipients in both groups showed similar body weight changes from postoperative day 0 to day 3 (85.6±1.8 vs. 85.0±2.2% of original weight on POD3; p=0.89; FIG. 18C). Gene expression analyses showed higher transgene (10.00±4.67 vs. 0.78±0.24-fold compared to untreated cases; p=0.0043; FIG. 18D) and IL-10 expressions (16.75±9.06 vs. 1.36±0.30-fold compared to untreated cases; p=0.0043; FIG. 18D) in the IL-10-activated and transplanted lung grafts after 3 days. Inflammatory gene expressions, which that are represented by IL-6, TNFα, and IL-1B, were not significantly increased at the transcriptional level in the IL-10-activated grafts compared to the diluent control (FIG. 18E).

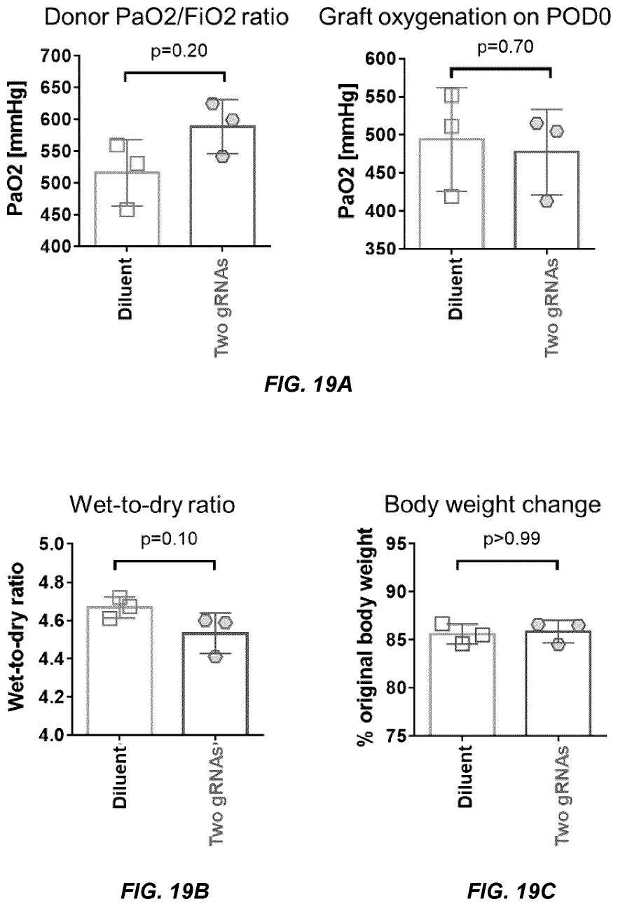

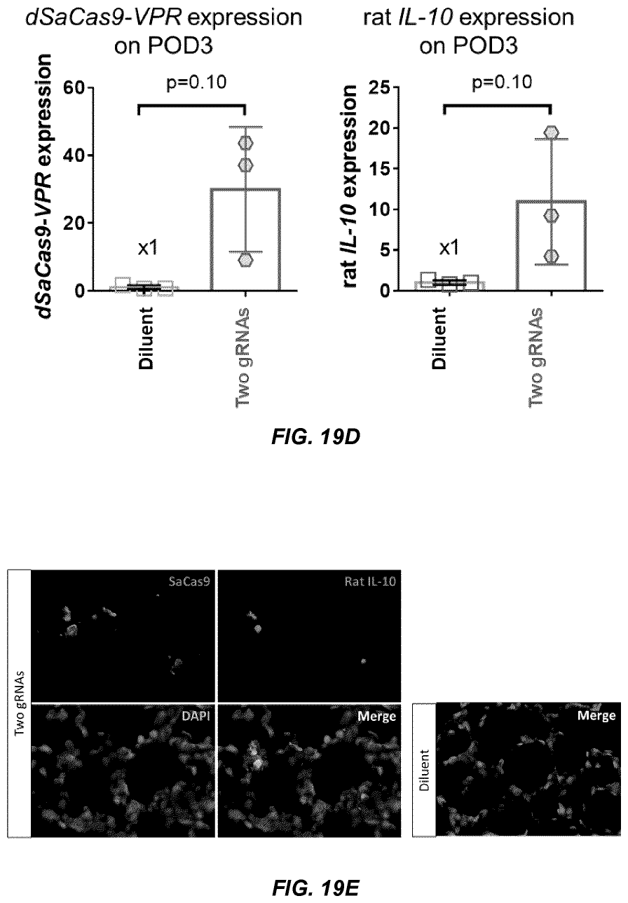

Next, it was examined whether IL-10-activated lung grafts were acceptable in an allogeneic transplant model. Transplant models using mismatched strain combinations permit the evaluation of the impact of interventions on allo-immune responses and have served as an important tool in transplant research. Using the same experimental methods, donor lungs from Brown Norway rats were treated using the same experimental methods, followed by transplantation into Lewis rats. Similar to syngeneic transplantation, the two gRNA group showed comparable donor PaO₂/FiO₂ ratios, graft oxygenation on POD0, W/D weight ratios, and recipient body weight changes to the diluent group (FIG. 19A to 19C). Sustained dSaCas9-VPR expression and IL-10 upregulation was detected, although they were not statistically significant, presumably because of the small sample size (FIG. 19D). Fluorescent in situ hybridization showed cells positive for both SaCas9 and IL-10 staining (FIG. 19E), supporting retained IL-10 activation in the graft after implantation in allorecipients.

Overall, these findings demonstrate that gene-activated donor lungs can be safely transplanted, maintain targeted gene activation in recipients, and do not exhibit signs of worsening of graft conditions after transplantation compared to the diluent control. These results show that a broad range of transcriptional modulations can be assessed in transplant models using both syngeneic and allogeneic strain combinations.

### Example 19: Human Organs for Transplantation

Organs can be taken from any donor as is known to those skilled in the art, including donors without a heartbeat, for example Maastricht category III and/or Maastricht category IV. The organ can be healthy or injured. An injured organ is one that is deemed sub-optimal or too high risk to be successfully transplanted into a human. For example, an injured organ may have been indicated as such because of the presence of pneumonia, edema, or inflammation. Also, the injured organ may have an injury such as a disruption of the blood-alveolar barrier, a severe injury, such as a mechanical injury, or an injury that is difficult to determine, such as a non-specific injury. In addition, or in other cases, the injured organ may have sub-optimal physiologic criteria, such as sub-optimal PaO₂/FIO₂, pulmonary vascular resistance, peak inspiratory pressure, or dynamic compliance. Categorization of injured organ can occur as part of standard evaluation, or as a separate step once donor organ has been determined to be an injured organ. Categories of injury of injured organ include, but are not limited to, severe injury, pneumonia, edema, inflammation, and non-specific injury. Generally, any organ that is considered “high risk” for transplantation into a human can fall under a category of injury.

Each injured organ can be treated, with the intent to repair, according to the category of injury it falls. It will be recognized by the skilled person that injured organ may fall under more than one category and may therefore benefit from a mixture of treatments. Ideally, every injured organ can undergo ex vivo organ perfusion before or concurrently with treatment. An ex vivo perfusion system is known to those skilled in the art, see for example, U.S. patent application Ser. No. 14/658,456, which is incorporated herein by reference.

Treatment of injured organ can involve any suitable treatment. For example, if injured organ falls under category severe injury, it can be treated with suitable stem cells or a de-cellularization and re-cellularization regimen, or mixtures thereof, according to methods known to those with skill in the art. If injured organ falls under category pneumonia, it can be treated with antibiotics, steroids, alveolar lavage, or mixtures thereof, according to methods known to those with skill in the art. If injured organ falls under category edema, it can be treated with antibiotics, hyperperfusion techniques, hyper-osmotic agents, beta-agonists, or flow techniques, where, for example, optimal perfusion flow is about 40% of estimated cardiac output of the donor, or mixtures thereof, according to methods known to those with skill in the art. If injured organ falls under category inflammation, it can be treated with anti-inflammatory agent, gene therapy, stem cell therapy, anti-coagulation therapy, or mixtures thereof, according to methods known to those with skill in the art. If injured organ falls under category non-specific injury, it can be evaluated by techniques known to the skilled person to determine the best treatment options.

Transplantable organs can also benefit from being subjected to an EVOP system (described, for example, in U.S. patent application Ser. No. 14/658,456). Such a system can preserve and maintain transplantable organs in order to ensure that they are in optimum condition for transplantation into a recipient. The EVOP system can also improve transplantable organs to make them even better candidates for transplantation.

The transplantable organ can also undergo gene therapy as described herein and/or endogenous genetic or epigenetic modulation of gene expression as described herein. For example, the cassettes or vectors provided herein (comprising a polynucleotide that encodes gRNA(s), Cas enzymes or derivatives thereof, and optionally, cDNA(s) encoding gene(s)-of-interest (along with promoter and termination signal) can be delivered through the airway using a bronchoscope or through circuit by adding to the perfusate during EVOP to suppress inflammation by modulating the expression of one or more inflammation-regulating and/or immune-regulating genes, such as interleukin-10 (IL-10), interleukin-1 receptor antagonist (IL1RN), Interleukin-4 (IL-4), Interleukin-6 (IL-6), Interleukin-8 (IL-8), tumor necrosis factor binding protein (TNFBP), cytotoxic T-lymphocyte associated protein 4 (CTLA4), NFKB inhibitor alpha (Ikba), Programmed cell death-1 (PD-1), Programmed cell death-ligand 1 (PD-L1), Interleukin-10 receptor, CD47, CD200, Fas Ligand (FasL), MHC molecule (MHC-Ib) H2-M3, Serine protease inhibitor 6 (Spi6), C-C Motif Chemokine Ligand 21 (Ccl21) and Milk Fat Globule-EGF Factor 8 Protein (Mfge8).

Additionally, organs with persistent or permanent immunomodulatory capacity can be transplanted into recipients. The methods provided herein for reducing inflammation by modifying the expression of inflammation-regulating and/or immune-regulating genes may be used to replace systemic immunosuppression regimens after organ transplantation.

Numerous specific details are set forth in order to provide a thorough understanding of the exemplary aspects of the present application. However, it will be understood by those of ordinary skilled in the art that the exemplary aspects described herein may be practiced without these specific details. In other instances, well-known methods, procedures and components have not been described in detail so as not to obscure the exemplary aspects described herein. Also, the description is not to be considered as limiting the scope of the exemplary aspects described herein. Any systems, method steps, method blocks, components, parts of components, and the like described herein in the singular are to be interpreted as also including a description of such systems, method steps or tasks, components, parts of components, and the like in the plural, and vice versa.

Persons of skill will appreciate that there are yet more alternative implementations and modifications possible, and that the above examples are only illustrations of one or more implementations. The scope, therefore, is only to be limited by the claims appended hereto.

The disclosures of all documents cited herein are incorporated herein by reference as if set forth in their entirety.

### REFERENCES

1 de Perrot, M., Liu, M., Waddell, T. K. & Keshavjee, S. Ischemia-reperfusion-induced lung injury. American journal of respiratory and critical care medicine 167, 490-511, doi: 10.1164/rccm.200207-670SO (2003).
2 Hsiao, H. M., Scozzi, D., Gauthier, J. M. & Kreisel, D. Mechanisms of graft rejection after lung transplantation. Current opinion in organ transplantation 22, 29-35, doi: 10.1097/mot.0000000000000371 (2017).
3 Chambers, D. C. et al. The International Thoracic Organ Transplant Registry of the International Society for Heart and Lung Transplantation: Thirty-sixth adult lung and heart-lung transplantation Report-2019; Focus theme: Donor and recipient size match. The Journal of heart and lung transplantation: the official publication of the International Society for Heart Transplantation 38, 1042-1055, doi: 10.1016/j.healun.2019.08.001 (2019).
4 Israni, A. K. et al. OPTN/SRTR 2018 Annual Data Report: Deceased Organ Donation. American journal of transplantation: official journal of the American Society of Transplantation and the American Society of Transplant Surgeons 20 Suppl s1, 509-541, doi: 10.1111/ajt. 15678 (2020).
5 Yeung, J. C. & Keshavjee, S. Overview of clinical lung transplantation. Cold Spring Harbor perspectives in medicine 4, a015628, doi: 10.1101/cshperspect.a015628 (2014).
6 Fischer, S. et al. Interleukin 10 gene transfection of donor lungs ameliorates posttransplant cell death by a switch from cellular necrosis to apoptosis. The Journal of thoracic and cardiovascular surgery 126, 1174-1180, doi: 10.1016/s0022 (2003).
7 Fischer, S. et al. In vivo donor adenoviral-mediated transtracheal transfection of human IL-10 (HIL-10) gene ameliorates ischemia-reperfusion (IR) injury and enhances transplanted lung function. The Journal of heart and lung transplantation: the official publication of the International Society for Heart Transplantation 20, 152-153 (2001).
8 de Perrot, M. et al. Impact of human interleukin-10 on vector-induced inflammation and early graft function in rat lung transplantation. American journal of respiratory cell and molecular biology 28, 616-625, doi: 10.1165/rcmb.2002-0109OC (2003).
9 Itano, H. et al. Adenovirus-mediated gene transfer of human interleukin 10 ameliorates reperfusion injury of rat lung isografts. The Journal of thoracic and cardiovascular surgery 120, 947-956, doi: 10.1067/mtc.2000.109240 (2000).
10 Machuca, T. N. et al. Safety and Efficacy of Ex vivo Donor Lung Adenoviral IL-10 Gene Therapy in a Large Animal Lung Transplant Survival Model. Human gene therapy 28, 757-765, doi: 10.1089/hum.2016.070 (2017).
11 Martins, S. et al. Transbronchial administration of adenoviral-mediated interleukin-10 gene to the donor improves function in a pig lung transplant model. Gene therapy 11, 1786-1796, doi: 10.1038/sj.gt.3302357 (2004).
12 Itano, H. et al. Lipid-mediated ex vivo gene transfer of viral interleukin 10 in rat lung allotransplantation. The Journal of thoracic and cardiovascular surgery 122, 29-38, doi: 10.1067/mtc.2001.114636 (2001).
13 Hirayama, S. et al. Lentivirus IL-10 gene therapy down-regulates IL-17 and attenuates mouse orthotopic lung allograft rejection. American journal of transplantation: official journal of the American Society of Transplantation and the American Society of Transplant Surgeons 13, 1586-1593, doi: 10.1111/ajt. 12230 (2013).
14 Cypel, M. et al. Technique for prolonged normothermic ex vivo lung perfusion. The Journal of heart and lung transplantation: the official publication of the International Society for Heart Transplantation 27, 1319-1325, doi: 10.1016/j.healun.2008.09.003 (2008).
15 Cypel, M. et al. Normothermic ex vivo lung perfusion in clinical lung transplantation. The New England journal of medicine 364, 1431-1440, doi: 10.1056/NEJMoa1014597 (2011).
16 Mariscal, A., Cypel, M. & Keshavjee, S. Ex vivo Lung Perfusion. Current transplantation reports 4, 149-158, doi: 10.1007/s40472-017-0145-x (2017).
17 Cypel, M. et al. Functional repair of human donor lungs by IL-10 gene therapy. Science translational medicine 1, 4ra9, doi: 10.1126/scitranslmed.3000266 (2009).
18 Yeung, J. C. et al. Ex vivo adenoviral vector gene delivery results in decreased vector-associated inflammation pre- and post-lung transplantation in the pig. Molecular therapy: the journal of the American Society of Gene Therapy 20, 1204-1211, doi: 10.1038/mt.2012.57 (2012).
19 Doudna, J. A. The promise and challenge of therapeutic genome editing. Nature 578, 229-236, doi: 10.1038/s41586-020-1978-5 (2020).
20 Doudna, J. A. & Charpentier, E. Genome editing. The new frontier of genome engineering with CRISPR-Cas9. Science (New York, N.Y.) 346, 1258096, doi: 10.1126/science. 1258096 (2014).
21 Jinek, M. et al. A programmable dual-RNA-guided DNA endonuclease in adaptive bacterial immunity. Science (New York, N.Y.) 337, 816-821, doi: 10.1126/science. 1225829 (2012).
22 Jiang, F. & Doudna, J. A. CRISPR-Cas9 Structures and Mechanisms. Annual review of biophysics 46, 505-529, doi: 10.1146/annurev-biophys-062215-010822 (2017).
23 Casas-Mollano, J. A., Zinselmeier, M. H., Erickson, S. E. & Smanski, M. J. CRISPR-Cas Activators for Engineering Gene Expression in Higher Eukaryotes. The CRISPR journal 3, 350-364, doi: 10.1089/crispr.2020.0064 (2020).
24 Hilton, I. B. et al. Epigenome editing by a CRISPR-Cas9-based acetyltransferase activates genes from promoters and enhancers. Nature biotechnology 33, 510-517, doi: 10.1038/nbt.3199 (2015).
25 Liu, X. S. et al. Editing DNA Methylation in the Mammalian Genome. Cell 167, 233-247.e217, doi: 10.1016/j.cell.2016.08.056 (2016).
26 Liu, X. S. et al. Rescue of Fragile X Syndrome Neurons by DNA Methylation Editing of the FMR1 Gene. Cell 172, 979-992.e976, doi: 10.1016/j.cell.2018.01.012 (2018).
27 Stephens, C. J., Kashentseva, E., Everett, W., Kaliberova, L. & Curiel, D. T. Targeted in vivo knock-in of human alpha-1-antitrypsin cDNA using adenoviral delivery of CRISPR/Cas9. Gene therapy 25, 139-156, doi: 10.1038/s41434-018-0003-1 (2018).
28 Brown, A. et al. Multiplexed and tunable transcriptional activation by promoter insertion using nuclease-assisted vector integration. Nucleic acids research 47, e67, doi: 10.1093/nar/gkz210 (2019).
29 Tak, Y. E. et al. Inducible and multiplex gene regulation using CRISPR-Cpf1-based transcription factors. Nature methods 14, 1163-1166, doi: 10.1038/nmeth.4483 (2017).
30 Nakamura, M. et al. Anti-CRISPR-mediated control of gene editing and synthetic circuits in eukaryotic cells. Nature communications 10, 194, doi: 10.1038/s41467-018-08158-x (2019).
31 Zetsche, B., Volz, S. E. & Zhang, F. A split-Cas9 architecture for inducible genome editing and transcription modulation. Nature biotechnology 33, 139-142, doi: 10.1038/nbt.3149 (2015).
32 Gao, Y. et al. Complex transcriptional modulation with orthogonal and inducible dCas9 regulators. Nature methods 13, 1043-1049, doi: 10.1038/nmeth.4042 (2016).
33 McCarty, N. S., Graham, A. E., Studená, L. & Ledesma-Amaro, R. Multiplexed CRISPR technologies for gene editing and transcriptional regulation. Nature communications 11, 1281, doi: 10.1038/s41467-020-15053-x (2020).
34 Chavez, A. et al. Highly efficient Cas9-mediated transcriptional programming. Nature methods 12, 326-328, doi: 10.1038/nmeth.3312 (2015).
35 Kurosaki, F. et al. AAV6-Mediated IL-10 Expression in the Lung Ameliorates Bleomycin-Induced Pulmonary Fibrosis in Mice. Human gene therapy 29, 1242-1251, doi: 10.1089/hum.2018.024 (2018).
36 Kurosaki, F. et al. Optimization of adeno-associated virus vector-mediated gene transfer to the respiratory tract. Gene therapy 24, 290-297, doi: 10.1038/gt.2017.19 (2017).
37 Fischer, S. et al. In vivo transtracheal adenovirus-mediated transfer of human interleukin-10 gene to donor lungs ameliorates ischemia-reperfusion injury and improves early posttransplant graft function in the rat. Human gene therapy 12, 1513-1526, doi: 10.1089/10430340152480249 (2001).
38 Tagawa, T. et al. Low-dose endobronchial gene transfer to ameliorate lung graft ischemia-reperfusion injury. The Journal of thoracic and cardiovascular surgery 123, 795-802 (2002).
39 Hirayama, S. et al. Local long-term expression of lentivirally delivered IL-10 in the lung attenuates obliteration of intrapulmonary allograft airways. Human gene therapy 22, 1453-1460, doi: 10.1089/hum.2010.225 (2011).
40 Oishi, H. et al. Transbronchial human interleukin-10 gene transfer reduces acute inflammation associated with allograft rejection and intragraft interleukin-2 and tumor necrosis factor-alpha gene expression in a rat model of lung transplantation. The Journal of heart and lung transplantation: the official publication of the International Society for Heart Transplantation 29, 360-367, doi: 10.1016/j.healun.2009.10.002 (2010).
41 Perez-Pinera, P. et al. RNA-guided gene activation by CRISPR-Cas9-based transcription factors. Nature methods 10, 973-976, doi: 10.1038/nmeth.2600 (2013).
42 Concordet, J.-P. & Haeussler, M. CRISPOR: intuitive guide selection for CRISPR/Cas9 genome editing experiments and screens. Nucleic acids research 46, W242-W245, doi: 10.1093/nar/gky354 (2018).
43 Cassivi, S. D. et al. Transplant immunosuppression increases and prolongs transgene expression following adenoviral-mediated transfection of rat lungs. The Journal of heart and lung transplantation: the official publication of the International Society for Heart Transplantation 19, 984-994, doi: 10.1016/s1053-2498 (00) 00166-2 (2000).
44 Maeder, M. L. et al. Development of a gene-editing approach to restore vision loss in Leber congenital amaurosis type 10. Nature medicine 25, 229-233, doi: 10.1038/s41591-018-0327-9 (2019).
45 Gillmore, J. D. et al. CRISPR-Cas9 In vivo Gene Editing for Transthyretin Amyloidosis. New England Journal of Medicine, doi: 10.1056/NEJMoa2107454 (2021).
46 Böhm, S. et al. A gene therapy for inherited blindness using dCas9-VPR-mediated transcriptional activation. Science advances 6, eaba5614, doi: 10.1126/sciadv.aba5614 (2020).
47 Sondhi, D., Stiles, K. M., De, B. P. & Crystal, R. G. Genetic Modification of the Lung Directed Toward Treatment of Human Disease. Human gene therapy 28, 3-84, doi: 10.1089/hum.2016.152 (2017).
48 Lee, C. S. et al. Adenovirus-Mediated Gene Delivery: Potential Applications for Gene and Cell-Based Therapies in the New Era of Personalized Medicine. Genes & diseases 4, 43-63, doi: 10.1016/j.gendis.2017.04.001 (2017).
49 Bulcha, J. T., Wang, Y., Ma, H., Tai, P. W. L. & Gao, G. Viral vector platforms within the gene therapy landscape. Signal transduction and targeted therapy 6, 53, doi: 10.1038/s41392-021-00487-6 (2021).
50 Shirley, J. L., de Jong, Y. P., Terhorst, C. & Herzog, R. W. Immune Responses to Viral Gene Therapy Vectors. Molecular therapy: the journal of the American Society of Gene Therapy 28, 709-722, doi: 10.1016/j.ymthe.2020.01.001 (2020).
51 Ahi, Y. S., Bangari, D. S. & Mittal, S. K. Adenoviral vector immunity: its implications circumvention strategies. Current gene and therapy 11, 307-320, doi: 10.2174/156652311796150372 (2011).
52 Carpentier, M. et al. Intrinsic transgene immunogenicity gears CD8 (+) T-cell priming after rAAV-mediated muscle gene transfer. Molecular therapy: the journal of the American Society of Gene Therapy 23, 697-706, doi: 10.1038/mt.2014.235 (2015).
53 Crudele, J. M. & Chamberlain, J. S. Cas9 immunity creates challenges for CRISPR gene editing therapies. Nature communications 9, 3497, doi: 10.1038/s41467-018-05843-9 (2018).
54 Charlesworth, C. T. et al. Identification of preexisting adaptive immunity to Cas9 proteins in humans. Nature medicine 25, 249-254, doi: 10.1038/s41591-018-0326-x (2019).
55 Chew, W. L. et al. A multifunctional AAV-CRISPR-Cas9 and its host response. Nature methods 13, 868-874, doi: 10.1038/nmeth.3993 (2016).
56 Suga, M., Gladdy, R., Xing, Z., Keshavjee, S. H. & Liu, M. Transplant immunosuppression enhances efficiency of adenoviral-mediated gene retransfection: inhibition of interferon-gamma and immunoglobin G. The Annals of thoracic surgery 73, 1092-1097, doi: 10.1016/s0003-4975 (01) 03574-3 (2002).
57 Cassivi, S. D. et al. Transgene expression after adenovirus-mediated retransfection of rat lungs is increased and prolonged by transplant immunosuppression. The Journal of thoracic and cardiovascular surgery 117, 1-7, doi: 10.1016/s0022-5223 (99) 70462-9 (1999).
58 Howe, K. L. et al. Ensembl 2021. Nucleic acids research 49, D884-D891, doi: 10.1093/nar/gkaa942 (2020).
59 Patro, R., Duggal, G., Love, M. I., Irizarry, R. A. & Kingsford, C. Salmon provides fast and bias-aware quantification of transcript expression. Nature methods 14, 417-419, doi: 10.1038/nmeth.4197 (2017).
60 Soneson, C., Love, M. & Robinson, M. Differential analyses for RNA-seq: transcript-level estimates improve gene-level inferences [version 1; peer review: 2 approved]. F1000Research 4, doi: 10.12688/f1000research.7563.1 (2015).
61 Love, M. I., Huber, W. & Anders, S. Moderated estimation of fold change and dispersion for RNA-seq data with DESeq2. Genome biology 15, 550, doi: 10.1186/s13059-014-0550-8 (2014).

1. A modified organ or tissue comprising at least one modified endogenous inflammation-regulating and/or immune-regulating gene, the at least one modified endogenous inflammation-regulating and/or immune-regulating gene is selected from the group consisting of: interleukin-10 (IL-10), interleukin-1 receptor antagonist (IL1RN), Interleukin-4 (IL-4), Interleukin-6 (IL-6), Interleukin-8 (IL-8), tumor necrosis factor binding protein (TNFBP), cytotoxic T-lymphocyte associated protein 4 (CTLA4), NFKB inhibitor alpha (Ikba), Programmed cell death-1 (PD-1), Programmed cell death-ligand 1 (PD-L1), Interleukin-10 receptor, CD47, CD200, Fas Ligand (FasL), MHC molecule (MHC-Ib) H2-M3, Serine protease inhibitor 6 (Spi6), C-C Motif Chemokine Ligand 21 (Ccl21) and Milk Fat Globule-EGF Factor 8 Protein (Mfge8).

2. (canceled)

3. (canceled)

4. The modified organ or tissue of claim 1, wherein the at least one modified endogenous inflammation-regulating and/or immune-regulating gene comprises at least one modification, and wherein the at least one modification is a genetic modification, an epigenetic modification, or a combination thereof.

5. The modified organ or tissue of claim 4, wherein;
(i) the genetic modification comprises insertion, deletion, or substitution of one or more nucleotides in a regulatory region of the at least one modified endogenous inflammation-regulating and/or immune-regulating gene;
(ii) the epigenetic modification comprises a modification of transcription, a histone modification, a modification of DNA methylation, or a combination thereof; and/or
(iii) the at least one modification comprises transcriptional activation or transcriptional derepression.

6. (canceled)

7. (canceled)

8. The modified organ or tissue of claim 4, wherein the modified organ or tissue comprises a CRISPR-associated (Cas) protein, wherein the Cas protein:
(i) has nuclease, nickase, transposase, base editing, or prime editing activity:
(ii) is nuclease-deficient or nuclease-dead:
(iii) is Cas9; or
(iv) any combination of (i) to (iii).

9. (canceled)

10. (canceled)

11. (canceled)

12. The modified organ or tissue of claim 3, wherein the at least one modified endogenous inflammation-regulating and/or immune-regulating gene is IL-10 and/or IL1RN, and the organ is a lung.

13. The modified organ or tissue of claim 1, wherein the organ comprises a polynucleotide encoding at least one guide RNA (gRNA), and wherein the polynucleotide comprises one or more of the nucleotide sequences set forth in SEQ ID NOs: 1-10, 12-42, 44-59 and 61-79, or a complement thereof.

14. (canceled)

15. A method of preparing an organ for transplantation into an animal, said method comprising the steps of:
(i) introducing into the organ (a) a polynucleotide encoding at least one guide RNA (gRNA) that binds to a regulatory region of at least one inflammation-regulating and/or immune-regulating gene selected from the group consisting of interleukin-10 (IL-10), interleukin-1 receptor antagonist (IL1RN), Interleukin-4 (IL-4), Interleukin-6 (IL-6), Interleukin-8 (IL-8), tumor necrosis factor binding protein (TNFBP), cytotoxic T-lymphocyte associated protein 4 (CTLA4), NFKB inhibitor alpha (Ikba), Programmed cell death-1 (PD-1), Programmed cell death-ligand 1 (PD-L1), Interleukin-10 receptor, CD47, CD200, Fas Ligand (FasL), MHC molecule (MHC-Ib) H2-M3, Serine protease inhibitor 6 (Spi6), C-C Motif Chemokine Ligand 21 (Ccl21) and Milk Fat Globule-EGF Factor 8 Protein (Mfge8); and (b) a polynucleotide encoding a CRISPR-associated (Cas) protein; and
(ii) modifying at least one endogenous inflammation-regulating and/or immune-regulating gene in the organ.

16. The method of claim 15, wherein the step of modifying the at least one endogenous inflammation-regulating and/or immune-regulating gene in the organ comprises CRISPR/Cas-mediated modification.

17. The method of claim 16, wherein the CRISPR/Cas-mediated modification comprises;
(i) genetic modification, epigenetic modification, or a combination thereof;
(ii) insertion, deletion, or substitution of one or more nucleotides in a regulatory region of the at least one modified endogenous inflammation-regulating and/or immune-regulating gene; and/or
(iii) a modification of transcription, a histone modification, a modification of DNA methylation, or a combination thereof.

18. (canceled)

19. (canceled)

20. (canceled)

21. The method of claim 15, further comprising the step of immunosuppression before, during and/or after the transplantation.

22. The method of claim 21, wherein the step of immunosuppression comprises administering an agent to a recipient of the organ, wherein the agent is;
(i) a steroid, corticosteroid, calcineurine inhibitor, cell cycle inhibitor, IL-2R antagonist, mTOR inhibitor, or a combination thereof;
(ii) methylprednisolone, cyclosporin, cyclosporin A, azathioprine, anti-thymocyte globulin, rapamycin, tacrolimus, mycophenolate mofetil, mycophenolic acid, sirolimus, everolimus, prednisone, or a combination thereof;
(iii) a combination of methylprednisolone, cyclosporin and azathioprine; or
(iv) a combination of methylprednisolone and cyclosporin.

23. (canceled)

24. (canceled)

25. (canceled)

26. The method of claim 15, wherein introducing into the organ the polypeptide comprises subjecting the organ to a perfusate, the perfusate comprises a polynucleotide encoding at least one gRNA, and wherein the polynucleotide comprises one or more of the nucleotide sequences set forth in SEQ ID NOs: 1-10, 12-42, 44-59 and 61-79, or a complement thereof.

27. (canceled)

28. The method of claim 26, wherein the organ is a lung and the step of subjecting the organ to a perfusate comprises ex vivo lung perfusion (EVLP) or in vivo lung perfusion (IVLP).

29. An expression cassette comprising:
(i) a polynucleotide encoding at least one guide RNA (gRNA) that binds to a regulatory region of at least one inflammation-regulating and/or immune-regulating gene selected from the group consisting of interleukin-10 (IL-10), interleukin-1 receptor antagonist (IL1RN), Interleukin-4 (IL-4), Interleukin-6 (IL-6), Interleukin-8 (IL-8), tumor necrosis factor binding protein (TNFBP), cytotoxic T-lymphocyte associated protein 4 (CTLA4), NFKB inhibitor alpha (Ikba), Programmed cell death-1 (PD-1), Programmed cell death-ligand 1 (PD-L1), Interleukin-10 receptor, CD47, CD200, Fas Ligand (FasL), MHC molecule (MHC-Ib) H2-M3, Serine protease inhibitor 6 (Spi6), C-C Motif Chemokine Ligand 21 (Ccl21) and Milk Fat Globule-EGF Factor 8 Protein (Mfge8); and
(ii) a polynucleotide encoding a CRISPR-associated (Cas) protein.

30.-60. (canceled)

61. A kit comprising the expression cassette of claim 29 and instructions for using same.

62. The kit of claim 61, further comprising a perfusate.

63.-72. (canceled)

73. The method of claim 15, wherein the organ is selected from the group consisting of lung, heart, kidney, liver, pancreas, stomach and intestine.

74. The method of claim 15, wherein the organ is a lung.

75. The method of claim 15, wherein step (i) further comprises introducing into the organ a polynucleotide encoding a transcriptional activator, the transcriptional activator comprises (i) VP64 from herpes simplex virus, p65 from human NF-κB, Rta from Epstein-Barr virus, or a combination thereof; or (ii) p300 core domain of human histone acetyltransferase.

76. The modified organ or tissue of claim 8, wherein the Cas protein is Staphylococcus aureus Cas9 (saCas9), Streptococcus pyogenes Cas9 (spCas9), or a variant thereof.
